(19) United States
(12) Patent Application Publication
(10) Pub. No.: US 2026/0027108 A1
(43) Pub. Date: Jan. 29, 2026

(54) PHARMACEUTICAL COMPOSITION OF HETEROARYL DERIVATIVE AND MEDICAL USE THEREOF

(71) Applicant: Haisco Pharmaceutical Group Co., Ltd., Shannan City, Tibet (CN)

(72) Inventors: Yao LI, Lhoka, Tibet (CN); Li WANG, Lhoka, Tibet (CN); Hongjun LIN, Lhoka, Tibet (CN); Yan YU, Lhoka, Tibet (CN)

(21) Appl. No.: 18/994,476

(22) PCT Filed: Jul. 14, 2023
(86) PCT No.: PCT/CN2023/107476

§ 371 Date: Jan. 14, 2025

(30) Foreign Application Priority Data
Jul. 14, 2022 (CN) 202210838605.2
Jul. 15, 2022 (CN) 202210840146.1
Nov. 10, 2022 (CN) 202211404496.X

(51) Int. Cl. A61K 31/496; A61K 9/20; A61K 31/497; A61K 31/4985; A61K 31/5025; A61P 35/00

(52) U.S. Cl. CPC A61K 31/496; A61K 9/2009; A61K 9/2013; A61K 9/2018; A61K 9/2027; A61K 9/2054; A61K 31/497; A61K 31/4985; A61K 31/5025; A61P 35/00

(57) ABSTRACT

A pharmaceutical composition or a pharmaceutical preparation, the pharmaceutical composition or the pharmaceutical preparation comprising a therapeutically effective amount of an active ingredient M and a pharmaceutically acceptable excipient; the active ingredient M is selected from among a compound having general formula (I) or a stereoisomer, a tautomer, a deuterated substance, a solvate, a prodrug, a metabolite, a pharmaceutically acceptable salt or a co-crystal thereof; and the pharmaceutical composition or the pharmaceutical preparation comprises 1-600 mg of the active ingredient M. Further provided is a use of the pharmaceutical composition or pharmaceutical preparation in preparing a pharmaceutical for treating cancer.

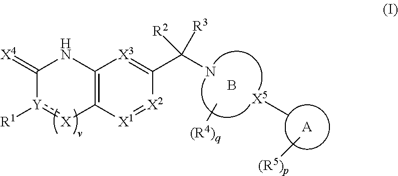

### CROSS-REFERENCE TO RELATED APPLICATION

This application is a National Stage of International Application No. PCT/CN2023/107476, filed Jul. 14, 2023, which is hereby incorporated by reference in its entirety.

### TECHNICAL FIELD

The present disclosure belongs to the field of pharmaceutical preparations, and in particular relates to a pharmaceutical composition or a pharmaceutical preparation, wherein the pharmaceutical composition or the pharmaceutical preparation comprises a therapeutically effective amount of an active ingredient M and a pharmaceutically acceptable excipient; the active ingredient M is selected from among a compound having general formula (I) or a stereoisomer, a tautomer, a deuterated substance, a solvate, a prodrug, a metabolite, a pharmaceutically acceptable salt or a co-crystal thereof; and the pharmaceutical composition or the pharmaceutical preparation comprises 1-600 mg of the active ingredient M. The present disclosure further provides a use of the pharmaceutical composition or pharmaceutical preparation in preparing a pharmaceutical for treating cancer.

### BACKGROUND

Approximately 5% of breast cancer patients are associated with germline mutations in the BRCA1/2 genes (3% in the BRCA1 gene and 2% in the BRCA2 gene). Most breast cancers caused by BRCA1 mutations are triple-negative breast cancers (70%), while BRCA2 mutations are more likely to cause estrogen receptor-positive breast cancers (70%). The BRCA1/2 genes are tumor suppressor genes and play an important role in DNA damage repair, normal cell growth, etc. Mutations in the genes can inhibit the normal repair ability after DNA damage and cause homologous recombination deficiency (HRD), that is, loss of BRCA function or mutation or loss of function in other homologous recombination-related genes, making repair of DNA double-strand breaks impossible through homologous recombination repair (HRR), eventually leading to cancer.

Poly(ADP-ribose) polymerase (PARP) is a DNA repair enzyme that plays a key role in the DNA repair pathway. PARP is activated by DNA damage and breakage. As a molecular sensor of DNA damage, it has the function of identifying and binding to the DNA break location, thereby activating and catalyzing the polyADP ribosylation of the receptor protein and participating in the DNA repair process. PARP plays a key role in the process of DNA single-strand base excision and repair. In HRD tumor cells, DNA double-strand breaks cannot be repaired, and PARP inhibitors block the single-strand repair, resulting in a “synthetic lethal” effect and leading to tumor cell death.

PARP inhibitors have a “trapping” effect on the PARP protein, causing the PARP protein that binds to damaged DNA to be trapped on the DNA, directly causing other DNA repair proteins to be unable to bind, eventually leading to cell death. At present, several PARP inhibitors have been successfully developed, such as olaparib, rucaparib and niraparib. However, adverse reactions limit their ability to be used in combination with chemotherapy drugs. This may be related to the lack of selectivity of marketed PARP inhibitors against the PARP family. The side effects include intestinal toxicity caused by tankyrase inhibition and hematological toxicity caused by PARP-2 inhibition. Therefore, it is of great clinical significance to develop highly selective PARP-1 inhibitors and reduce the toxic and side effects associated with non-selective PARP inhibitors.

### SUMMARY

The objective of the present disclosure is to provide a pharmaceutical composition or a pharmaceutical preparation, wherein the pharmaceutical composition or the pharmaceutical preparation comprises a therapeutically effective amount of an active ingredient M and a pharmaceutically acceptable excipient; the active ingredient M is selected from among a compound having general formula (I) or a stereoisomer, a tautomer, a deuterated substance, a solvate, a prodrug, a metabolite, a pharmaceutically acceptable salt or a co-crystal thereof; and the preparation specification of the pharmaceutical composition or the pharmaceutical preparation is 1-600 mg. The present disclosure further provides a use of the pharmaceutical composition or pharmaceutical preparation in preparing a pharmaceutical for treating cancer.

The compounds of the present disclosure have advantages of good oral properties, good efficacy, low toxic side effects, good safety, high selectivity, good pharmacokinetics, high bioavailability, and no inhibition of CYP enzyme.

The present disclosure relates to a pharmaceutical composition or a pharmaceutical preparation, comprising a therapeutically effective amount of an active ingredient M and a pharmaceutically acceptable excipient. The pharmaceutical composition can be in a unit preparation form.

The present disclosure relates to a pharmaceutical composition or a pharmaceutical preparation, wherein the pharmaceutical composition or the pharmaceutical preparation comprises an active ingredient M and a pharmaceutically acceptable excipient, and the active ingredient M is selected from among a compound having general formula (I), (II), (III), (IV), (V), (VI), (II-1) or (II-2) or a stereoisomer, a tautomer, a deuterated substance, a solvate, a prodrug, a metabolite, a pharmaceutically acceptable salt or a co-crystal thereof.

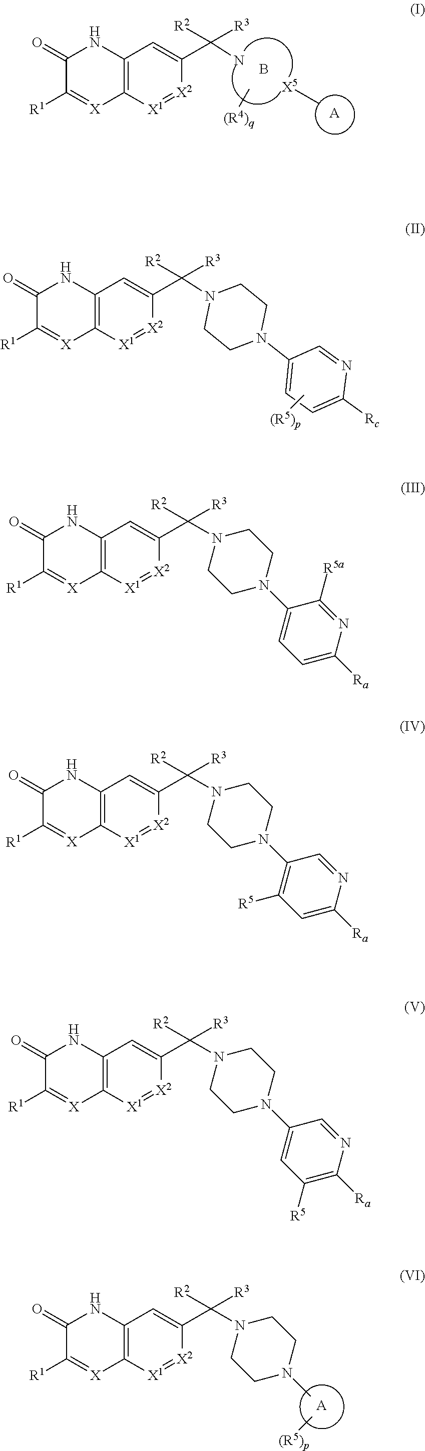

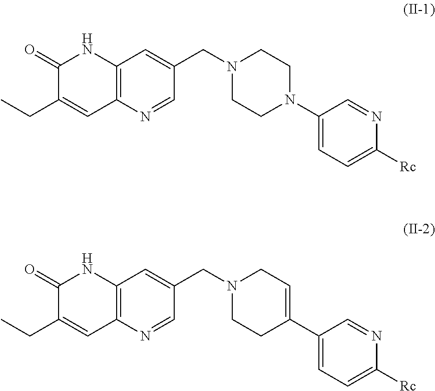

wherein X is selected from CR^{x}, C(R^{x})₂, O, N, or NR^{x}; in some embodiments, X is selected from CR^{x};
Y is selected from N, C or CH; in some embodiments, Y is selected from N or C; in some embodiments, Y is selected from C;
[symbol] represents a single bond or a double bond;
v is selected from 1, 2 or 3; in some embodiments, v is selected from 1 or 2; in some embodiments, v is selected from 1;
X¹, X² and X³ are each independently selected from N or CR^{x}; in some embodiments, X¹ is selected from N, and X² and X³ are selected from CR^{x}; in some embodiments, X¹, X² and X³ are selected from N; in some embodiments, X¹ is selected from N, X² is selected from N, and X³ is selected from CR^{x}; in some embodiments, X¹ is selected from N, X² is selected from CR^{x}, and X³ is selected from N; in some embodiments, X¹, X² and X³ are selected from CR^{x}; in some embodiments, X¹ is selected from CR^{x}, and X² and X³ are selected from N; in some embodiments, X¹ is selected from CR^{x}, X² is selected from N, and X³ is selected from CR^{x}; in some embodiments, X¹ is selected from CR^{x}, X² is selected from CR^{x}, and X³ is selected from N;
provided that when [symbol] represents a double bond, and v is selected from 1, X, X¹, X² and X³ are not all selected from CR^{x};
X⁴ is selected from O or S; in some embodiments, X⁴ is selected from O; in some embodiments, X⁴ is selected from S;
X⁵ is selected from N or CR^{x}; in some embodiments, X⁵ is selected from N; in some embodiments, X⁵ is selected from CR^{x}; in some embodiments, X⁵ is selected from CH;
each R^{x} is independently selected from H, D, halogen, cyano, amino, hydroxyl, —SF₅, C₁₋₆ alkyl, halo C₁₋₆ alkyl, halo C₁₋₆ alkoxy, deuterated C₁₋₆ alkyl, deuterated C₁₋₆ alkoxy, C₁₋₆ alkoxy, C₂₋₆ alkenyl, C₂₋₆ alkynyl, C₂₋₆ alkyl-O—C₁₋₆ alkyl, —(CH₂)ᵣ—C₃₋₁₂ cycloalkyl or —(CH₂)ᵣ-(3- to 12-membered heterocycloalkyl); or two R^{x} on the same carbon atom together form ═O; in some embodiments, each R^{x} is independently selected from H, D, halogen, cyano, amino, hydroxyl, C₁₋₄ alkyl, halo C₁₋₄ alkyl, halo C₁₋₄ alkoxy, deuterated C₁₋₄ alkyl, deuterated C₁₋₄ alkoxy, C₁₋₄ alkoxy, C₂₋₄ alkenyl, C₂₋₄ alkynyl, C₁₋₂ alkyl-O—C₁₋₂ alkyl, —(CH₂)ᵣ—C₃₋₆ monocyclic cycloalkyl, —(CH₂)ᵣ—C₅₋₇ bicyclic spirt cycloalkyl, —(CH₂)ᵣ(4- to 6-membered heterocycloalkyl), or —(CH₂)ᵣ-(7- to 9-membered bicyclic spiro heterocycloalkyl); or two R^{x} on the same carbon atom together form ═O; in some embodiments, each R^{x} is independently selected from H, D, F, Cl, cyano, amino, hydroxyl, C₁₋₂ alkyl, halo C₁₋₂ alkyl, deuterated C₁₋₂ alkyl, C₁₋₂ alkoxy, —(CH₂)ᵣ—C₃₋₄ monocyclic cycloalkyl, —(CH₂)ᵣ—C₅₋₆ bicyclic spiro cycloalkyl, —(CH₂)ᵣ(4- to 5-membered heterocycloalkyl), or —(CH₂)ᵣ-(7- to 8-membered bicyclic spiro heterocycloalkyl); or two R^{x} on the same carbon atom together form ═O; in some embodiments, each R^{x} is independently selected from H, D, F, Cl, cyano, hydroxyl, C₁₋₂ alkyl, halo C₁₋₂ alkyl, or deuterated C₁₋₂ alkyl; or two R^{x} on the same carbon atom together form ═O; in some embodiments, each R^{x} is independently selected from H, D, C₁₋₂ alkyl, halo C₁₋₂ alkyl, or deuterated C₁₋₂ alkyl; or two R^{x} on the same carbon atom together form ═O; in some embodiments, each R^{x} is independently selected from H or D, or two R^{x} on the same carbon atom together form ═O; in some embodiments, each R^{x} is independently selected from H or D;
R¹ is selected from halogen, nitro, cyano, amino, hydroxyl, —SF₅, C₁₋₆ alkyl, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl), wherein the alkyl, alkoxy, alkenyl, alkynyl, cycloalkyl or heterocycloalkyl is optionally further substituted with 1, 2 or 3 groups selected from D, halogen, cyano, amino, hydroxyl, C₁₋₆ alkyl or C₁₋₆ alkoxy; in some embodiments, R¹ is selected from halogen, nitro, cyano, amino, hydroxyl, —SF₅, C₁₋₄ alkyl, C₁₋₄ alkoxy, C₂₋₄ alkenyl, C₂₋₄ alkynyl, C₁₋₂ alkyl-O—C₁₋₂ alkyl, —(CH₂)ᵣ—C₃₋₆ monocyclic cycloalkyl, —(CH₂)ᵣ—C₅₋₇ bicyclic spiro cycloalkyl, —(CH₂)ᵣ-(4- to 6-membered heterocycloalkyl), or —(CH₂)ᵣ-(6- to 9-membered bicyclic spiro heterocycloalkyl), wherein the alkyl, alkoxy, alkenyl, alkynyl, cycloalkyl, or heterocycloalkyl is optionally further substituted with 1, 2 or 3 groups selected from D, halogen, cyano, amino, hydroxyl, C₁₋₂ alkyl, or C₁₋₂ alkoxy; in some embodiments, R¹ is selected from F, Cl, cyano, amino, hydroxyl, C₁₋₂ alkyl, C₁₋₂ alkoxy, C₂₋₃ alkenyl, C₁₋₂ alkyl-O—C₁₋₂ alkyl, —(CH₂)—C₃₋₄ monocyclic cycloalkyl, —(CH₂)ᵣ—C₅₋₇ bicyclic spiro cycloalkyl, —(CH₂)ᵣ-(4- to 5-membered heterocycloalkyl), or —(CH₂)ᵣ-(6- to 8-membered bicyclic spiro heterocycloalkyl), wherein the alkyl, alkoxy, alkenyl, alkynyl, cycloalkyl, or heterocycloalkyl is optionally further substituted with 1, 2 or 3 groups selected from D, halogen, cyano, amino, hydroxyl, C₁₋₂ alkyl, or C₁₋₂ alkoxy; in some embodiments, R¹ is selected from cyano, C₁₋₂ alkyl, C₂₋₃ alkenyl, C₁₋₂ alkyl-O—C₁₋₂ alkyl, C₃₋₄ monocyclic cycloalkyl, or 4- to 5-membered heterocycloalkyl, wherein the alkyl, alkenyl, cycloalkyl, or hetercycloalkyl is optionally further substituted with 1, 2 or 3 groups selected from D, F, Cl, cyano, amino, hydroxyl, C₁₋₂ alkyl, or C₁₋₂ alkoxy; in some embodiments, R¹ is selected from cyano, C₁₋₂ alkyl, C₂₋₃ alkenyl, C₁₋₂ alkyl-O—C₁₋₂ alkyl, or C₃₋₄ monocyclic cycloalkyl;
each r is independently selected from 0, 1, 2 or 3; in some embodiments, each r is independently selected from 0 or 1; in some embodiments, r is selected from 0;
R² and R³ are each independently selected from H, D, halogen, cyano, amino, hydroxyl, C₁₋₆ alkyl-O—C₁₋₆ alkyl, hydroxy C₁₋₆ alkyl, C₁₋₆ alkoxy, halo C₁₋₆ alkyl, halo C₁₋₆ alkoxy, deuterated C₁₋₆ alkyl, deuterated C₁₋₆ alkoxy or C₁₋₆ alkyl; or R² and R³ together with the carbon atom to which they are attached form C₃₋₅ cycloalkyl or 4- to 5-membered heterocycloalkyl;
in some embodiments, R² and R³ are each independently selected from H, D, halogen, cyano, amino, hydroxyl, C₁₋₂ alkyl-O—C₁₋₂ alkyl, hydroxy C₁₋₄ alkyl, C₁₋₄ alkoxy, halo C₁₋₄ alkyl, halo C₁₋₄ alkoxy, deuterated C₁₋₄ alkyl, deuterated C₁₋₄ alkoxy or C₁₋₄ alkyl; or R² and R³ together with the carbon atom to which they are attached form C₃₋₅ cycloalkyl or 4- to 5-membered heterocycloalkyl; in some embodiments, R² and R³ are each independently selected from H, D, halogen, cyano, amino, hydroxyl, C₁₋₂ alkyl-O—C₁₋₂ alkyl, hydroxy C₁₋₂ alkyl, C₁₋₂ alkoxy, halo C₁₋₂ alkyl, halo C₁₋₂ alkoxy, deuterated C₁₋₂ alkyl, deuterated C₁₋₂ alkoxy or C₁₋₂ alkyl; or R² and R³ together with the carbon atom to which they are attached form C₁₋₄ cycloalkyl or 4-membered heterocycloalkyl; in some embodiments, R² and R³ are each independently selected from H, D, F, hydroxyl, halo C₁₋₂ alkyl, deuterated C₁₋₂ alkyl or C₁₋₂ alkyl; or R² and R³ together with the carbon atom to which they are attached form C₃₋₄ cycloalkyl; in some embodiments, R² and R³ are each independently selected from H, D, or C₁₋₂ alkyl; or R² and R³ together with the carbon atom to which they are attached form C₃₋₄ cycloalkyl; in some embodiments, R² and R³ are each independently selected from H, D, or C₁₋₂ alkyl; or R² and R³ together with the carbon atom to which they are attached form C₃₋₄ cycloalkyl; in some embodiments, R² and R³ are each independently selected from H or D;
each R⁴ is independently selected from D, halogen, cyano, amino, hydroxyl, —SF₅, C₁₋₆ alkyl, C₁₋₆ alkoxy, halo C₁₋₆ alkyl, halo C₁₋₆ alkoxy, deuterated C₁₋₆ alkyl or deuterated C₁₋₆ alkoxy; or two R⁴ on the same carbon atom together with the carbon atom to which they are attached form ═O; in some embodiments, each R⁴ is independently selected from D, halogen, cyano, amino, hydroxyl, C₁₋₄ alkyl, C₁₋₄ alkoxy, halo C₁₋₄ alkyl, halo C₁₋₄ alkoxy, deuterated C₁₋₄ alkyl, or deuterated C₁₋₄ alkoxy; or two R⁴ on the same carbon atom together with the carbon atom to which they are attached form ═O; in some embodiments, each R⁴ is independently selected from D, F, Cl, cyano, amino, hydroxyl, C₁₋₂ alkyl, C₁₋₂ alkoxy, halo C₁₋₂ alkyl, halo C₁₋₂ alkoxy, deuterated C₁₋₂ alkyl, or deuterated C₁₋₂ alkoxy; or two R⁴ on the same carbon atom together with the carbon atom to which they are attached form ═O; in some embodiments, each R⁴ is independently selected from D, F, Cl, methyl, ethyl, methoxy, ethoxy, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂D, —CHD₂, —CD₃, —CH₂CH₂D, —CH₂CHD₂, —CH₂CD₃, —CHDCH₂D, —CHDCHD₂, —CHDCD₃, —CD₂CH₂D, —CD₂CHD₂, or —CD₂CD₃; or two R⁴ on the same carbon atom together with the carbon atom to which they are attached form ═O;
each R⁵ is independently selected from D, halogen, cyano, amino, hydroxyl, —SF₅, C₁₋₆ alkyl, C₁₋₆ alkoxy, halo C₁₋₆ alkyl, halo C₁₋₆ alkoxy, deuterated C₁₋₆ alkyl or deuterated C₁₋₆ alkoxy; in some embodiments, each R⁵ is independently selected from D, halogen, cyano, amino, hydroxyl, C₁₋₄ alkyl, C₁₋₄ alkoxy, halo C₁₋₄ alkyl, halo C₁₋₄ alkoxy, deuterated C₁₋₄ alkyl, or deuterated C₁₋₄ alkoxy; in some embodiments, each R⁵ is independently selected from D, F, Cl, cyano, amino, hydroxyl, C₁₋₂ alkyl, C₁₋₂ alkoxy, halo C₁₋₂ alkyl, halo C₁₋₂ alkoxy, deuterated C₁₋₂ alkyl, or deuterated C₁₋₂ alkoxy; in some embodiments, each R⁵ is independently selected from D, F, Cl, C₁₋₂ alkyl, halo C₁₋₂ alkyl, or deuterated C₁₋₂ alkyl; in some embodiments, each R⁵ is independently selected from D, F, Cl, methyl, ethyl, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂D, —CHD₂, —CD₃, —CH₂CH₂D, —CH₂CHD₂, —CH₂CD₃, —CHDCH₂D, —CHDCHD₂, —CHDCD₃, —CD₂CH₂D, —CD₂CHD₂, or —CD₂CD₃;
q is selected from 0, 1, 2 or 3; in some embodiments, q is selected from 0, 1 or 2; in some embodiments, q is selected from 0 or 1; in some embodiments, q is selected from 0;
p is selected from 0, 1, 2 or 3; in some embodiments, p is selected from 0, 1 or 2; in some embodiments, p is selected from 0 or 1; in some embodiments, p is selected from 0;
ring B is 5- to 6-membered saturated monocyclic heterocycloalkane containing 1-2 nitrogen atoms, 5- to 6-membered partially unsaturated monocyclic heterocycloalkane containing 1-2 nitrogen atoms, 6- to 8-membered saturated bridged heterocycle containing 1-4 nitrogen atoms, 5- to 10-membered saturated fused heterocycle containing 1-4 nitrogen atoms, or 5- to 11-membered saturated spiro heterocycle containing 1-4 nitrogen atoms; in some embodiments, ring B is 5-membered saturated monocyclic heterocycloalkane containing 1-2 nitrogen atoms, 6-membered saturated monocyclic heterocycloalkane containing 1-2 nitrogen atoms, 6-membered saturated bridged heterocycle containing 1, 2, 3 or 4 nitrogen atoms, 7-membered saturated bridged heterocycle containing 1, 2, 3 or 4 nitrogen atoms, 8-membered saturated bridged heterocycle containing 1, 2, 3 or 4 nitrogen atoms, 8-membered saturated fused heterocycle containing 1, 2, 3 or 4 nitrogen atoms, 9-membered saturated fused heterocycle containing 1, 2, 3 or 4 nitrogen atoms, 10-membered saturated fused heterocycle containing 1, 2, 3 or 4 nitrogen atoms, 7-membered saturated spiro heterocycle containing 1, 2, 3 or 4 nitrogen atoms, 8-membered saturated spiro heterocycle containing 1, 2, 3 or 4 nitrogen atoms, 9-membered saturated spiro heterocycle containing 1, 2, 3 or 4 nitrogen atoms, 10-membered saturated spiro heterocycle containing 1, 2, 3 or 4 nitrogen atoms, or 11-membered saturated spiro heterocycle containing 1, 2, 3 or 4 nitrogen atoms; in some embodiments, ring B is 6-membered saturated monocyclic heterocycloalkane containing 1-2 nitrogen atoms, 6-membered saturated bridged heterocycle containing 1, 2, 3 or 4 nitrogen atoms, 7-membered saturated bridged heterocycle containing 1, 2, 3 or 4 nitrogen atoms, 8-membered saturated bridged heterocycle containing 1, 2, 3 or 4 nitrogen atoms, 7-membered saturated spiro heterocycle containing 1, 2, 3 or 4 nitrogen atoms, 9-membered saturated spiro heterocycle containing 1, 2, 3 or 4 nitrogen atoms, or 11-membered saturated spiro heterocycle containing 1, 2, 3 or 4 nitrogen atoms; in some embodiments, ring B is 6-membered saturated monocyclic heterocycloalkane containing 1-2 nitrogen atoms;
ring A is selected from 5-membered monocyclic heteroaromatic ring containing 1-5 nitrogen, oxygen or sulfur atoms, 6-membered monocyclic heteroaromatic ring containing 2-5 nitrogen, oxygen or sulfur atoms, or 2-pyridyl, wherein the heteroaromatic ring or 2-pyridyl is further substituted with 1 substituent selected from Rₐ; in some embodiments, ring A is selected from 5-membered monocyclic heteroaromatic ring containing 1, 2, 3, 4 or 5 nitrogen, oxygen or sulfur atoms, 6-membered monocyclic heteroaromatic ring containing 2, 3, 4 or 5 nitrogen, oxygen or sulfur atoms, or 2-pyridyl, wherein the heteroaromatic ring or 2-pyridyl is further substituted with 1 substituent selected from Rₐ; in some embodiments, ring A is selected from 5-membered monocyclic heteroaromatic ring containing 1, 2, 3 or 4 nitrogen, oxygen or sulfur atoms, 6-membered monocyclic heteroaromatic ring containing 2 or 3 nitrogen, oxygen or sulfur atoms, or 2-pyridyl, wherein the heteroaromatic ring or 2-pyridyl is further substituted with 1 substituent selected from Rₐ; in some embodiments,

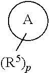

is selected from

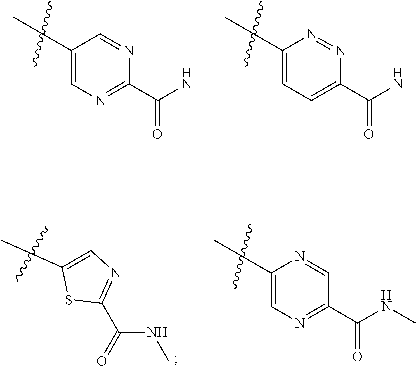

or
ring A is selected from 7- to 10-membered bicyclic heteroaromatic ring containing 1-5 nitrogen, oxygen or sulfur atoms, or 7- to 10-membered bicyclic aromatic ring, wherein the heteroaromatic ring or aromatic ring is optionally further substituted with 1-3 substituents selected from R_{b}; in some embodiments, ring A is selected from 8- to 10-membered bicyclic fused heteroaromatic ring containing 1, 2, 3, 4 or 5 nitrogen, oxygen or sulfur atoms, or 8- to 10-membered bicyclic fused aromatic ring, wherein the heteroaromatic ring or aromatic ring is optionally further substituted with 1, 2 or 3 substituents selected from R_{b}; in some embodiments, ring A is selected from 8-membered bicyclic fused heteroaromatic ring containing 1, 2, 3, 4 or 5 nitrogen, oxygen or sulfur atoms, 9-membered bicyclic fused heteroaromatic ring containing 1, 2, 3, 4 or 5 nitrogen, oxygen or sulfur atoms, 10-membered bicyclic fused heteroaromatic ring containing 1, 2, 3, 4 or 5 nitrogen, oxygen or sulfur atoms, 8-membered bicyclic fused aromatic ring, 9-membered bicyclic fused aromatic ring, or 10-membered bicyclic fused aromatic ring, wherein the heteroaromatic ring or aromatic ring is optionally further substituted with 1 substituent selected from R_{b}; in some embodiments, ring A is selected from 8-membered bicyclic fused heteroaromatic ring containing 1, 2, 3, 4 or 5 nitrogen, oxygen or sulfur atoms, 9-membered bicyclic fused heteroaromatic ring containing 1, 2, 3, 4 or 5 nitrogen, oxygen or sulfur atoms, 10-membered bicyclic fused heteroaromatic ring containing 1, 2, 3, 4 or 5 nitrogen, oxygen or sulfur atoms, 8-membered bicyclic fused aromatic ring, 9-membered bicyclic fused aromatic ring, or 10-membered bicyclic fused aromatic ring, wherein the heteroaromatic ring or aromatic ring is optionally further substituted with 1 substituent selected from R_{b}; in some embodiments,

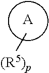

is selected from

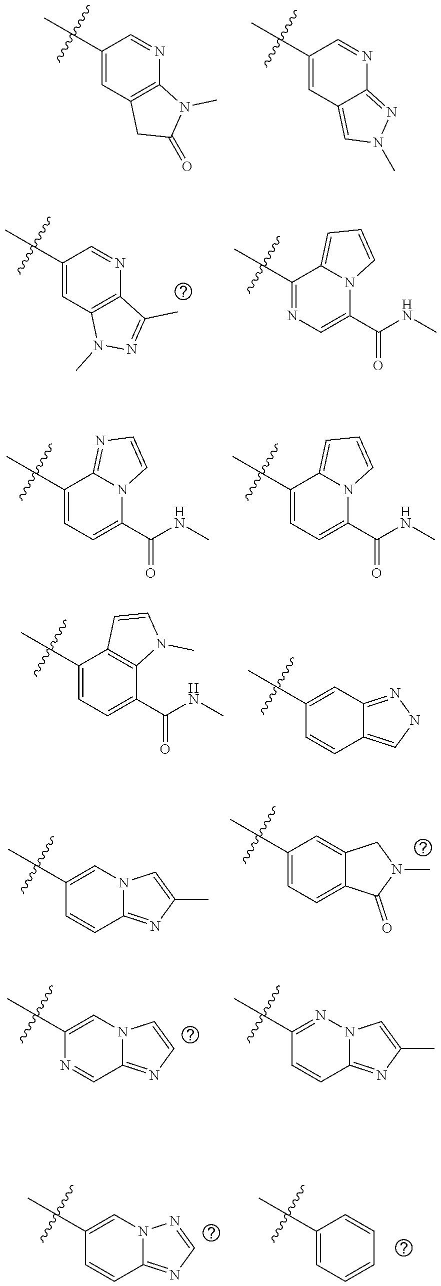

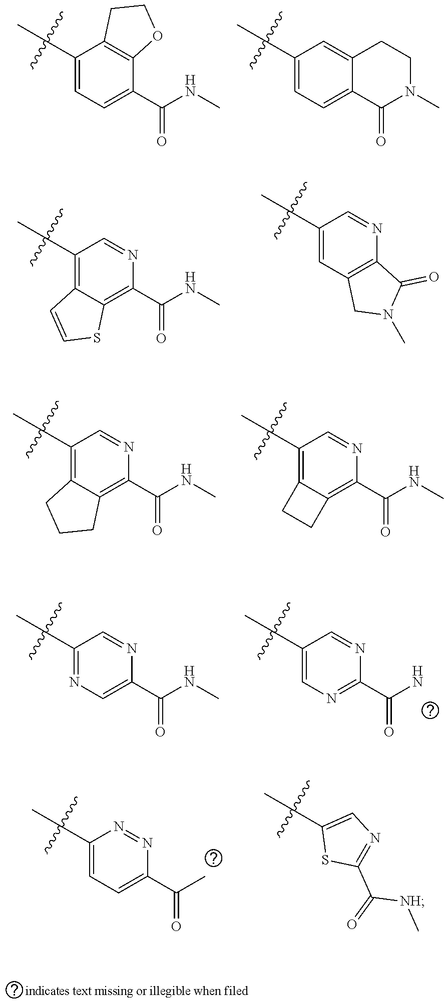

in some embodiments,

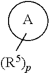

is selected from

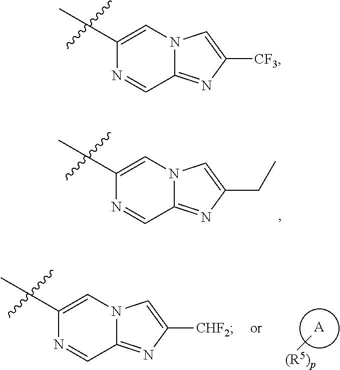

is selected from

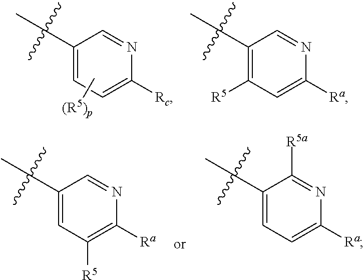

in some embodiments,

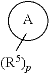

is selected from

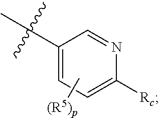

in some embodiments,

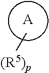

is selected from

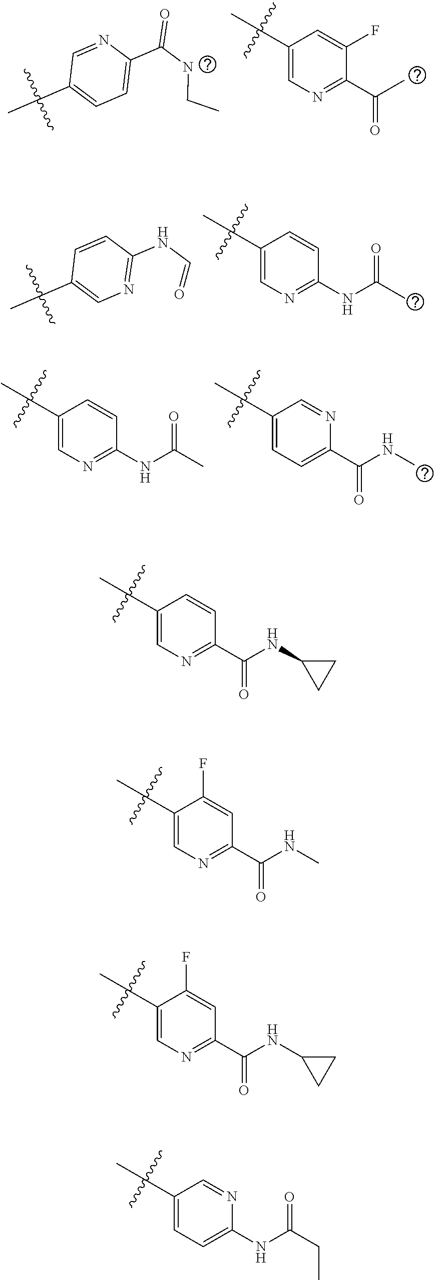

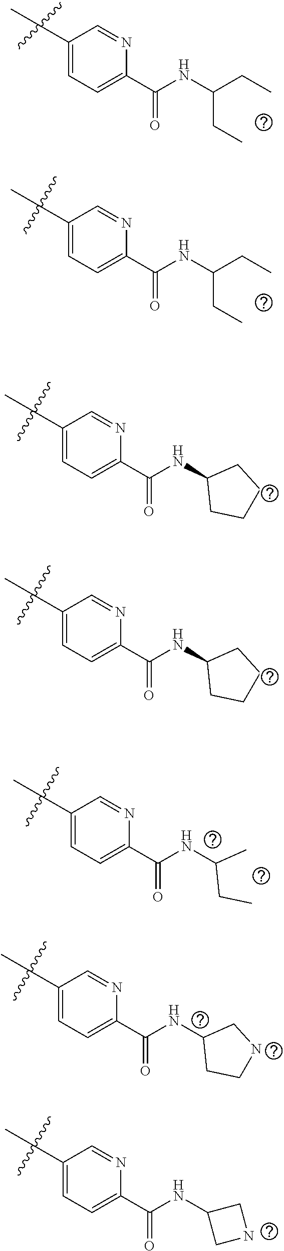

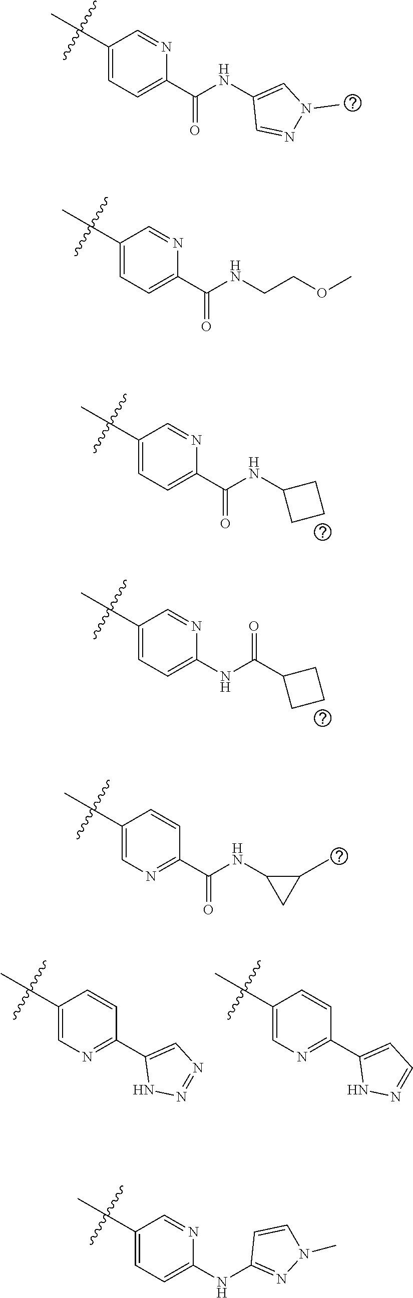

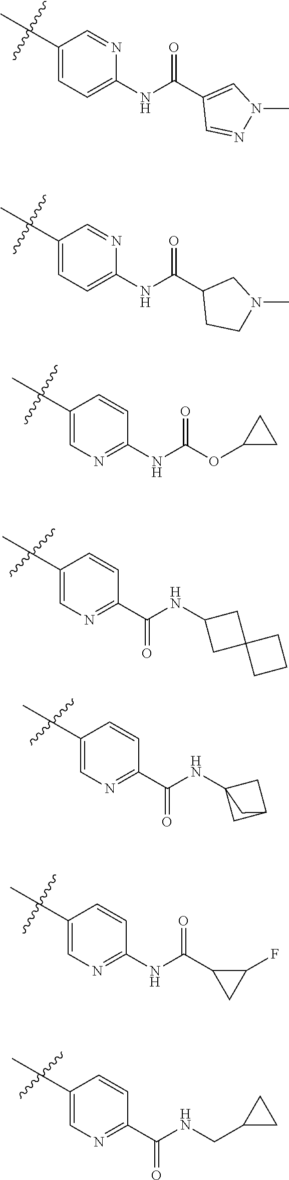

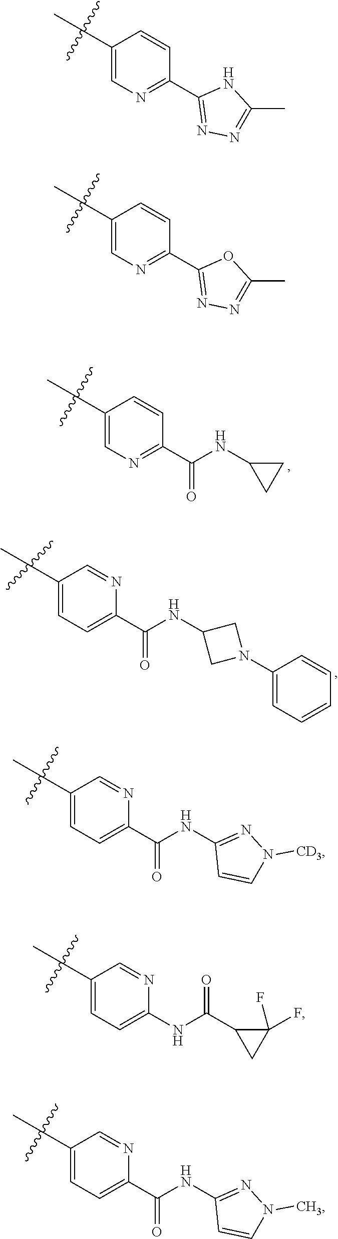

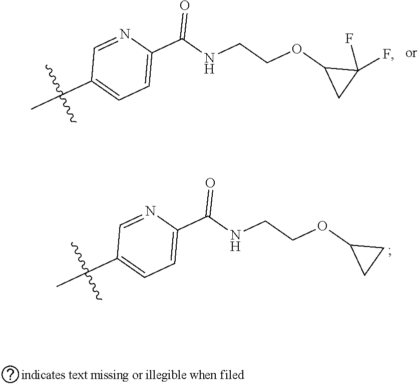 [SMILES: *c1ccc(C(=O)NCCOC2CC2(F)F)nc1 ; "or" ; *c1ccc(C(=O)NCCOC2CC2)nc1]

in some embodiments,

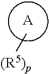

is selected from

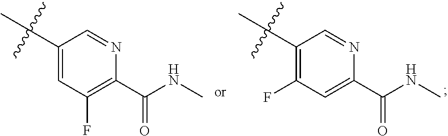 [SMILES: *c1cnc(C(=O)NC)c(F)c1 ; *c1cnc(C(=O)NC)cc1F]

R^{5a} is selected from cyano, amino, hydroxyl, —SF₅, C₁₋₆ alkoxy, halo C₁₋₆ alkoxy, deuterated C₁₋₆ alkyl, or deuterated C₁₋₆ alkoxy; in some embodiments, R^{5a} is selected from cyano, amino, hydroxyl, —SF₅, C₁₋₄ alkoxy, halo C₁₋₄ alkoxy, deuterated C₁₋₄ alkyl, or deuterated C₁₋₄ alkoxy; in some embodiments. R^{5a} is selected from cyano, amino, hydroxyl, C₁₋₂ alkoxy, halo C₁₋₂ alkoxy, deuterated C₁₋₂ alkyl, or deuterated C₁₋₂ alkoxy; in some embodiments, R^{5a} is selected from cyano or deuterated C₁₋₂ alkyl;
Rₐ is selected from —C(O)N(R^{a1})₂, —NR^{a1}C(O)OR^{a1}, —NR^{a1}C(O)R^{a1}, —NR^{a1}C(O)N(R^{a1})₂, —C(═S)N(R^{a1})₂, —S(O)₂N(R^{a1})₂, 5- to 6-membered monocyclic heteroaryl containing 1-5 nitrogen, oxygen or sulfur atoms, 4- to 7-membered monocyclic heterocycloalkyl containing 1-4 nitrogen, oxygen or sulfur atoms, or 3- to 7-membered monocyclic cycloalkyl, wherein the heteroaryl, heterocycloalkyl, or cycloalkyl is optionally further substituted with 1-3 substituents selected from D, halogen, cyano, hydroxyl, amino, —NHC₁₋₆ alkyl, —N(C₁₋₆ alkyl)₂, C₁₋₆ alkyl, halo C₁₋₆ alkyl, C₁₋₆ alkoxy, halo C₁₋₆ alkoxy, deuterated C₁₋₆ alkyl, or deuterated C₁₋₆ alkoxy; in some embodiments, Rₐ is selected from —C(O)N(R^{a1})₂, —NR^{a1}C(O)R^{a1}, —NR^{a1}C(O)OR^{a1}, —NR^{a1}C(O)N(R^{a1})₂, —C(═S)N(R^{a1})₂, —S(O)₂N(R^{a1})₂, 5-membered monocyclic heteroaryl containing 1, 2, 3, 4 or 5 nitrogen, oxygen or sulfur atoms, 6-membered monocyclic heteroaryl containing 1, 2, 3, 4 or 5 nitrogen, oxygen or sulfur atoms, 4- to 7-membered monocyclic heterocycloalkyl containing 1, 2, 3 or 4 nitrogen, oxygen or sulfur atoms, or 3- to 7-membered monocyclic cycloalkyl, wherein the heteroaryl, heterocycloalkyl, or cycloalkyl is optionally further substituted with 1, 2 or 3 substituents selected from D, halogen, cyano, hydroxyl, amino, —NHC₁₋₂ alkyl, —N(C₁₋₂ alkyl)₂, C₁₋₂ alkyl, halo C₁₋₂ alkyl, C₁₋₂ alkoxy, halo C₁₋₂ alkoxy, deuterated C₁₋₂ alkyl, or deuterated C₁₋₂ alkoxy; in some embodiments, Rₐ is selected from —C(O)N(R^{a1})₂, —NR^{a1}C(O)OR^{a1}, —NR^{a1}C(O)R^{a1}, —NR^{a1}C(O)N(R^{a1})₂, —C(═S)N(R^{a1})₂, —S(O)₂N(R^{a1})₂, 5-membered monocyclic heteroaryl containing 1, 2, 3 or 4 nitrogen, oxygen or sulfur atoms, 4-membered monocyclic heterocycloalkyl containing 1 or 2 nitrogen, oxygen or sulfur atoms, 5-membered monocyclic heterocycloalkyl containing 1 or 2 nitrogen, oxygen or sulfur atoms, 6-membered monocyclic heterocycloalkyl containing 1 or 2 nitrogen, oxygen or sulfur atoms, 4-membered monocyclic cycloalkyl, 5-membered monocyclic cycloalkyl, or 6-membered monocyclic cycloalkyl, wherein the heteroaryl, heterocycloalkyl, or cycloalkyl is optionally further substituted with 1, 2 or 3 substituents selected from D, halogen, cyano, hydroxyl, amino, —NHCH₃, —NHCH₂CH₃, —N(CH₃)₂, methyl, ethyl, methoxy, ethoxy, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —OCHF₂, —OCH₂F, —OCF₃, —OCH₂CH₂F, —OCH₂CHF₂, —OCH₂CF₃, —OCHFCH₂F, —OCHFCHF₂, —OCHFCF₃, —OCF₂CH₂F, —OCF₂CHF₂, —OCF₂CF₃, —CH₂D, —CHD₂, —CD₃, —CH₂CH₂D, —CH₂CHD₂, —CH₂CD₃, —CHDCH₂D, —CHDCHD₂, —CHDCD₃, —CD₂CH₂D, —CD₂CHD₂, —CD₂CD₃, —OCHD₂, —OCH₂D, —OCD₃, —OCH₂CH₂D, —OCH₂CHD₂, —OCH₂CD₃, —OCHDCH₂D, —OCHDCHD₂, —OCHDCD₃, —OCD₂CH₂D, —OCD₂CHD₂, or —OCD₂CD₃;
R_{b} is selected from —C(O)N(R^{a1})₂, —NR^{a1}C(O)OR^{a1}, —NR^{a1}C(O)R^{a1}, —NR^{a1}C(O)N(R^{a1})₂, —C(═S)N(R^{a1})₂, —S(O)₂N(R^{a1})₂, ═O, D, halogen, cyano, hydroxyl, amino, —NHC₁₋₆ alkyl, —N(C₁₋₆ alkyl)₂, C₁₋₆ alkyl, C₃₋₁₂ cycloalkyl, 3- to 12-membered heterocycloalkyl, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl, or deuterated C₁₋₆ alkoxy; in some embodiments, R_{b} is selected from —C(O)N(R^{a1})₂, —NR^{a1}C(O)OR^{a1}, —NR^{a1}C(O)R^{a1}, —NR^{a1}C(O)N(R^{a1})₂, —C(═S)N(R^{a1})₂, —S(O)₂N(R^{a1})₂, ═O, D, halogen, cyano, hydroxyl, amino, —NHC₁₋₂ alkyl, —N(C₁₋₂ alkyl)₂, C₁₋₂ alkyl, C₁₋₂ alkoxy, C₁₋₂ alkyl-O—C₁₋₂ alkyl, halo C₁₋₂ alkyl, halo C₁₋₂ alkoxy, deuterated C₁₋₂ alkyl, or deuterated C₁₋₂ alkoxy; in some embodiments, R_{b} is selected from —C(O)N(R^{a1})₂, —NR^{a1}C(O)R^{a1}, ═O, D, halogen, cyano, hydroxyl, amino, —NHC₁₋₂ alkyl, —N(C₁₋₂ alkyl)₂, C₁₋₂ alkyl, C₁₋₂ alkoxy, C₁₋₂ alkyl-O—C₁₋₂ alkyl, halo C₁₋₂ alkyl, halo C₁₋₂ alkoxy, deuterated C₁₋₂ alkyl, or deuterated C₁₋₂ alkoxy; in some embodiments, R_{b} is selected from —C(O)N(R^{a1})₂, ═O, D, halogen, cyano, hydroxyl, amino, —NHC₁₋₂ alkyl, —N(C₁₋₂ alkyl)₂, C₁₋₂ alkyl, or halo C₁₋₂ alkyl; in some embodiments, R_{b} is selected from —C(O)N(R^{a1})₂, —NR^{a1}C(O)R^{a1}, ═O, D, halogen, cyano, hydroxyl, amino, —NHCH₃, —NHCH₂CH₃, —N(CH₃)₂, methyl, ethyl, —CH₂F, —CHF₂, —CF₃, —CH₂CH₂F, —CH₂CHF₂, —CH₂CF₃, —CHFCH₂F, —CHFCHF₂, or —CHFCF₃;
R_{c} is selected from —C(O)R^{a2}, —NHR^{a2}, —C(O)N(R^{a2})₂, —C(O)NHR^{a2}, —NR^{a1}C(O)OR^{a1}, —NR^{a1}C(O)R^{a1}, —NR^{a1}C(O)R^{a2}, —NR^{a1}R^{a2}, —NR^{a1}C(O)N(R^{a1})₂, —C(═S)N(R^{a1})₂, —S(O)₂N(R^{a1})₂, 5- to 6-membered monocyclic heteroaryl containing 1-5 nitrogen, oxygen or sulfur atoms, 4- to 7-membered monocyclic heterocycloalkyl containing 1-4 nitrogen, oxygen or sulfur atoms, or 3- to 7-membered monocyclic cycloalkyl, wherein the heteroaryl, heterocycloalkyl, or cycloalkyl is optionally further substituted with 1-3 substituents selected from D, halogen, cyano, hydroxyl, amino, —NHC₁₋₆alkyl, —N(C₁₋₆ alkyl)₂, C₁₋₆ alkyl, halo C₁₋₆ alkyl, C₁₋₆ alkoxy, halo C₁₋₆ alkoxy, deuterated C₁₋₆ alkyl, or deuterated C₁₋₆ alkoxy; or R_{c} is selected from —C(O)N(R^{a2})₂, —C(O)NHR^{a2}, —NR^{a1}C(O)OR^{a1}, —NR^{a1}C(O)R^{a1}, —NR^{a1}C(O)R^{a2}, —NR^{a1}R^{a2}, —NR^{a1}C(O)N(R^{a1})₂, —C(═S)N(R^{a1})₂, —S(O)₂N(R^{a1})—, 5- to 6-membered monocyclic heteroaryl containing 1-5 nitrogen, oxygen or sulfur atoms, 4- to 7-membered monocyclic heterocycloalkyl containing 1-4 nitrogen, oxygen or sulfur atoms, or 3- to 7-membered monocyclic cycloalkyl, wherein the heteroaryl, heterocycloalkyl, or cycloalkyl is optionally further substituted with 1-3 substituents selected from D, halogen, cyano, hydroxyl, amino, —NHC₁₋₆ alkyl, —N(C₁₋₆ alkyl)₂, C₁₋₆ alkyl, halo C₁₋₆ alkyl, C₁₋₆ alkoxy, halo C₁₋₆ alkoxy, deuterated C₁₋₆ alkyl, or deuterated C₁₋₆ alkoxy; in some embodiments, R_{c} is selected from —C(O)N(R^{a2})₂, —C(O)NHR^{a2}, —NR^{a1}C(O)OR^{a1}, —NR^{a1}C(O)N(R^{a1})₂, —NR^{a1}C(O)R^{a1}, —NR^{a1}(O)R^{a2}, —NR^{a1}R^{a2}, —C(═S)N(R^{a1})₂, —S(O)₂N(R^{a1})₂, 5-membered monocyclic heteroaryl containing 1, 2, 3, 4 or 5 nitrogen, oxygen or sulfur atoms, 6-membered monocyclic heteroaryl containing 1, 2, 3, 4 or 5 nitrogen, oxygen or sulfur atoms, 4- to 7-membered monocyclic heterocycloalkyl containing 1, 2, 3 or 4 nitrogen, oxygen or sulfur atoms, or 3- to 7-membered monocyclic cycloalkyl, wherein the heteroaryl, heterocycloalkyl, or cycloalkyl is optionally further substituted with 1, 2 or 3 substituents selected from D, halogen, cyano, hydroxyl, amino, —NHC₁₋₂ alkyl, —N(C₁₋₂ alkyl)₂, C₁₋₂ alkyl, halo C₁₋₂ alkyl, C₁₋₂ alkoxy, halo C₁₋₂ alkoxy, deuterated C₁₋₂ alkyl, or deuterated C₁₋₂ alkoxy; in some embodiments, R_{c} is selected from —C(O)N(R^{2a})₂, —C(O)NHR^{a2}, —NR^{a1}C(O)OR^{a1}, —NR^{a1}C(O)R^{a1}, —NR^{a1}C(O)R^{a2}, —NR^{a1}R^{a2}, —NR^{a1}C(O)N(R^{a1})₂, —C(═S)N(R^{a1})₂, —S(O)₂N(R^{a1})₂, 5-membered monocyclic heteroaryl containing 1, 2, 3 or 4 nitrogen, oxygen or sulfur atoms, 4-membered monocyclic heterocycloalkyl containing 1 or 2 nitrogen, oxygen or sulfur atoms, 5-membered monocyclic heterocycloalkyl containing 1 or 2 nitrogen, oxygen or sulfur atoms, 6-membered monocyclic heterocycloalkyl containing 1 or 2 nitrogen, oxygen or sulfur atoms, 4-membered monocyclic cycloalkyl, 5-membered monocyclic cycloalkyl, or 6-membered monocyclic cycloalkyl, wherein the heteroaryl, heterocycloalkyl, or cycloalkyl is optionally further substituted with 1, 2 or 3 substituents selected from D, halogen, cyano, hydroxyl, amino, —NHCH₃, —NHCH₂CH₃, —N(CH₃)₂, methyl, ethyl, methoxy, ethoxy, —CH₂F, —CHF₂, —CF₃—CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —OCHF₂, —OCH₂F, —OCF₃, —OCH₂CH₂F, —OCH₂CHF₂, —OCH₂CF₃, —OCHFCH₂F, —OCHFCHF₂, —OCHFCF₃, —OCF₂CH₂F, —OCF₂CHF₂, —OCF₂CF₃, —CH₂D, —CHD₂, —CD₃, —CH₂CH₂D, —CH₂CHD₂, —CH₂CD₃, —CHDCH₂D, —CHDCHD₂, —CHDCD₃, —CD₂CH₂D, —CD₂CHD₂, —CD₂CD, —OCHD₂, —OCH₂D, —OCD₃, —OCH₂CH₂D, —OCH₂CHD₂, —OCH₂CD₃, —OCHDCH₂D, —OCHDCHD₂, —OCHDCD₃, —OCD₂CH₂D, —OCD₂CHD₂, or —OCD₂CD₃;
each R^{a1} is independently selected from H, D, C₁₋₆ alkyl, C₃₋₁₂ cycloalkyl, 3- to 12-membered heterocycloalkyl, 5- to 6-membered monocyclic heteroaryl containing 1-5 nitrogen, oxygen or sulfur atoms, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl, or deuterated C₁₋₆ alkoxy, wherein the cycloalkyl, heterocycloalkyl, or heteroaryl is optionally substituted with 1-3 substituents selected from halogen, deuterium or C₁₋₆ alkyl; or each R^{a1} is independently selected from H, D, C₁₋₆ alkyl, C₃₋₁₂ cycloalkyl, 3- to 12-membered heterocycloalkyl, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl, or deuterated C₁₋₆ alkoxy, wherein the cycloalkyl or heterocycloalkyl is optionally substituted with 1-3 substituents selected from halogen, deuterium, or C₁₋₆ alkyl; in some embodiments, each R^{a1} is independently selected from H, D, C₁₋₄ alkyl, C₃₋₆ monocyclic cycloalkyl, C₅₋₁₁ bicyclic spiro cycloalkyl, C₆₋₈ bicyclic bridged cycloalkyl, C₇₋₁₀ bicyclic fused cycloalkyl, 4- to 6-membered heterocycloalkyl, 6- to 9-membered bicyclic spiro heterocycloalkyl, C₆₋₈ bicyclic bridged heterocycloalkyl, C₇₋₁₀ bicyclic fused heterocycloalkyl, C₁₋₄ alkoxy, C₁₋₂ alkyl-O—C₁₋₂ alkyl, halo C₁₋₄ alkyl, halo C₁₋₄ alkoxy, deuterated C₁₋₄ alkyl, or deuterated C₁₋₄ alkoxy, wherein the cycloalkyl or heterocycloalkyl is optionally substituted with 1-3 substituents selected from halogen, deuterium, or C₁₋₂ alkyl; in some embodiments, each R^{a1} is independently selected from H, C₁₋₄ alkyl, C₃₋₆ monocyclic cycloalkyl, C₅₋₇ bicyclic spiro cycloalkyl, 4- to 6-membered heterocycloalkyl, C₁₋₂ alkyl-O—C₁₋₂ alkyl, halo C₁₋₄ alkyl, or deuterated C₁₋₄ alkyl, wherein the cycloalkyl or heterocycloalkyl is optionally substituted with 1, 2 or 3 substituents selected from F, Cl, deuterium, or C₁₋₂ alkyl; in some embodiments, each R^{a1} is independently selected from H, C₁₋₄ alkyl, C₃₋₄ monocyclic cycloalkyl, C₁₋₂ alkyl-O—C₁₋₂ alkyl, halo C₁₋₄ alkyl, or deuterated C₁₋₄ alkyl, wherein the cycloalkyl is optionally substituted with 1, 2 or 3 substituents selected from F, Cl, deuterium, or C₁₋₂ alkyl; in some embodiments, each R^{a1} is independently selected from H, methyl, ethyl, propyl, isopropyl, tert-butyl, cyclopropyl, cyclobutyl,

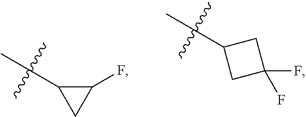 [SMILES: *C1CC1F ; *C1CC(F)(F)C1]

methoxymethyl, ethoxymethyl, methoxyethyl, ethoxyethyl, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂CH₂CH₂F, —CH₂CH₂CHF₂, or —CH₂CH₂CF₃;
each R^{a2} is independently selected from C₃₋₁₂ cycloalkyl, 3- to 12-membered heterocycloalkyl, C₁₋₆ alkyl-C₃₋₁₂ cycloalkyl, 5- to 6-membered monocyclic heteroaryl containing 1-5 nitrogen, oxygen or sulfur atoms, C₁₋₆ alkoxy, C₁₋₆ alkyl-O—C₁₋₆ alkyl, C₁₋₆ alkyl-O—C₃₋₆ cycloalkyl, halo C₁₋₆ alkyl, halo C₁₋₆ alkoxy, deuterated C₁₋₆ alkyl, or deuterated C₁₋₆ alkoxy, wherein the cycloalkyl, heterocycloalkyl, or heteroaryl is optionally substituted with 1-3 substituents selected from halogen, deuterium, C₁₋₆ alkyl, deuterated C₁₋₆ alkyl or phenyl; in some embodiments, each R^{a2} is independently selected from C₃₋₅ monocyclic cycloalkyl, C₁₋₂ alkyl-C₃₋₅ monocyclic cycloalkyl, C₅₋₉ spiro cycloalkyl, C₅₋₉ bridged cycloalkyl, 4- to 6-membered monocyclic heterocycloalkyl, 5- to 9-membered spiro heterocycloalkyl, 5- to 6-membered monocyclic heteroaryl containing 1-5 nitrogen, oxygen or sulfur atoms, C₁₋₄ alkoxy, C₁₋₂ alkyl-O—C₁₋₂ alkyl, C₁₋₄ alkyl-O—C₃₋₄ cycloalkyl, halo C₁₋₄ alkyl, halo C₁₋₄ alkoxy, deuterated C₁₋₄ alkyl, or deuterated C₁₋₄ alkoxy, wherein the cycloalkyl, heterocycloalkyl, or heteroaryl is optionally substituted with 1-3 substituents selected from halogen, deuterium, C₁₋₂ alkyl, deuterated C₁₋₂ alkyl, or phenyl; in some embodiments, each R^{a2} is independently selected from C₃₋₅ monocyclic cycloalkyl, C₁₋₂ alkyl-C₃₋₅ monocyclic cycloalkyl, C₅₋₉ spiro cycloalkyl, C₅₋₉ bridged cycloalkyl, 4- to 6-membered monocyclic heterocycloalkyl, 5- to 9-membered spiro heterocycloalkyl, 5- to 6-membered monocyclic heteroaryl containing 1-5 nitrogen, oxygen or sulfur atoms, C₁₋₄ alkoxy, C₁₋₂ alkyl-O—C₁₋₂ alkyl, C₁₋₃ alkyl-O—C₃₋₄ cycloalkyl, halo C₁₋₄ alkyl, halo C₁₋₄ alkoxy, deuterated C₁₋₄ alkyl, or deuterated C₁₋₄ alkoxy, wherein the cycloalkyl, heterocycloalkyl, or heteroaryl is optionally substituted with 1-3 substituents selected from halogen, deuterium, C₁₋₂ alkyl, deuterated C₁₋₂ alkyl, or phenyl; in some embodiments, each R^{a2} is independently selected from C₃₋₁₂ cycloalkyl, 3- to 12-membered heterocycloalkyl, C₁₋₆ alkyl-C₃₋₁₂ cycloalkyl, 5- to 6-membered monocyclic heteroaryl containing 1-5 nitrogen, oxygen or sulfur atoms, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl, or deuterated C₁₋₆ alkoxy, wherein the cycloalkyl, heterocycloalkyl, or heteroaryl is optionally substituted with 1-3 substituents selected from halogen, deuterium, C₁₋₆ alkyl, deuterated C₁₋₆ alkyl, or phenyl; in some embodiments, each R^{a2} is independently selected from C₃₋₁₂ cycloalkyl, 3- to 12-membered heterocycloalkyl, C₁₋₆ alkyl-C₃₋₁₂ cycloalkyl, 5- to 6-membered monocyclic heteroaryl containing 1-5 nitrogen, oxygen or sulfur atoms, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl, or deuterated C₁₋₆ alkoxy, wherein the cycloalkyl, heterocycloalkyl, or heteroaryl is optionally substituted with 1-3 substituents selected from halogen, deuterium, or C₁₋₆ alkyl; in some embodiments, each R^{a2} is independently selected from C₃₋₆ monocyclic cycloalkyl, C₁₋₄ alkyl-C₃₋₁₂ cycloalkyl, 5- to 6-membered monocyclic heteroaryl containing 1-5 nitrogen, oxygen or sulfur atoms, C₅₋₉ spiro cycloalkyl, C₅₋₉ bridged cycloalkyl, C₅₋₁₁ bicyclic spiro cycloalkyl, C₆₋₈ bicyclic bridged cycloalkyl, C₇₋₁₀ bicyclic fused cycloalkyl, 4- to 6-membered monocyclic heterocycloalkyl, 6- to 9-membered bicyclic spiro heterocycloalkyl, C₆₋₈ bicyclic bridged heterocycloalkyl, C₇₋₁₀ bicyclic fused heterocycloalkyl, C₁₋₄ alkoxy, C₁₋₂ alkyl-O—C₁₋₂ alkyl, halo C₁₋₄ alkyl, halo C₁₋₄ alkoxy, deuterated C₁₋₄ alkyl, or deuterated C₁₋₄ alkoxy, wherein the cycloalkyl, heterocycloalkyl, or heteroaryl is optionally substituted with 1-3 substituents selected from halogen, deuterium, or C₁₋₄ alkyl; in some embodiments, each R^{a2} is independently selected from C₃₋₆ monocyclic cycloalkyl, C₁₋₂ alkyl-C₃₋₅ monocyclic cycloalkyl, C₅₋₉ spiro cycloalkyl, C₅₋₉ bridged cycloalkyl, 5- to 6-membered monocyclic heteroaryl containing 1-5 nitrogen, oxygen or sulfur atoms, C₅₋₇ bicyclic spiro cycloalkyl, 4- to 6-membered monocyclic heterocycloalkyl, C₁₋₂ alkyl-O—C₁₋₂ alkyl, halo C₁₋₄ alkyl, or deuterated C₁₋₄ alkyl, wherein the cycloalkyl, heterocycloalkyl, or heteroaryl is optionally substituted with 1, 2 or 3 substituents selected from halogen, deuterium, C₁₋₂ alkyl, deuterated C₁₋₂ alkyl, or phenyl; in some embodiments, each R^{a2} is independently selected from C₃₋₆ monocyclic cycloalkyl, C₁₋₂ alkyl-C₃₋₅ monocyclic cycloalkyl, C₅₋₉ spiro cycloalkyl. C₅₋₉ bridged cycloalkyl, 5- to 6-membered monocyclic heteroaryl containing 1-5 nitrogen, oxygen or sulfur atoms, C₅₋₇ bicyclic spiro cycloalkyl, 4- to 6-membered monocyclic heterocycloalkyl, C₁₋₂ alkyl-O—C₁₋₂ alkyl, halo C₁₋₄ alkyl, or deuterated C₁₋₄ alkyl, wherein the cycloalkyl, heterocycloalkyl, or heteroaryl is optionally substituted with 1, 2 or 3 substituents selected from halogen, deuterium, or C₁₋₂ alkyl; in some embodiments, each R² is independently selected from C₃₋₄ monocyclic cycloalkyl, —CH₂—C₃₋₄ monocyclic cycloalkyl, C₆₋₉ spiro cycloalkyl, C₅₋₈ bridged cycloalkyl, 5- to 6-membered monocyclic heteroaryl containing 1-5 nitrogen, oxygen or sulfur atoms, 4- to 6-membered monocyclic heterocycloalkyl, C₁₋₂ alkyl-O—C₁₋₂ alkyl, halo C₁₋₄ alkyl, or deuterated C₁₋₄ alkyl, wherein the cycloalkyl, heterocycloalkyl, or heteroaryl is optionally substituted with 1, 2 or 3 substituents selected from F, Cl, deuterium, C₁₋₂ alkyl, deuterated C₁₋₂ alkyl, or phenyl; in some embodiments, each R^{a2} is independently selected from C₃₋₄ monocyclic cycloalkyl, —CH₂—C₃₋₄ monocyclic cycloalkyl, C₆₋₉ spiro cycloalkyl, C₅₋₈ bridged cycloalkyl, 5- to 6-membered monocyclic heteroaryl containing 1-5 nitrogen, oxygen or sulfur atoms, 4- to 6-membered monocyclic heterocycloalkyl, C₁₋₂ alkyl-O—C₁₋₂ alkyl, halo C₁₋₄ alkyl, or deuterated C₁₋₄ alkyl, wherein the cycloalkyl, heterocycloalkyl, or heteroaryl is optionally substituted with 1, 2 or 3 substituents selected from F, Cl, deuterium, or C₁₋₂ alkyl; in some embodiments, each R² is independently selected from cyclopropyl, cyclobutyl,

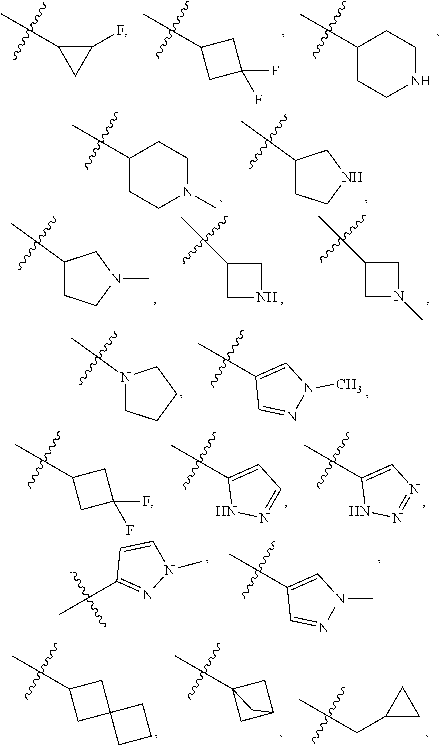

methoxymethyl, ethoxymethyl, methoxyethyl, ethoxyethyl, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂CH₂CH₂F, —CH₂CH₂CHF₂, —CH₂CH₂CF₃,

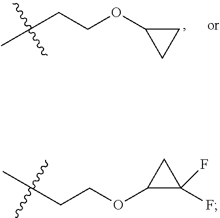 [SMILES: *CCOC1CC1 ; *CCOC1CC1(F)F]

in some embodiments, each R^{a2} is independently selected from

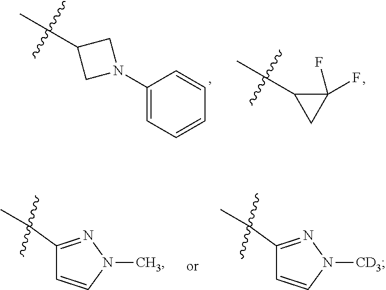 [SMILES: *C1CN(c2ccccc2)C1 ; *C1CC1(F)F ; *c1ccn(C)n1 ; *c1ccn(C([2H])([2H])[2H])n1]

in some embodiments, each R^{a2} is independently selected from cyclopropyl,

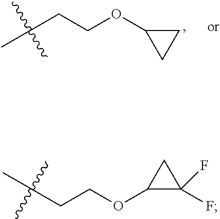 [SMILES: *CCOC1CC1 ; *CCOC1CC1(F)F]

alternatively, two R^{a2} together with the nitrogen atom to which they are attached form 4- to 6-membered heterocycloalkyl, wherein the heterocycloalkyl is optionally substituted with 1-3 substituents selected from halogen, deuterium, or C₁₋₆ alkyl; in some embodiments, two R² together with the nitrogen atom to which they are attached form 4-, 5- or 6-membered heterocycloalkyl, wherein the heterocycloalkyl is optionally substituted with 1, 2 or 3 substituents selected from F, Cl, deuterium, or C₁₋₂ alkyl;
unless otherwise specified, the above-mentioned heterocycloalkane, heterocycloalkyl, heteroaryl, or heteroaromatic ring contains 1, 2, 3, 4 or 5 heteroatoms selected from nitrogen, oxygen or sulfur; in some embodiments, the heterocycloalkane, heterocycloalkyl, heteroaryl, or heteroaromatic ring contains 1, 2, 3 or 4 heteroatoms selected from nitrogen, oxygen or sulfur; the heterocycloalkane, heterocycloalkyl, heteroaryl, or heteroaromatic ring contains 1, 2 or 3 heteroatoms selected from nitrogen, oxygen or sulfur; the heterocycloalkane, heterocycloalkyl, heteroaryl, or heteroaromatic ring contains 1 or 2 heteroatoms selected from nitrogen, oxygen or sulfur.

In some embodiments, the compound has a structure selected from one of the structures shown in Table S-1:

TABLE S-1
Compound structure

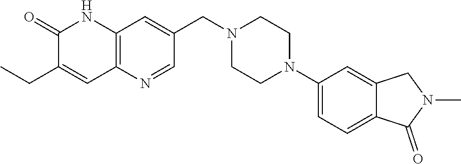 [SMILES: CCc1cc2ncc(CN3CCN(c4ccc5c(c4)CN(C)C5=O)CC3)cc2[nH]c1=O]

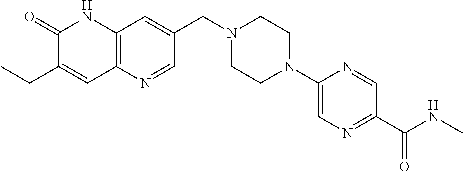 [SMILES: CCc1cc2ncc(CN3CCN(c4cnc(C(=O)NC)cn4)CC3)cc2[nH]c1=O]

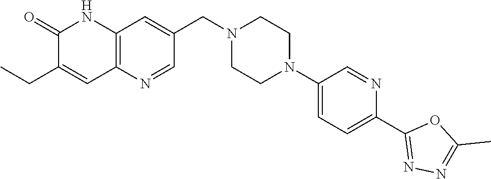 [SMILES: CCc1cc2ncc(CN3CCN(c4ccc(-c5nnc(C)o5)nc4)CC3)cc2[nH]c1=O]

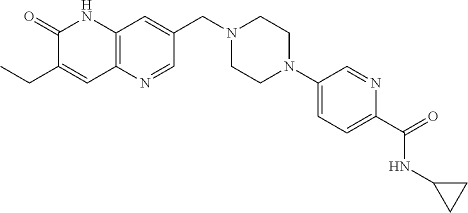 [SMILES: CCc1cc2ncc(CN3CCN(c4ccc(C(=O)NC5CC5)nc4)CC3)cc2[nH]c1=O]

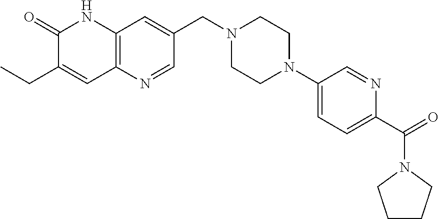 [SMILES: CCc1cc2ncc(CN3CCN(c4ccc(C(=O)N5CCCC5)nc4)CC3)cc2[nH]c1=O]

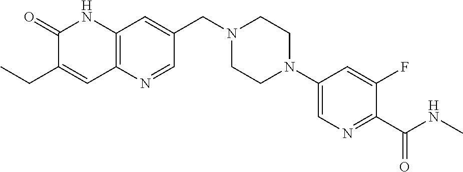 [SMILES: CCc1cc2ncc(CN3CCN(c4cnc(C(=O)NC)c(F)c4)CC3)cc2[nH]c1=O]

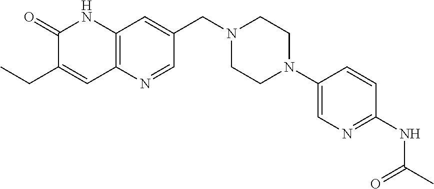 [SMILES: CCc1cc2ncc(CN3CCN(c4ccc(NC(C)=O)nc4)CC3)cc2[nH]c1=O]

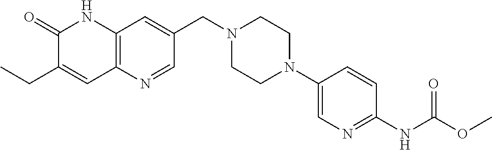 [SMILES: CCc1cc2ncc(CN3CCN(c4ccc(NC(=O)OC)nc4)CC3)cc2[nH]c1=O]

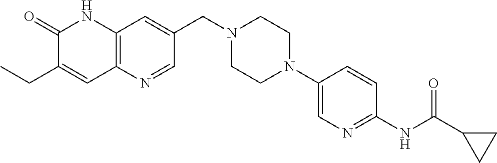 [SMILES: CCc1cc2ncc(CN3CCN(c4ccc(NC(=O)C5CC5)nc4)CC3)cc2[nH]c1=O]

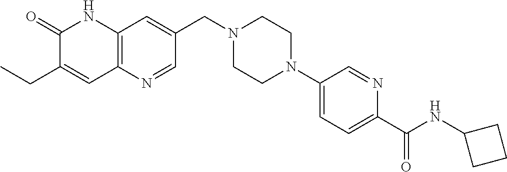 [SMILES: CCc1cc2ncc(CN3CCN(c4ccc(C(=O)NC5CCC5)nc4)CC3)cc2[nH]c1=O]

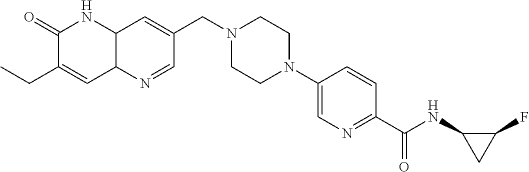 [SMILES: CCC1=CC2N=CC(CN3CCN(c4ccc(C(=O)N[C@@H]5C[C@@H]5F)nc4)CC3)=CC2NC1=O]

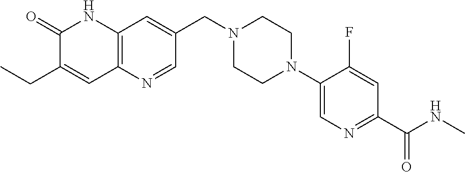 [SMILES: CCc1cc2ncc(CN3CCN(c4cnc(C(=O)NC)cc4F)CC3)cc2[nH]c1=O]

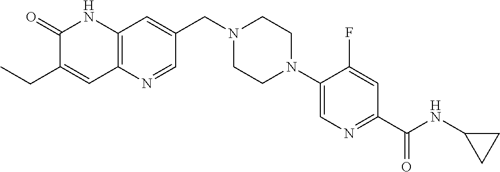 [SMILES: CCc1cc2ncc(CN3CCN(c4cnc(C(=O)NC5CC5)cc4F)CC3)cc2[nH]c1=O]

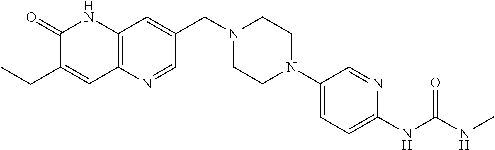 [SMILES: CCc1cc2ncc(CN3CCN(c4ccc(NC(=O)NC)nc4)CC3)cc2[nH]c1=O]

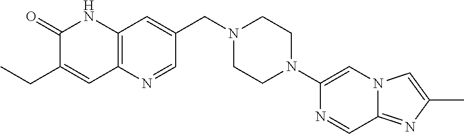 [SMILES: CCc1cc2ncc(CN3CCN(c4cn5cc(C)nc5cn4)CC3)cc2[nH]c1=O]

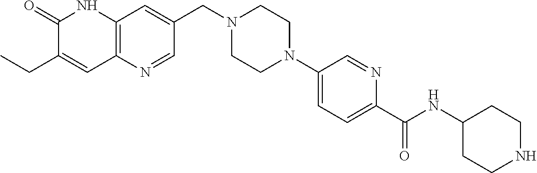 [SMILES: CCc1cc2ncc(CN3CCN(c4ccc(C(=O)NC5CCNCC5)nc4)CC3)cc2[nH]c1=O]

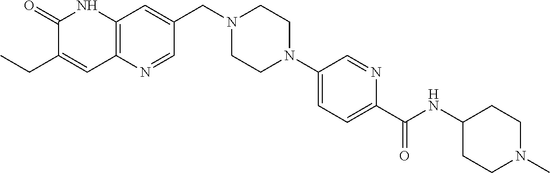 [SMILES: CCc1cc2ncc(CN3CCN(c4ccc(C(=O)NC5CCN(C)CC5)nc4)CC3)cc2[nH]c1=O]

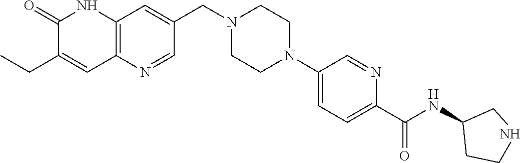 [SMILES: CCc1cc2ncc(CN3CCN(c4ccc(C(=O)N[C@@H]5CCNC5)nc4)CC3)cc2[nH]c1=O]

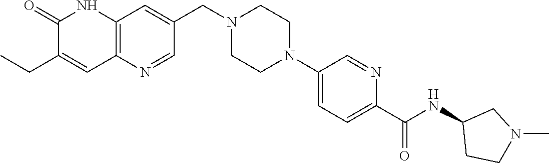 [SMILES: CCc1cc2ncc(CN3CCN(c4ccc(C(=O)N[C@@H]5CCN(C)C5)nc4)CC3)cc2[nH]c1=O]

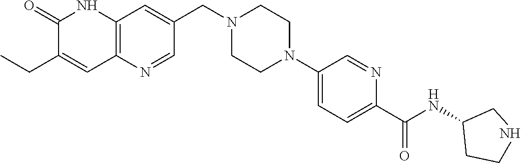 [SMILES: CCc1cc2ncc(CN3CCN(c4ccc(C(=O)N[C@H]5CCNC5)nc4)CC3)cc2[nH]c1=O]

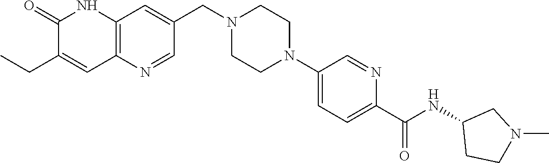 [SMILES: CCc1cc2ncc(CN3CCN(c4ccc(C(=O)N[C@H]5CCN(C)C5)nc4)CC3)cc2[nH]c1=O]

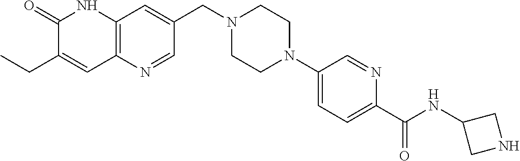 [SMILES: CCc1cc2ncc(CN3CCN(c4ccc(C(=O)NC5CNC5)nc4)CC3)cc2[nH]c1=O]

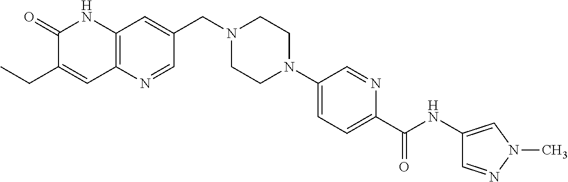 [SMILES: CCc1cc2ncc(CN3CCN(c4ccc(C(=O)Nc5cnn(C)c5)nc4)CC3)cc2[nH]c1=O]

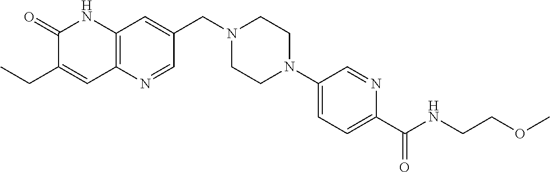 [SMILES: CCc1cc2ncc(CN3CCN(c4ccc(C(=O)NCCOC)nc4)CC3)cc2[nH]c1=O]

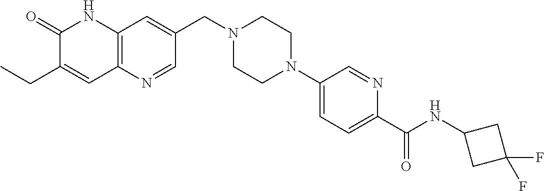 [SMILES: CCc1cc2ncc(CN3CCN(c4ccc(C(=O)NC5CC(F)(F)C5)nc4)CC3)cc2[nH]c1=O]

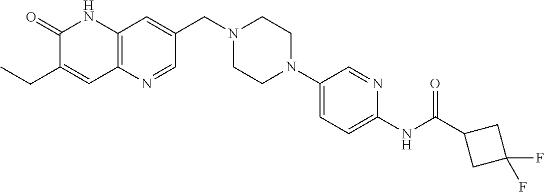 [SMILES: CCc1cc2ncc(CN3CCN(c4ccc(NC(=O)C5CC(F)(F)C5)nc4)CC3)cc2[nH]c1=O]

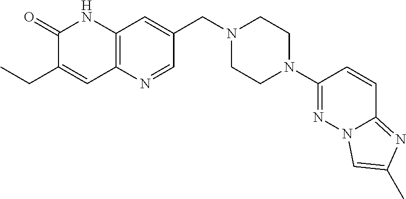 [SMILES: CCc1cc2ncc(CN3CCN(c4ccc5nc(C)cn5n4)CC3)cc2[nH]c1=O]

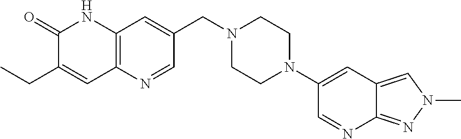 [SMILES: CCc1cc2ncc(CN3CCN(c4cnc5nn(C)cc5c4)CC3)cc2[nH]c1=O]

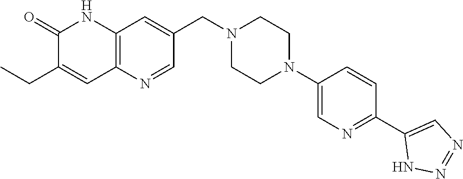 [SMILES: CCc1cc2ncc(CN3CCN(c4ccc(-c5cnn[nH]5)nc4)CC3)cc2[nH]c1=O]

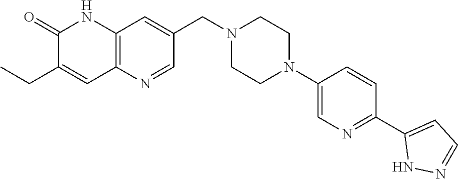 [SMILES: CCc1cc2ncc(CN3CCN(c4ccc(-c5ccn[nH]5)nc4)CC3)cc2[nH]c1=O]

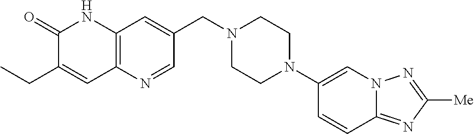 [SMILES: CCc1cc2ncc(CN3CCN(c4ccc5nc(C)nn5c4)CC3)cc2[nH]c1=O]

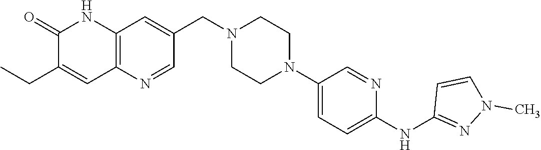 [SMILES: CCc1cc2ncc(CN3CCN(c4ccc(Nc5ccn(C)n5)nc4)CC3)cc2[nH]c1=O]

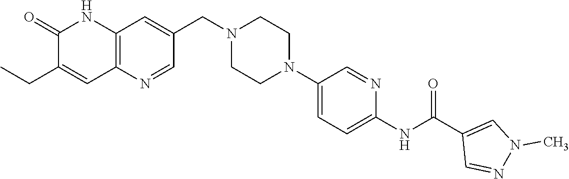 [SMILES: CCc1cc2ncc(CN3CCN(c4ccc(NC(=O)c5cnn(C)c5)nc4)CC3)cc2[nH]c1=O]

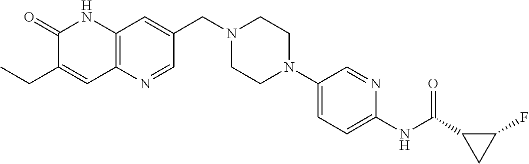

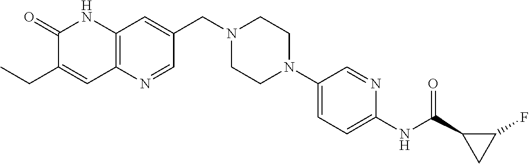

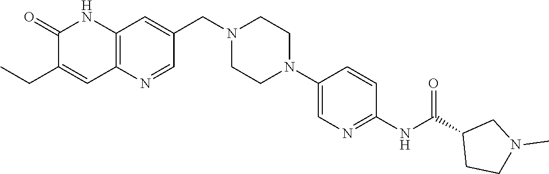 [SMILES: CCc1cc2ncc(CN3CCN(c4ccc(NC(=O)[C@H]5CCN(C)C5)nc4)CC3)cc2[nH]c1=O]

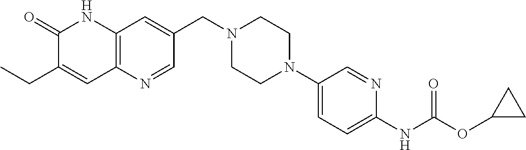 [SMILES: CCc1cc2ncc(CN3CCN(c4ccc(NC(=O)OC5CC5)nc4)CC3)cc2[nH]c1=O]

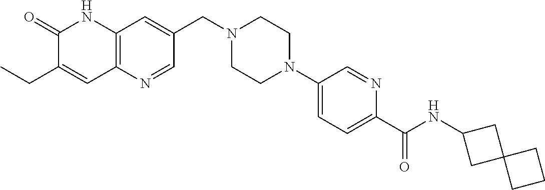 [SMILES: CCc1cc2ncc(CN3CCN(c4ccc(C(=O)NC5CC6(CCC6)C5)nc4)CC3)cc2[nH]c1=O]

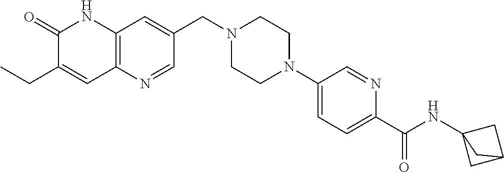 [SMILES: CCc1cc2ncc(CN3CCN(c4ccc(C(=O)NC56CC(C5)C6)nc4)CC3)cc2[nH]c1=O]

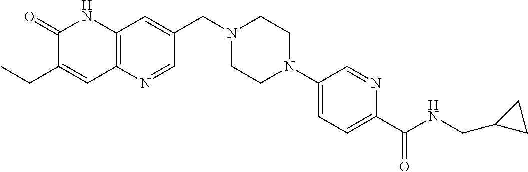 [SMILES: CCc1cc2ncc(CN3CCN(c4ccc(C(=O)NCC5CC5)nc4)CC3)cc2[nH]c1=O]

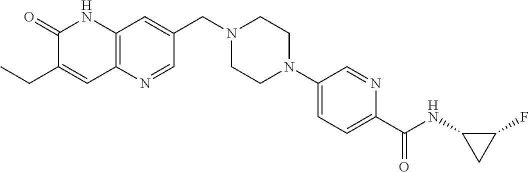 [SMILES: CCc1cc2ncc(CN3CCN(c4ccc(C(=O)N[C@H]5C[C@H]5F)nc4)CC3)cc2[nH]c1=O]

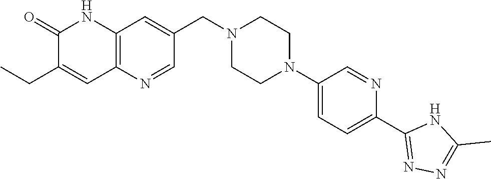 [SMILES: CCc1cc2ncc(CN3CCN(c4ccc(-c5nnc(C)[nH]5)nc4)CC3)cc2[nH]c1=O]

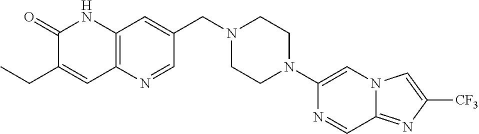 [SMILES: CCc1cc2ncc(CN3CCN(c4cn5cc(C(F)(F)F)nc5cn4)CC3)cc2[nH]c1=O]

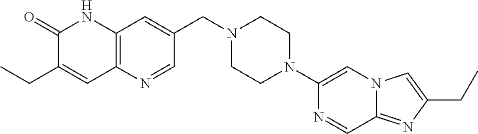 [SMILES: CCc1cn2cc(N3CCN(Cc4cnc5cc(CC)c(=O)[nH]c5c4)CC3)ncc2n1]

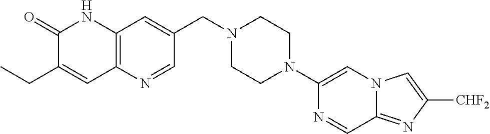 [SMILES: CCc1cc2ncc(CN3CCN(c4cn5cc(C(F)F)nc5cn4)CC3)cc2[nH]c1=O]

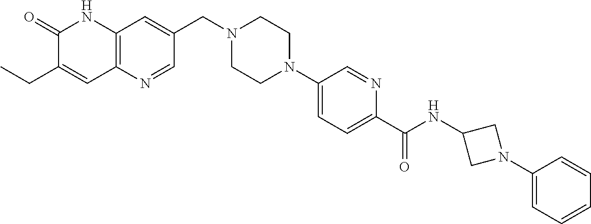 [SMILES: CCc1cc2ncc(CN3CCN(c4ccc(C(=O)NC5CN(c6ccccc6)C5)nc4)CC3)cc2[nH]c1=O]

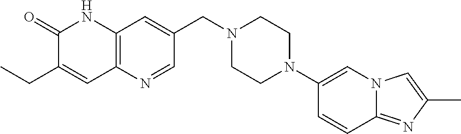 [SMILES: CCc1cc2ncc(CN3CCN(c4ccc5nc(C)cn5c4)CC3)cc2[nH]c1=O]

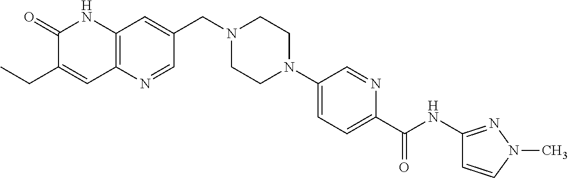 [SMILES: CCc1cc2ncc(CN3CCN(c4ccc(C(=O)Nc5ccn(C)n5)nc4)CC3)cc2[nH]c1=O]

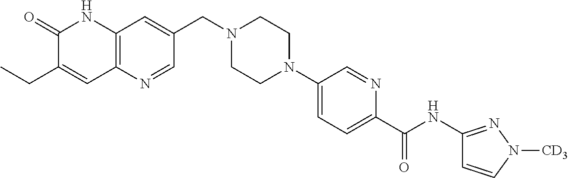 [SMILES: [2H]C([2H])([2H])n1ccc(NC(=O)c2ccc(N3CCN(Cc4cnc5cc(CC)c(=O)[nH]c5c4)CC3)cn2)n1]

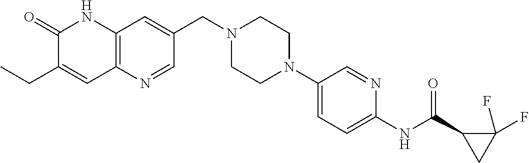 [SMILES: CCc1cc2ncc(CN3CCN(c4ccc(NC(=O)[C@@H]5CC5(F)F)nc4)CC3)cc2[nH]c1=O]

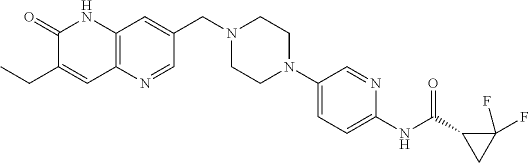 [SMILES: CCc1cc2ncc(CN3CCN(c4ccc(NC(=O)[C@H]5CC5(F)F)nc4)CC3)cc2[nH]c1=O]

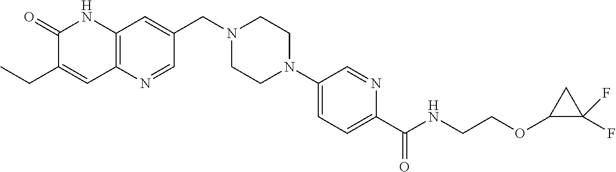 [SMILES: CCc1cc2ncc(CN3CCN(c4ccc(C(=O)NCCOC5CC5(F)F)nc4)CC3)cc2[nH]c1=O]

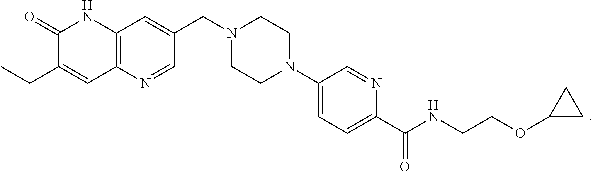 [SMILES: CCc1cc2ncc(CN3CCN(c4ccc(C(=O)NCCOC5CC5)nc4)CC3)cc2[nH]c1=O]

In some embodiments, the active ingredient M is selected from the following structure

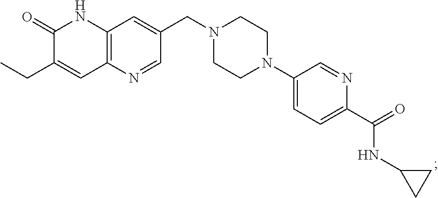 [SMILES: CCc1cc2ncc(CN3CCN(c4ccc(C(=O)NC5CC5)nc4)CC3)cc2[nH]c1=O]

the pharmaceutical composition or the pharmaceutical preparation comprises 1-600 mg of the active ingredient M;
in some embodiments, the pharmaceutical composition or the pharmaceutical preparation of the present disclosure comprises 5-300 mg of the active ingredient M;
in some embodiments, the pharmaceutical composition or the pharmaceutical preparation of the present disclosure comprises 5-200 mg of the active ingredient M;
in some embodiments, the pharmaceutical composition or the pharmaceutical preparation of the present disclosure comprises 5-100 mg of the active ingredient M;
in some embodiments, the pharmaceutical composition or the pharmaceutical preparation of the present disclosure comprises 5 mg of the active ingredient M;
in some embodiments, the pharmaceutical composition or the pharmaceutical preparation of the present disclosure comprises 10 mg of the active ingredient M;
in some embodiments, the pharmaceutical composition or the pharmaceutical preparation of the present disclosure comprises 20 mg of the active ingredient M;
in some embodiments, the pharmaceutical composition or the pharmaceutical preparation of the present disclosure comprises 50 mg of the active ingredient M;
in some embodiments, the pharmaceutical composition or the pharmaceutical preparation of the present disclosure comprises 60 mg of the active ingredient M;
in some embodiments, the pharmaceutical composition or the pharmaceutical preparation of the present disclosure comprises 70 mg of the active ingredient M;
in some embodiments, the pharmaceutical composition or the pharmaceutical preparation of the present disclosure comprises 80 mg of the active ingredient M;
in some embodiments, the pharmaceutical composition or the pharmaceutical preparation of the present disclosure comprises 90 mg of the active ingredient M;
in some embodiments, the pharmaceutical composition or the pharmaceutical preparation of the present disclosure comprises 100 mg of the active ingredient M;
in some embodiments, the pharmaceutical composition or the pharmaceutical preparation of the present disclosure comprises 150 mg of the active ingredient M;
in some embodiments, the pharmaceutical composition or the pharmaceutical preparation of the present disclosure comprises 200 mg of the active ingredient M.

The present disclosure relates to a pharmaceutical composition or a pharmaceutical preparation, wherein the pharmaceutical composition or the pharmaceutical preparation comprises an active ingredient M and a pharmaceutically acceptable excipient, and the active ingredient M is selected from among a compound having general formula (I) or a stereoisomer, a tautomer, a deuterated substance, a solvate, a prodrug, a metabolite, a pharmaceutically acceptable salt or a co-crystal thereof, having a structure of formula (II), (III), (IV), (V) or (VI):

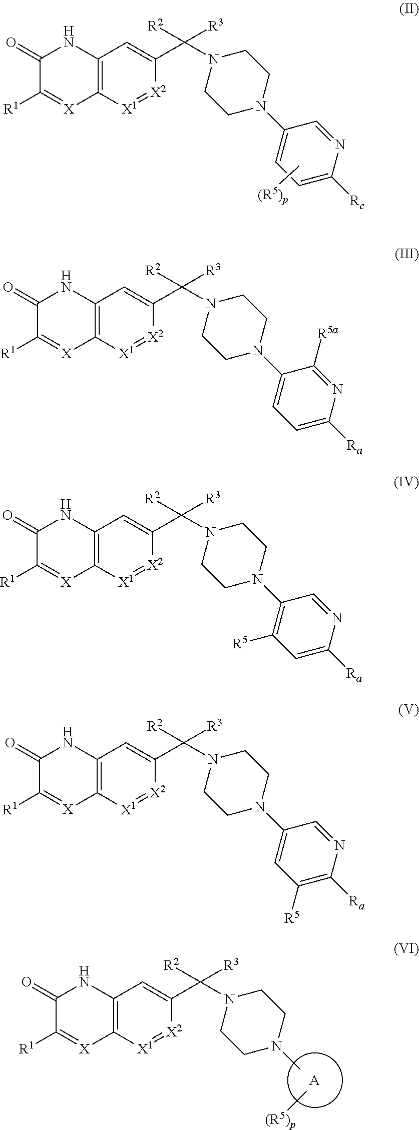

wherein X is selected from CR^{x} or N, provided that X, X¹ and X² are not all selected from CR^{x};
the definitions of other groups are consistent with those mentioned above.

The present disclosure relates to a pharmaceutical composition or a pharmaceutical preparation, wherein the pharmaceutical composition or the pharmaceutical preparation comprises an active ingredient M and a pharmaceutically acceptable excipient, and the active ingredient M is selected from among a compound having general formula (I), (II), (III), (IV), (V), or (VI) or a stereoisomer, a tautomer, a deuterated substance, a solvate, a prodrug, a metabolite, a pharmaceutically acceptable salt or a co-crystal thereof.

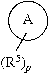

is selected from

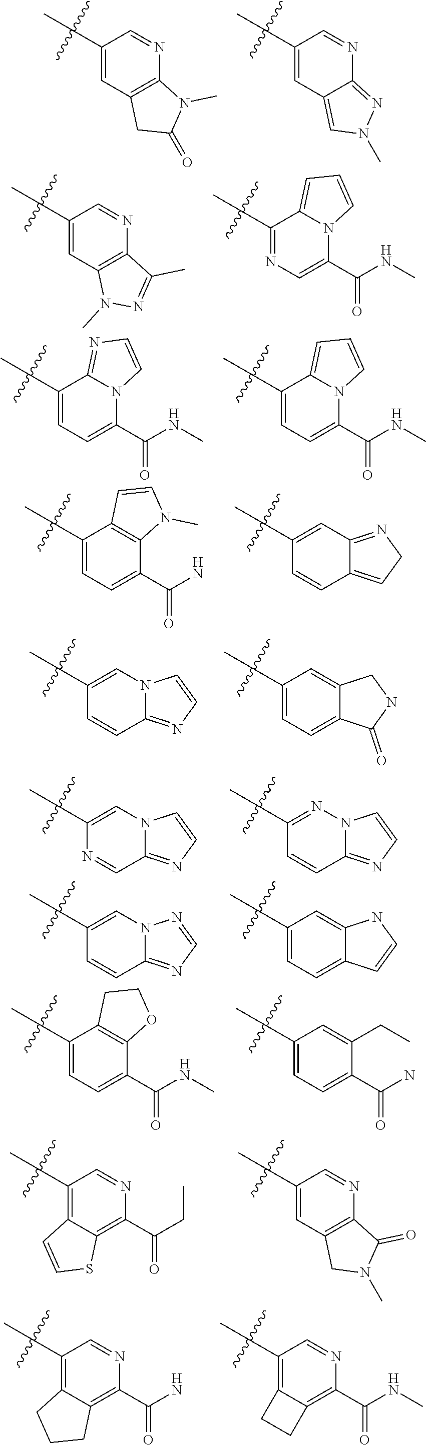

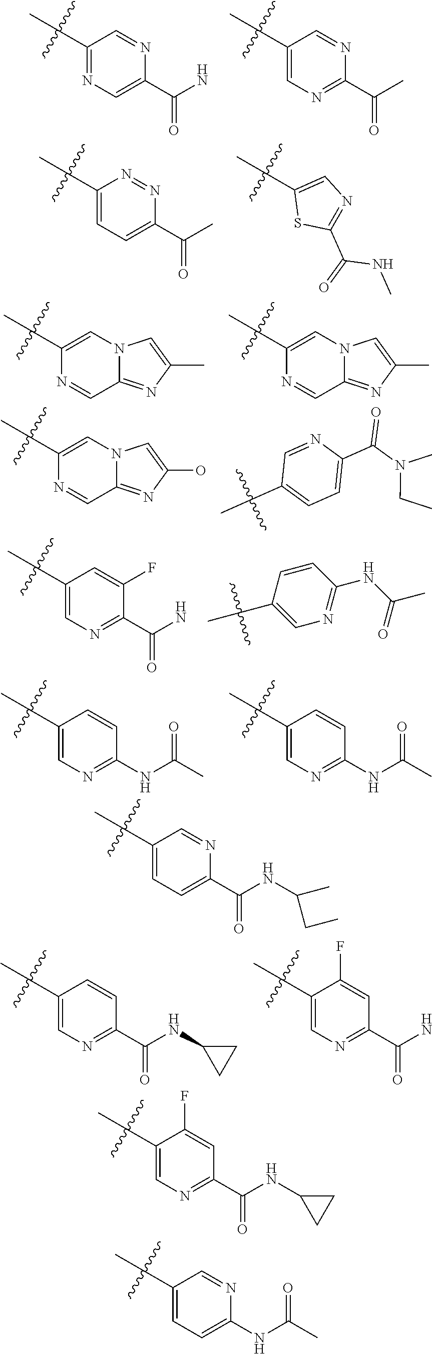

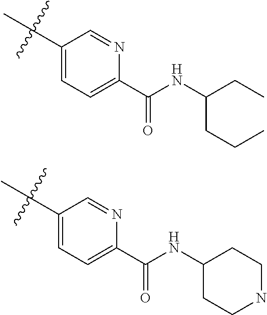 [SMILES: *c1ccc(C(=O)NC(CC)CCC)nc1 ; *c1ccc(C(=O)NC2CCNCC2)nc1]

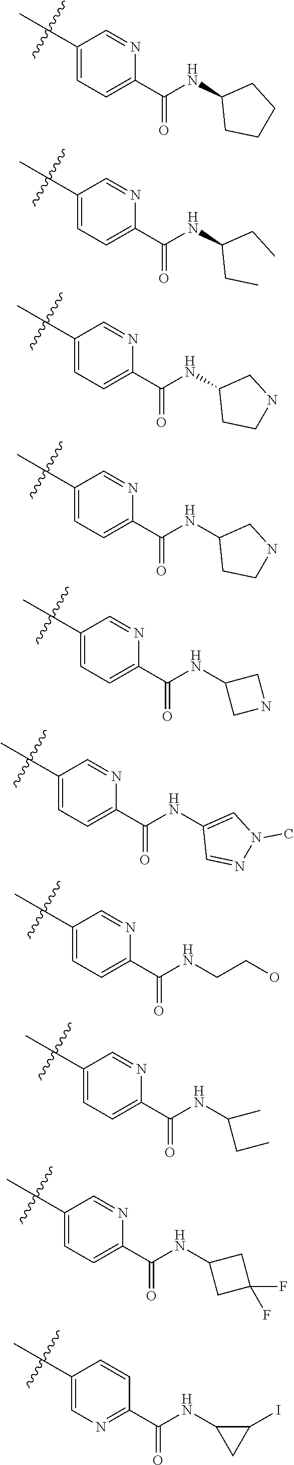

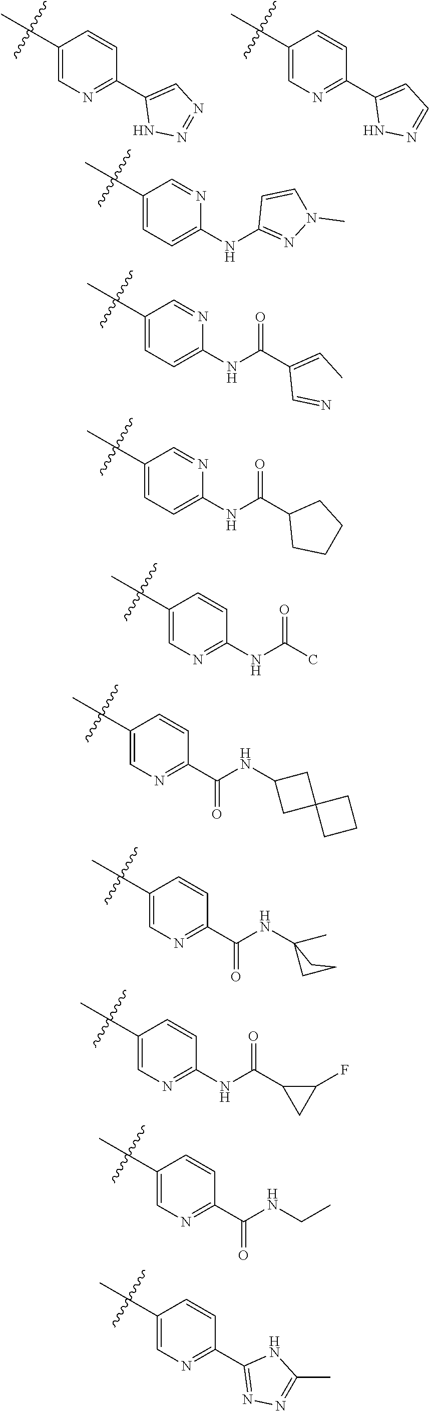

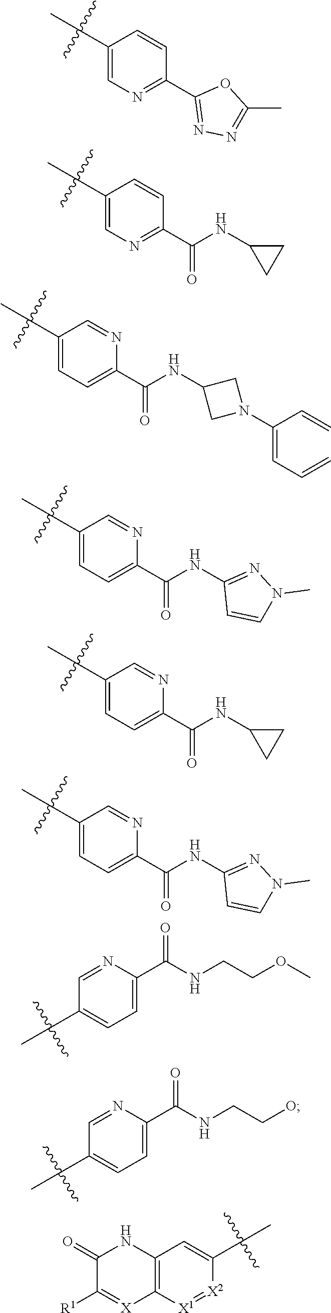

is selected from

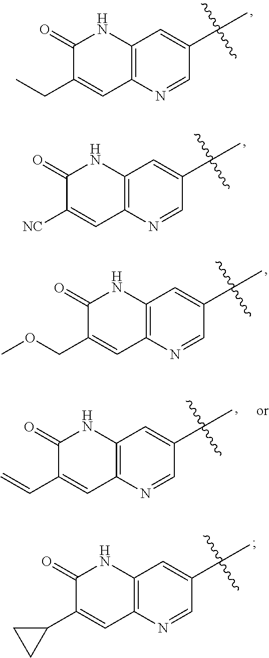 [SMILES: *c1cnc2cc(CC)c(=O)[nH]c2c1 ; *c1cnc2cc(C#N)c(=O)[nH]c2c1 ; *c1cnc2cc(COC)c(=O)[nH]c2c1 ; *c1cnc2cc(C=C)c(=O)[nH]c2c1 ; *c1cnc2cc(C3CC3)c(=O)[nH]c2c1]

the definitions of other groups are consistent with those mentioned above.

The present disclosure relates to a pharmaceutical composition or a pharmaceutical preparation comprising the active ingredient M and the pharmaceutically acceptable excipient of any of the preceding embodiments, wherein the pharmaceutical composition or the pharmaceutical preparation comprises 1-600 mg of the active ingredient M; in some embodiments, the pharmaceutical composition or the pharmaceutical preparation of the present disclosure comprises 5-300 mg of the active ingredient M; in some embodiments, the pharmaceutical composition or the pharmaceutical preparation of the present disclosure comprises 5-200 mg of the active ingredient M; in some embodiments, the pharmaceutical composition or the pharmaceutical preparation of the present disclosure comprises 5-100 mg of the active ingredient M; in some embodiments, the pharmaceutical composition or the pharmaceutical preparation of the present disclosure comprises 5 mg of the active ingredient M; in some embodiments, the pharmaceutical composition or the pharmaceutical preparation of the present disclosure comprises 10 mg of the active ingredient M; in some embodiments, the pharmaceutical composition or the pharmaceutical preparation of the present disclosure comprises 20 mg of the active ingredient M; in some embodiments, the pharmaceutical composition or the pharmaceutical preparation of the present disclosure comprises 50 mg of the active ingredient M; in some embodiments, the pharmaceutical composition or the pharmaceutical preparation of the present disclosure comprises 60 mg of the active ingredient M; in some embodiments, the pharmaceutical composition or the pharmaceutical preparation of the present disclosure comprises 70 mg of the active ingredient M; in some embodiments, the pharmaceutical composition or the pharmaceutical preparation of the present disclosure comprises 80 mg of the active ingredient M; in some embodiments, the pharmaceutical composition or the pharmaceutical preparation of the present disclosure comprises 90 mg of the active ingredient M; in some embodiments, the pharmaceutical composition or the pharmaceutical preparation of the present disclosure comprises 100 mg of the active ingredient M; in some embodiments, the pharmaceutical composition or the pharmaceutical preparation of the present disclosure comprises 150 mg of the active ingredient M; in some embodiments, the pharmaceutical composition or the pharmaceutical preparation of the present disclosure comprises 200 mg of the active ingredient M.

The pharmaceutical composition or the pharmaceutical preparation of the present disclosure, comprising the active ingredient M and the pharmaceutically acceptable excipient of any one of the preceding embodiments, wherein the active ingredient M is present in an amount of 0.5%-90%; in some embodiments, in an amount of 1%-90%; in some embodiments, in an amount of 1%-80%; in some embodiments, in an amount of %-70%; in some embodiments, in an amount of 1%-60%; in some embodiments, in an amount of 1%-50%; in some embodiments, in an amount of 1%-40%; in some embodiments, in an amount of 1%-30%; in some embodiments, in an amount of 1%-20%; in some embodiments, in an amount of 1%-10%; in some embodiments, in an amount of 5%-90%; in some embodiments, in an amount of 5%-80%; in some embodiments, in an amount of 5%-70%; in some embodiments, in an amount of 5%-60%; in some embodiments, in an amount of 5%-50%; in some embodiments, in an amount of 5%-40%; in some embodiments, in an amount of 5%-30%; in some embodiments, in an amount of 5%-20%; in some embodiments, in an amount of 5%-10%; in some embodiments, in an amount of 10%-90%; in some embodiments, in an amount of 10%-80%; in some embodiments, in an amount of 10%-70%; in some embodiments, in an amount of 10%-60%; in some embodiments, in an amount of 10%-50%; in some embodiments, in an amount of 10%-40%; in some embodiments, in an amount of 10%-30%; in some embodiments, in an amount of 10%-20%; in some embodiments, in an amount of 10%.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes one or both of a filler and a disintegrant.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes one or both of a filler and a disintegrant, and the active ingredient M is present in an amount of 0.5%-90%; in some embodiments, in an amount of 1%-90%; in some embodiments, in an amount of 1%-80%; in some embodiments, in an amount of 1%-70%; in some embodiments, in an amount of 1%-60%; in some embodiments, in an amount of 1%-50%; in some embodiments, in an amount of 1%-40%; in some embodiments, in an amount of 1%-30%; in some embodiments, in an amount of 1%-20%; in some embodiments, in an amount of 1%-10%; in some embodiments, in an amount of 5%-90%; in some embodiments, in an amount of 5%-80%; in some embodiments, in an amount of 5%-70%; in some embodiments, in an amount of 5%-60%; in some embodiments, in an amount of 5%-50%; in some embodiments, in an amount of 5%-40%; in some embodiments, in an amount of 5%-30%; in some embodiments, in an amount of 5%-20%; in some embodiments, in an amount of 5%-10%; in some embodiments, in an amount of 10%-90%; in some embodiments, in an amount of 10%-80%; in some embodiments, in an amount of 10%-70%; in some embodiments, in an amount of 10%-60%; in some embodiments, in an amount of 10%-50%; in some embodiments, in an amount of 10%-40%; in some embodiments, in an amount of 10%-30%; in some embodiments, in an amount of 10%-20%; in some embodiments, in an amount of 10%.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler and a disintegrant, and further includes one or more of a binder, a glidant, and a lubricant.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler and a disintegrant, and further includes one or more of a binder, a glidant, a lubricant, and a pH regulator.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler, a disintegrant, a binder, a glidant, and a lubricant, and further includes a pH regulator.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler and a disintegrant, and further includes one or more of a binder, a glidant, a lubricant, and a pH regulator; the active ingredient M is present in an amount of 0.5%-99%; and in some embodiments, the active ingredient M is present in an amount of 0.5%-90%; in some embodiments, in an amount of 1%-90%; in some embodiments, in an amount of 1%-80%; in some embodiments, in an amount of 1%-70%; in some embodiments, in an amount of 1%-60%; in some embodiments, in an amount of 1%-50%; in some embodiments, in an amount of 1%-40%; in some embodiments, in an amount of %-30%; in some embodiments, in an amount of 1%-20%; in some embodiments, in an amount of 1%-10%; in some embodiments, in an amount of 5%-90%; in some embodiments, in an amount of 5%-80%; in some embodiments, in an amount of 5%-70%; in some embodiments, in an amount of 5%-60%; in some embodiments, in an amount of 5%-50%; in some embodiments, in an amount of 5%-40%; in some embodiments, in an amount of 5%-30%; in some embodiments, in an amount of 5%-20%; in some embodiments, in an amount of 5%-10%; in some embodiments, in an amount of 10%-90%; in some embodiments, in an amount of 10%-80%; in some embodiments, in an amount of 10%-70%; in some embodiments, in an amount of 10%-60%; in some embodiments, in an amount of 10%-50%; in some embodiments, in an amount of 10%-40%; in some embodiments, in an amount of 10%-30%; in some embodiments, in an amount of 10%-20%; in some embodiments, in an amount of 10%.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler and a disintegrant, and preferably, further includes one or more of a binder, a glidant, a lubricant, and a pH regulator, and the filler is present in an amount of 50%-90%; in some embodiments, in an amount of 60%-90%; in some embodiments, in an amount of 70%-90%; in some embodiments, in an amount of 50%-80%; in some embodiments, in an amount of 60%-80%; in some embodiments, in an amount of 70%-85%; in some embodiments, in an amount of 75%-82%.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler and a disintegrant, and preferably, further includes one or more of a binder, a glidant, a lubricant, and a pH regulator, and the filler is a combination of microcrystalline cellulose and mannitol; in some embodiments, the content ratio of microcrystalline cellulose to mannitol is 1:1-1:2; in some embodiments, the content ratio of microcrystalline cellulose to mannitol is 1:1-1:1.5.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler and a disintegrant, and preferably, further includes one or more of a binder, a glidant, a lubricant, and a pH regulator, and the filler is a combination of microcrystalline cellulose and mannitol; in some embodiments, microcrystalline cellulose accounts for 30%-50% of the total content; in some embodiments, microcrystalline cellulose accounts for 30%-40% of the total content; in some embodiments, microcrystalline cellulose accounts for 30% of the total content; in some embodiments, mannitol accounts for 30%-60% of the total content; in some embodiments, mannitol accounts for 40%-60% of the total content; in some embodiments, mannitol accounts for 45%-52% of the total content.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler and a disintegrant, and preferably, further includes one or more of a binder, a glidant, a lubricant, and a pH regulator, the disintegrant is selected from croscarmellose sodium, and in some embodiments, the disintegrant accounts for 1%-5% of the total content; in some embodiments, accounting for 1%-3% of the total content; in some embodiments, accounting for 3%-5% of the total content; in some embodiments, accounting for 3% of the total content.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler and a disintegrant, and preferably, further includes one or more of a binder, a glidant, a lubricant, and a pH regulator, the disintegrant is selected from crospovidone, and in some embodiments, the disintegrant accounts for 1%-5% of the total content; in some embodiments, accounting for 5% of the total content.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler and a disintegrant, and preferably, further includes one or more of a binder, a glidant, a lubricant, and a pH regulator, and the binder is selected from copovidone; in some embodiments, accounting for 1%-5% of the total content; in some embodiments, accounting for 1%-3% of the total content; in some embodiments, accounting for 3%-5% of the total content; in some embodiments, accounting for 3% of the total content.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler and a disintegrant, and preferably, further includes one or more of a binder, a glidant, a lubricant, and a pH regulator, and the binder is selected from povidone K30; in some embodiments, accounting for 1%-5% of the total content; in some embodiments, accounting for 5% of the total content.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler and a disintegrant, and preferably, further includes one or more of a binder, a glidant, a lubricant, and a pH regulator, and the glidant is selected from silica; in some embodiments, accounting for 0.1%-3% of the total content; in some embodiments, accounting for 0.1%-2% of the total content; in some embodiments, accounting for 0.1%-1% of the total content; in some embodiments, accounting for 0.5%-2% of the total content; in some embodiments, accounting for 0.5%-1% of the total content; in some embodiments, accounting for 1%-3% of the total content; in some embodiments, accounting for 1% of the total content.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler and a disintegrant, and preferably, further includes one or more of a binder, a glidant, a lubricant, and a pH regulator, and the lubricant is selected from sodium stearyl fumarate; in some embodiments, accounting for 0.1%-3% of the total content; in some embodiments, accounting for 0.1%-2% of the total content; in some embodiments, accounting for 0.1%-1% of the total content; in some embodiments, accounting for 0.5%-2% of the total content; in some embodiments, accounting for 0.5%-1% of the total content; in some embodiments, accounting for 1%-3% of the total content; in some embodiments, accounting for 1% of the total content.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes copovidone, silica, crosscarmellose sodium, microcrystalline cellulose, mannitol, and sodium stearate fumarate; in some embodiments, fumaric acid is further included.

The pharmaceutical composition or the pharmaceutical preparation of the present disclosure, comprising the active ingredient M and the pharmaceutically acceptable excipient of any one of the preceding embodiments, wherein the pharmaceutically acceptable excipient includes a filler and a disintegrant, and preferably, further includes one or more of a binder, a glidant, a lubricant, and a pH regulator, and the pharmaceutical composition or the pharmaceutical preparation comprises
(i) the active ingredient M in an amount of 0.5%-99%; in some embodiments, the active ingredient M is present in an amount of 0.5%-90%; in some embodiments, in an amount of 1%-90%; in some embodiments, in an amount of %-80%; in some embodiments, in an amount of %-70%; in some embodiments, in an amount of %-60%; in some embodiments, in an amount of 1%-50%; in some embodiments, in an amount of 1%-40%; in some embodiments, in an amount of 1%-30%; in some embodiments, in an amount of 1%-20%; in some embodiments, in an amount of 1%-10%; in some embodiments, in an amount of 5%-90%; in some embodiments, in an amount of 5%-80%; in some embodiments, in an amount of 5%-70%; in some embodiments, in an amount of 5%-60%; in some embodiments, in an amount of 5%-50%; in some embodiments, in an amount of 5%-40%; in some embodiments, in an amount of 5%-30%; in some embodiments, in an amount of 5%-20%; in some embodiments, in an amount of 5%-10%; in some embodiments, in an amount of 10%-90%; in some embodiments, in an amount of 10%-80%; in some embodiments, in an amount of 10%-70%; in some embodiments, in an amount of 10%-60%; in some embodiments, in an amount of 10%-50%; in some embodiments, in an amount of 10%-40%; in some embodiments, in an amount of 10%-30%; in some embodiments, in an amount of 10%-20%; in some embodiments, in an amount of 10%;
(ii) the filler in an amount of 50%-90% which is a combination of microcrystalline cellulose and mannitol; in some embodiments, the filler is present in an amount of 60%-90%; in some embodiments, in an amount of 70%-90%; in some embodiments, in an amount of 50%-80%; in some embodiments, in an amount of 60%-80%; in some embodiments, in an amount of 70%-85%; in some embodiments, in an amount of 75%-82%; further, in some embodiments, the content ratio of microcrystalline cellulose to mannitol is 1:1-1:2; in some embodiments, the content ratio of microcrystalline cellulose to mannitol is 1:1-1:1.5;
(iii) the disintegrant, croscarmellose sodium, in an amount of 1%-5%; in some embodiments, accounting for 1%-3% of the total content; in some embodiments, accounting for 3%-5% of the total content; in some embodiments, accounting for 3% of the total content;
(iv) the binder, copovidone, in an amount of 1%-5%; in some embodiments, accounting for 1%-3% of the total content; in some embodiments, accounting for 3%-5% of the total content; in some embodiments, accounting for 3% of the total content;
(v) the lubricant, sodium stearyl fumarate, in an amount of 0.1%-3%; in some embodiments, accounting for 0.1%-2% of the total content; in some embodiments, accounting for 0.1%-1% of the total content; in some embodiments, accounting for 0.5%-2% of the total content; in some embodiments, accounting for 0.5%-1% of the total content; in some embodiments, accounting for 1%-3% of the total content; in some embodiments, accounting for 1% of the total content;
(vi) the glidant, silica, in an amount of 0.1%-3%; in some embodiments, accounting for 0.1%-2% of the total content; in some embodiments, accounting for 0.1%-1% of the total content; in some embodiments, accounting for 0.5%-2% of the total content; in some embodiments, accounting for 0.5%-1% of the total content; in some embodiments, accounting for 1%-3% of the total content; in some embodiments, accounting for 1% of the total content.

The pharmaceutical composition or the pharmaceutical preparation of the present disclosure, comprising the active ingredient M and the pharmaceutically acceptable excipient of any one of the preceding embodiments, wherein the pharmaceutical composition or the pharmaceutical preparation comprises:
(i) the active ingredient M which may be present in an amount of 0.5%-99%; and optionally,
(ii) the pharmaceutically acceptable excipient including one or more of a filler, a binder, a wetting agent, a disintegrant, a glidant, and a lubricant;
(iii) the filler including, but not limited to, one or more of microcrystalline cellulose, mannitol, lactose, sucrose, sorbitol, dextran, pregelatinized starch, calcium dihydrogen phosphate, and starch;
(iv) the binder including, but not limited to, one or more of povidone, hydroxypropylcellulose, hypromellose, and methylcellulose;
(v) the wetting agent including, but not limited to, one or more of water and ethanol;
(vi) the disintegrant including, but not limited to, one or more of sodium carboxymethyl starch, low-substituted hydroxypropyl cellulose, crospovidone, croscarmellose sodium, and carboxymethylcellulose calcium;
(vii) the glidant including, but not limited to, one or more of talc, silica, colloidal silicon dioxide, polyethylene glycol, and dodecyl magnesium sulfate;
(viii) the lubricant including, but not limited to, magnesium stearate, calcium stearate, stearic acid, and sodium stearyl fumarate;
(ix) and may further comprise one or more of a flavoring agent, an antioxidant, a preservative, an opacifier, and a film coating premixer.

Optionally, for the pharmaceutical preparation as described above, the binder can be added in solution or powder form; The disintegrant can be added internally, externally, or both.

In some embodiments, the pharmaceutical composition or the pharmaceutical preparation comprises 1-600 mg of the active ingredient M.

In some embodiments, the pharmaceutical composition or the pharmaceutical preparation comprises 5-300 mg of the active ingredient M.

In some embodiments, the pharmaceutical composition or the pharmaceutical preparation comprises 5-200 mg of the active ingredient M.

In some embodiments, the pharmaceutical composition or the pharmaceutical preparation comprises 5-100 mg of the active ingredient M.

In some embodiments, the pharmaceutical composition or the pharmaceutical preparation comprises 5 mg of the active ingredient M.

In some embodiments, the pharmaceutical composition or the pharmaceutical preparation comprises 10 mg of the active ingredient M.

In some embodiments, the pharmaceutical composition or the pharmaceutical preparation comprises 20 mg of the active ingredient M.

In some embodiments, the pharmaceutical composition or the pharmaceutical preparation comprises 25 mg of the active ingredient M.

In some embodiments, the pharmaceutical composition or the pharmaceutical preparation comprises 30 mg of the active ingredient M.

In some embodiments, the pharmaceutical composition or the pharmaceutical preparation comprises 40 mg of the active ingredient M.

In some embodiments, the pharmaceutical composition or the pharmaceutical preparation comprises 50 mg of the active ingredient M.

In some embodiments, the pharmaceutical composition or the pharmaceutical preparation comprises 75 mg of the active ingredient M.

In some embodiments, the pharmaceutical composition or the pharmaceutical preparation comprises 100 mg of the active ingredient M.

In some embodiments, the pharmaceutical composition or the pharmaceutical preparation comprises 125 mg of the active ingredient M.

In some embodiments, the pharmaceutical composition or the pharmaceutical preparation comprises 150 mg of the active ingredient M.

In some embodiments, the pharmaceutical composition or the pharmaceutical preparation comprises 200 mg of the active ingredient M.

In some embodiments, the pharmaceutical composition or the pharmaceutical preparation comprises 250 mg of the active ingredient M.

In some embodiments, the pharmaceutical composition or the pharmaceutical preparation comprises 350 mg of the active ingredient M.

The present disclosure further provides a method for the treatment of a disease in a mammal, comprising administering to the mammal a therapeutically effective amount of the compound or pharmaceutical composition disclosed in the present application, preferably 1-600 mg, wherein preferably, the disease is cancer.

The term “effective amount” or “therapeutically effective amount” according to the present application refers to a sufficient amount of the compound disclosed in the present application that is administered to ameliorate, to some extent, one or more symptoms of a disease or condition (such as cancer) being treated. In some embodiments, the outcome is the reduction and/or remission of signs, symptoms or causes of the disease, or any other desired change in the biological system. For example, an “effective amount” in terms of the therapeutic use is an amount of the composition comprising the compound disclosed in the present application that is required to provide clinically significant reduction of the symptoms of the disease. Examples of therapeutically effective amounts include, but are not limited to 1-600 mg, 2-600 mg, 3-600 mg, 4-600 mg, 5-600 mg, 6-600 mg, 10-600 mg, 20-60 mg, 25-600 mg, 30-600 mg, 40-600 mg, 50-600 mg, 60-600 mg, 70-600 mg, 75-600 mg, 80-600 mg, 90-600 mg, 100-600 mg, 200-600 mg, 1-500 mg, 2-500 mg, 3-500 mg, 4-500 mg, 5-500 mg, 6-500 mg, 10-500 mg, 20-500 mg, 25-500 mg, 30-500 mg, 40-500 mg, 50-500 mg, 60-500 mg, 70-500 mg, 75-500 mg, 80-500 mg, 90-500 mg, 100-500 mg, 125-500 mg, 150-500 mg, 200-500 mg, 250-500 mg, 300-500 mg, 400-500 mg, 5-400 mg, 10-400 mg, 20-400 mg, 25-400 mg, 30-400 mg, 40-400 mg, 50-400 mg, 60-400 mg, 70-400 mg, 75-400 mg, 80-400 mg, 90-400 mg, 100-400 mg, 125-400 mg, 150-400 mg, 200-400 mg, 250-400 mg, 300-400 mg, 1-300 mg, 2-300 mg, 5-300 mg, 10-300 mg, 20-300 g, 25-300 mg, 30-300 mg, 40-300 mg, 50-300 mg, 60-300 mg, 70-300 mg, 75-300 mg, 80-300 mg, 90-300 mg, 100-300 mg, 125-300 mg, 150-300 mg, 200-300 mg, 250-300 mg, 1-200 mg, 2-200 mg, 5-200 mg, 10-200 mg, 20-200 mg, 25-200 mg, 30-200 mg, 40-200 mg, 50-200 mg, 60-200 mg, 70-200 mg, 75-200 mg, 80-200 mg, 90-200 mg, 100-200 mg, 125-200 mg, 150-200 mg, 1-100 mg, 2-100 mg, 5-100 mg, 10-100 mg, 15-100 mg, 20-100 mg, 25-100 mg, 30-100 mg, 40-100 mg, 50-100 mg, 60-100 mg, 70-100 mg, 75-100 mg, 80-100 mg, and 90-100 mg. In some embodiments, examples of therapeutically effective amounts include, but are not limited to 1 mg, 5 mg, 10 mg, 15 mg, 20 mg, 25 mg, 30 mg, 35 mg, 40 mg, 45 mg, 50 mg, 55 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, 240 mg, 250 mg, and 300 mg.

In some embodiments, the preparation specifications of the pharmaceutical composition or the pharmaceutical preparation of the present disclosure include, but are not limited to 1-600 mg, 2-600 mg, 3-600 mg, 4-600 mg, 5-600 mg, 6-600 mg, 10-600 mg, 20-600 mg, 25-600 mg, 30-600 mg, 40-600 mg, 50-600 mg, 60-600 mg, 70-600 mg, 75-600 mg, 80-600 mg, 90-600 mg, 100-600 mg, 200-600 mg, 1-500 mg, 2-500 mg, 3-500 mg, 4-500 mg, 5-500 mg, 6-500 mg, 10-500 mg, 20-500 mg, 25-500 mg, 30-500 mg, 40-500 mg, 50-500 mg, 60-500 mg, 70-500 mg, 75-500 mg, 80-500 mg, 90-500 mg, 100-500 mg, 125-500 mg, 150-500 mg, 200-500 mg, 250-500 mg, 300-500 mg, 400-500 mg, 5-400 mg, 10-400 mg, 20-400 mg, 25-400 mg, 30-400 mg, 40-400 mg, 50-400 mg, 60-400 mg, 70-400 mg, 75-400 mg, 80-400 mg, 90-400 mg, 10-400 mg, 125400 mg, 150-400 mg, 200-400 mg, 250-400 mg, 300-400 mg, 1-300 mg, 2-300 mg, 5-300 mg, 10-300 mg, 20-300 mg, 25-300 mg, 30-300 mg, 40-300 mg, 50-300 mg, 60-300 mg, 70-300) mg, 75-300 mg, 80-300 mg, 90-300 mg, 100-300 mg, 125-300 mg, 150-300 mg. 200-300 mg, 250-300 mg, 1-200 mg, 2-200 mg, 5-200 mg, 10-200 mg, 20-200 mg, 25-200 mg, 30-200 mg, 40-200 mg, 50-200 mg, 60-200 mg, 70-200 mg, 75-200 mg, 80-200 mg, 90-200 mg, 100-200 mg, 125-200 mg, 150-200 mg, 1-100 mg, 2-100 mg, 5-100 mg, 10-100 mg, 15-100 mg, 20-100 mg, 25-100 mg, 30-100 mg, 40-100 mg, 50-100 mg, 60-100 mg, 70-100 mg, 75-100 mg, 80-100 mg, and 90-100 mg. In some embodiments, the preparation specifications of the pharmaceutical composition include, but are not limited to 1 mg, 5 mg, 10 mg, 15 mg, 20 mg, 25 mg, 30 mg, 35 mg, 40 mg, 45 mg, 50 mg, 55 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, 240 mg, 250 mg, and 300 mg.

In some embodiments, the amount of the active ingredient M in the unit preparation of the pharmaceutical composition or the pharmaceutical preparation of the present disclosure includes but is not limited to 1-600 mg, 2-600 mg, 3-600 mg, 4-600 mg, 5-600 mg, 6-600 mg, 10-600 mg, 20-600 mg, 25-600 mg, 30-600 mg, 40-600 mg, 50-600 mg, 60-600 mg, 70-600 mg, 75-600 mg, 80-600 mg, 90-600 mg, 100-600 mg, 200-600 mg, 1-500 mg, 2-500 mg, 3-500 mg, 4-500 mg, 5-500 mg, 6-500 mg, 10-500 mg, 20-500 mg, 25-500 mg, 30-500 mg, 40-500 mg, 50-500 mg, 60-500 mg, 70-500 mg, 75-500 mg, 80-500 mg, 90-500 mg, 100-500 mg, 125-500 mg, 150-500 mg, 200-500 mg, 250-500 mg, 300-500 mg, 400-500 mg, 5-400 mg, 10-400 mg, 20-400 mg, 25-400 mg, 30-400 mg, 40-400 mg, 50-400 mg, 60-400 mg, 70-400 mg, 75-400 mg, 80-400 mg, 90-400 mg, 100-400 mg, 125-400 mg, 150-400 mg, 200-400 mg, 250-400 mg, 300-400 mg, 1-300 mg, 2-300 mg, 5-300 mg, 10-300 mg, 20-300 mg, 25-300 mg, 30-300 mg, 40-300 mg, 50-300 mg, 60-300 mg, 70-300 mg, 75-300 mg, 80-300 mg, 90-300 mg, 100-300 mg, 125-300 mg, 150-300 mg, 200-300 mg, 250-300 mg, 1-200 mg, 2-200 mg, 5-200 mg, 10-200 mg, 20-200 mg, 25-200 mg, 30-200 mg, 40-200 mg, 50-200 mg, 60-200 mg, 70-200 mg, 75-200 mg, 80-200 mg, 90-200 mg, 100-200 mg, 125-200 mg, 150-200 mg, 1-100 mg, 2-100 mg, 5-100 mg, 10-100 mg, 15-100 mg, 20-100 mg, 25-100 mg, 30-100 mg, 40-100 mg, 50-100 mg, 60-100 mg, 70-100 mg, 75-100 mg, 80-100 mg, and 90-100 mg. In some embodiments, the amount of the active ingredient M in the unit preparation of the pharmaceutical composition or the pharmaceutical preparation includes but is not limited to 1 mg, 5 mg, 10 mg, 15 mg, 20 mg, 25 mg, 30 mg, 35 mg, 40 mg, 45 mg, 50 mg, 55 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, 240 mg, 250 mg, and 300 mg.

A method for the treatment of a disease in a mammal, comprising administering the active ingredient M to a subject at a daily dose of 1-800 mg/day, which may be a single dose or divided doses; in some embodiments, the daily dose includes but is not limited to 10-800 mg/day, 25-800 mg/day, 50-800 mg/day, 100-800 mg/day, 200-800 mg/day, 25-400 mg/day, 50-400 mg/day, 100-400 mg/day, and 200-400 mg/day; in some embodiments, the daily dose includes but is not limited to 10 mg/day, 20 mg/day, 25 mg/day, 50 mg/day, 100 mg/day, 120 mg/day, 125 mg/day, 150 mg/day, 200 mg/day, 240 mg/day, 400 mg/day, 600 mg/day, and 800 mg/day.

The present disclosure relates to a kit, which may include the pharmaceutical composition or the pharmaceutical preparation in the form of a single dose or multiple doses, and which comprises the active ingredient M in the pharmaceutical composition of the present disclosure, in an amount including but not limited to 1 mg, 5 mg, 10 mg, 15 mg, 20 mg, 25 mg, 30 mg, 35 mg, 40 mg, 45 mg, 50 mg, 55 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, 240 mg, 250 mg, and 300 mg.

Unless stated to the contrary, the terms used in the description and claims of the present application have the following meanings.

The term “preparation specification” refers to the weight of the active drug (the active ingredient M) contained in each vial, tablet or other unit preparation.

Unless otherwise specified, the terms of the present disclosure have the following meanings.

The carbon, hydrogen, oxygen, sulfur, nitrogen and halogen involved in the groups and compounds of the present disclosure all include isotopes thereof, and are optionally further replaced by one or more of the corresponding isotopes thereof, wherein the isotopes of carbon include ¹²C. ¹³C and ¹⁴C; the isotopes of hydrogen include protium (H), deuterium (D, also known as heavy hydrogen) and tritium (T, also known as superheavy hydrogen); the isotopes of oxygen include ¹⁶O, ¹⁷O and ¹⁸O; the isotopes of sulfur include ³²S, ³³S, ³⁴S and ³⁶S; the isotopes of nitrogen include ¹⁴N and ¹⁵N; the isotope of fluorine includes ¹⁹F; the isotopes of chlorine include ³⁵Cl and ³⁷Cl; and the isotopes of bromine include ⁷⁹Br and ⁸¹Br.

The term “halogen” herein refers to F, Cl, Br, I, or isotopes thereof.

The term “halo” or “substituted with halogen” refers to being substituted with one or more groups selected from F, Cl, Br, I, or isotopes thereof, wherein the upper limit of the number of halogen substituents is equal to the sum of the number of hydrogens that can be substituted in the group to be substituted. Without particular limitation, the number of halogen substituents is any integer between 1 and the upper limit, and when the number of halogen substituents is greater than 1, the group to be substituted can be substituted with the same or different halogen. Generally, the circumstances of being substituted with 1-5 halogen, 1-3 halogen, 1-2 halogen, and 1 halogen are included.

The term “deuterium” refers to the isotope deuterium of hydrogen (H).

The term “deuterated” or “deuterated substance” refers to the case where a hydrogen atom on a group, such as alkyl, cycloalkyl, alkylene, aryl, heteroaryl, mercapto, heterocycloalkyl, alkenyl and alkynyl is substituted with at least one deuterium atom, wherein the upper limit of the number of deuterium substituents is equal to the sum of the number of hydrogens that can be substituted in the group to be substituted. Without particular limitation, the number of deuterium substituents is any integer between 1 and the upper limit, for example, 1-20 deuterium atoms, 1-10 deuterium atoms, 1-6 deuterium atoms, 1-3 deuterium atoms, 1-2 deuterium atoms or 1 deuterium atom.

Group “C_{x-y}” refers to a group comprising x to y carbon atoms, for example, “C₁₋₆ alkyl” refers to alkyl comprising 1-6 carbon atoms.

The term “alkyl” refers to a monovalent straight or branched saturated aliphatic hydrocarbon group, usually an alkyl group with 1 to 20 carbon atoms, or an alkyl group with 1 to 8 carbon atoms, or an alkyl group with 1 to 6 carbon atoms, or an alkyl group with 1 to 4 carbon atoms, such as “C₁₋₆ alkyl”, “C₁₋₅ alkyl”, “C₁₋₄ alkyl”, “C₁₋₃ alkyl”, “C₁₋₂ alkyl”, “C₂₋₆ alkyl”, “C₂₋₅ alkyl”. “C₂₋₄ alkyl”, “C₂₋₃ alkyl”. “C₃₋₆ alkyl”, “C₃₋₅ alkyl”, “C₃₋₄ alkyl”, “C₄₋₆ alkyl”, “C₄₋₅ alkyl”, and “C₅₋₆ alkyl”. Non-limiting examples of alkyl include methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1,2-dimethylpropyl, etc. The alkyl can be further substituted with any substituent.

The term “alkylene” refers to a divalent straight or branched saturated alkyl group. Examples of alkylene include, but are not limited to, methylene, ethylidene, etc. The alkylene can be optionally further substituted with a substituent.

The term “haloalkyl” refers to an alkyl group in which one or more hydrogens are replaced by one or more halogen atoms (e.g., fluorine, chlorine, bromine, iodine, or isotopes thereof), wherein the upper limit of the number of halogen substituents is equal to the sum of the number of hydrogens that can be substituted in the alkyl group. Without particular limitation, the number of halogen substituents is any integer between 1 and the upper limit. Generally, the alkyl group is substituted with 1-5 halogen, 1-3 halogen, 1-2 halogen or 1 halogen; and when the number of halogen substituents is greater than 1, the group to be substituted can be substituted with the same or different halogen. Specific examples include, but are not limited to —CF₃, —CH₂Cl, —CH₂CF₃, —CCl₂, CF₃, etc.

The term “alkoxy” or “alkyloxy” refers to —O-alkyl, such as —O—C₁₋₈ alkyl, —O—C₁₋₆ alkyl, —O—C₁₋₄ alkyl or —O—C₁₋₂ alkyl. Non-limiting and specific examples of alkoxy or alkyloxy include methoxy, ethoxy, n-propoxy, isopropoxy, n-butoxy, sec-butoxy, tert-butoxy, n-pentoxy, n-hexyloxy, cyclopropoxy, cyclobutoxy, etc. The alkoxy may be optionally substituted with a substituent.

The term “haloalkoxy” refers to —O-haloalkyl, such as —O-halo C₁₋₈ alkyl, —O-halo C₁₋₆ alkyl, —O-halo C₁₋₄ alkyl or —O-halo C₁₋₂ alkyl; the upper limit of the number of halogen substituents is equal to the sum of the number of hydrogens that can be substituted in the group to be substituted. Without particular limitation, the number of halogen substituents is any integer between 1 and the upper limit, preferably 1-5 halogen, 1-3 halogen, 1-2 halogen, and 1 halogen; and when the number of halogen substituents is greater than 1, the group to be substituted can be substituted with the same or different halogen. Non-limiting examples of haloalkoxy include monofluoromethoxy, difluoromethoxy, trifluoromethoxy, difluoroethyloxy, etc.

The term “alkenyl” refers to a straight or branched hydrocarbon group comprising at least one carbon-carbon double bond (C═C) and generally comprises 2 to 18 carbon atoms, such as 2 to 8 carbon atoms, further such as 2 to 6 carbon atoms, and still further such as 2 to 4 carbon atoms. Examples of alkenyl include, but are not limited to ethenyl, allyl, 1-propenyl, 2-propenyl, 1-butenyl, 2-butenyl, 3-butenyl, I-pentenyl, 2-pentenyl, 3-pentenyl, 4-pentenyl, I-methyl-1-butenyl, 2-methyl-1-butenyl, 2-methyl-3-butenyl, 1-hexenyl, 2-hexenyl, 3-hexenyl, 4-hexenyl, 5-hexenyl, 1-methyl-1-pentenyl, 2-methyl-1-pentenyl, 1-heptenyl, 2-heptenyl, 3-heptenyl, 4-heptenyl, 1-octenyl, 3-octenyl, 1-nonenyl, 3-nonenyl, 1-decenyl, 4-decenyl, 1,3-butadiene, 1,3-pentadiene, 1,4-pentadiene, 1,4-hexadiene, etc.; and the alkenyl may further be optionally substituted with a substituent.

The term “alkenylene” refers to a straight or branched divalent unsaturated hydrocarbon group comprising at least one carbon-carbon double bond (C═C). Unless otherwise specified, the alkynylene contains 2-6 carbon atoms, preferably 2-4 carbon atoms. Non-limiting examples of alkynylene include ethynylene, and the alkenylene may be optionally substituted with a substituent.

The term “alkynyl” refers to a straight or branched hydrocarbon group comprising at least one carbon-carbon triple bond (CEC) and generally comprises 2 to 18 carbon atoms, further comprises 2 to 8 carbon atoms, further comprises 2 to 6 carbon atoms, and still further comprises 2 to 4 carbon atoms. Examples of alkynyl include, but are not limited to ethynyl, I-propynyl, 2-propynyl, butynyl, 2-butynyl, 3-butynyl, 1-methyl-2-propynyl, 4-pentynyl, 3-pentynyl, 1-methyl-2-butynyl, 2-hexynyl, 3-hexynyl, 2-heptynyl, 3-heptynyl, 4-heptynyl, 3-octynyl, 3-nonynyl, 4-decynyl, etc.; and the alkynyl may be optionally substituted with a substituent.

The term “alkynylene” refers to a straight or branched divalent unsaturated hydrocarbon group comprising a carbon-carbon triple bond (C≡C) and generally comprises 2-6 carbon atoms, and further comprises 2-4 carbon atoms. Non-limiting examples of alkynylene include ethynylene, propynylene and butynylene; and the alkynylene may be optionally substituted with a substituent.

The term “cycloalkyl” refers to a saturated or partially unsaturated, non-aromatic carbocyclic hydrocarbon group containing no ring heteroatoms. The cycloalkyl may be monocyclic, bicyclic or polycyclic; the bicyclic or polycyclic cycloalkyl may be in the form of a fused ring, a spiro ring, a bridged ring or a combination thereof, and may comprise one or more aromatic rings, but the ring system is non-aromatic as a whole; and the connection site is on a non-aromatic ring. Generally, the cycloalkyl contains 3 to 20 carbon atoms, further contains 3-8 carbon atoms, and still further contains 3-6 carbon atoms; when the cycloalkyl is monocyclic cycloalkyl, the cycloalkyl contains 3-15 carbon atoms, or 3-10 carbon atoms, or 3-8 carbon atoms, or 3-6 carbon atoms; when the cycloalkyl is bicyclic or polycyclic cycloalkyl, the cycloalkyl contains 5-12 carbon atoms, or 5-11 carbon atoms, or 6-10 carbon atoms. Non-limiting examples of cycloalkyl include cyclopropyl, cyclobutyl, cyclopentyl, cyclohexyl, cycloheptyl, butenyl, cyclopentenyl, cyclohexenyl,

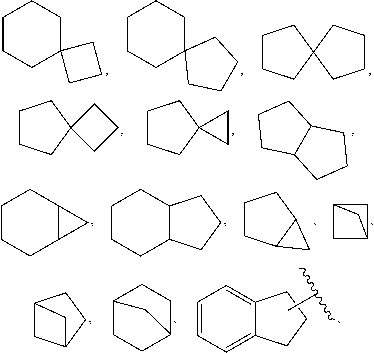

etc., and the cycloalkyl may be optionally substituted with a substituent.

The term “cycloalkylene” refers to a divalent group of cycloalkyl.

The term “aryl” refers to a substituted or unsubstituted aromatic 6- to 15-membered carbocycle, and includes monocyclic aryl and fused aryl. Aryl is preferably a 6- to 10-membered aromatic ring, further preferably a 6- to 9-membered aromatic ring, and further preferably a 6- to 8-membered aromatic ring. Aryl ring can be fused to an aryl ring and a non-aryl ring (such as a heteroaryl, heterocycloalkyl, or cycloalkyl ring), wherein the aryl ring is the connection site. The “x- to y-membered aryl” means that the aryl has a total number of ring atoms of x to y, and can be a phenyl fused non-aromatic ring in which the aromatic ring is the connection site. For example, “7- to 12-membered aryl” represents that the aryl, as the connection site, has a total number of ring atoms of 7-12, e.g., benzocyclobutyl and benzocyclopentyl. Non-limiting examples of aryl include phenyl, naphthyl, anthryl, phenanthryl

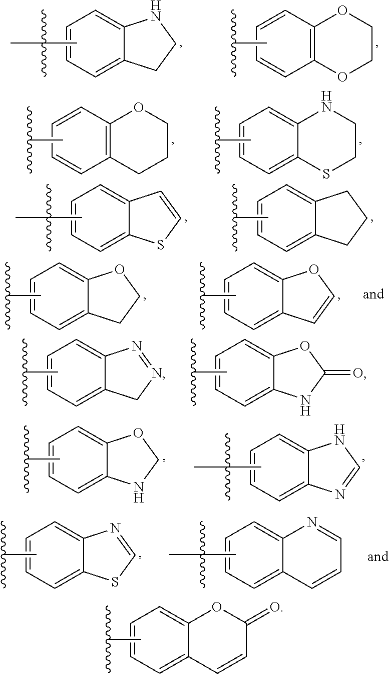

and the aryl may be optionally further substituted with any substituent.

The term “heterocycloalkyl” refers to a saturated or partially unsaturated, non-aromatic carbocycle comprising 1, 2, 3, 4 or 5 heteroatoms selected from N, S, O, P or Si. The heterocycloalkyl may be monocyclic, bicyclic or polycyclic; the bicyclic or polycyclic heterocycloalkyl may be in the form of a bridged ring, a fused ring, a spiro ring or a combination thereof, and may comprise one or more aromatic rings or heteroaromatic rings, but the ring system is non-aromatic as a whole; and the connecting site is on a non-aromatic ring. Generally, the heterocycloalkyl is a 3- to 20-membered ring. When the heterocycloalkyl is monocyclic heterocycloalkyl, the heterocycloalkyl is usually a 3- to 15-membered ring, or a 3- to 10-membered ring, or a 3- to 8-membered ring, or a 3- to 6-membered ring; when the heterocycloalkyl is bicyclic or polycyclic heterocycloalkyl, the heterocycloalkyl is usually a 5- to 12-membered ring, or a 5- to 11-membered ring, or a 6- to 9-membered ring. The heteroatoms N, S and P include their oxidation states C═O, N—O, S═O, S(═O), P═O, and P(═O)₂. When heterocycloalkyl is a bicyclic or polycyclic ring, at least one ring contains at least one heteroatom, and the heterocycloalkyl can be a bicyclic or polycyclic ring formed by a ring containing the heteroatom(s) and a ring containing no heteroatom, or a bicyclic or polycyclic ring formed by a ring containing the heteroatom(s) and a ring containing the heteroatom(s). When heterocycloalkyl is connected to other groups, a connection point can be at a heteroatom or a carbon atom. Non-limiting examples of heterocycloalkyl include azetidinyl, morpholinyl, piperazinyl, piperidyl, tetrahydropyranyl, oxetanyl, pyranyl, azacyclopentenyl, azacyclohexenyl, oxacyclopentenyl, oxacyclohexenyl, etc., and the heterocycloalkyl may be optionally substituted with a substituent.

The term “heteroaromatic ring” or “heteroaryl”, unless otherwise specified, refers to a substituted or unsubstituted aromatic ring containing 1 to 5 heteroatoms selected from N, O, S, P, Si and their oxidation states, which can be monocyclic, bicyclic, or polycyclic, wherein the bicyclic or polycyclic heteroaromatic ring or heteroaryl can be bridged rings, fused rings, spiro rings and combinations thereof. Bicyclic or polycyclic heteroaromatic ring or heteroaryl can be formed by fusion of heteroaryl to aryl, of heteroaryl to heteroaryl, or of heteroaryl to cycloalkyl or heterocycloalkyl, wherein the heteroaryl is the connection site. The “x- to y-membered heteroaryl” means that the heteroaryl has a total number of ring atoms of x to y, which can be 5- to 6-membered heteroaryl, and can also be 5- to 6-membered heteroaryl fused to other rings (e.g., cycloalkyl, heterocycloalkyl, and aromatic rings) in which the heteroaromatic ring is the connection site. For example, “5- to 12-membered heteroaryl” represents that the heteroaryl, as the connection site, has a total number of ring atoms of 5-12, e.g., pyridocyclobutyl and pyridocyclopentyl. Non-limiting examples of heteroaromatic ring or heteroaryl include furyl, thienyl, pyrrolyl, oxazolyl, thiazolyl, imidazolyl, pyrazolyl, pyridyl, pyrimidyl, pyridazinyl, pyrazinyl, indolyl, purinyl,

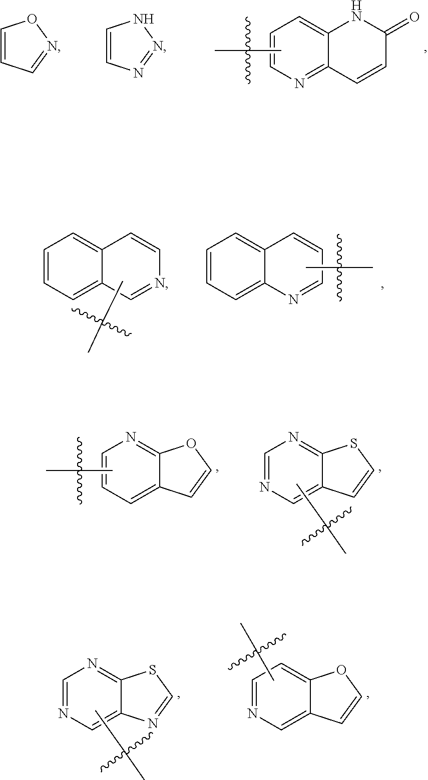

etc. The heteroaryl may be optionally substituted with a substituent.

The term “aromatic ring” refers to an aromatic ring system containing or containing no N, S, O, P, Si and other heteroatoms, and its definition includes aryl and heteroaryl. The aromatic ring may be optionally substituted with a substituent.

The term “heterocycle” or “heterocyclyl” refers to a saturated or unsaturated, aromatic or non-aromatic ring containing 1 to 5 heteroatoms selected from N, O, S, P, Si and their oxidation states, and its meaning includes heteroaryl and heterocycloalkyl. The heterocycle may be in the form of a monocyclic heterocycle, a bicyclic bridged heterocycle, a bicyclic fused heterocycle, a bicyclic spiro heterocycle or a combination thereof. The heterocycle is usually a 3- to 12-membered heterocycle, or a 5- to 12-membered heterocycle, or a 5- to 7-membered heterocycle. Heterocyclyl can be connected to a heteroatom or a carbon atom. Non-limiting examples of heterocyclyl include oxiranyl, azacyclopropyl, oxetanyl, azetidinyl, 1,3-dioxolanyl, 1,4-dioxolanyl, 1,3-dioxanyl, piperazinyl, azacycloheptyl, pyridyl, furyl, thienyl, pyranyl. N-alkylpyrrolyl, pyrimidyl, pyrazinyl, pyrazolyl, pyridazinyl, imidazolyl, piperidyl, piperadinyl, morpholinyl, thiomorpholinyl, 1,3-dithianyl, dihydrofuryl, dihydropyranyl, dithiolanyl, tetrahydrofuryl, tetrahydropyrrolyl, tetrahydroimidazolyl, oxazolyl, dihydrooxazolyl, tetrahydrooxazolyl, tetrahydrothiazolyl, tetrahydropyranyl, benzoimidazolyl, benzopyridyl, pyrrolopyridyl, benzodihydrofuryl, azabicyclo[3.2.1]octanyl, azabicyclo[5.2.0]nonanyl, oxatricyclo[5.3.1.1]dodecyl, azaadamantyl and oxaspiro[3.3]heptanyl,

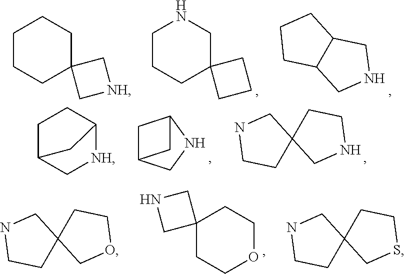

etc., and the heterocycle may be optionally substituted with a substituent.

The term “spiro ring” refers to a polycyclic group sharing one carbon atom (referred to as a spiro atom) between rings, which may contain 0 or at least 1 double or triple bond, and may contain 0 to 5 heteroatoms selected from N, O, S, P, Si and their oxidation states. Generally, a spiro ring is a 5- to 14-membered ring, or a 5- to 12-membered ring, or a 5- to 10-membered ring. Generally, a spiro ring is a spiro ring formed by a three-membered ring and a three-membered ring, a three-membered ring and a four-membered ring, a three-membered ring and a five-membered ring, a three-membered ring and a six-membered ring, a four-membered ring and a four-membered ring, a four-membered ring and a five-membered ring, a four-membered ring and a six-membered ring, a five-membered ring and a five-membered ring or a five-membered ring and a six-membered ring. The spiro ring may be spirocyclic, non-limiting examples of which include

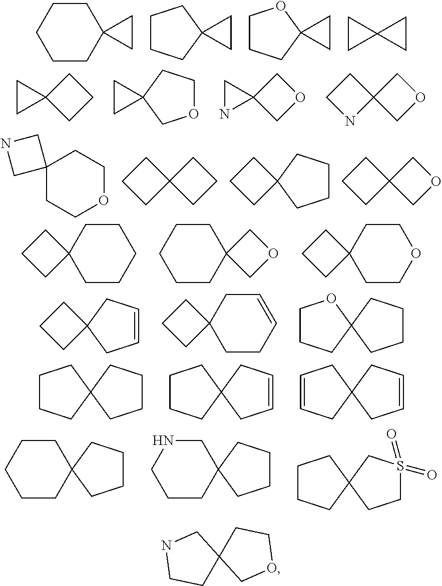

and the spiro ring may be optionally substituted with a substituent.

The term “bicyclic spiro cycloalkyl” means that both rings forming the spiro ring are cycloalkyl.

The term “bicyclic spiro heterocycloalkyl” means that at least one of the two rings forming the spiro ring is heterocycloalkyl.

The term “fused ring” refers to a polycyclic group in which the rings share two adjacent ring atoms and one chemical bond, which may contain one or more double or triple bonds, and may contain 0 to 5 heteroatoms selected from N, S, O, P. Si and their oxidation states. Generally, a fused ring is a 5- to 20-membered ring, or a 5- to 14-membered ring, or a 5- to 12-membered ring or a 5- to 10-membered ring. Generally, a fused ring is in the form of a three-membered ring fused a four-membered ring (indicating a fused ring formed by a three-membered ring and a four-membered ring, and either the three-membered ring or the four-membered ring may be possibly used as the basic ring according to the IUPC nomenclature; similarly hereinafter), a three-membered ring fused a five-membered ring, a three-membered ring fused a six-membered ring, a four-membered ring fused a four-membered ring, a four-membered ring fused a five-membered ring, a four-membered ring fused a six-membered ring, a five-membered ring fused a five-membered ring, a five-membered ring fused a six-membered ring, and a six-membered ring fused a six-membered ring. Non-limiting examples of fused ring include purine, quinoline, isoquinoline, benzopyran, benzofuran, benzothiophene, and

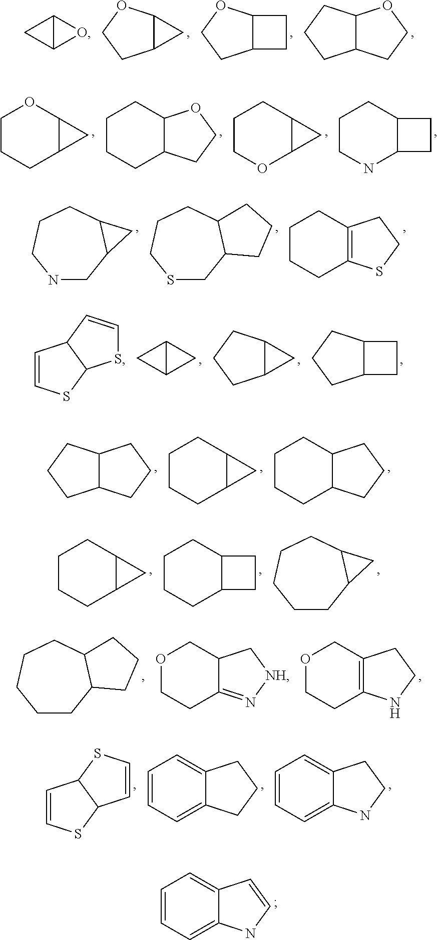

and the fused ring may be optionally substituted with a substituent.

The term “bridged ring” refers to a ring system in which two non-adjacent ring atoms are shared between two rings, which may contain one or more double or triple bonds. The bridged ring may contain 0 to 5 heteroatoms selected from N, S, O, P, Si and their oxidation states. Generally, the bridged ring has 5 to 20, or 5 to 14, or 5 to 12, or 5 to 10 ring atoms. Non-limiting examples of the bridged ring include adamantane,

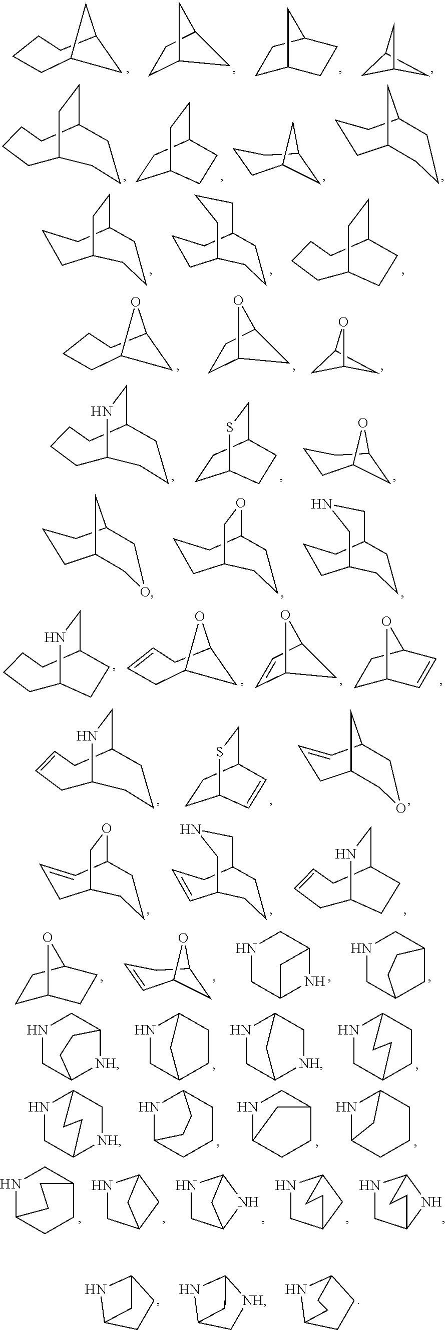

Unless otherwise specified, the term “substitution” or “substituent” refers to any substitution at a position allowed by chemical theory, and the number of substituents conforms to the rules of chemical bonding. Exemplary substituents include, but are not limited to: C₁₋₆ alkyl, C₂₋₆ alkenyl, C₂₋₆ alkynyl, C₃₋₈ heteroalkyl, C₅₋₁₂ aryl, 5- to 12-membered heteroaryl, hydroxyl, C₁₋₆ alkoxy, C₅₋₁₂ aryloxy, thiol, C₁₋₆ alkylthio, cyano, halogen, C₁₋₆ alkylthiocarbonyl, C₁₋₆ alkylcarbamoyl, N-carbamoyl, nitro, silyl, sulfinyl, sulfonyl, sulfoxide, halo C₁₋₆ alkyl, halo C₁₋₆ alkoxy, amino, phosphonic acid, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂Cl₆ alkyl, etc.

“ [symbol] ” represents the connection site.

The term “optional” or “optionally” refers to that the events or circumstances subsequently described may but not necessarily occur, and the description includes the occasions where the events or circumstances occur or do not occur. For example, “alkyl optionally substituted with F” means that the alkyl may but not necessarily be substituted with F, and the description includes the case where the alkyl is substituted with F and the case where the alkyl is not substituted with F.

The term “pharmaceutically acceptable salt” refers to a salt of the compound of the present disclosure, which salt maintains the biological effectiveness and characteristics of a free acid or a free base and is obtained by reacting the free acid with a non-toxic inorganic base or organic base, or reacting the free base with a non-toxic inorganic acid or organic acid.

The term “pharmaceutical composition” represents a mixture of one or more compounds or stereoisomers, solvates, pharmaceutically acceptable salts or co-crystals thereof as described herein and other components including physiologically/pharmaceutically acceptable carriers and/or excipients.

The term “preparation specification” refers to the weight of the active drug contained in each vial, tablet or other unit preparation.

The term “carrier” refers to: a system that does not cause significant irritation to the organism and does not eliminate the biological activity and characteristics of the administered compound and can change the way the drug enters the human body and the distribution of the drug in the body, control the release rate of the drug and delivery the drug to targeted organs. Non-limiting examples of the carrier include microcapsule, microsphere, nanoparticle, liposome, etc.

The term “excipient” refers to: a substance that is not a therapeutic agent per se, but used as a diluent, adjuvant, binder and/or vehicle for addition to a pharmaceutical composition, thereby improving the disposal or storage properties thereof, or allowing to or promoting the formation of a compound or a pharmaceutical composition into a unit dosage form for administration. As is known to those skilled in the art, pharmaceutically acceptable excipients can provide various functions and can be described as a wetting agent, a buffer, a suspending agent, a lubricant, an emulsifier, a disintegrating agent, an absorbent, a preservative, a surfactant, a colorant, a flavoring agent, and a sweetening agent. Examples of pharmaceutically acceptable excipients include, but are not limited to: (1) sugars, such as lactose, glucose and sucrose; (2) starch, such as corn starch and potato starch; (3) cellulose and its derivatives, such as sodium carboxymethyl cellulose, ethyl cellulose, cellulose acetate, hydroxypropyl methylcellulose, hydroxypropyl cellulose, microcrystalline cellulose and croscarmellose (such as croscarmellose sodium); (4) tragacanth powder; (5) malt; (6) gelatin; (7) talc; (8) excipients, such as cocoa butter or suppository wax; (9) oils, such as peanut oil, cottonseed oil, safflower oil, sesame oil, olive oil, corn oil and soybean oil; (10) diols, such as propylene glycol; (11) polyols, such as glycerin, sorbitol, mannitol and polyethylene glycol; (12) esters, such as ethyl oleate and ethyl laurate; (13) agar; (14) buffers, such as magnesium hydroxide and aluminum hydroxide; (15) alginic acid; (16) pyrogen-free water, (17) isotonic saline; (18) Ringer's solution; (19) ethanol; (20) pH buffered solution; (21) polyester, polycarbonate and/or polyanhydride; and (22) other non-toxic compatible substances used in a pharmaceutical preparation.

The term “stereoisomer” refers to an isomer produced as a result of different spatial arrangement of atoms in molecules, including cis-trans isomers, enantiomers and conformational isomers.

The term “solvate” refers to a substance formed by the compound of the present disclosure or the salt thereof and a stoichiometric or non-stoichiometric solvent bound by intermolecular non-covalent forces. When the solvent is water, the solvate is a hydrate.

The term “co-crystal” refers to a crystal formed by the combination of active pharmaceutical ingredient (API) and co-crystal former (CCF) under the action of hydrogen bonds or other non-covalent bonds. The pure state of API and CCF are both solid at room temperature, and there is a fixed stoichiometric ratio between various components. The co-crystal is a multi-component crystal, which includes both a binary co-crystal formed between two neutral solids and a multi-element co-crystal formed between a neutral solid and a salt or solvate.

### BRIEF DESCRIPTION OF THE DRAWINGS

FIG. 1 shows the tumor growth curve of the mouse MDA-MB-436 subcutaneous in vivo transplanted tumor model.

FIG. 2 shows the animal body weight change curve of the mouse MDA-MB-436 subcutaneous in vivo transplanted tumor model.

### DETAILED DESCRIPTION

The technical solutions of the present disclosure will be described in detail by the following examples, but the scope of protection of the present disclosure includes but is not limited thereto.

The compounds used in the reactions described herein are prepared according to organic synthesis techniques known to those skilled in the art, and starting from commercially available chemicals and(or) compounds described in chemical documents. “Commercially available chemicals” are obtained from regular commercial sources, and suppliers include: Titan Technology Co., Ltd., Energy Chemical Co., Ltd., Shanghai Demo Co., Ltd., Chengdu Kelong Chemical Co., Ltd., Accela ChemBio Co., Ltd., PharmaBlock Sciences (Nanjing), Inc., WuXi Apptec Co., Ltd., J&K Scientific Co., Ltd., etc.

The structures of the compounds are determined by nuclear magnetic resonance (NMR) or (and) mass spectrometry (MS). The NMR shift (δ) is given in the unit of 10⁻⁶ (ppm). NMR is measured with (Bruker Avance II 400 and Bruker Avance 300) NMR instrument, and the solvent for determination is deuterated dimethyl sulfoxide (DMSO-d₆), deuterated chloroform (CDCl₃), deuterated methanol (CD₃OD), and the internal standard is tetramethylsilane (TMS);
MS is determined with Agilent 6120B (ESI) and Agilent 6120B (APCI); and
HPLC is determined with Agilent 1260DAD high pressure liquid chromatograph (Zorbax SB-C18 100×4.6 mm, 3.5 μM);
Yantai Huanghai HSGF₂₅₄ or Qingdao GF₂₅₄ silica gel plate is used as a thin layer chromatography silica plate, and the silica gel plate for the thin layer chromatography (TLC) is of the specification of 0.15 mm-0.20 mm, and the specification when separating and purifying a product by thin layer chromatography is 0.4 mm-0.5 mm;
for the column chromatography, Yantai Huanghai silica gel of 200-300 mesh silica gel is generally used as a carrier;
RuPhos-Pd-G3: catalyst with CAS No. 1445085-77-7.

### Example 1

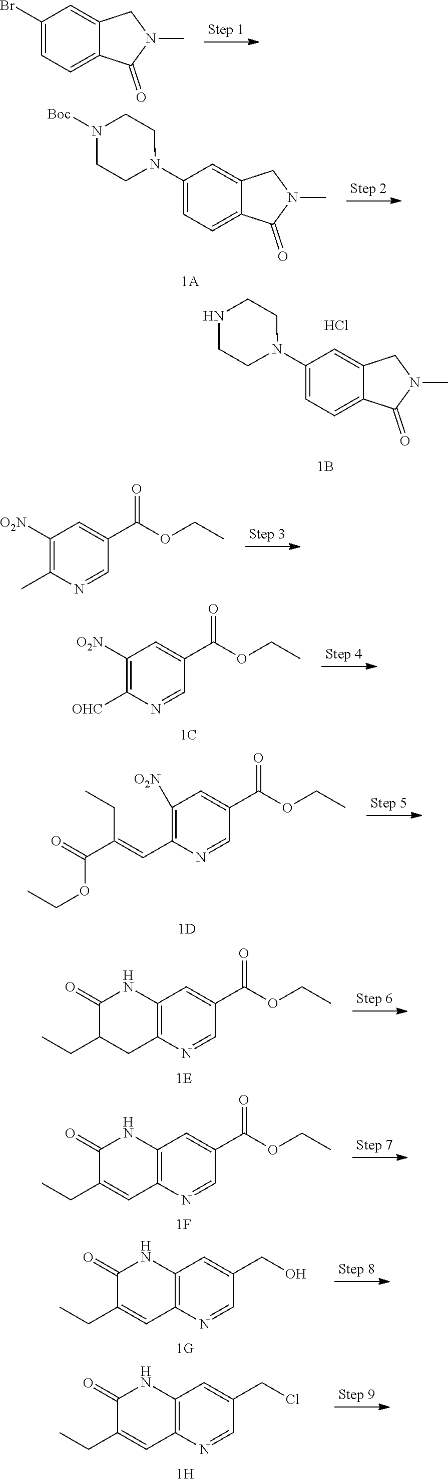

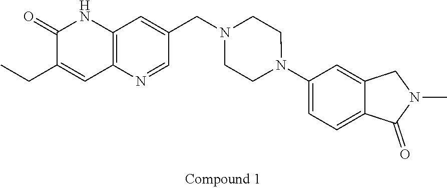 [SMILES: CCc1cc2ncc(CN3CCN(c4ccc5c(c4)CN(C)C5=O)CC3)cc2[nH]c1=O]

### Step 1:

5-Bromo-2-methyl-1,3-dihydroisoindol-1-one (600 mg, 2.65 mmol) and N-Boc-piperazine (593 mg, 3.18 mmol) were dissolved in 1,4-dioxane (10 mL), Cs₂CO₃ (0.73 g, 5.31 mmol) and RuPhos-Pd-G3 (89 mg, 0.11 mmol) were added, and the mixture was reacted overnight at 100° C. under nitrogen protection. Subsequently, the reaction liquid was quenched with water (15 mL) and extracted with ethyl acetate (20 mL×3). The organic phases were combined, dried over anhydrous Na₂SO₄, filtered, spun to dryness, and separated by silica gel chromatographic column (PE:EA (v/v)=1:0-1:1), to afford title compound 1A (720 mg, 81.9%) as a yellow solid.

LC-MS (ESI): m/z=332.2, 276.1 [M+H]⁺.

### Step 2:

1A (720 mg, 2.17 mmol) was dissolved in methanol (5 mL), and a solution of hydrogen chloride in dioxane (5 mL, 4 M) was added. The mixture was reacted at room temperature for two hours and spun to dryness to obtain title compound 1B (580 mg, crude).

LC-MS (ESI): m/z=232.2 [M+H]⁺.

### Step 3:

Ethyl 6-methyl-5-nitronicotinate (10 g, 47.6 mmol) and selenium dioxide (21.14 g, 190.5 mmol) were dissolved in 1,4-dioxane (100 ml), and the mixture was refluxed overnight at 100° C. After the reaction was completed, the reaction liquid was filtered through a funnel lined with diatomaceous earth, the diatomaceous earth was washed with ethyl acetate, the filtrate was concentrated, and the resulting residue was separated and purified by silica gel column chromatography (eluent: ethyl acetate:petroleum ether (v/v)=0%-40%), to afford compound 1C (10.104 g, 94.7%) as a yellow oil.

LCMS(ESI) m/z=225.1 [M+1]⁺

### Step 4:

Sodium hydride (2.695 g, 112.3 mmol) was dissolved in anhydrous tetrahydrofuran (100 ml) and stirred at 0° C., and triethyl 2-phosphonobutyrate (28.3 g, 112.3 mmol) was added dropwise. After the dropwise addition was completed, the mixture was stirred at 0° C. for 20 min, warmed to 40° C. and stirred for 10 min, and then transferred to a dry ice ethanol bath. Compound 1C (10.48 g, 46.8 mmol) was dissolved in anhydrous tetrahydrofuran (100 ml) and added dropwise to a reaction flask. The mixture was kept in the dry ice ethanol bath and stirred for 1 h. After the reaction was completed, the reaction liquid was quenched with saturated ammonium chloride solution (100 ml) and extracted with ethyl acetate (200 ml). The organic phase was separated, and the aqueous phase was extracted with ethyl acetate (200 ml×2). The organic phases were combined, dried over anhydrous sodium sulfate and concentrated, and the resulting residue was purified by silica gel column chromatography (eluent: ethyl acetate:petroleum ether (v/v)=0-10%), to afford compound 1D (11.57 g, 76.8%), a mixture of two isomers as a yellow oil.

LC-MS(ESI) m/z=323.1 [M+H]⁺

### Step 5:

Compound 1D (11.57 g, 35.9 mmol) was dissolved in ethanol (50 ml), and 10% palladium on carbon catalyst (1 g) was added. The mixture was subjected to hydrogen replacement three times, stirred overnight at room temperature, and filtered through a funnel lined with diatomaceous earth, and the diatomaceous earth was washed with anhydrous ethanol. The filtrate was concentrated, to the resulting residue was added a solution of hydrogen chloride in dioxane (60 ml, 4M), and the mixture was stirred at mom temperature for 1 h and concentrated. To the resulting residue was added ethyl acetate (50 ml), the mixture was stirred and filtered, and the filter cake was washed with ethyl acetate and dried, to afford compound 1E (4.28 g, 42.0%) as a white solid.

¹H NMR (400 MHz, DMSO-d₆) δ 10.39 (s, 1H), 8.62 (d, 1H), 7.75 (s, 1H), 4.38-4.29 (m, 2H), 3.24 (dd, 1H), 2.97 (dd, 1H), 2.62-2.53 (m, 1H), 1.83-1.64 (m, 1H), 1.55-1.35 (m, 1H), 1.33 (dd, 3H), 0.94 (t, 3H).

### Step 6:

Compound 1E (4.28 g, 17.3 mmol) and 2,3-dichloro-5,6-dicyanobenzoquinone (4.309 g, 19.0 mmol) were dissolved in dioxane (86 ml), and the mixture was reacted at 100° C. for 3.5 h at reflux. After the reaction was completed, saturated sodium bicarbonate solution (40 ml) and ethyl acetate (120 ml) were added. The organic phase was separated, and the aqueous phase was extracted with ethyl acetate (120 ml×2). The organic phases were combined, dried over anhydrous sodium sulfate and concentrated, and the resulting residue was purified by silica gel column chromatography (eluent: ethyl acetate:petroleum ether=0-50%), to afford compound 1F (3.375 g, 79.5%) as a pale yellow solid.

LC-MS(ESI) m/z=247.1 [M+1]⁺

### Step 7:

Compound 1F (3.375 g, 13.72 mmol) was dissolved in anhydrous tetrahydrofuran (150 ml), and the mixture was stirred at −78° C. Lithium aluminum hydride (1.564 g, 41.16 mmol) was added portionwise, and the mixture was stirred at −78° C. for 20 min, warmed to −40° C. and stirred for 20 min. After the reaction was completed. 1M hydrochloric acid was added to adjust the system to a neutral pH, the solvent was removed by distillation under reduced pressure, to the resulting residue was added methanol/dichloromethane (1:10, 100 ml) for dissolution, and the mixture was subjected to ultrasonic vibration for 10 min and filtered. The filtrate was collected, the filter cake was redissolved in methanol/dichloromethane (1:10, 100 ml), and this process was repeated 8 times. The filtrate was combined and concentrated, to afford compound 1G (2.8 g, 100%) as a pale yellow solid.

¹H NMR (400 MHz, DMSO) δ 11.86 (s, 1H), 8.37 (d, 1H), 7.72 (d, 1H), 7.62 (d, 1H), 5.44 (t, 1H), 4.61 (d, 2H), 2.57-2.51 (m, 2H), 1.18 (t, 3H).

### Step 8:

1G (100 mg, 0.49 mmol) was added to dichloromethane (2.5 mL), DMF (1 mL) was added to assist dissolution, thionyl chloride (350 mg, 2.94 mmol) was added dropwise at 0° C. and the mixture was reacted at room temperature for 1 hour. Upon complete depletion of raw materials monitored by LCMS, a product was generated, and the system was directly spun to dryness, to afford title compound 1H (109 mg, crude) which was used in the next reaction.

LC-MS (ESI): m/z=223.1, 225.1 [M+H]⁺.

### Step 9:

1H (109 mg, 0.49 mmol) and 1B (131 mg, 0.49 mmol) were dissolved in anhydrous acetonitrile (5 mL), potassium iodide (8 mg, 0.05 mmol) and DIPEA (316 mg, 2.45 mmol) were added, and the mixture was subjected to nitrogen replacement and reacted at 80° C. for 2 hours. Upon complete depletion of raw materials monitored by LCMS, a product was generated, and the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, passed through column (DCM:MeOH (v/v)=1:0-10:1), and separated with a preparative silica gel plate (DCM:MeOH=10:1), to afford compound 1 (39 mg, 19.1%).

¹H NMR (400 MHz, DMSO-d₆) δ 11.82 (s, 1H), 8.41 (s, 1H), 7.75 (s, 1H), 7.63 (s, 1H), 7.45 (d, 1H), 7.12-6.93 (m, 2H), 4.33 (s, 2H), 3.65 (s, 2H), 3.30-3.25 (m, 4H), 3.01 (s, 3H), 2.60-2.52 (m, 6H), 1.19 (t, 3H).

LC-MS (ESI): m/z=418.3 [M+H]⁺.

### Example 2

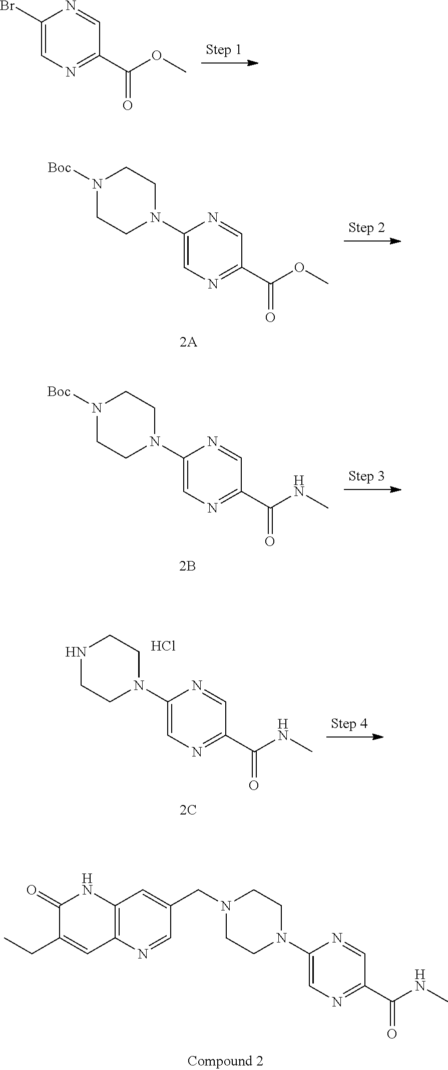 [SMILES: COC(=O)c1cnc(Br)cn1 ; COC(=O)c1cnc(N2CCN(C(=O)OC(C)(C)C)CC2)cn1 ; CNC(=O)c1cnc(N2CCN(C(=O)OC(C)(C)C)CC2)cn1 ; CNC(=O)c1cnc(N2CCNCC2)cn1 ; Cl ; CCc1cc2ncc(CN3CCN(c4cnc(C(=O)NC)cn4)CC3)cc2[nH]c1=O]

### Step 1:

Methyl 5-bromopyrazine-2-carboxylate (600 mg, 2.76 mmol) and N-Boc-piperazine (618 mg, 3.32 mmol) were dissolved in 1,4-dioxane (10 mL), Cs₂CO₃ (1.8 g, 5.53 mmol) and RuPhos-Pd-G3 (93 mg, 0.11 mmol) were added, and the mixture was reacted overnight at 100° C. under nitrogen protection. Subsequently, the reaction liquid was quenched with water (15 mL) and extracted with ethyl acetate (20 mL×3). The organic phases were combined, dried over anhydrous Na₂SO₄, filtered, spun to dryness, and separated by silica gel chromatographic column (PE:EA (v/v)=1:0-1:1), to afford title compound 2A (779 mg, 87.7%) as a white solid.

LC-MS (ESI): m/z=323.1, 267.1 [M+H]⁺.

### Step 2:

2A (779 mg, 2.42 mmol) was dissolved in methanol (10 mL), methylamine aqueous solution (750 mg, 40%) was added, and the mixture was reacted at room temperature for 4 hours. The suspension was concentrated, saturated ammonium chloride solution was added, and the mixture was extracted with DCM. The organic phases were combined, dried over anhydrous sodium sulfate, filtered and spun to dryness, to afford title compound 2B (760 mg, 97.9%).

LC-MS (ESI): m/z=322.2 [M+H]⁺.

### Step 3:

2B (760 mg, 2.37 mmol) was dissolved in methanol (5 mL), and a solution of hydrogen chloride in dioxane (5 mL, 4 M) was added. The mixture was reacted at room temperature for two hours and spun to dryness to obtain title compound 2C (523 mg, crude).

LC-MS (ESI): m/z=222.1 [M+H]⁺.

### Step 4:

1H (109 mg, 0.49 mmol) and 2C (127 mg, 0.49 mmol) were dissolved in anhydrous acetonitrile (5 mL), potassium iodide (8 mg, 0.05 mmol) and DIPEA (316 mg, 2.45 mmol) were added, and the mixture was subjected to nitrogen replacement and reacted at 80° C. for 2 hours. Upon complete depletion of raw materials monitored by LCMS, a product was generated, and the system was concentrated, saturated sodium bicarbonate solution (20 mL) was added, and the mixture was extracted with a mixed solution of DCM:MeOH (v/v)=10:1 (10 mL×3). The organic phases were combined, dried over anhydrous sodium sulfate, concentrated, and passed through column (DCM:MeOH (v/v)=1:0-10:1), to afford compound 2 (80 mg, 40.1%).

¹H NMR (400 MHz, DMSO-d₆) δ 11.84 (s, 1H), 8.59 (s, 1H), 8.43-8.38 (m, 1H), 8.32 (q, 1H), 8.26 (s, 1H), 7.75 (s, 1H), 7.63 (s, 1H), 3.70 (s, 4H), 3.65 (s, 2H), 2.77 (d, 3H), 2.60-2.52 (m, 4H), 1.18 (t, 3H).

LC-MS (ESI): m/z=408.2 [M+H]⁺.

### Example 3

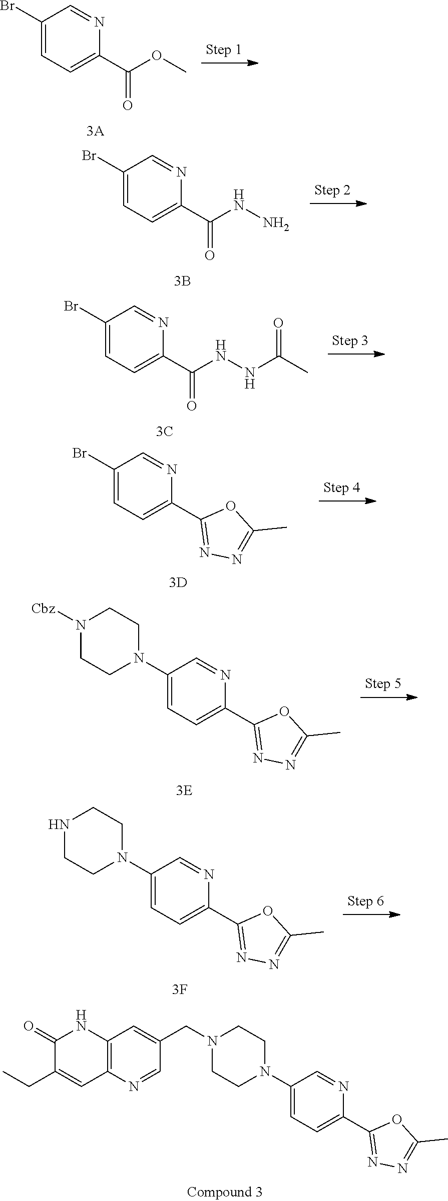

### Step 1:

Hydrazine hydrate (694 mg, 13.89 mmol) was added to a solution of methyl 5-bromopyridine-2-carboxylate (1 g, 4.63 nmol) in methanol (10 mL), and the mixture was heated to reflux for 1 hour. The reaction liquid was concentrated under reduced pressure, and the solid was filtered off, washed with methanol and dried, to afford title compound 3B (880 mg, 88.0%).

LC-MS (ESI): m/z=216.1, 218.1 [M+H]⁺.

### Step 2:

3B (880 mg, 4.07 mmol) and triethylamine (1.14 mL, 8.15 mmol) were added to DCM (15 mL), Ac₂O (0.44 mL, 4.48 mmol) was added dropwise at 25° C., and the reaction liquid was stirred for 1.5 hours. Subsequently, the reaction liquid was poured into ice water, and the solid was filtered off, washed with water and dried, to afford title compound 3C (0 g, 95.1%).

LC-MS (ESI): m/z=257.1, 259.1 [M+H]⁺.

### Step 3:

3C (1 g, 3.88 mmol) and triethylamine (3.2 mL, 23.3 mmol) were added to DCM (15 mL), TsCl (884 mg, 4.65 mmol) was then added, and the mixture was reacted at room temperature for 3 hours. After the reaction was completed as monitored by TLC, the reaction liquid was quenched with saturated NaHCO₃ solution (20 mL) and extracted with DCM (20 mL×2). The organic layers were combined, dried over anhydrous sodium sulfate, concentrated, and purified by silica gel chromatographic column (EA:PE (v/v)=0:1-1:0), to afford title compound 3D (810 mg, 87.1%).

LC-MS (ESI): m/z=223.1 [M+H]⁺.

### Step 4:

3D (400 mg, 1.67 mmol), benzyl-1-piperazine carbonate (440 mg, 2.00 mmol), cesium carbonate (1.63 g, 5.00 mmol) and RuPhos-Pd-G3 (56 mg, 0.04 mmol) were added to 1,4-dioxane (10 mL), and the mixture was subjected to nitrogen replacement and reacted overnight at 100° C. Upon complete depletion of raw materials monitored by TLC, the reaction liquid was filtered, concentrated, and separated and purified by silica gel chromatographic column (EA:PE (v/v)=0:1-1:0), to afford title compound 3E (494 mg, 78.2%).

LC-MS(ESI): m/z=380.2 [M+H]⁺

### Step 5:

3E (250 mg, 0.66 mmol) was dissolved in methanol, and palladium on carbon catalyst (10%, 100 mg) was added. The reaction was carried out under hydrogen conditions for 2 hours, and the reaction liquid was filtered and spun to dryness, to afford title compound 3F (160 mg, 99.0%).

### Step 6:

1H (50 mg, 0.22 mmol) and 3F (66 mg, 0.27 mmol) were dissolved in anhydrous acetonitrile (5 mL), potassium iodide (4 mg, 0.02 mmol) and DIPEA (144 mg, 1.12 mmol) were added, and the mixture was subjected to nitrogen replacement and reacted at 80° C. for 2 hours. Upon complete depletion of raw materials monitored by LCMS, a product was generated, and the system was concentrated, saturated sodium bicarbonate solution (20 mL) was added, and the mixture was extracted with DCM (10 mL×3). The organic phases were combined, dried over anhydrous sodium sulfate, concentrated, and passed through column (DCM:MeOH (v/v)=1:0-10:1), to afford compound 3 (56 mg, 57.9%).

¹H NMR (400 MHz, DMSO-d₆) δ 11.83 (s, 1H), 8.45-8.37 (m, 2H), 7.91 (d, 1H), 7.75 (s, 1H), 7.63 (s, 1H), 7.45 (dd, 1H), 3.66 (s, 2H), 3.45-3.34 (m, 4H), 2.60-2.52 (m, 9H), 1.19 (t, 3H).

LC-MS (ESI): m/z=432.2 [M+H]⁺.

### Example 4

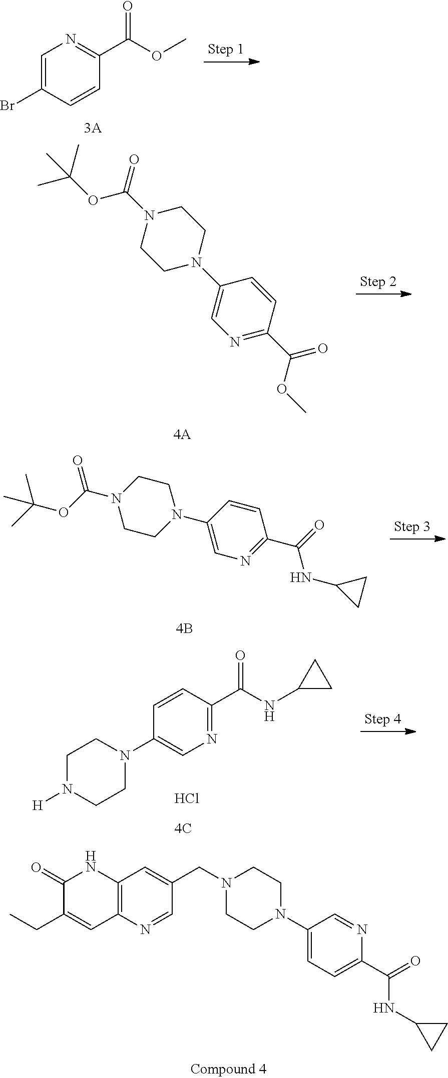

### Step 1:

Methyl 5-bromopyridine-2-carboxylate (2.16 g, 10 mmol) and N-Boc-piperazine (2.03 g, 11 mmol) were dissolved in 1,4-dioxane (100 mL), Cs₂CO₃ (6.5 g, 20 mmol) and RuPhos-Pd-G3 (253 mg, 0.3 mmol) were added, and the mixture was reacted overnight at 100° C. under nitrogen protection. After the reaction was completed as monitored by LCMS, the reaction was terminated, cooled to room temperature, and filtered. The filtrate was collected, and the filter residue was washed with ethyl acetate (20 mL×3). The filtrate was concentrated, a small amount of anhydrous ethanol was added, and the mixture was heated and dissolved. A large amount of petroleum ether was then added, and the mixture was cooled to collect the precipitated crystals, to afford title compound 4A (2.37 g, 73.4%) as a pale yellow solid.

LC-MS (ESI): m/z=321.1 [M+H]⁺.

### Step 2:

Compound 4A (400 mg, 1.24 mmol) was dissolved in THF (10 mL) and H₂O (1 mL), LiOH (30 mg, 1.24 mmol) was added, and the mixture was stirred and reacted at room temperature for 2 h. The solvent was removed by distillation under reduced pressure, water was added for dilution, and the mixture was extracted with ethyl acetate (20 mL×3). The organic phases were combined, dried over anhydrous Na₂SO₄, filtered, and spun to dryness. To the resulting solid was added DMF (10 mL), HATU (565 mg, 1.49 mmol) was added under stirring, and the mixture was stirred at room temperature. After the solid was completely dissolved, DIEPA (2 mL) was added, excess cyclopropylamine was finally added, and the mixture was stirred overnight at room temperature. After the reaction was completed as monitored by LCMS, to the system was added ethyl acetate (50 mL), and the mixture was washed with water (50 mL×4). The organic phase was collected, dried over anhydrous sodium sulfate, filtered, evaporated to dryness, and separated by silica gel chromatographic column (PE:EA (v/v)=1:0-1:1), to afford title compound 4B (309 mg, 71.5%) as a pale yellow solid.

LC-MS (ESI): m/z=347.2 [M+H]⁺.

### Step 3:

4B (309 mg, 0.89 mmol) was dissolved in methanol (5 mL), and a solution of hydrogen chloride in dioxane (5 mL, 4 M) was added. The mixture was reacted at room temperature for two hours and spun to dryness to obtain title compound 4C (200 mg, crude).

LC-MS (ESI): m/z=247.1 [M+H]⁺.

### Step 4:

1H (100 mg, 0.44 mmol) and 4C (200 mg, 0.81 mmol) were dissolved in anhydrous acetonitrile (10 mL), potassium iodide (8 mg, 0.05 mmol) and DIPEA (0.5 mL) were added, and the mixture was subjected to nitrogen replacement and reacted at 80° C. for 8 hours. Upon complete depletion of raw materials monitored by LCMS, a product was generated, and the system was concentrated, saturated sodium bicarbonate solution (20 mL) was added, and the mixture was extracted with a mixed solution of DCM:MeOH (v/v)=10:1 (10 mL×3). The organic phases were combined, dried over anhydrous sodium sulfate, concentrated, and passed through column (DCM:MeOH (v/v)=1:0-10:1), to afford compound 4 (76 mg, 38.1%).

¹H NMR (400 MHz, DMSO-d₆) δ 11.84 (s, 1H), 8.40 (d, 1H), 8.32 (d, 1H), 8.23 (d, 1H), 7.83 (d, 1H), 7.75 (s, 1H), 7.63 (d, 1H), 7.39 (dd, 1H), 3.65 (s, 2H), 2.90-2.80 (m, 1H), 2.56 (d, 4H), 2.54 (d, 4H), 1.19 (t, 3H), 0.66 (dd, 2H), 0.63 (q, 2H).

LC-MS (ESI): m/z=433.2 [M+H]⁺.

### Example 5

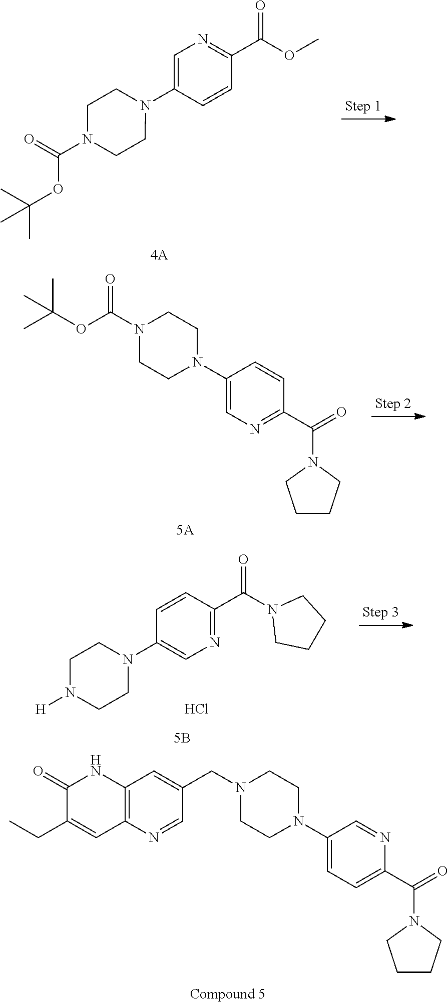

### Step 1:

Compound 4A (400 mg, 1.24 mmol) was dissolved in THF (10 mL) and H₂O (1 mL), LiOH (30 mg, 1.24 mmol) was added, and the mixture was stirred and reacted at room temperature for 2 h. The solvent was removed by distillation under reduced pressure, water was added for dilution, and the mixture was extracted with ethyl acetate (20 mL×3). The organic phases were combined, dried over anhydrous Na₂SO₄, filtered, and spun to dryness. To the resulting solid was added DMF (10 mL), HATU (565 mg, 1.49 mmol) was added under stirring, and the mixture was stirred at room temperature. After the solid was completely dissolved, DIEPA (2 mL) was added, excess pyrrolidine was finally added, and the mixture was stirred overnight at room temperature. After the reaction was completed as monitored by LCMS, to the system was added ethyl acetate (50 mL), and the mixture was washed with water (50 mL×4). The organic phase was collected, dried over anhydrous sodium sulfate, filtered, evaporated to dryness, and separated by silica gel chromatographic column (PE:EA (v/v)=1:0-1:1), to afford title compound 5A (362 mg, 80.5%) as a pale yellow solid.

LC-MS (ESI): m/z=361.2 [M+H]⁺.

### Step 3:

5A (360 mg, 1 mmol) was dissolved in methanol (5 mL), and a solution of hydrogen chloride in dioxane (5 mL, 4 M) was added. The mixture was reacted at rom temperature for four hours and spun to dryness to obtain title compound 5B (243 mg, crude).

LC-MS (ESI): m/z=261.1 [M+H]⁺.

### Step 4:

1H (100 mg, 0.44 mmol) and 5B (243 mg, 0.93 mmol) were dissolved in anhydrous acetonitrile (10 mL), potassium iodide (8 mg, 0.05 mmol) and DIPEA (0.5 mL, 2.45 mmol) were added, and the mixture was subjected to nitrogen replacement and reacted at 80° C. overnight. Upon complete depletion of raw materials monitored by LCMS, a product was generated, and the system was concentrated, saturated sodium bicarbonate solution (20 mL) was added, and the mixture was extracted with a mixed solution of DCM:MeOH (v/v)=10:1 (10 mL×3). The organic phases were combined, dried over anhydrous sodium sulfate, concentrated, and passed through column (DCM:MeOH (v/v)=1:0-10:1), to afford compound 5 (64 mg, 34.6%).

¹H NMR (400 MHz, DMSO-d₆) δ 11.84 (s, 1H), 8.40 (d, 1H), 8.26 (d, 1H), 7.75 (s, 1H), 7.65 (d, 1H), 7.63 (d, 1H), 7.36 (dd, 1H), 3.77-3.68 (m, 2H), 3.65 (s, 2H), 3.47 (t, 2H), 3.31 (s, 2H), 2.56 (dt, 4H), 2.54 (d, 2H), 1.89-1.75 (m, 4H), 1.18 (t, 3H).

LC-MS (ESI): m/z=447.2 [M+H]⁺.

### Example 6

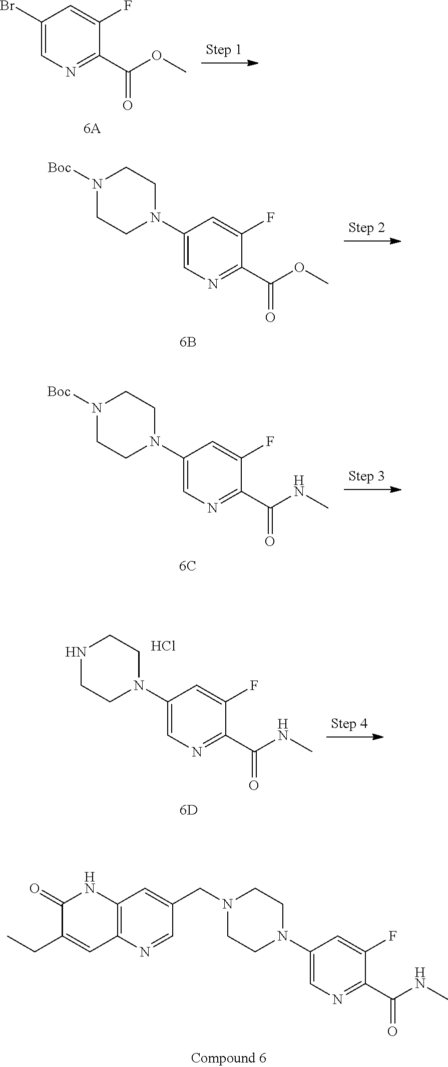 [SMILES: COC(=O)c1ncc(Br)cc1F ; COC(=O)c1ncc(N2CCN(C(=O)OC(C)(C)C)CC2)cc1F ; CNC(=O)c1ncc(N2CCN(C(=O)OC(C)(C)C)CC2)cc1F ; CNC(=O)c1ncc(N2CCNCC2)cc1F ; Cl ; CCc1cc2ncc(CN3CCN(c4cnc(C(=O)NC)c(F)c4)CC3)cc2[nH]c1=O]

### Step 1:

Methyl 5-bromo-3-fluoropyridine-2-carboxylate (2.34 g, 10 mmol) and N-Boc-piperazine (2.03 g, 11 mmol) were dissolved in 1,4-dioxane (100 mL), Cs₂CO₃ (6.5 g, 20 mmol) and RuPhos-Pd-G3 (253 mg, 0.3 mmol) were added, and the mixture was reacted overnight at 100° C. under nitrogen protection. After the reaction was completed as monitored by LCMS, the reaction was terminated, cooled to room temperature, and filtered. The filtrate was collected, and the filter residue was washed with ethyl acetate (20 mL×3). The filtrate was concentrated, a small amount of anhydrous ethanol was added, and the mixture was heated and dissolved. A large amount of petroleum ether was then added, and the mixture was cooled to collect the precipitated crystals, to afford title compound 6B (1.89 g, 56.5%) as a white solid.

LC-MS (ESI): m/z=340.2 [M+H]⁺.

### Step 2:

6B (400 mg, 1.18 mmol) was dissolved in methanol (10 mL), methylamine aqueous solution (0.5 mL, 40%) was added, and the mixture was reacted at room temperature for 4 hours. The system was concentrated, saturated ammonium chloride solution was added, and the mixture was extracted with DCM. The organic phases were combined, dried over anhydrous sodium sulfate, filtered and spun to dryness, to afford title compound 6C (384 mg, 96.7%).

LC-MS (ESI): m/z=339.2 [M+H]⁺.

### Step 3:

6C (380 mg, 1.12 mmol) was dissolved in methanol (5 mL), and a solution of HCl in dioxane (5 mL, 4 M) was added. The mixture was reacted at room temperature for two hours and spun to dryness to obtain title compound 6D (255 mg, crude).

LC-MS (ESI): m/z=239.1 [M+H]⁺.

### Step 4:

1H (100 mg, 0.44 mmol) and 6D (255 mg, 1.07 mmol) were dissolved in anhydrous acetonitrile (10 mL), potassium iodide (8 mg, 0.05 mmol) and DIPEA (316 mg, 2.45 mmol) were added, and the mixture was subjected to nitrogen replacement and reacted at 80° C. for 2 hours. Upon complete depletion of raw materials monitored by LCMS, a product was generated, and the system was concentrated, saturated sodium bicarbonate solution (20 mL) was added, and the mixture was extracted with a mixed solution of DCM:MeOH (v/v)=10:1 (10 mL×3). The organic phases were combined, dried over anhydrous sodium sulfate, concentrated, and passed through column (DCM:MeOH (v/v)=1:0-10:1), to afford compound 6 (78 mg, 41.1%).

¹H NMR (400 MHz, DMSO-d₆) δ 11.84 (s, 1H), 8.40 (d, 1H), 8.25 (q, 1H), 8.13 (t, 1H), 7.75 (s, 1H), 7.62 (d, 1H), 7.21 (dd, 1H), 3.65 (s, 2H), 3.39 (t, 4H), 2.75 (d, 3H), 2.56 (d, 2H), 2.54 (s, 4H), 1.19 (t, 3H).

LC-MS (ESI): m/z=425.3 [M+H]⁺.

### Example 7

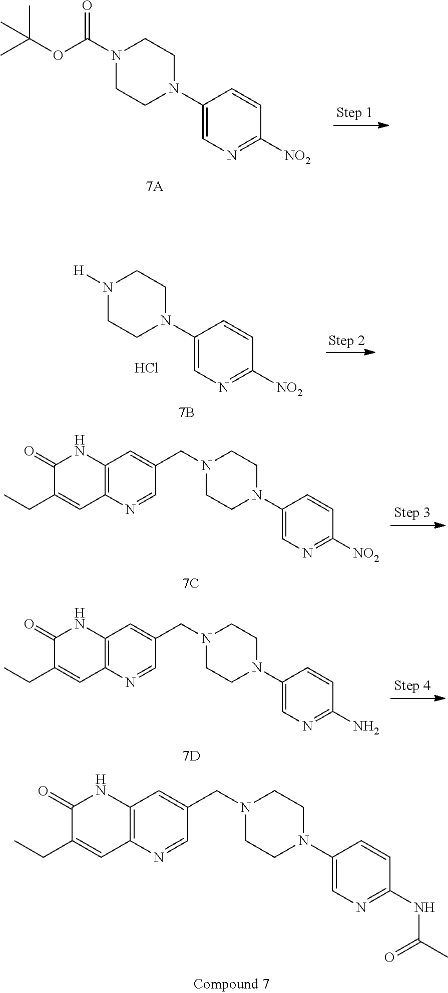 [SMILES: CC(C)(C)OC(=O)N1CCN(c2ccc([N+](=O)[O-])nc2)CC1 ; O=[N+]([O-])c1ccc(N2CCNCC2)cn1 ; Cl ; CCc1cc2ncc(CN3CCN(c4ccc([N+](=O)[O-])nc4)CC3)cc2[nH]c1=O ; CCc1cc2ncc(CN3CCN(c4ccc(N)nc4)CC3)cc2[nH]c1=O ; CCc1cc2ncc(CN3CCN(c4ccc(NC(C)=O)nc4)CC3)cc2[nH]c1=O]

### Step 1:

Tert-butyl 4-(6-nitropyridin-3-yl)piperazine-1-carboxylate (800 mg, 2.60 mmol) was dissolved in methanol (10 mL), and a solution of hydrogen chloride in dioxane (5 mL, 4 M) was added. The mixture was reacted at room temperature for four hours and spun to dryness to obtain title compound 7B (602 mg, crude).

LC-MS (ESI): m/z=209.1 [M+H]⁺.

### Step 2:

1H (300 mg, 1.34 mmol) and 7B (600 mg, 2.87 mmol) were dissolved in anhydrous acetonitrile (20 mL), potassium iodide (15 mg, 0.05 mmol) and DIPEA (i mL) were added, and the mixture was subjected to nitrogen replacement and reacted at 80° C. for 8 hours, at which time a large amount of yellow solid was observed to be generated. Upon complete depletion of raw materials monitored by LCMS, the system was concentrated, ethyl acetate (20 mL) was added, and the mixture was subjected to ultrasonic vibration and filtered to collect the filter residue, which was compound 7C (247 mg, 46.7%).

LC-MS (ESI): m/z=395.2 [M+H]⁺.

### Step 3:

7C (247 mg, 0.62 mmol) was dissolved in anhydrous methanol (20 mL), palladium on carbon (50 mg, 10%) and hydrazine hydrate (0.5 mL) were added, and the mixture was reacted at 75° C. for 4 hours. After the reaction was completed as monitored by LCMS, the reaction liquid was filtered to remove palladium on carbon and spun to dryness, to afford target compound 7D (204 mg, 89.8%) as a white solid.

LC-MS (ESI): m/z=365.2 [M+H]⁺.

### Step 4:

Compound 7D (100 mg, 0.27 mmol) was dissolved in THF (10 mL), acetic anhydride (27.5 mg, 0.27 mmol) and two drops of pyridine were added, and the mixture was stirred overnight at room temperature. After the reaction was completed as monitored by LCMS, the system was concentrated, saturated sodium bicarbonate solution (20 mL) was added, and the mixture was extracted with a mixed solution of DCM:MeOH (v/v)=10:1 (10 mL×3). The organic phases were combined, dried over anhydrous sodium sulfate, concentrated, and passed through column (DCM:MeOH (v/v)=1:0-10:1), to afford compound 7 (81 mg, 72.9%).

¹H NMR (400 MHz, DMSO-d₆) δ 12.17 (s, 1H), 10.29 (s, 1H), 8.55 (d, 1H), 8.04 (d, 1H), 7.95 (d, 1H), 7.81 (d, 1H), 7.78 (d, 1H), 7.46 (dd, 1H), 4.53 (s, 2H), 3.81 (s, 4H), 3.00 (s, 4H), 2.65-2.51 (m, 2H), 2.05 (s, 3H), 1.20 (t, 3H).

LC-MS (ESI): m/z=407.2 [M+H]⁺.

### Example 8

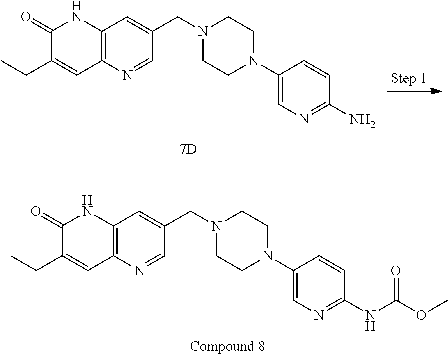 [SMILES: CCc1cc2ncc(CN3CCN(c4ccc(N)nc4)CC3)cc2[nH]c1=O ; CCc1cc2ncc(CN3CCN(c4ccc(NC(=O)OC)nc4)CC3)cc2[nH]c1=O]

Compound 7D (100 mg, 0.27 mmol) was dissolved in methanol (10 mL), (Boc)₂O (70.6 mg, 0.32 mmol) was added, and the mixture was stirred at room temperature for 24 h. After the reaction was completed as monitored by LCMS, the system was concentrated, saturated sodium bicarbonate solution (10 mL) was added, and the mixture was extracted with a mixed solution of DCM:MeOH (v/v)=10:1 (10 mL×3). The organic phases were combined, dried over anhydrous sodium sulfate, concentrated, and passed through column (DCM:MeOH (v/v)=1:0-10:1), to afford compound 8 (82 mg, 71.3%).

¹H NMR (400 MHz, DMSO-d₆) δ 12.17 (s, 1H), 9.92 (s, 1H), 8.55 (d, 1H), 8.01 (d, 1H), 7.81 (s, 1H), 7.78 (d, 1H), 7.70 (d, 1H), 7.48 (dd, 1H), 4.53 (s, 2H), 3.65 (s, 3H), 3.43 (s, 4H), 3.13 (d, 4H), 2.64-2.53 (m, 2H), 1.20 (t, 3H).

LC-MS (ESI): m/z=423.2 [M+H]⁺.

### Example 9

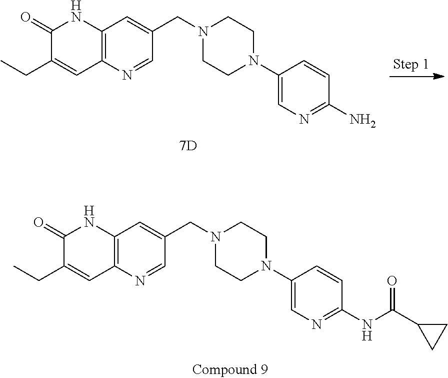 [SMILES: CCc1cc2ncc(CN3CCN(c4ccc(N)nc4)CC3)cc2[nH]c1=O ; CCc1cc2ncc(CN3CCN(c4ccc(NC(=O)C5CC5)nc4)CC3)cc2[nH]c1=O]

Cyclopropanecarboxylic acid (86.1 mg, 1 mmol) was dissolved in DMF (10 mL). HATU (570 mg, 1.5 mmol) was added under stirring, 7D (240 mg, 0.65 mmol) and DIPEA (420 mg, 3.25 mmol) were added after 30 min, and the mixture was reacted at room temperature for 4 h. After the reaction was completed as monitored by LCMS, to the system was added ethyl acetate (50 mL), and the mixture was washed with water (50 mL×4). The organic phase was collected, dried over anhydrous sodium sulfate, filtered, evaporated to dryness, and separated by silica gel chromatographic column (DCM:MeOH (v/v)=1:0-1:1), to afford title compound 9 (181 mg, 63.7%).

¹H NMR (400 MHz, DMSO-d₆) δ 11.83 (s, 1H), 10.49 (s, 1H), 8.40 (d, 1H), 7.98 (d, 1H), 7.91 (d, 1H), 7.75 (s, 1H), 7.63 (d, 1H), 7.36 (dd, 1H), 3.64 (s, 2H), 3.32 (s, 3H), 3.14 (t, 4H), 2.56 (m, 4H), 2.54-2.52 (m, 2H), 2.01-1.89 (m, 1H), 1.19 (t, 3H), 0.79 (t, 2H), 0.77-0.70 (in, 2H).

LC-MS (ESI): m/z=433.2 [M+H]⁺.

### Example 10

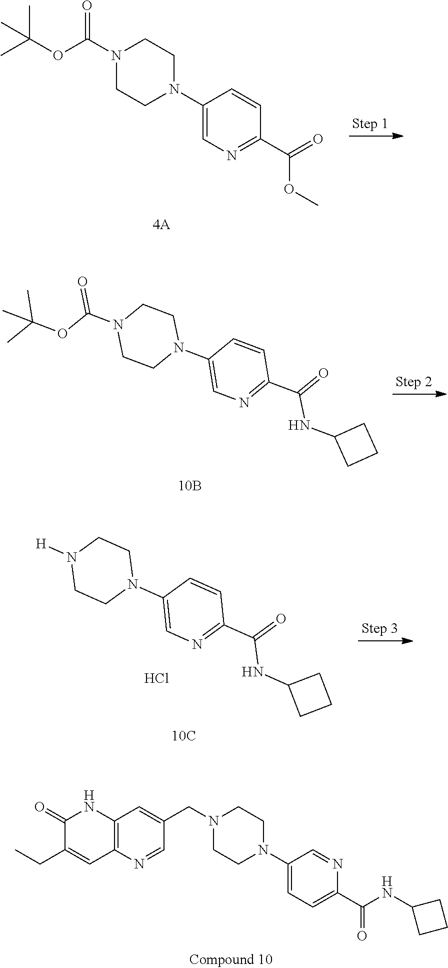

### Step 1:

Compound 4A (321 mg, 1 mmol) was dissolved in THF (10 mL) and H₂O (1 mL), LiOH·H₂O (45 mg, 1.1 mmol) was added, and the mixture was stirred and reacted at room temperature for 2 h. The solvent was removed by distillation under reduced pressure, to the resulting solid was added DMF (10 mL), HATU (570 mg, 1.5 mmol) was added under stirring, and the mixture was stirred at room temperature. After the solid was completely dissolved, DIEPA (1 mL) was added, excess cyclobutylamine was finally added, and the mixture was stirred overnight at room temperature. After the reaction was completed as monitored by LCMS, to the system was added ethyl acetate (50 mL), and the mixture was washed with water (50 mL×4). The organic phase was collected, dried over anhydrous sodium sulfate, filtered, evaporated to dryness, and separated by silica gel chromatographic column (PE:EA=1:0-1:1), to afford title compound 10B (336 mg, 93.3%) as a pale yellow solid.

LC-MS (ESI): m/z=361.2 [M+H]⁺.

### Step 2:

10B (336 mg, 0.93 mmol) was dissolved in methanol (5 mL), and a solution of hydrogen chloride in dioxane (5 mL, 4 M) was added. The mixture was reacted at room temperature for two hours and spun to dryness to obtain title compound 10C (251 mg, crude).

LC-MS (ESI): m/z=261.2 [M+H]⁺.

### Step 3:

1H (150 mg, 0.67 mmol) and 10C (251 mg, 0.85 mmol) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, a product was generated, the system was concentrated, saturated sodium bicarbonate solution (20 mL) was added, and the mixture was extracted with a mixed solution of DCM:MeOH (v/v)=10:1 (10 mL×3). The organic phases were combined, dried over anhydrous sodium sulfate, concentrated, and passed through column (DCM:MeOH (v/v)=1:0-10:1), to afford compound 10 (128 mg, 42.8%).

¹H NMR (400 MHz, DMSO-d₆) δ 11.83 (s, 1H), 8.51 (d, 1H), 8.41 (d, 1H), 8.27 (d, 1H), 7.81 (d, 1H), 7.75 (q, 1H), 7.68-7.60 (m, 1H), 7.39 (dd, 1H), 4.41 (h, 1H), 3.66 (s, 2H), 3.39-3.32 (m, 4H), 2.56 (dd, 4H), 2.54 (d, 2H), 2.22-2.16 (m, 2H), 2.15-2.10 (m, 2H), 1.69-1.58 (m, 2H), 1.18 (t, 3H).

LC-MS (ESI): m/z=447.2 [M+H]⁺.

### Example 11

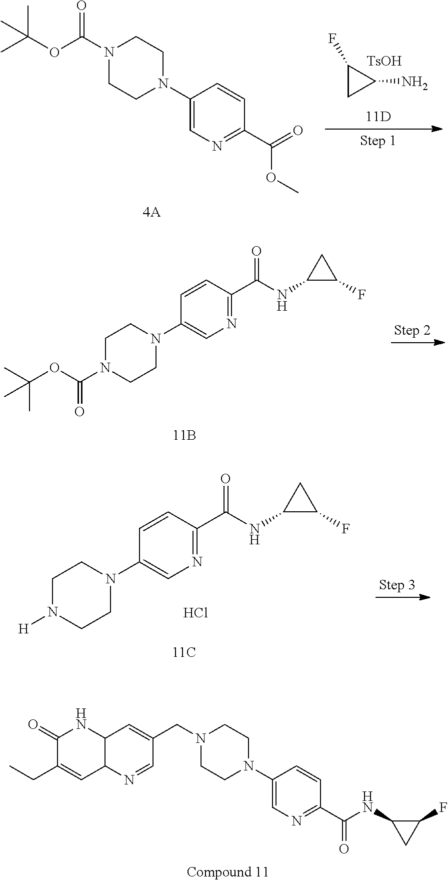

### Step 1:

Compound 4A (321 mg, 1 mmol) was dissolved in THF (10 mL) and H₂O (1 mL), LiOH·H₂O (45 mg, 1.1 mmol) was added, and the mixture was stirred and reacted at room temperature for 2 h. The solvent was removed by distillation under reduced pressure, to the resulting solid was added DMF (10 mL), HATU (570 mg, 1.5 mmol) was added under stirring, and the mixture was stirred at room temperature. After the solid was completely dissolved, DIEPA (1 mL) was added, excess 11D was finally added, and the mixture was stirred overnight at room temperature. After the reaction was completed as monitored by LCMS, to the system was added ethyl acetate (50 mL), and the mixture was washed with water (50 mL×4). The organic phase was collected, dried over anhydrous sodium sulfate, filtered, evaporated to dryness, and separated by silica gel chromatographic column (PE:EA(v/v)=1:0-1:1), to afford title compound 11B (312 mg, 85.7%) as a pale yellow solid.

LC-MS (ESI): m/z=365.2 [M+H]⁺.

### Step 2:

11B (312 mg, 0.86 mmol) was dissolved in methanol (5 mL), and a solution of hydrogen chloride in dioxane (5 mL, 4 M) was added. The mixture was reacted at room temperature for two hours and spun to dryness to obtain title compound 11C (215 mg, crude).

LC-MS (ESI): m/z=265.2 [M+H]⁺.

### Step 3:

1H (150 mg, 0.67 mmol) and 11C (215 mg, 0.72 mmol) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, a product was generated, the system was concentrated, saturated sodium bicarbonate solution (20 mL) was added, and the mixture was extracted with a mixed solution of DCM:MeOH (v/v)=10:1 (10 mL×3). The organic phases were combined, dried over anhydrous sodium sulfate, concentrated, and passed through column (DCM:MeOH (v/v)=1:0-10:1), to afford compound 11 (128 mg, 42.8%).

¹H NMR (400 MHz, DMSO-d₆) δ 11.81 (s, 1H), 8.40 (d, 1H), 8.31 (d, 1H), 8.27 (d, 1H), 7.85 (d, 1H), 7.75 (s, 1H), 7.63 (d, 1H), 7.41 (dd, 1H), 4.90-4.65 (m, 1H), 3.65 (s, 2H), 3.35 (t, 4H), 2.94-2.80 (m, 1H), 2.56 (dd, 4H), 2.54 (d, 2H), 1.29-1.19 (m, 1H), 1.17 (t, 3H), 1.15-1.03 (m, 1H).

LC-MS (ESI): m/z=451.2 [M+H]⁺.

### Example 12

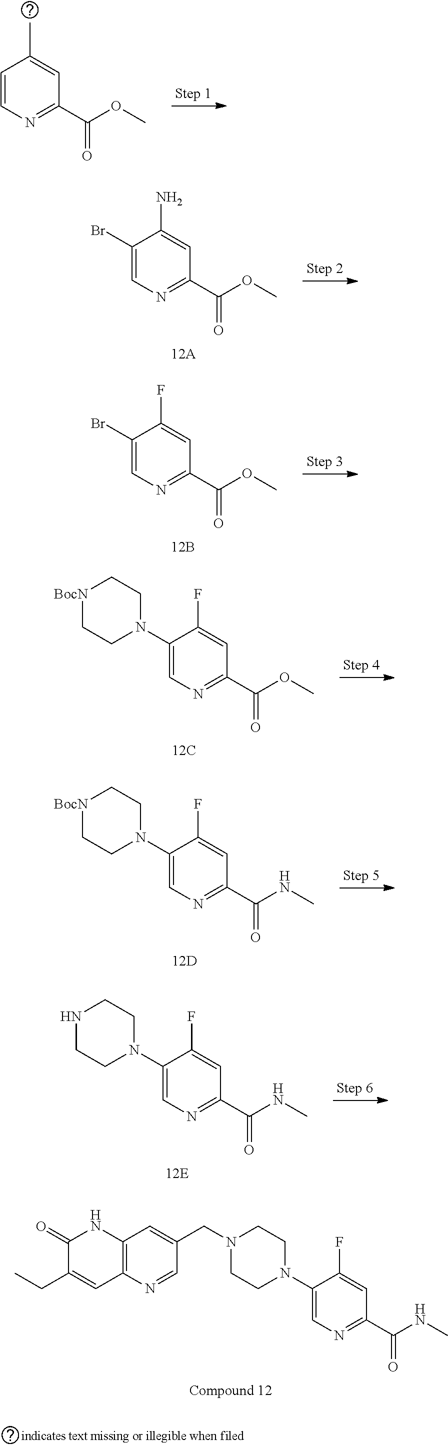

### Step 1:

Methyl 4-aminopyridine-2-carboxylate (1.52 g, 10 mmol) was dissolved in dichloroethane (50 mL), NBS (1.78 g, 10 mmol) was added under stirring, and the mixture was reacted overnight at room temperature. After the reaction was completed as monitored by LCMS, water (50 mL) was added for dilution, and the mixture was extracted with ethyl acetate (50 mL×3). The organic phases were combined, washed with aqueous saturated NaCl solution, dried over anhydrous sodium sulfate, and subjected to solvent removal, to afford title compound 12A (1.76 g, 76%) as a white solid.

LC-MS (ESI): m/z=231.0/233.0 [M+H]⁺.

### Step 2:

To the hydrogen fluoride pyridine solution (50 mL, 65%-70% w/w) in a plastic bottle was added NaNO₂ solid (3.15 g, 46 mmol) in an ice-water bath, 12A (1.76 g, 7.6 mmol) was added under stirring, and the mixture was stirred overnight at 30° C. After the reaction was completed, the reaction liquid was cooled to room temperature, quenched with 200 mL of water, and extracted with dichloromethane (200 mL×3). The organic phases were combined, washed with aqueous saturated NaCl solution, dried over anhydrous sodium sulfate, evaporated to remove the solvent, and separated by silica gel chromatographic column (DCM:MeOH (v/v)=1:0-10:1), to afford title compound 12B (747 mg, 42.1%) as a pale yellow solid.

LC-MS (ESI): m/z=233.9/235.9 [M+H]⁺.

### Step 3:

12B (747 mg, 3.19 mmol) and N-Boc-piperazine (653 mg, 3.51 mmol) were dissolved in 1,4-dioxane (30 mL), Cs₂CO₃ (2.07 g, 6.38 mmol) and RuPhos-Pd-G3 (86 mg, 0.1 mmol) were added, and the mixture was reacted overnight at 100° C. under nitrogen protection. After the reaction was completed as monitored by LCMS, the reaction was terminated, cooled to room temperature, and filtered. The filtrate was collected, and the filter residue was washed with ethyl acetate (20 mL×3). The filtrate was concentrated, filtered, spun to dryness, and separated by silica gel chromatographic column (PE:EA (v/v)=1:0-1:1), to afford title compound 12C (706 mg, 67.2%) as a pale yellow solid.

LC-MS (ESI): m/z=340.1 [M+H]⁺.

### Step 4:

12C (706 mg, 2.08 mmol) was dissolved in methanol (20 mL), methylamine aqueous solution (1 mL, 40%) was added, and the mixture was reacted at room temperature for 4 hours. The system was concentrated, saturated ammonium chloride solution was added, and the mixture was extracted with DCM. The organic phases were combined, dried over anhydrous sodium sulfate, filtered and spun to dryness, to afford title compound 12D (384 mg, 96.7%).

LC-MS (ESI): m/z=339.2 [M+H]⁺.

### Step 5:

12D (338 mg, 1.0 mmol) was dissolved in methanol (5 mL), and a solution of hydrogen chloride in dioxane (2 mL, 4 M) was added. The mixture was reacted at room temperature for two hours and spun to dryness to obtain title compound 12E (243 mg, crude).

LC-MS (ESI): m/z=239.1 [M+H]⁺.

### Step 6:

1H (100 mg, 0.44 mmol) and 12E (243 mg, 0.89 mmol) were dissolved in anhydrous acetonitrile (10 mL), potassium iodide (8 mg, 0.05 mmol) and DIPEA (316 mg, 2.45 mmol) were added, and the mixture was subjected to nitrogen replacement and reacted at 80° C. for 8 hours. Upon complete depletion of raw materials monitored by LCMS, a product was generated, and the system was concentrated, saturated sodium bicarbonate solution (20 mL) was added, and the mixture was extracted with a mixed solution of DCM:MeOH (v/v)=10:1 (10 mL×3). The organic phases were combined, dried over anhydrous sodium sulfate, concentrated, and passed through column (DCM:MeOH (v/v)=1:0-10:1), to afford compound 12 (80 mg, 42.1%).

¹H NMR (400 MHz, DMSO-d₆) δ 12.15 (s, 1H), 8.59 (d, 1H), 8.55-8.53 (m, 1H), 8.36 (d, 1H), 7.81 (s, 1H), 7.77 (s, 1H), 7.74 (d, 1H), 4.50 (s, 2H), 3.88-3.55 (m, 4H), 3.41-3.13 (m, 4H), 2.81 (d, 3H), 2.62-2.53 (m, 2H), 1.20 (t, 3H).

LC-MS (ESI): m/z=425.2 [M+H]⁺.

### Example 13

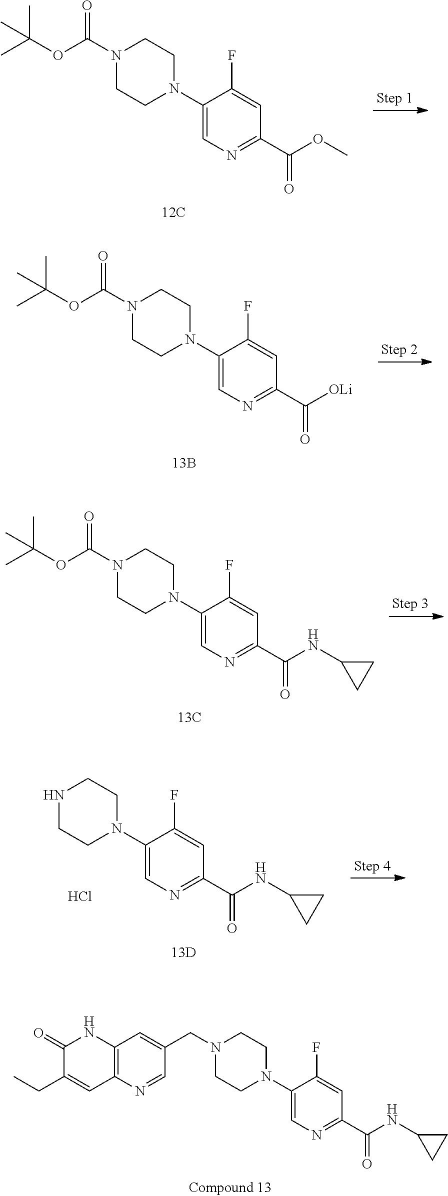

### Step 1:

Compound 12C (340 mg, 1 mmol) was dissolved in THE (10 mL) and H₂O (1 mL), LiOH·H₂O (45 mg, 1.1 mmol) was added, and the mixture was stirred and reacted at room temperature for 2 h. The solvent was removed by distillation under reduced pressure, to afford compound 13B (331 mg, 100%) as a pale yellow solid.

LC-MS (ESI): m/z=332.2 [M+H]⁺.

### Step 2:

Compound 13B (331 mg, 1 mmol) was dispersed in DMF (10 mL), HATU (570 mg, 1.5 mmol) was added under stirring, and the mixture was stirred at room temperature. After the solid was completely dissolved, DIEPA (1 mL) was added, excess cyclopropylamine was finally added, and the mixture was reacted at room temperature for 4 h. After the reaction was completed as monitored by LCMS, to the system was added ethyl acetate (50 mL), and the mixture was washed with water (50 mL×4). The organic phase was collected, dried over anhydrous sodium sulfate, filtered, evaporated to dryness, and separated by silica gel chromatographic column (PE:EA (v/v)=1:0-1:1), to afford title compound 13C (321 mg, 87.9%) as a pale yellow solid.

LC-MS (ESI): m/z=365.2 [M+H]⁺.

### Step 3:

13C (321 mg, 0.88 mmol) was dissolved in methanol (5 mL), and a solution of hydrogen chloride in dioxane (5 mL, 4 M) was added. The mixture was reacted at room temperature for two hours and spun to dryness to obtain title compound 13D (275 mg, crude).

LC-MS (ESI): m/z=265.2 [M+H]⁺.

### Step 4:

1H (150 mg, 0.67 mmol) and 13D (275 mg, 0.92 mmol) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH=1:0-10:1), to afford compound 13 (134 mg, 44.4%).

¹H NMR (400 MHz, DMSO-d₆) δ 11.83 (s, 1H), 8.53 (d, 1H), 8.47-8.37 (m, 1H), 8.24 (d, 1H), 7.75 (s, 1H), 7.69 (d, 1H), 7.62 (s, 1H), 3.66 (s, 2H), 3.23 (t, 4H), 2.90-2.83 (m, 1H), 2.61-2.56 (m, 4H), 2.54 (d, 2H), 1.18 (t, 3H), 0.72-0.66 (m, 2H), 0.66-0.59 (m, 2H).

LC-MS (ESI): m/z=451.2 [M+H]⁺.

### Example 14

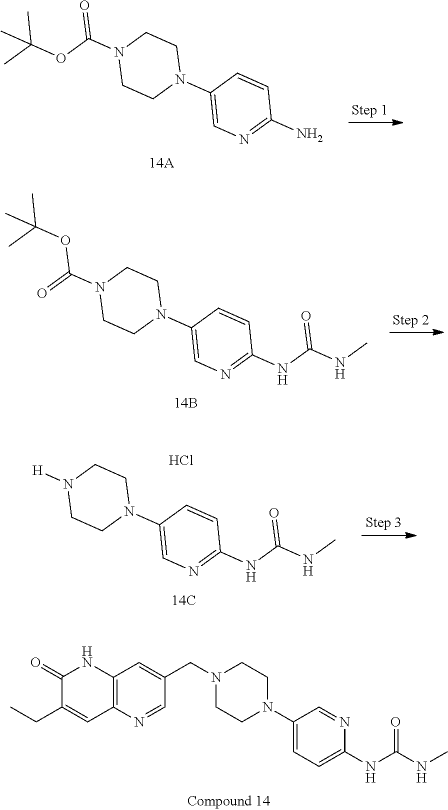

### Step 1:

Compound 14A (558 mg, 2 mmol) was dissolved in DMF (20 mL) under N₂ protection and cooled in an ice-water bath, sodium hydride (320 mg, 60%) was then added, and the mixture was stirred and reacted for 1 h in the ice bath. Subsequently, CDI (486 mg, 3 mmol) was added, and the mixture was stirred and reacted for another 30 min, at which time the color of the system was observed to become lighter. Excess methylamine tetrahydrofuran solution was finally added, and the mixture was reacted at room temperature for 2 h. After the reaction was completed, to the system was added ethyl acetate (100 mL), and the mixture was washed with water (100 mL×4). The organic phase was collected, dried over anhydrous sodium sulfate, filtered, evaporated to dryness, and separated by silica gel chromatographic column (PE:EA (v/v)=1:0-1:1), to afford title compound 14B (233 mg, 34.9%).

LC-MS (ESI): m/z=336.2 [M+H]⁺.

### Step 2:

14B (233 mg, 0.69 mmol) was dissolved in methanol (10 mL), and a solution of hydrogen chloride in dioxane (5 mL, 4 M) was added. The mixture was reacted at room temperature for two hours and spun to dryness to obtain title compound 14C (175 mg, crude).

LC-MS (ESI): m/z=236.2 [M+H]⁺.

### Step 3:

1H (120 mg, 0.54 mmol) and 14C (175 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, a product was generated, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH (v/v)=1:0-10:1), to afford compound 14 (104 mg, 45.8%).

¹H NMR (400 MHz, DMSO-d₆) δ 11.72 (s, 1H), 8.92 (s, 1H), 8.39 (d, 1H), 7.95-7.85 (m, 1H), 7.81 (d, 1H), 7.74 (s, 1H), 7.62 (d, 1H), 7.39 (dd, 1H), 7.21 (d, 1H), 3.64 (s, 2H), 3.07 (t, 4H), 2.70 (d, 3H), 2.57-2.54 (m, 4H), 2.54-2.51 (m, 2H), 1.18 (t, 3H).

LC-MS (ESI): m/z=422.2 [M+H]⁺.

### Example 15

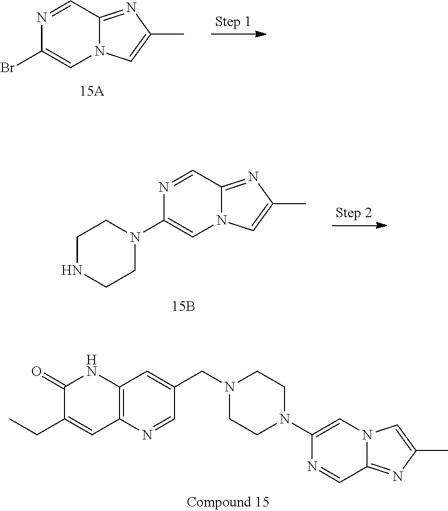 [SMILES: Cc1cn2cc(Br)ncc2n1 ; Cc1cn2cc(N3CCNCC3)ncc2n1 ; CCc1cc2ncc(CN3CCN(c4cn5cc(C)nc5cn4)CC3)cc2[nH]c1=O]

### Step 1:

Under nitrogen protection, 6-bromo-2-methylimidazolo[1,2-a]pyrazine (15A) (300 mg, 1.42 mmol), piperazine (610 mg, 7.08 mmol), tris(dibenzylideneacetone)dipalladium (129.6 mg, 0.142 mmol), 2-(di-tert-butylphosphine)biphenyl (63.3 mg, 0.212 mmol) and sodium tert-butoxide (271.7 mg, 2.83 mmol) were all added to a reaction flask, toluene (20 mL) was added, and the mixture was warmed to 110° C. and reacted overnight. After the reaction was completed as monitored by TLC, the reaction liquid was filtered through diatomaceous earth and washed with ethyl acetate. The organic phase was concentrated, and the resulting residue was separated and purified by silica gel column chromatography (dichloromethane:methanol:concentrated ammonia water (v/v/v)=100:10:0.5), to afford 15B (270 mg, 87.8%) as a pale yellow solid.

LC-MS (ESI): 218.2 [M+H]⁺.

### Step 2:

Compound 1H (50 mg, 0.225 mmol), 15B (53.7 mg, 0.247 mmol), N,N-diisopropylethylamine (145 mg, 2.24 mmol), and potassium iodide (3.71 mg, 0.0225 mmol) were all added to a reaction tube, dry acetonitrile (3 mL) was added, and the mixture was warmed to 80° C. and reacted for about 5 hours. After the reaction was completed as monitored by TLC, water (5 mL) was added, and the mixture was extracted with ethyl acetate (3 mL×10). The organic phases were combined, dried over anhydrous sodium sulfate, filtered and concentrated, to afford a crude. The crude was separated and purified by preparative HPLC with methods: 1. Instruments: waters 2767 preparative liquid chromatographic instrument; chromatographic column: SunFirm@ Prep C18 (19 mm×250 mm); 2. The sample was filtered with a 0.45 μm filter to prepare a sample liquid; 3. Preparative chromatography conditions: a. composition of mobile phases A and B: mobile phase A: acetonitrile; mobile phase B: water (containing 0.5% ammonium acetate); b. gradient elution, mobile phase A: 5%-50%; c. flow rate: 12 mL/min; d. elution time: 10 min; to afford compound 15 (22 mg, 24.3%).

¹H NMR (400 MHz, CD₃OD): δ 8.59 (s, 1H), 8.49 (d, 1H), 7.82 (s, 1H), 7.76 (d, 1H), 7.74 (d, 1H), 7.62 (s, 1H), 3.74 (s, 2H), 3.42-3.29 (m, 5H), 2.76-2.62 (m, 5H), 2.41 (s, 3H), 1.27 (t, 3H).

LC-MS (ESI): 404.1 [M+H]⁺

### Example 16

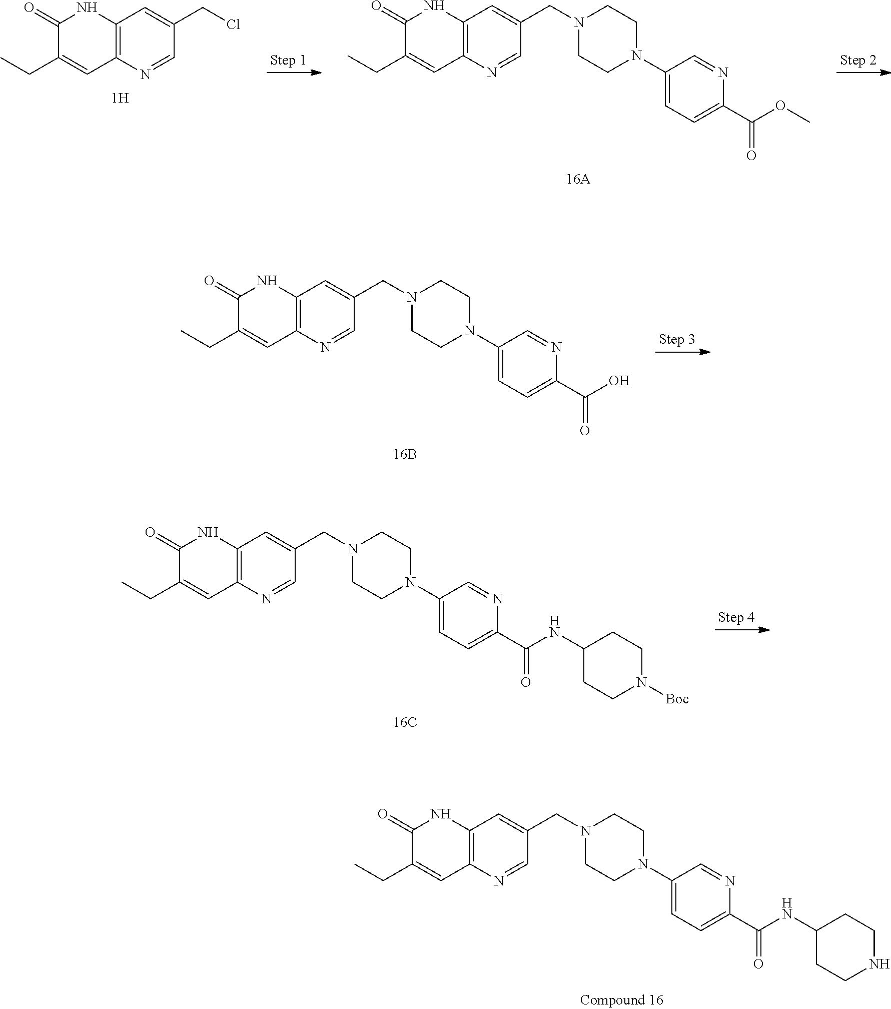 [SMILES: CCc1cc2ncc(CCl)cc2[nH]c1=O ; CCc1cc2ncc(CN3CCN(c4ccc(C(=O)OC)nc4)CC3)cc2[nH]c1=O ; CCc1cc2ncc(CN3CCN(c4ccc(C(=O)O)nc4)CC3)cc2[nH]c1=O ; CCc1cc2ncc(CN3CCN(c4ccc(C(=O)NC5CCN(C(=O)OC(C)(C)C)CC5)nc4)CC3)cc2[nH]c1=O ; CCc1cc2ncc(CN3CCN(c4ccc(C(=O)NC5CCNCC5)nc4)CC3)cc2[nH]c1=O]

### Step 1:

Intermediate 1H (3.20 g, 14.4 mmol), methyl 5-(piperazin-1-yl)picolinate (4.23 g, 14.4 mmol), potassium iodide (478 mg, 2.88 mmol) and N,N-diisopropylethylamine (12.5 ml, 71.96 mmol) were dissolved in acetonitrile (150 ml), and the mixture was reacted at 80° C. for 2 h at reflux. After the reaction was completed, the reaction liquid was concentrated, and extracted with water (80 ml), dichloromethane (150 ml) and methanol (15 ml). The organic phase was separated, and the aqueous phase was extracted with dichloromethane (150 ml×3). The organic phases were combined and concentrated, and the resulting residue was purified by silica gel column chromatography (eluent: methanol:dichloromnethane (v/v)=0%-15%), to afford compound 16A (4.24 g, 72.1%).

LCMS m/z=408.2 [M+H]⁺

### Step 2:

Compound 16A (4.24 g, 10.4 mmol) was dissolved in tetrahydrofuran (80 ml) and water (80 ml), lithium hydroxide (750 mg, 31.2 mmol) was added, and the mixture was stirred and reacted at room temperature for 2 h. After the reaction was completed, 1M hydrochloric acid was added to adjust the system to pH 4-5, the mixture was concentrated, and the resulting residue was purified by C18 column chromatography (eluent: methanol:water containing 0.1% trifluoroacetic acid=30%), to afford compound 16B (4.01 g, 100%) as a pale yellow solid.

LCMS m/z=394.21 [M+1]⁺

### Step 3:

Compound 16B (1 g, 2.54 mmol) and tert-butyl 4-aminopiperidine-1-carboxylate (609 mg, 3.05 mmol) were dissolved in DMF (5 mL), benzotriazole-N,N,N′,N′-tetramethyluronium hexafluorophosphate (962 mg, 2.54 mmol) and N,N-diisopropylethylamine (1.31 ml, 7.54 mmol) were added, and the mixture was stirred and reacted at room temperature for 1 h. After the reaction was completed, water (10 ml) and ethyl acetate (12 mL) were added. The organic phase was separated, and the aqueous phase was extracted with ethyl acetate (10 mL×2). The organic phases were combined, dried over anhydrous sodium sulfate and concentrated, and the resulting residue was separated and purified by silica gel column chromatography (eluent: methanol:dichloromethane=0%-15%), to afford product 16C (563 mg, 38.5%).

LCMS m/z=576.3 [M+1]⁺

### Step 4:

Compound 16C (563 mg, 0.98 mmol) was dissolved in dichloromethane (3 mL), trifluoroacetic acid (1 mL) was added, and the mixture was reacted at room temperature for 30 min. After the reaction was completed, the reaction liquid was concentrated, adjusted to pH>7 with triethylamine, and separated and purified by liquid phase preparative column after concentration (liquid phase preparative conditions: C18 reverse-phase preparative column, mobile phase: deionized water containing 0.1% aqueous ammonia (A) and acetonitrile (B), gradient elution, mobile phase B=5%-50%, elution time: 15 min, flow rate: 12 mL/min, and column temperature: 30° C.) to obtain the title compound 16 (351 mg, yield: 75.5%, retention time: about 6.5 min).

¹H NMR (400 MHz, CD3OD) δ 8.49 (d, 1H), 8.27 (d, 1H), 7.91 (d, 1H), 7.83 (s, 1H), 7.76 (d, 1H), 7.37 (d, 1H), 4.06-3.90 (m, 1H), 3.74 (s, 2H), 3.44-3.37 (m, 4H), 3.22-3.06 (m, 2H), 2.84-2.73 (m, 2H), 2.72-2.55 (m, 6H), 2.10-1.83 (m, 2H), 1.75-1.50 (m, 2H), 1.36-1.20 (m, 3H).

MS M/Z (ESI): m/z=476.2 [M+1]⁺

### Example 17

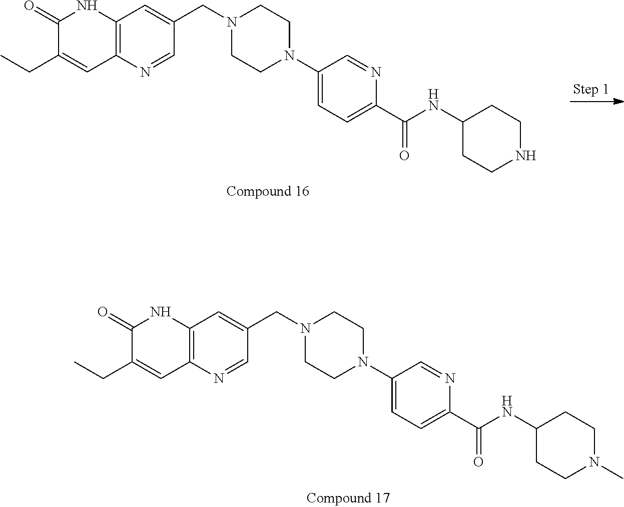 [SMILES: CCc1cc2ncc(CN3CCN(c4ccc(C(=O)NC5CCNCC5)nc4)CC3)cc2[nH]c1=O ; CCc1cc2ncc(CN3CCN(c4ccc(C(=O)NC5CCN(C)CC5)nc4)CC3)cc2[nH]c1=O]

### Step 1:

Compound 16 (50 mg, 0.11 mmol) and paraformaldehyde (50 mg) were added to methanol (2 mL), 1,2-dichloroethane (2 mL) was then added, three drops of glacial acetic acid were added dropwise, and the mixture was reacted at 60° C. for 12 hours. Subsequently, sodium cyanoborohydride (50 mg, 0.8 mmol) was added, and the mixture was reacted at room temperature for 1 h. The reaction liquid was spun to dryness, and then separated and purified by liquid phase preparative column (liquid phase preparative conditions: C18 reverse-phase preparative column, mobile phase: deionized water containing 0.1% ammonium bicarbonate (A) and acetonitrile (B), gradient elution, mobile phase B=5%-50%, elution time: 15 min, flow rate: 12 mL/min, and column temperature: 30° C.) to obtain the title compound 17 (30 mg, yield: 58%, retention time: about 8.1 min).

¹H NMR (400 MHz, DMSO-d₆) β 8.39 (s, 1H), 8.26 (s, 1H), 8.11 (d, 1H), 7.83 (d, 1H), 7.74 (s, 1H), 7.62 (s, 1H), 7.39 (d, 1H), 3.77-3.67 (m, 1H), 3.65 (s, 2H), 3.35-3.31 (m, 4H), 2.70 (d, 2H), 2.60-2.51 (m, 6H), 2.15 (s, 3H), 2.04-1.89 (m, 2H), 1.79-1.67 (m, 2H), 1.66-1.52 (m, 2H), 1.27-1.11 (m, 3H).

MS M/Z (ESI): m/z=490.3 [M+1]⁺

### Example 18

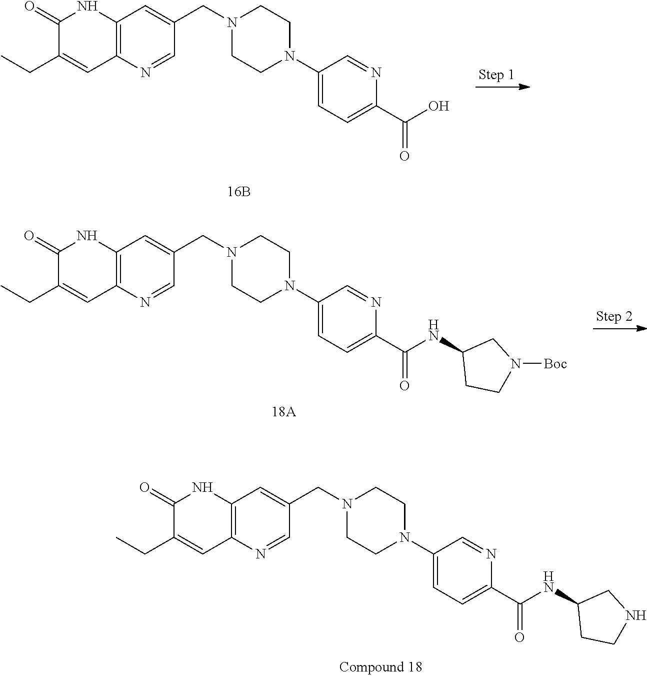 [SMILES: CCc1cc2ncc(CN3CCN(c4ccc(C(=O)O)nc4)CC3)cc2[nH]c1=O ; CCc1cc2ncc(CN3CCN(c4ccc(C(=O)N[C@@H]5CCN(C(=O)OC(C)(C)C)C5)nc4)CC3)cc2[nH]c1=O ; CCc1cc2ncc(CN3CCN(c4ccc(C(=O)N[C@@H]5CCNC5)nc4)CC3)cc2[nH]c1=O]

### Step 1:

Compound 16B (0.5 g, 1.27 mmol) and tert-butyl (R)-3-aminopyrrolidine-1-carboxylate (283 mg, 1.5 mmol) were dissolved in DMF (5 mL), benzotriazole-N,N,N′,N′-tetramethyluronium hexafluorophosphate (570 mg, 1.5 mmol) and N,N-diisopropylethylamine (330 mg, 2.54 mmol) were added, and the mixture was stirred and reacted at room temperature for 1 h. After the reaction was completed, water (10 ml) and ethyl acetate (10 mL) were added. The organic phase was separated, and the aqueous phase was extracted with ethyl acetate (10 mL×2). The organic phases were combined, dried over anhydrous sodium sulfate and concentrated, to afford crude product 18A (700 mg, 98%).

LCMS m/z=562.3 [M+1]⁺

### Step 2:

Compound 18A (700 mg, 1.27 mmol) was dissolved in dichloromethane (6 mL), trifluoroacetic acid (2 mL) was added, and the mixture was reacted at room temperature for 30 min. After the reaction was completed, the reaction liquid was concentrated, adjusted to pH>7 with triethylamine, and separated and purified by liquid phase preparative column (liquid phase preparative conditions: C18 reverse-phase preparative column, mobile phase: deionized water containing 0.1% aqueous ammonia (A) and acetonitrile (B), gradient elution, mobile phase B=5%-50%, elution time: 15 min, flow rate: 12 mL/min. and column temperature: 30° C.) to afford title compound 18 (400 ng, yield: 68%, retention time: about 6.6 min).

¹H NMR (400 MHz, DMSO-d₆) δ 8.40 (d, 1H), 8.26 (d, 2H), 7.83 (d, 1H), 7.75 (s, 1H), 7.63 (s, 1H), 7.39 (d, 1H), 4.33 (s, 1H), 3.65 (s, 2H), 3.37-3.31 (m, 4H), 3.06-2.79 (m, 3H), 2.77-2.62 (m, 2H), 2.60-2.52 (m, 6H), 2.04-1.95 (m, 1H), 1.70-1.51 (m, 1H), 1.22-1.14 (m, 3H).

MS M/Z (ESI): m/z=462.2 [M+1]⁺

### Example 19

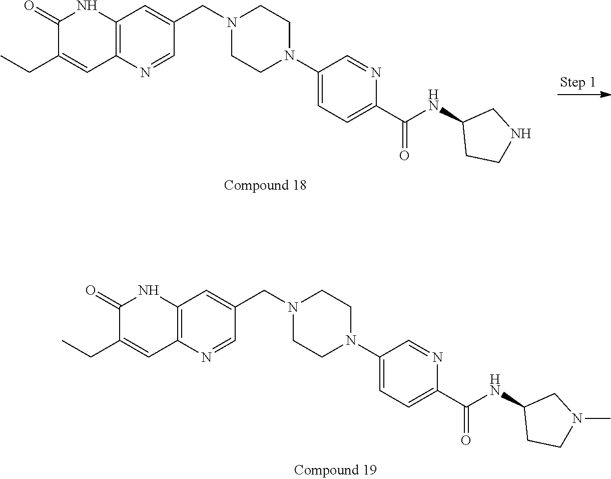 [SMILES: CCc1cc2ncc(CN3CCN(c4ccc(C(=O)N[C@@H]5CCNC5)nc4)CC3)cc2[nH]c1=O ; CCc1cc2ncc(CN3CCN(c4ccc(C(=O)N[C@@H]5CCN(C)C5)nc4)CC3)cc2[nH]c1=O]

### Step 1:

Compound 18 (50 mg, 0.11 mmol) and paraformaldehyde (50 mg) were added to methanol (2 mL), 1,2-dichloroethane (2 mL) was then added, three drops of glacial acetic acid were added dropwise, and the mixture was reacted at 60° C. for 12 hours. Subsequently, sodium cyanoborohydride (50 mg, 0.8 mmol) was added, and the mixture was reacted at room temperature for 1 h. The reaction liquid was spun to dryness, and then separated and purified by liquid phase preparative column (liquid phase preparative conditions: C18 reverse-phase preparative column, mobile phase: deionized water containing 0.1% ammonium acetate (A) and acetonitrile (B), gradient elution, mobile phase B=5%-50%, elution time: 15 min, flow rate: 12 mL/min, and column temperature: 30° C.) to afford title compound 19 (32 mg, yield: 58%, retention time: about 8.1 min).

¹H NMR (400 MHz, DMSO-d₆) δ 8.40 (d, 1H), 8.27 (d, 1H), 8.19 (d, 1H), 7.82 (d, 1H), 7.75 (s, 1H), 7.63 (s, 1H), 7.40 (d, 1H), 4.46-4.29 (m, 1H), 3.65 (s, 2H), 3.45-3.24 (m, 4H), 2.71-2.58 (m, 2H), 2.59-2.52 (m, 6H), 2.47-2.41 (m, 1H), 2.35-2.29 (m, 1H), 2.26 (s, 3H), 2.22-2.11 (m, 1H), 1.72-1.61 (m, 1H), 1.22-1.15 (m, 3H).

LCMS m/z (ESI): m/z=476.2 [M+1]⁺

### Example 20

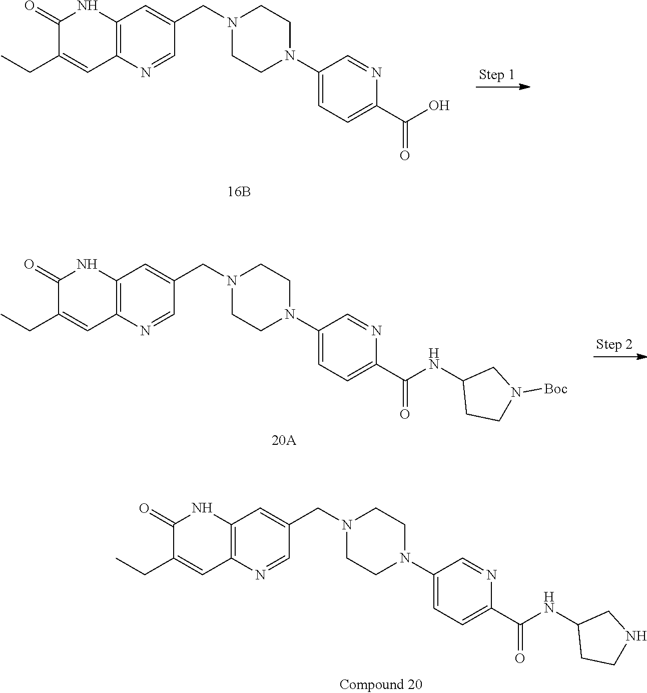 [SMILES: CCc1cc2ncc(CN3CCN(c4ccc(C(=O)O)nc4)CC3)cc2[nH]c1=O ; CCc1cc2ncc(CN3CCN(c4ccc(C(=O)NC5CCN(C(=O)OC(C)(C)C)C5)nc4)CC3)cc2[nH]c1=O ; CCc1cc2ncc(CN3CCN(c4ccc(C(=O)NC5CCNC5)nc4)CC3)cc2[nH]c1=O]

### Step 1:

Compound 16B (0.5 g, 1.27 mmol) and tert-butyl (S)-3-aminopyrrolidine-1-carboxylate (283 mg, 1.5 mmol) were dissolved in DMF (5 mL), benzotriazole-N,N,N′,N′-tetramethyluronium hexafluorophosphate (570 mg, 1.5 mmol) and N,N-diisopropylethylamine (330 mg, 2.54 mmol) were added, and the mixture was stirred and reacted at room temperature for 1 h. After the reaction was completed, water (10 ml) and ethyl acetate (10 mL) were added. The organic phase was separated, and the aqueous phase was extracted with ethyl acetate (10 mL×2). The organic phases were combined, dried over anhydrous sodium sulfate and concentrated, to afford crude product 20A (700 mg, 98%).

LCMS m/z=562.3 [M+1]⁺

### Step 2:

Compound 20A (700 mg, 1.27 mmol) was dissolved in dichloromethane (6 mL), trifluoroacetic acid (2 mL) was added, and the mixture was reacted at room temperature for 30 min. After the reaction was completed, the reaction liquid was concentrated, adjusted to pH>7 with triethylamine, and separated and purified by liquid phase preparative column (liquid phase preparative conditions: C18 reverse-phase preparative column, mobile phase: deionized water containing 0.1% aqueous ammonia (A) and acetonitrile (B), gradient elution, mobile phase B=5%-50%, elution time: 15 min. flow rate: 12 mL/min, and column temperature: 30° C.) to afford title compound 20 (401 mg, yield: 68%, retention time: about 6.6 min).

¹H NMR (400 MHz, DMSO-d6) δ 8.40 (d, 1H), 8.27 (s, 2H), 7.83 (d, 1H), 7.75 (s, 1H), 7.64 (s, 1H), 7.40 (d, 1H), 4.32 (s, 1H), 3.65 (s, 2H), 3.38-3.30 (m, 4H), 2.98-2.82 (m, 3H), 2.76-2.59 (m, 2H), 2.58-2.52 (m, 6H), 2.08-1.89 (m, 1H), 1.70-1.57 (m, 1H), 1.21-1.13 (m, 3H).

MS M/Z (ESI): m/z=462.2 [M+1]⁺

### Example 21

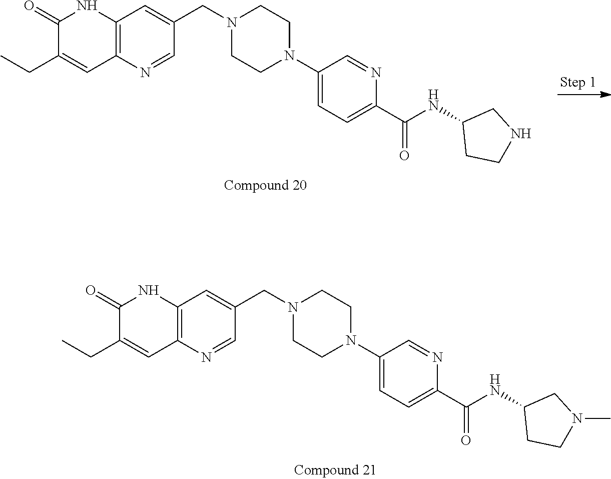 [SMILES: CCc1cc2ncc(CN3CCN(c4ccc(C(=O)N[C@H]5CCNC5)nc4)CC3)cc2[nH]c1=O ; CCc1cc2ncc(CN3CCN(c4ccc(C(=O)N[C@H]5CCN(C)C5)nc4)CC3)cc2[nH]c1=O]

### Step 1:

Compound 20 (50 mg, 0.11 mmol) and paraformaldehyde (50 mg) were added to methanol (2 mL), 1,2-dichloroethane (2 mL) was then added, three drops of glacial acetic acid were added dropwise, and the mixture was reacted at 60° C. for 12 hours. Subsequently, sodium cyanoborohydride (50 mg, 0.8 mmol) was added, and the mixture was reacted at room temperature for 1 h. The reaction liquid was spun to dryness, and then separated and purified by liquid phase preparative column (liquid phase preparative conditions: C18 reverse-phase preparative column, mobile phase: deionized water containing 0.1% ammonium acetate (A) and acetonitrile (B), gradient elution, mobile phase B=5%-50%, elution time: 15 min, flow rate: 12 mL/min, and column temperature: 30° C.) to obtain the title compound 21 (33 mg, yield: 58%, retention time: about 8.1 min).

¹H NMR (400 MHz, DMSO-d₆) δ 8.40 (d, 1H), 8.27 (d, 1H), 8.19 (d, 1H), 7.82 (d, 1H), 7.75 (s, 1H), 7.63 (d, 1H), 7.40 (d, 1H), 4.46-4.32 (m, 1H), 3.65 (s, 2H), 3.37-3.31 (m, 4H), 2.70-2.59 (m, 2H), 2.59-2.52 (m, 6H), 2.47-2.42 (m, 1H), 2.35-2.28 (m, 1H), 2.26 (s, 3H), 2.22-2.12 (m, 1H), 1.76-1.60 (m, 1H), 1.22-1.14 (m, 3H).

MS M/Z (ESI): m/z=476.2 [M+1]⁺

### Example 22

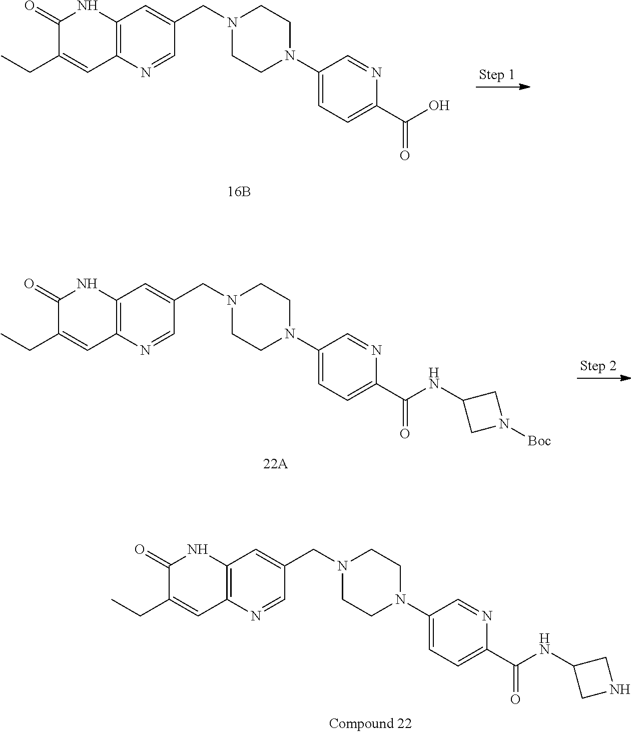 [SMILES: CCc1cc2ncc(CN3CCN(c4ccc(C(=O)O)nc4)CC3)cc2[nH]c1=O ; CCc1cc2ncc(CN3CCN(c4ccc(C(=O)NC5CN(C(=O)OC(C)(C)C)C5)nc4)CC3)cc2[nH]c1=O ; CCc1cc2ncc(CN3CCN(c4ccc(C(=O)NC5CNC5)nc4)CC3)cc2[nH]c1=O]

### Step 1:

Compound 16B (1 g, 2.54 mmol) and tert-butyl 3-aminoazetidine-1-carboxylate (525 mg, 3.05 mmol) were dissolved in DMF (5 mL), benzotriazole-N,N,N′,N′-tetramethyluronium hexafluorophosphate (962 mg, 2.54 mmol) and N,N-diisopropylethylamine (1.31 ml, 7.54 mmol) were added, and the mixture was stirred and reacted at room temperature for 1 h. After the reaction was completed, water (10 ml) and ethyl acetate (12 mL) were added. The organic phase was separated, and the aqueous phase was extracted with ethyl acetate (10 mL×2). The organic phases were combined, dried over anhydrous sodium sulfate and concentrated, and the resulting residue was separated and purified by silica gel column chromatography (eluent: methanol:dichloromethane=0%-15%), to afford product 22A (500 mg, 36%).

LCMS m/z=548.3 [M+1]+

### Step 2:

Compound 22A (500 mg, 0.91 mmol) was dissolved in dichloromethane (3 mL), trifluoroacetic acid (0 mL) was added, and the mixture was reacted at room temperature for 30 min. After the reaction was completed, the reaction liquid was concentrated, adjusted to pH>7 with triethylamine, and separated and purified by liquid phase preparative column after concentration (liquid phase preparative conditions: C18 reverse-phase preparative column, mobile phase: deionized water containing 0.1% aqueous ammonia (A) and acetonitrile (B), gradient elution, mobile phase B=5%-50%, elution time: 15 min. flow rate: 12 mL/min, and column temperature: 30° C.) to obtain the title compound 22 (300 mg, yield: 73%, retention time: about 6.5 min).

¹H NMR (400 MHz, CD₃OD) δ 8.49 (d, 1H), 8.30 (d, 1H), 7.90 (d, 1H), 7.83 (s, 1H), 7.76 (s, 1H), 7.37 (d, 1H), 3.95-3.90 (m, 1H), 3.88-3.83 (m, 1H), 3.74 (s, 2H), 3.44-3.39 (m, 3H), 3.31-3.30 (m, 4H), 2.75-2.56 (m, 6H), 1.32-1.20 (m, 3H).

MS M/Z (ESI): m/z=448.2 [M+1]⁺

### Example 23

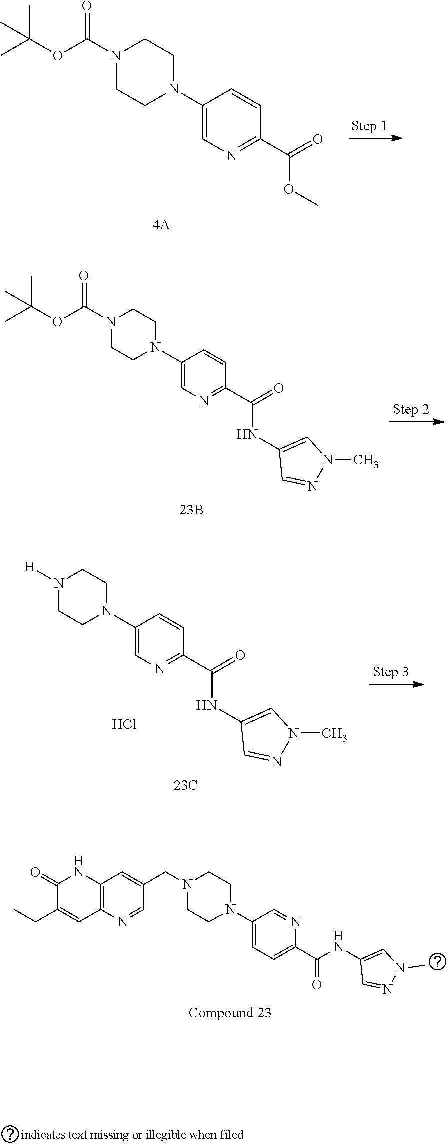

### Step 1:

Compound 4A (321 mg, 1 mmol) was dissolved in THF (10 mL) and H₂O (1 mL), LiOH·H₂O (45 mg, 1.1 mmol) was added, and the mixture was stirred and reacted at room temperature for 2 h. The solvent was removed by distillation under reduced pressure, to the resulting solid was added DMF (10 mL), HATU (570 mg, 1.5 mmol) was added under stirring, and the mixture was stirred at room temperature. After the solid was completely dissolved, DIEPA (1 mL) was added, 1-methyl-4-aminopyrazole hydrochloride (336 mg, 2 mmol was finally added, and the mixture was stirred overnight at room temperature. After the reaction was completed as monitored by LCMS, to the system was added ethyl acetate (50 mL), and the mixture was washed with water (50 mL×4). The organic phase was collected, dried over anhydrous sodium sulfate, filtered, evaporated to dryness, and separated by silica gel chromatographic column (PE:EA=1:0-1:1), to afford title compound 23B (351 mg, 90.6%).

LC-MS (ESI): m/z=387.2 [M+H]⁺.

### Step 2:

23B (351 mg, 0.90 mmol) was dissolved in methanol (5 mL), and a solution of hydrogen chloride in dioxane (5 mL, 4 M) was added. The mixture was reacted at room temperature for 4 hours and spun to dryness to obtain title compound 23C (274 mg, crude).

LC-MS (ESI): m/z=287.2 [M+H]⁺.

### Step 3:

1H (150 mg, 0.67 mmol) and 23C (274 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH (v/v)=1:0-10:1), to afford compound 23 (141 mg, 48.4%).

¹H NMR (400 MHz, DMSO-d₆) δ 12.19 (s, 1H), 10.55 (s, 1H), 8.56 (d, 1H), 8.39 (d, 1H), 8.04 (s, 1H), 7.98 (d, 1H), 7.81 (s, 1H), 7.79 (d, 1H), 7.71 (s, 1H), 7.53 (dd, 1H), 4.55 (s, 2H), 3.81 (s, 3H), 3.81-3.64 (m, 4H), 3.24-3.04 (m, 4H), 2.62-2.54 (m, 2H), 1.20 (t, 3H).

LC-MS (ESI): m/z=473.2 [M+H]⁺.

### Example 24

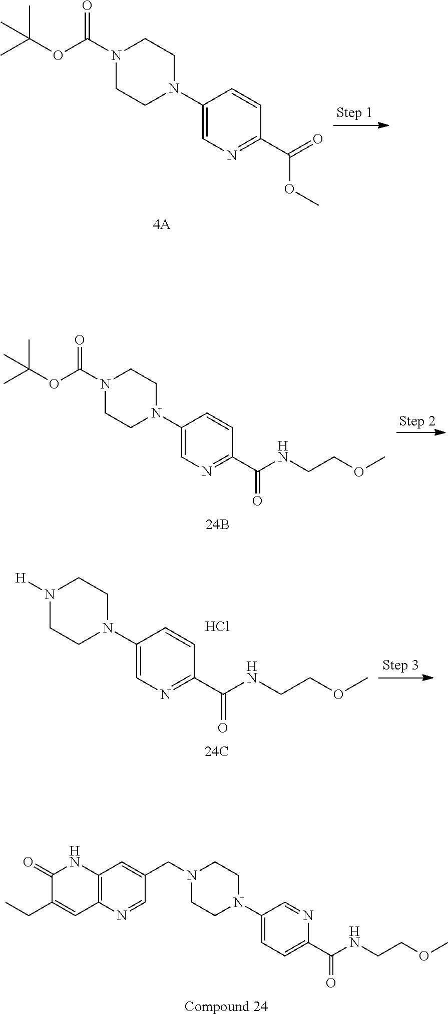

Compound 4A (321 mg, 1 mmol) was dissolved in THF (10 mL) and H₂O (1 mL), LiOH·H₂O (45 mg, 1.1 mmol) was added, and the mixture was stirred and reacted at room temperature for 2 h. The solvent was removed by distillation under reduced pressure, to the resulting solid was added DMF (10 mL) HATU (570 mg, 1.5 mmol) was added under stirring, and the mixture was stirred at room temperature. After the solid was completely dissolved, DIEPA (1 mL) was added, excess 2-methoxyethylamine was finally added, and the mixture was stirred overnight at room temperature. After the reaction was completed as monitored by LCMS, to the system was added water (50 mL) under stirring, at which time a large amount of white solid was observed to be precipitated out, and the mixture was stirred for another 10 min, filtered and dried under vacuum, to afford target compound 24B (282 mg, 77.5%) as a white plate-like crystal.

LC-MS (ESI): m/z=365.2 [M+H]⁺.

### Step 2:

24B (282 mg, 0.77 mmol) was dissolved in methanol (5 mL), and a solution of hydrogen chloride in dioxane (5 mL, 4 M) was added. The mixture was reacted at room temperature for 4 hours and spun to dryness to obtain title compound 24C (225 mg, crude).

LC-MS (ESI): m/z=265.2 [M+H]⁺.

### Step 3:

1H (120 mg, 0.54 mmol) and 24C (225 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH (v/v)=1:0-10:1), to afford compound 24 (91 mg, 37.6%).

¹H NMR (400 MHz, DMSO-d₆) δ 11.73 (s, 1H), 8.40 (d, 1H), 8.32 (d, 1H), 8.29 (s, 1H), 7.84 (d, 1H), 7.75 (s, 1H), 7.63 (d, 1H), 7.40 (dd, 1H), 3.65 (s, 2H), 3.47-3.40 (m, 4H), 3.35 (t, 4H), 3.27 (s, 3H), 2.61-2.51 (m, 4H), 2.55-2.50 (m, 2H), 1.19 (t, 3H).

LC-MS (ESI): m/z=451.2 [M+H]⁺.

### Example 25

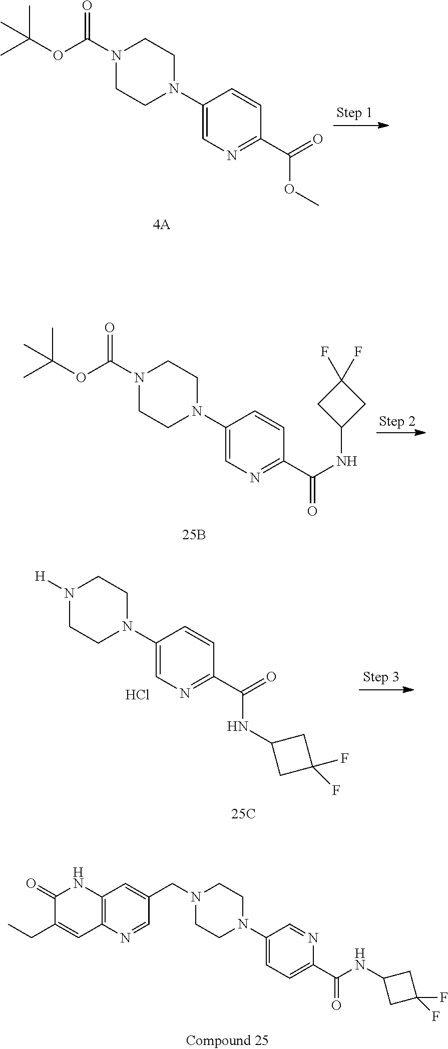 [SMILES: COC(=O)c1ccc(N2CCN(C(=O)OC(C)(C)C)CC2)cn1 ; CC(C)(C)OC(=O)N1CCN(c2ccc(C(=O)NC3CC(F)(F)C3)nc2)CC1 ; O=C(NC1CC(F)(F)C1)c1ccc(N2CCNCC2)cn1 ; Cl ; CCc1cc2ncc(CN3CCN(c4ccc(C(=O)NC5CC(F)(F)C5)nc4)CC3)cc2[nH]c1=O]

### Step 1:

Compound 4A (321 mg, 1 mmol) was dissolved in THF (10 mL) and H₂O (1 mL), LiOH·H₂O (45 mg, 1.1 mmol) was added, and the mixture was stirred and reacted at room temperature for 2 h. The solvent was removed by distillation under reduced pressure, to the resulting solid was added DMF (10 mL), HATU (570 mg, 1.5 mmol) was added under stirring, and the mixture was stirred at room temperature. After the solid was completely dissolved, DIEPA (1 mL) was added, 3,3-difluorocyclobutylamine (214 mg, 2 mmol) was finally added, and the mixture was stirred overnight at room temperature. After the reaction was completed as monitored by LCMS, to the system was added water (50 mL) under stirring, at which time a large amount of white solid was observed to be precipitated out, and the mixture was stirred for another 10 min, filtered and dried under vacuum, to afford target compound 25B (332 mg, 83.6%) as a white solid.

LC-MS (ESI): m/z=397.2 [M+H]⁺.

### Step 2:

25B (332 mg, 0.83 mmol) was dissolved in methanol (5 mL), and a solution of hydrogen chloride in dioxane (5 mL, 4 M) was added. The mixture was reacted at room temperature for 4 hours and spun to dryness to obtain title compound 25C (275 mg, crude).

LC-MS (ESI): m/z=297.2 [M+H]⁺.

### Step 3:

1H (160 mg, 0.72 mmol) and 25C (275 mg, crude) were dispersed in anhydrous acetonitrile (10 mL), potassium iodide (8 mg, 0.05 nmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH=1:0-10:1), to afford compound 25 (114 mg, 23.6%).

¹H NMR (400 MHz, DMSO-d₆) δ 11.82 (s, 1H), 8.91 (d, 1H), 8.41 (d, 1H), 8.27 (s, 1H), 7.83 (d, 1H), 7.75 (d, 1H), 7.63 (d, 1H), 7.40 (dd, 1H), 4.36-4.21 (m, 1H), 3.66 (s, 2H), 3.36 (t, 4H), 2.93-2.80 (m, 4H), 2.64-2.55 (m, 4H), 2.55-2.51 (m, 2H), 1.18 (t, 3H).

LC-MS (ESI): m/z=483.2 [M+H]⁺.

### Example 26

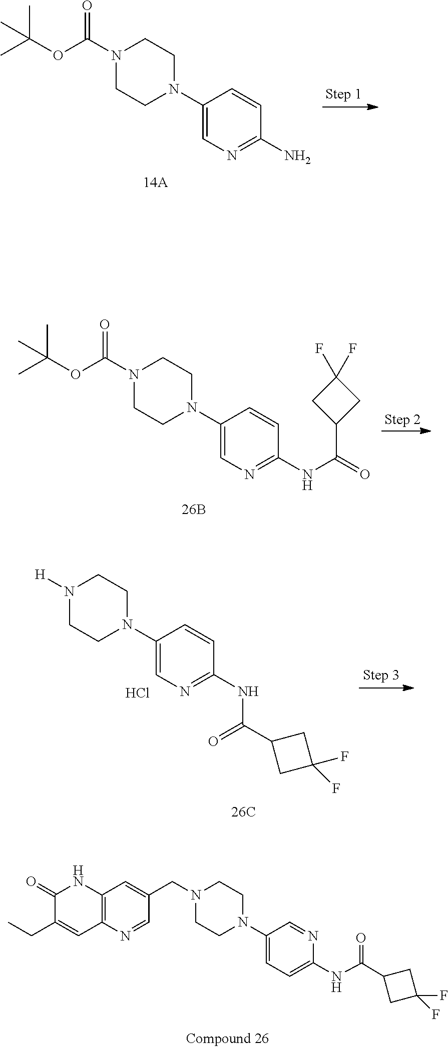 [SMILES: CC(C)(C)OC(=O)N1CCN(c2ccc(N)nc2)CC1 ; CC(C)(C)OC(=O)N1CCN(c2ccc(NC(=O)C3CC(F)(F)C3)nc2)CC1 ; O=C(Nc1ccc(N2CCNCC2)cn1)C1CC(F)(F)C1 ; Cl ; CCc1cc2ncc(CN3CCN(c4ccc(NC(=O)C5CC(F)(F)C5)nc4)CC3)cc2[nH]c1=O]

### Step 1:

3,3-Difluorocyclobutane-1-carboxylic acid (214 mg, 2 mmol) was dissolved in DMF (10 mL), HATU (1140 mg, 3 mmol) was added under stirring, and the mixture was stirred at room temperature. After the solid was completely dissolved, DIEPA (1 mL) was added, 14A (278 mg, 1 mmol) was finally added, and the mixture was stirred overnight at room temperature. After the reaction was completed as monitored by LCMS, to the system was added 50 mL of ethyl acetate, and the mixture was washed with water (50 mL×4). The organic phase was collected, dried over anhydrous sodium sulfate, filtered, evaporated to dryness, and separated by silica gel chromatographic column (PE:EA=1:0-1:1), to afford title compound 26B (254 mg, 64.1%) as a pale yellow solid.

LC-MS (ESI): m/z=397.2 [M+H]⁺.

### Step 2:

26B (254 mg, 0.64 mmol) was dissolved in methanol (5 mL), and a solution of hydrogen chloride in dioxane (5 mL, 4 M) was added. The mixture was reacted at room temperature for two hours and spun to dryness to obtain title compound 26C (197 mg, crude).

LC-MS (ESI): m/z=297.2 [M+H]⁺.

### Step 3:

1H (120 mg, 0.54 mmol) and 26C (197 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, a product was generated, the system was concentrated, saturated sodium bicarbonate solution (20 mL) was added, and the mixture was extracted with a mixed solution of DCM:MeOH (v/v)=10:1 (10 mL×3). The organic phases were combined, dried over anhydrous sodium sulfate, concentrated, and passed through column (DCM:MeOH (v/v)=1:0-10:1), to afford compound 26 (84 mg, 32.3%).

¹H NMR (400 MHz, DMSO-d₆) δ 11.83 (s, 1H), 10.41 (s, 1H), 8.40 (d, 1H), 8.00 (d, 1H), 7.96 (d, 1H), 7.75 (s, 1H), 7.63 (d, 1H), 7.40 (dd, 1H), 3.64 (s, 2H), 3.26-3.17 (m, 1H), 3.14 (t, 4H), 2.86-2.68 (m, 4H), 2.58-2.54 (m, 4H), 2.54-2.50 (m, 2H), 1.18 (t, 3H).

LC-MS (ESI): m/z=483.2 [M+H]⁺.

### Example 27

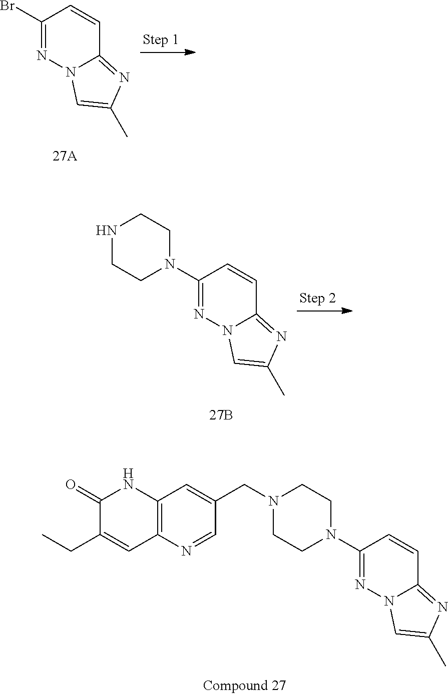 [SMILES: Cc1cn2nc(Br)ccc2n1 ; Cc1cn2nc(N3CCNCC3)ccc2n1 ; CCc1cc2ncc(CN3CCN(c4ccc5nc(C)cn5n4)CC3)cc2[nH]c1=O]

### Step 1:

6-Bromo-2-methylimidazolo[1,2-b]pyridazine (200 mg, 0.94 mmol), piperazine (89 mg, 1.07 mmol), Pd₂(dba)₃ (26 mg, 0.028 mmol), JohnPhos (12.45 mg, 0.028 mmol), sodium tert-butoxide (226 mg, 2.35 mmol) and toluene (10 mL) were added to a reaction flask, and the mixture was stirred at 100° C. under N₂ protection for 5 h. After the reaction was completed, the reaction liquid was concentrated under reduced pressure, extracted three times with ethyl acetate, washed with saturated sodium chloride solution, dried over anhydrous sodium sulfate, and concentrated under reduced pressure, to afford a crude. The crude was subjected to flash column chromatography (MeOH:DCM=9%), to afford light brown 27B (119 mg, 0.55 mmol, yield: 58.3%).

### Step 2:

27B (119 mg, 0.55 mmol), 7-(chloromethyl)-3-ethyl-1,5-naphthyridin-2(1H)-one (122.47 mg, 0.55 mmol), diisopropylethylamine (213 mg, 1.65 mmol), and KI (46 mg, 0.28 mmol) were added to a reaction flask, and the mixture was stirred at 80° C. for 5 h. After the reaction was completed, the reaction system was directly subjected to preparative HPLC with preparative HPLC separation methods: 1. Instruments: waters 2767 preparative liquid chromatographic instrument; chromatographic column: SunFire@ Prep C18 (19 mm×250 mm); 2. The sample was filtered with a 0.45 μm filter to prepare a sample liquid; 3. Preparative chromatography conditions: a. composition of mobile phases A and B: mobile phase A: acetonitrile; mobile phase B: water (containing 0.1% ammonium acetate); b. gradient elution, mobile phase A: 10%-55%; c. flow rate: 12 mL/min; retention time: 7.0 min, to obtain title compound 27 (17 mg, 8%).

LCMS m/z=404.2 [M+H]⁺

¹H NMR (400 MHz, DMSO-d₆) δ 11.82 (s, 1H), 8.40 (d, 1H), 7.75 (s, 1H), 7.70 (d, 1H), 7.64 (d, 2H), 7.07 (d, 1H), 3.65 (s, 2H), 3.45 (s, 4H), 2.57-2.52 (m, 6H), 2.28 (s, 3H), 1.18 (t, 3H).

### Example 28

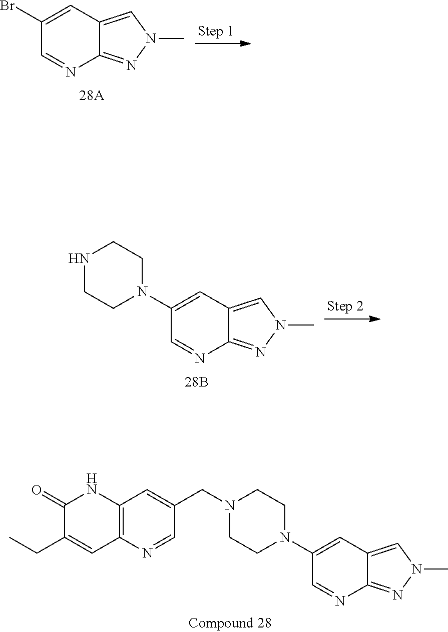 [SMILES: Cn1cc2cc(Br)cnc2n1 ; Cn1cc2cc(N3CCNCC3)cnc2n1 ; CCc1cc2ncc(CN3CCN(c4cnc5nn(C)cc5c4)CC3)cc2[nH]c1=O]

### Step 1:

5-Bromo-2-methyl-2H-pyrazolo[3,4-b]pyridine (200 mg, 0.94 mmol), piperazine (178 mg, 2.07 mmol), Pd₂(dba)₃ (26 mg, 0.028 mmol), JohnPhos (12.45 mg, 0.028 mmol), sodium tert-butoxide (226 mg, 2.35 mmol) and toluene (10 mL) were added to a reaction flask, and the mixture was stirred at 100° C. under N₂ protection for 5 h. After the reaction was completed, the reaction liquid was concentrated under reduced pressure, extracted three times with ethyl acetate, washed with saturated sodium chloride solution, dried over anhydrous sodium sulfate, and concentrated under reduced pressure, to afford a crude. The crude was subjected to flash column chromatography (MeOH:DCM=0.9%), to afford light brown 28B (130 mg, 0.55 mmol, yield: 63.7%).

### Step 2:

28B (140 mg, 0.64 mmol), 7-(chloromethyl)-3-ethyl-1,5-naphthyridine-2(1H)-one (143 mg, 0.64 mmol), diisopropylethylamine (248 mg, 1.92 mmol), and KI (53 mg, 0.32 mmol) were added to a reaction flask, and the mixture was stirred at 100° C. for 5 h. After the reaction was completed, the reaction system was directly subjected to preparative HPLC with preparative HPLC separation methods: 1. Instruments: waters 2767 preparative liquid chromatographic instrument; chromatographic column: SunFire@ Prep C18 (19 mm×250 mm); 2. The sample was filtered with a 0.45 μm filter to prepare a sample liquid; 3. Preparative chromatography conditions: a. composition of mobile phases A and B: mobile phase A: acetonitrile; mobile phase B: water (containing 0.1% ammonium acetate); b. gradient elution, mobile phase A: 10%-55%; c. flow rate: 12 mL/min; retention time: 7.0 min, to obtain title compound 28 (17 mg, 7%).

LCMS m/z=404.2 [M+1]⁺

¹H NMR (400 MHz, DMSO-d₆) δ 11.82 (s, 1H), 8.53 (d, 1H), 8.41 (d, 1H), 8.14 (s, 1H), 7.75 (s, 1H), 7.63 (s, 1H), 7.34 (d, 1H), 4.12 (s, 3H), 3.66 (s, 2H), 3.12 (s, 4H), 2.61 (d, 4H), 2.58-2.52 (m, 2H), 1.18 (t, 3H).

### Example 29

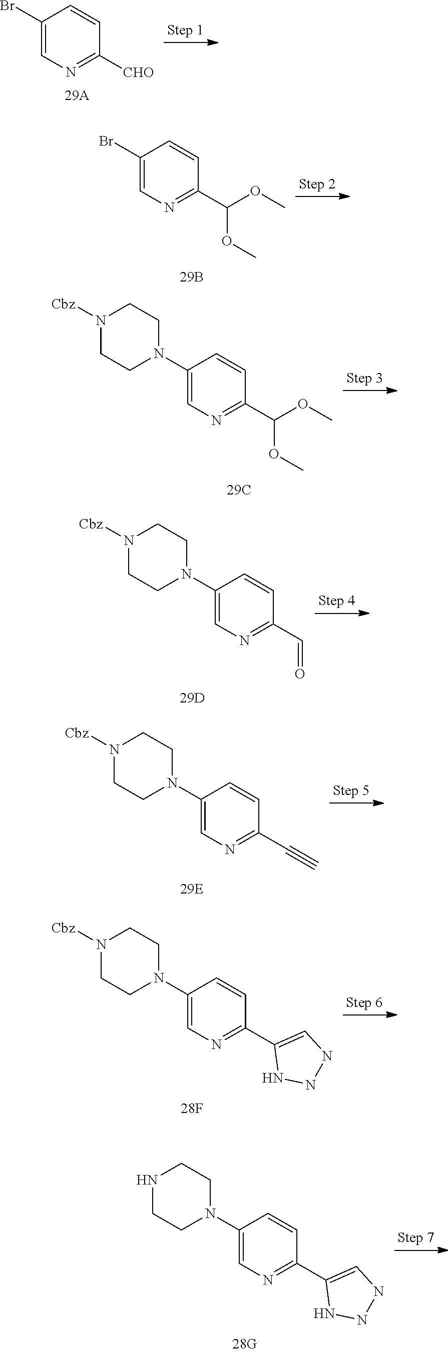

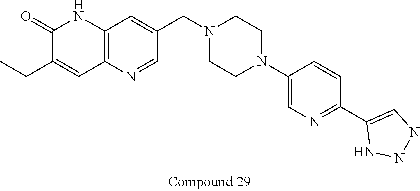 [SMILES: CCc1cc2ncc(CN3CCN(c4ccc(C5=CNNN5)nc4)CC3)cc2[nH]c1=O]

### Step 1:

Compound 29A (1.5 g, 8.04 mmol) was dissolved in methanol (30 mL), trimethoxymethane (3.41 g, 32.16 mmol) and p-toluenesulfonic acid (0.028 g, 0.16 mmol) were added, and the mixture was warmed to 75° C. and reacted for 3 h. After the reaction was completed, the reaction system was diluted with ethyl acetate (50 ml). The organic phase was washed with water (10 ml), dried over anhydrous sodium sulfate, filtered and concentrated, and the residue was separated and purified by flash column chromatography (eluent: MeOH:DCM=0%-10%), to afford compound 29B (1.5 g, 80%).

LC-MS (ESI): m/z=232.1 [M+1]⁺

### Step 2:

Under nitrogen protection, compound 29B (1.5 g, 6.46 mmol) and benzyl piperazine-1-carboxylate (1.71 g, 7.75 mmol) were dissolved in 1,4-dioxane (30 mL), methanesulfonic acid (2-dicyclohexylphosphino-2′,6′-diisopropoxy-1,1′-biphenyl)(2-amino-1,1′-biphenyl-2-yl)palladium (II) (0.27 g, 0.32 mmol) and cesium carbonate (6.31 g, 19.38 mmol) were added, and the mixture was warmed to 100° C. and reacted overnight. After the reaction was completed, the reaction system was diluted with ethyl acetate (50 ml). The organic phase was washed with water (10 ml), dried over anhydrous sodium sulfate, filtered and concentrated, and the residue was separated and purified by flash column chromatography (eluent: MeOH:DCM=0%-10%), to afford compound 29C (1.3 g, 54%).

LC-MS (ESI): m/z=372.3 [M+1]⁺

### Step 3:

Compound 29C (1.3 g, 3.50 mmol) was dissolved in tetrahydrofuran (10 mL), 4M hydrochloric acid (0.64 g, 17.57 mmol) was added, and the mixture was warmed to 50° C. and reacted for 5 h. After the reaction was completed, the reaction system was adjusted to pH 6 with sodium bicarbonate and diluted with ethyl acetate (50 ml). The organic phase was washed with water (10 ml), dried over anhydrous sodium sulfate, filtered and concentrated, and the residue was separated and purified by flash column chromatography (eluent: MeOH:DCM=0%-10%), to afford compound 29D (1.1 g, 97%).

LC-MS (ESI): m/z=326.2 [M+1]⁺

### Step 4:

Compound 29D (800 mg, 2.46 mmol) was dissolved in methanol (10 mL), dimethyl l-diazo-2-oxopropylphosphonate (0.71 g, 3.70 mmol) and potassium carbonate (0.68 g, 4.92 mmol) were added, and the mixture was reacted at room temperature for 12 h. After the reaction was completed, the reaction liquid was filtered, the organic phase was collected and concentrated, and the residue was separated and purified by flash column chromatography (eluent: MeOH:DCM=0%-10%), to afford compound 29E (0.75 g, 95%).

LC-MS (ESI): m/z=322.2 [M+1]⁺

### Step 5:

Compound 29E (750 mg, 2.33 mmol) and azidotrimethylsilane (0.54 g, 4.66 mmol) were added to DMF (8 mL), copper sulfate pentahydrate (II) (0.12 g, 0.47 mmol), L-sodium ascorbate (0.18 g, 0.93 mmol) and water (2 mL) were added, and the mixture was warmed to 100° C. and reacted for 2 h. After the reaction was completed, the reaction system was diluted with ethyl acetate (20 ml). The organic phase was washed with water (5 ml), dried over anhydrous sodium sulfate, filtered and concentrated, and the residue was separated and purified by flash column chromatography (eluent: MeOH:DCM=0%-10%), to afford compound 29F (0.75 g, 88%).

LC-MS (ESI): m/z=365.2 [M+1]⁺

### Step 6:

Under hydrogen protection, compound 29F (0.75 g, 2.06 mmol) was dissolved in methanol (20 mL), palladium on carbon (0.033 g, 0.31 mmol) and palladium hydroxide (0.043 g, 0.31 mmol) were added, and the mixture was reacted at room temperature for 5 h. After the reaction was completed, the reaction liquid was filtered, the organic phase was collected and concentrated, and the residue was separated and purified by flash column chromatography (eluent: MeOH:DCM=0%-10%), to afford compound 29G (0.35 g, 74%).

LC-MS (ESI): m/z=231.2 [M+1]⁺

### Step 7:

Under nitrogen protection, compound 29G (0.12 g, 0.54 mmol) was added to a flask, DMSO (2 mL) was added, followed by 7-(chloromethyl)-3-ethyl-1,2-dihydro-1,5-naphthyridin-2-one (100 mg, 0.45 mmol), ethyl diisopropylamine (0.35 g, 2.7 mmol) and potassium iodide (0.015 g, 0.090 mmol), and the mixture was warmed to 100° C. and reacted at this temperature for 1 h. After the reaction was completed as monitored by LCMS, the reaction system was directly subjected to preparative HPLC with preparative HPLC separation methods: 1. Instruments: waters 2767 preparative liquid chromatographic instrument; chromatographic column: SunFire@ Prep C18 (19 mm×250 mm); 2. The sample was filtered with a 0.45 μm filter to prepare a sample liquid; 3. Preparative chromatography conditions: a. composition of mobile phases A and B: mobile phase A: acetonitrile; mobile phase B: water (containing 0.1% ammonium acetate); b. gradient elution, mobile phase A: 10%-55%; c. flow rate: 12 mL/min. d. elution time: 20 min; retention time: 7.0 min, to afford compound 29 (40 mg, 21%).

LC-MS (ESI): m/z=417.3 [M+1]⁺

¹H NMR (400 MHz, DMSO-d₆) δ 14.98 (s, 1H), 11.82 (s, 1H), 8.41 (d, 1H), 8.32 (d, 1H), 8.15 (s, 1H), 7.84-7.76 (m, 1H), 7.75 (d, 1H), 7.64 (d, 1H), 7.42 (dd, 1H), 3.66 (s, 2H), 3.27 (d, 4H), 2.62-2.56 (m, 4H), 2.54 (dd, 2H), 1.18 (t, 3H).

### Example 30

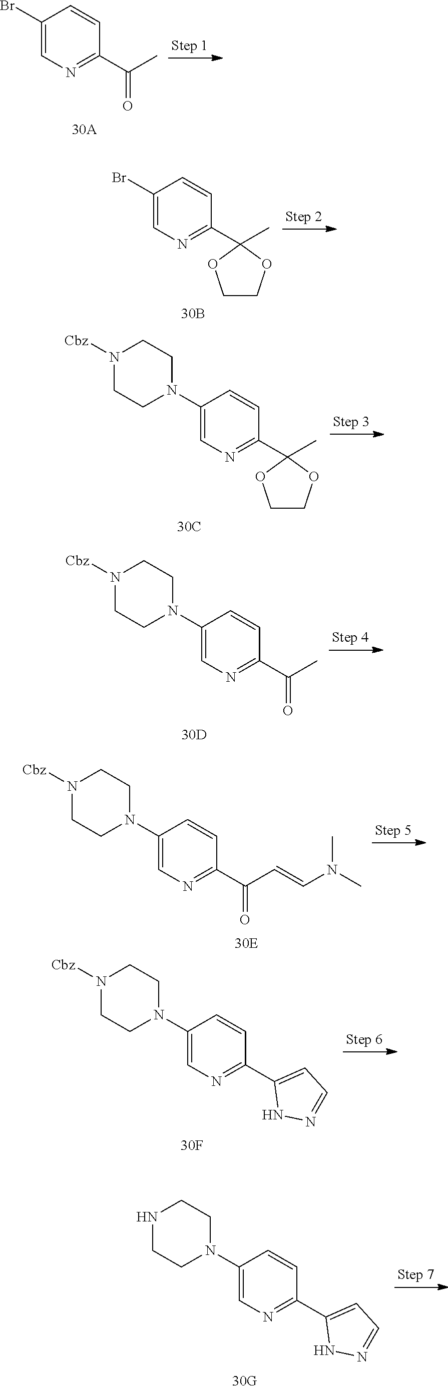

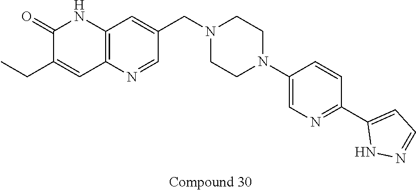 [SMILES: CCc1cc2ncc(CN3CCN(c4ccc(-c5ccn[nH]5)nc4)CC3)cc2[nH]c1=O]

### Step 1:

Compound 30A (1.5 g, 7.50 mmol) and ethylene glycol (1.40 g, 22.5 mmol) were dissolved in toluene (25 mL), p-toluenesulfonic acid (2.58 g, 15 mol) was added, and the mixture was heated to reflux for 10 h. After the reaction was completed, the reaction system was diluted with ethyl acetate (30 ml). The organic phase was washed with saturated sodium bicarbonate (10 ml), dried over anhydrous sodium sulfate, filtered and concentrated, and the residue was separated and purified by flash column chromatography (eluent: MeOH:DCM=0%-10%), to afford compound 30B (1.5 g, 82%).

LC-MS (ESI): m/z=244.0 [M+1]⁺

### Step 2:

Under nitrogen protection, compound 30B (1.5 g, 6.15 mmol) and benzyl piperazine-1-carboxylate (1.63 g, 7.38 mmol) were dissolved in 1,4-dioxane (20 mL), methanesulfonic acid (2-dicyclohexylphosphino-2,6′-diisopropoxy-1,1′-biphenyl)(2-amino-1,1-biphenyl-2-yl)palladium (II) (0.26 g, 0.31 mmol) was added, and the mixture was warmed to 100° C. and reacted overnight. After the reaction was completed, the reaction system was diluted with ethyl acetate (30 ml). The organic phase was washed with water (10 ml), dried over anhydrous sodium sulfate, filtered and concentrated, and the residue was separated and purified by flash column chromatography (eluent: MeOH:DCM=0%-10%), to afford compound 30C (1.8 g, 76%).

LC-MS (ESI): m/z=384.2 [M+1]⁺

### Step 3:

Compound 30C (1.8 g, 4.69 mmol) was dissolved in tetrahydrofuran (20 mL), 4M hydrochloric acid (0.17 g, 4.69 mmol) was added, and the mixture was warmed to 100° C. and reacted for 2 h. After the reaction was completed, the reaction system was adjusted to pH 6 with sodium bicarbonate and diluted with ethyl acetate (50 ml). The organic phase was washed with water (10 ml), dried over anhydrous sodium sulfate, filtered and concentrated, and the residue was separated and purified by flash column chromatography (eluent: MeOH:DCM=0%-10%), to afford compound 30D (1.3 g, 82%).

LC-MS (ESI): m/z=340.1 [M+1]⁺

### Step 4:

Compound 30D (500 mg, 1.47 mmol) was added to (dimethoxymethyl)dimethylamine (9.00 g, 75.53 mmol), and the mixture was warmed to 110° C. and reacted for 15 h. After the reaction was completed, the reaction liquid was directly concentrated, to afford crude compound 30E (0.5 g).

LC-MS (ESI): m/z=395.2 [M+1]⁺

### Step 5:

Compound 30E (500 mg, 1.27 mmol) was dissolved in ethanol (10 mL), hydrazine hydrate (0.64 g, 12.79 mmol) was added, and the mixture was warmed to 85° C. and reacted for 2 h. After the reaction was completed, the reaction liquid was directly concentrated, and the residue was separated and purified by flash column chromatography (eluent: MeOH:DCM=0%-10%), to afford compound 30F (0.42 g, 91%).

LC-MS (ESI): m/z=364.1 [M+1]⁺

### Step 6:

Under hydrogen protection, compound 30F (300 mg, 0.83 mmol) was dissolved in methanol (10 mL), palladium on carbon (0.013 g, 0.12 mmol) and palladium hydroxide (0.017 g, 0.12 mmol) were added, and the mixture was reacted overnight at room temperature. After the reaction was completed, the reaction liquid was filtered, the organic phase was collected and concentrated, and the residue was separated and purified by flash column chromatography (eluent: MeOH:DCM=0%-10%), to afford compound 30G (0.15 g, 79%).

LC-MS (ESI): m/z=230.1 [M+1]⁺

### Step 7:

Under nitrogen protection, compound 30G (0.07 g, 0.31 mmol) was added to a flask, DMSO (2 mL) was added, followed by 7-(chloromethyl)-3-ethyl-1,2-dihydro-1,5-naphthyridin-2-one (0.07 g, 0.31 mmol), ethyl diisopropylamine (0.24 g, 1.86 mmol) and potassium iodide (0.01 g, 0.062 mmol), and the mixture was warmed to 100° C. and reacted at this temperature for 1 h. After the reaction was completed as monitored by LCMS, the reaction system was directly subjected to preparative HPLC with preparative HPLC separation methods: 1. Instruments: waters 2767 preparative liquid chromatographic instrument; chromatographic column: SunFire@ Prep C18 (19 mm×250 mm); 2. The sample was filtered with a 0.45 μm filter to prepare a sample liquid; 3. Preparative chromatography conditions: a. composition of mobile phases A and B: mobile phase A: acetonitrile; mobile phase B: water (containing 0.1% ammonium acetate); b. gradient elution, mobile phase A: 10%-55%; c. flow rate: 12 mL/min. d. elution time: 20 min; retention time: 7.0 min, to afford compound 30 (30 mg, 23%).

LC-MS (ESI): m/z=416.2 [M+1]⁺

¹H NMR (400 MHz, DMSO-d₆) δ 12.79 (s, 1H), 11.83 (s, 1H), 8.41 (d, 1H), 8.28 (d, 1H), 7.88-7.53 (m, 4H), 7.38 (dd, 1H), 6.68 (d, 1H), 3.67 (s, 2H), 3.28-3.12 (m, 4H), 2.68-2.56 (m, 4H), 2.55 (dd, 2H), 1.19 (t, 3H).

### Example 31

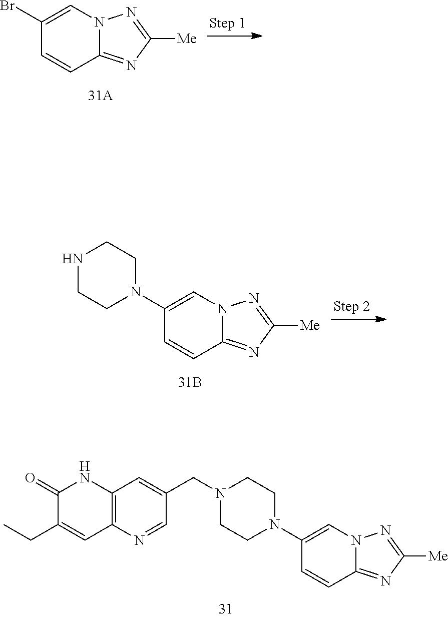 [SMILES: Cc1nc2ccc(Br)cn2n1 ; Cc1nc2ccc(N3CCNCC3)cn2n1 ; CCc1cc2ncc(CN3CCN(c4ccc5nc(C)nn5c4)CC3)cc2[nH]c1=O]

### Step 1:

Under nitrogen protection, compound 31A (100 mg, 0.47 mmol) and piperazine (49 mg, 0.57 mmol) were dissolved in 1,4-dioxane (5 mL), cesium carbonate (460 mg, 1.41 mmol) and methanesulfonic acid (2-dicyclohexylphosphino-2′,6′-diisopropoxy-1,1′-biphenyl)(2-amino-1,1′-biphenyl-2-yl)palladium (II) (20 mg, 0.024 mmol) were added, and the mixture was warmed to 100° C. and reacted overnight. After the reaction was completed, the reaction system was diluted with ethyl acetate (10 ml). The organic phase was washed with water (2 ml), dried over anhydrous sodium sulfate, filtered and concentrated, and the residue was separated and purified by flash column chromatography (eluent: MeOH:DCM=0%-10%), to afford compound 31B (0.05 g, 49%).

LC-MS (ESI): m/z=218.1 [M+1]⁺

### Step 2:

Under nitrogen protection, compound 31B (50 mg, 0.22 mmol) was added to a flask, DMSO (2 mL) was added, followed by 7-(chloromethyl)-3-ethyl-1,2-dihydro-1,5-naphthyridin-2-one (47.8 mg, 0.22 mmol), ethyl diisopropylamine (170 mg, 1.32 mmol) and potassium iodide (7.3 mg, 0.043 mmol), and the mixture was warmed to 100° C. and reacted at this temperature for 1 h. After the reaction was completed as monitored by LCMS, the reaction system was directly subjected to preparative HPLC with preparative HPLC separation methods: 1. Instruments: waters 2767 preparative liquid chromatographic instrument; chromatographic column: SunFire@ Prep C18 (19 mm×250 mm); 2. The sample was filtered with a 0.45 μm filter to prepare a sample liquid; 3. Preparative chromatography conditions: a. composition of mobile phases A and B: mobile phase A: acetonitrile; mobile phase B: water (containing 0.1% ammonium acetate); b. gradient elution, mobile phase A: 10%-55%; c. flow rate: 12 mL/min. d. elution time: 20 min; retention time: 7.0 min, to afford compound 31 (23 mg, 26%).

LC-MS (ESI): m/z=404.2 [M+1]⁺

¹H NMR (400 MHz, DMSO-d₆) δ 11.83 (s, 1H), 8.40 (d, 1H), 8.19 (dd, 1H), 7.75 (d, 1H), 7.63 (d, 1H), 7.59-7.52 (m, 2H), 3.66 (s, 2H), 3.12 (t, 4H), 2.61-2.51 (m, 6H), 2.40 (s, 3H), 1.18 (t, 3H).

### Example 32

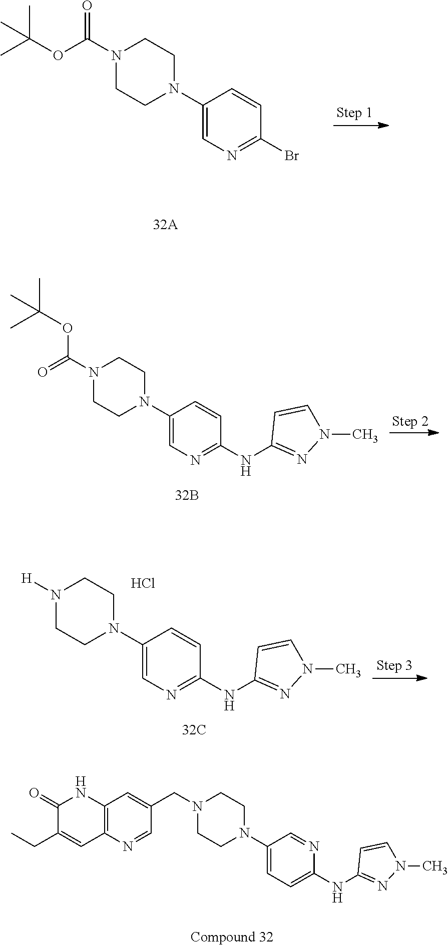

### Step 1:

Compound tert-butyl 4-(6-bromopyridin-3-yl)piperazine-1-carboxylate (321 mg, 1 mmol), 1-methyl-1H-pyrazole-3-amine (145 mg, 1.5 mmol), Brettphos Pd G₃ (45.3 mg, 0.05 mmol) and sodium tert-butoxide (192 mg, 2 mmol) were dissolved in 1,4-dioxane, and the mixture was reacted overnight at 95° C. under N₂ protection. After the reaction was completed, the reaction liquid was cooled to room temperature and filtered, and the filtrate was collected and separated by silica gel chromatographic column (DCM:MeOH=20:1), to afford title compound 32B (289 mg, 82.1%) as a yellow solid.

LC-MS (ESI): m/z=359.2 [M+H]⁺.

### Step 2:

32B (289 mg, 0.82 mmol) was dissolved in methanol (5 mL), and a solution of hydrogen chloride in dioxane (5 mL, 4 M) was added. The mixture was reacted at room temperature for 4 hours and spun to dryness to obtain title compound 32C (225 mg, crude).

LC-MS (ESI): m/z=259.2 [M+H]⁺.

### Step 3:

1H (100 mg, 0.45 mmol) and 32C (225 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH=1:0-10:1), to afford compound 32 (88 mg, 44.4%).

¹H NMR (400 MHz, DMSO-d₆) δ 11.81 (s, 1H), 8.77 (s, 1H), 8.40 (d, 1H), 7.79 (d, 1H), 7.75 (s, 1H), 7.63 (d, 1H), 7.44 (d, 1H), 7.30 (dd, 1H), 7.21 (d, 1H), 6.17 (d, 1H), 3.70 (s, 3H), 3.64 (s, 2H), 3.02 (t, 4H), 2.58-2.54 (m, 4H), 2.54-2.50 (m, 2H), 1.18 (t, 3H).

LC-MS (ESI): m/z=445.2 [M+H]⁺.

### Example 33

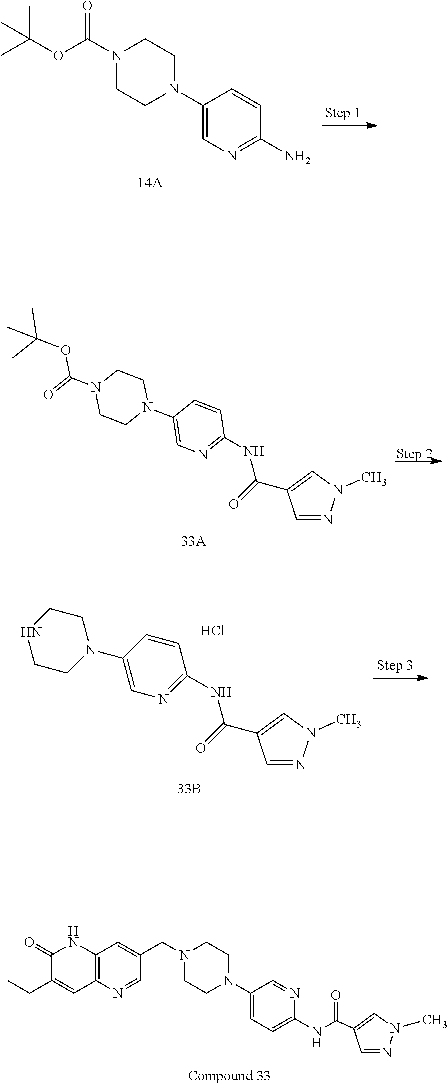

### Step 1:

Compound 1-methyl-1H-pyrazole-4-carboxylic acid (378 mg, 3 mmol) was dissolved in thionyl chloride (20 mL), the mixture was reacted at 70° C. for 1 h at reflux, and the solvent was removed by distillation under reduced pressure. The residue was dissolved in DCM (20 mL), tert-butyl 4-(6-aminopyridin-3-yl)piperazine-1-carboxylate (556 mg, 2 mmol) and triethylamine were added, and the mixture was stirred overnight at room temperature, 0.1M (20 mL) hydrochloric acid solution was added to quench the reaction, and the mixture was extracted with DCM (10 mL×3), washed with saturated NaHCO₃ solution, dried, and separated by silica gel chromatographic column (PE:EA=2:1), to afford title compound 33A (277 mg, 42.5%) as a yellow solid.

LC-MS (ESI): m/z=387.2 [M+H]⁺.

### Step 2:

33A (277 mg, 0.71 mmol) was dissolved in methanol (5 mL), and a solution of hydrogen chloride in dioxane (5 mL, 4 M) was added. The mixture was reacted at room temperature for 4 hours and spun to dryness to obtain title compound 33B (233 mg, crude).

LC-MS (ESI): m/z=287.2 [M+H]⁺.

### Step 3:

1H (120 mg, 0.54 mmol) and 33B (233 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and separated and purified by column chromatography (DCM:MeOH=1:0-10:1), to afford compound 33 (86 mg, 33.8%).

¹H NMR (400 MHz, DMSO-d₆) δ 11.83 (s, 1H), 10.22 (s, 1H), 8.41 (s, 1H), 8.37 (s, 1H), 8.08 (d, 1H), 8.02 (d, 1H), 7.98 (s, 1H), 7.75 (s, 1H), 7.63 (s, 1H), 7.42 (dd, 1H), 3.87 (s, 3H), 3.65 (s, 2H), 3.17 (s, 4H), 2.59-2.55 (m, 4H), 2.55-2.52 (m, 2H), 1.18 (t, 3H).

LC-MS (ESI): m/z=473.2 [M+H]⁺.

### Example 34

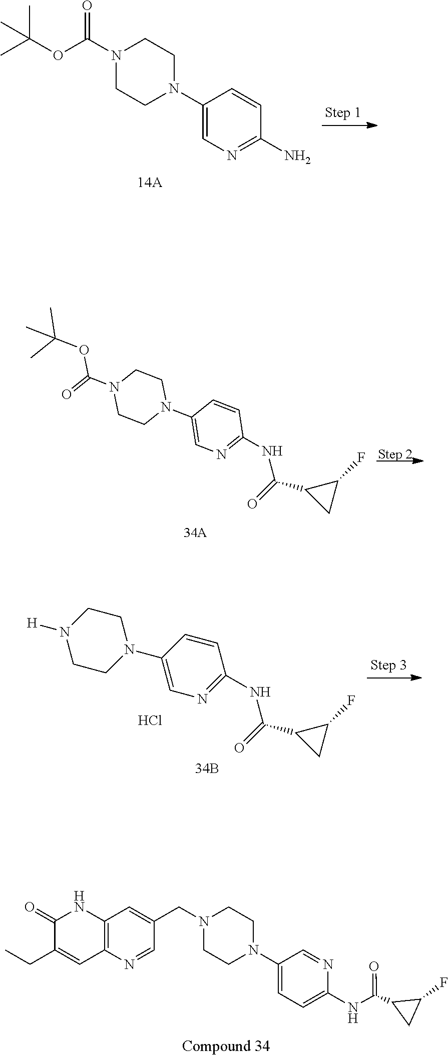

### Step 1:

Compound (1R,2R)-2-fluorocyclopropane-1-carboxylic acid (312 mg, 3 mmol) was dissolved in thionyl chloride (10 mL), the mixture was reacted at 70° C. for 1 h at reflux, and the solvent was removed by distillation under reduced pressure. The residue was dissolved in DCM (20 mL), tert-butyl 4-(6-aminopyridin-3-yl)piperazine-1-carboxylate (556 mg, 2 mmol) and triethylamine were added, and the mixture was stirred overnight at room temperature. 0.1M (20 mL) hydrochloric acid solution was added to quench the reaction, and the mixture was extracted with DCM (10 mL×3), washed with saturated NaHCO₃ solution, dried, and separated by silica gel chromatographic column (PE:EA=2:1), to afford title compound 34A (389 mg, 53.3%) as a pale yellow solid.

LC-MS (ESI): m/z=365.1 [M+H]⁺.

### Step 2:

34A (389 mg, 1.1 mmol) was dissolved in methanol (10 mL), and a solution of hydrogen chloride in dioxane (5 mL, 4 M) was added. The mixture was reacted at room temperature for 4 hours and spun to dryness to obtain title compound 34B (294 mg, crude).

LC-MS (ESI): m/z=365.2 [M+H]⁺.

### Step 3:

1H (180 mg, 0.8 mmol) and 34B (294 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH=1:0-10:1), to afford compound 34 (121 mg, 33.6%).

¹H NMR (400 MHz, DMSO-d₆) δ 11.82 (s, 1H), 10.65 (s, 1H), 8.40 (d, 1H), 8.00 (d, 1H), 7.86 (d, 1H), 7.75 (d, 1H), 7.62 (d, 1H), 7.37 (dd, 1H), 4.96-4.73 (m, 1H), 3.64 (s, 2H), 3.15 (t, 4H), 2.55 (t, 4H), 2.55-2.54 (m, 2H), 2.49-2.41 (m, 1H), 1.52-1.38 (m, 1H), 1.18 (t, 3H), 1.25-1.15 (m, 1H).

LC-MS (ESI): m/z=451.2 [M+H]⁺.

### Example 35

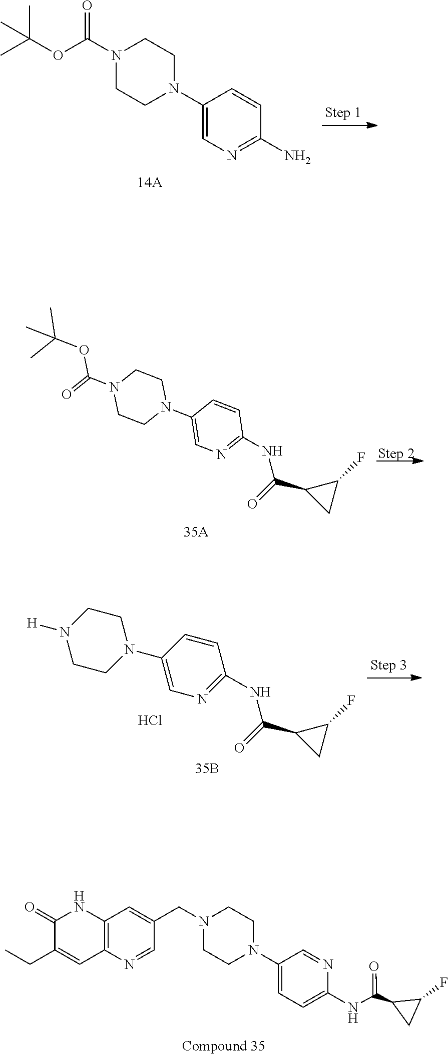 [SMILES: CC(C)(C)OC(=O)N1CCN(c2ccc(N)nc2)CC1 ; CC(C)(C)OC(=O)N1CCN(c2ccc(NC(=O)[C@@H]3C[C@H]3F)nc2)CC1 ; O=C(Nc1ccc(N2CCNCC2)cn1)[C@@H]1C[C@H]1F ; Cl ; CCc1cc2ncc(CN3CCN(c4ccc(NC(=O)[C@@H]5C[C@H]5F)nc4)CC3)cc2[nH]c1=O]

### Step 1:

Compound (1S,2R)-2-fluorocyclopropane-1-carboxylic acid (312 mg, 3 mmol) was dissolved in thionyl chloride (10 mL), the mixture was reacted at 70° C. for 1 h at reflux, and the solvent was removed by distillation under reduced pressure. The residue was dissolved in DCM (20 mL), tert-butyl 4-(6-aminopyridin-3-yl)piperazine-1-carboxylate (556 mg, 2 mmol) and triethylamine were added, and the mixture was stirred overnight at room temperature. 0.1M (20 mL) hydrochloric acid solution was added to quench the reaction, and the mixture was extracted with DCM (10 mL×3), washed with saturated NaHCO₃ solution, dried, and separated by silica gel chromatographic column (PE:EA=2:1), to afford title compound 35A (421 mg, 58.6%).

LC-MS (ESI): m/z=365.1 [M+H]⁺.

### Step 2:

35A (421 mg, 1.1 mmol) was dissolved in methanol (10 mL), and a solution of HCl in dioxane (5 mL, 4 M) was added. The mixture was reacted at room temperature for 4 hours and spun to dryness to obtain title compound 35B (314 mg, crude).

LC-MS (ESI): m/z=365.2 [M+H]⁺.

### Step 3:

1H (180 mg, 0.8 mmol) and 35B (300 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH=1:0-10:1), to afford compound 35 (111 mg, 32.4%).

¹H NMR (400 MHz, DMSO-d₆) δ 11.82 (s, 1H), 10.52 (s, 1H), 8.40 (d, 1H), 7.99 (d, 1H), 7.91 (d, 1H), 7.75 (s, 1H), 7.63 (d, 1H), 7.38 (dd, 1H), 2.74-5.01 (m, 1H), 3.65 (s, 2H), 3.15 (t, 4H), 2.58 (t, 4H), 2.54-2.53 (m, 2H), 2.27-2.03 (m, 1H), 1.68-1.54 (m, 1H), 1.18 (t, 3H), 1.15-1.08 (m, 1H).

LC-MS (ESI): m/z=451.2 [M+H]⁺.

### Example 36

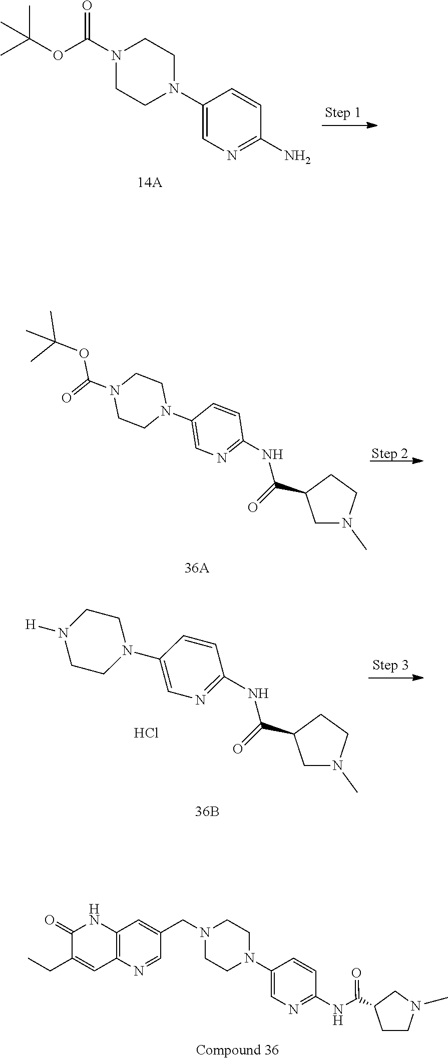

### Step 1:

Compound (S)-1-methylpyrrolidine-3-carboxylic acid (387 mg, 3 mmol) was dissolved in thionyl chloride (10 mL), the mixture was reacted at 70° C. for 1 h at reflux, and the solvent was removed by distillation under reduced pressure. The residue was dissolved in DCM (20 mL), tert-butyl 4-(6-aminopyridin-3-yl)piperazine-1-carboxylate (556 mg, 2 mmol) and triethylamine were added, and the mixture was stirred overnight at room temperature. 0.1M (20 mL) hydrochloric acid solution was added to quench the reaction, and the mixture was extracted with DCM (10 mL×3), washed with saturated NaHCO₃ solution, dried, and separated by silica gel chromatographic column (PE:EA=2:1), to afford title compound 36A (352 mg, 45.3%).

LC-MS (ESI): m/z=390.2 [M+H]⁺.

### Step 2:

36A (352 mg, 0.9 mmol) was dissolved in methanol (10 mL), and a solution of hydrogen chloride in dioxane (5 mL, 4 M) was added. The mixture was reacted at room temperature for 4 hours and spun to dryness to obtain title compound 36B (282 mg, crude).

LC-MS (ESI): m/z=290.2 [M+H]⁺.

### Step 3:

1H (180 mg, 0.8 mmol) and 36B (282 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH=1:0-10:1), to afford compound 36 (132 mg, 37.5%).

¹H NMR (400 MHz, DMSO-d₆) δ 11.83 (s, 1H), 10.15 (s, 1H), 8.40 (d, 1H), 7.98 (d, 1H), 7.92 (d, 1H), 7.75 (s, 1H), 7.63 (d, 1H), 7.38 (dd, 1H), 3.64 (s, 2H), 3.15 (t, 4H), 3.13-3.05 (m, 2H), 2.75 (t, 1H), 2.55 (t, 4H), 2.55-2.53 (m, 2H), 2.47 (dd, 1H), 2.40 (q, 1H), 2.24 (s, 3H), 2.04-1.89 (m, 2H), 1.18 (t, 3H).

LC-MS (ESI): m/z=476.2 [M+H]⁺.

### Example 37

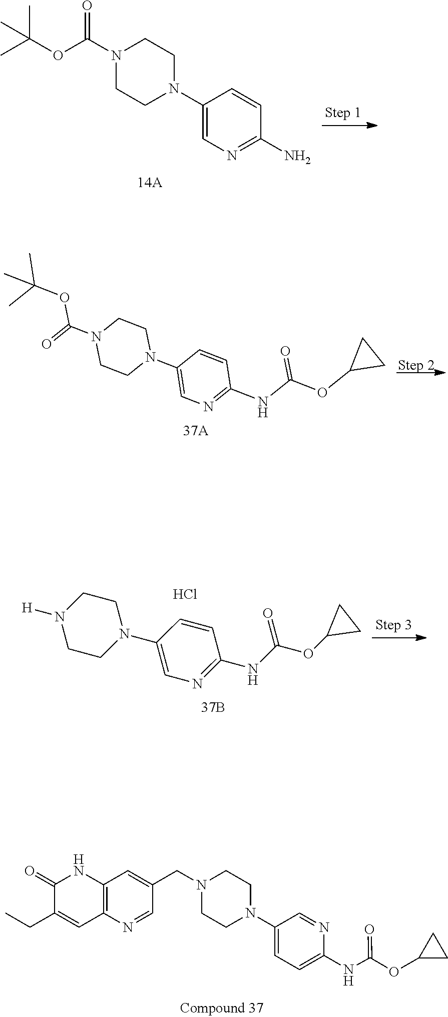 [SMILES: CC(C)(C)OC(=O)N1CCN(c2ccc(N)nc2)CC1 ; CC(C)(C)OC(=O)N1CCN(c2ccc(NC(=O)OC3CC3)nc2)CC1 ; O=C(Nc1ccc(N2CCNCC2)cn1)OC1CC1 ; Cl ; CCc1cc2ncc(CN3CCN(c4ccc(NC(=O)OC5CC5)nc4)CC3)cc2[nH]c1=O]

### Step 1:

Compound tert-butyl 4-(6-aminopyridin-3-yl)piperazine-1-carboxylate (556 mg, 2 mmol) was dissolved in DMF (20 mL) under N₂ protection and cooled in an ice-water bath, NaH (320 mg, 60%) was then added, and the mixture was stirred and reacted for 1 h in the ice bath. Subsequently, CDI (486 mg, 3 mmol) was added, and the mixture was stirred and reacted for another 30 min, at which time the color of the system was observed to become lighter. Excess cyclopropanol was finally added, and the mixture was reacted at room temperature for 2 h. After the reaction was completed, to the system was added ethyl acetate (100 mL), and the mixture was washed with water (100 mL×4). The organic phase was collected, dried over anhydrous sodium sulfate, filtered, evaporated to dryness, and separated by silica gel chromatographic column (PE:EA=1:0-1:1), to afford title compound 37A (253 mg, 35.1%).

LC-MS (ESI): m/z=363.2 [M+H]⁺.

### Step 2:

37A (253 mg, 0.7 mmol) was dissolved in methanol (10 mL), and a solution of hydrogen chloride in dioxane (5 mL, 4 M) was added. The mixture was reacted at room temperature for 4 hours and spun to dryness to obtain title compound 37B (200 mg, crude).

LC-MS (ESI): m/z=290.2 [M+H]⁺.

### Step 3:

1H (150 mg, 0.67 mmol) and 37B (200 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH=1:0-10:1), to afford compound 37 (76 mg, 25.3%).

¹H NMR (400 MHz, DMSO-d₆) δ 12.17 (s, 1H), 9.88 (d, 1H), 8.55 (d, 1H), 8.01 (d, 1H), 7.81 (s, 1H), 7.79 (d, 1H), 7.69 (d, 1H), 7.48 (dd, 1H), 4.53 (s, 2H), 4.05 (tt, 1H), 3.15 (t, 4H), 2.65 (t, 4H), 2.62-2.53 (m, 2H), 1.20 (t, 3H), 0.83-0.57 (m, 4H).

LC-MS (ESI): m/z=449.2 [M+H]⁺.

### Example 38

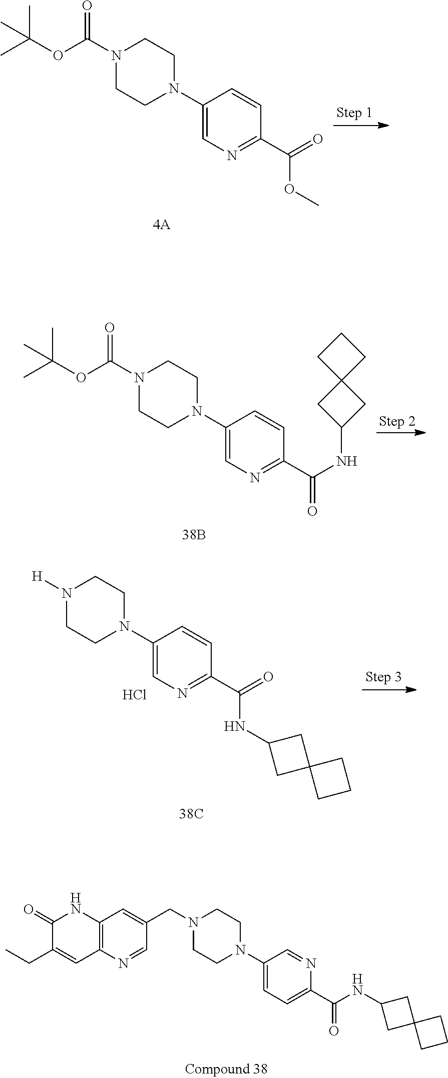 [SMILES: COC(=O)c1ccc(N2CCN(C(=O)OC(C)(C)C)CC2)cn1 ; CC(C)(C)OC(=O)N1CCN(c2ccc(C(=O)NC3CC4(CCC4)C3)nc2)CC1 ; O=C(NC1CC2(CCC2)C1)c1ccc(N2CCNCC2)cn1 ; Cl ; CCc1cc2ncc(CN3CCN(c4ccc(C(=O)NC5CC6(CCC6)C5)nc4)CC3)cc2[nH]c1=O]

### Step 1:

Compound 4A (321 mg, 1 mmol) was dissolved in THF (10 mL) and H₂O (0 mL), LiOH·H₂O (45 mg, 1.1 mmol) was added, and the mixture was stirred and reacted at room temperature for 2 h. The solvent was removed by distillation under reduced pressure, to the resulting solid was added DMF (10 mL), HATU (570 mg, 1.5 mmol) was added under stirring, and the mixture was stirred at room temperature. After the solid was completely dissolved, DIEPA (1 mL) was added, spiro[3.3]heptan-2-amine (222 mg, 2 mmol) was finally added, and the mixture was stirred overnight at room temperature. After the reaction was completed as monitored by LCMS, to the system was added water (50 mL) under stirring, at which time a large amount of white solid was observed to be precipitated out, and the mixture was stirred for another 10 min, filtered and dried under vacuum, to afford target compound 38B (322 mg, 80.5%) as a white solid.

LC-MS (ESI): m/z=401.2 [M+H]⁺.

### Step 2:

38B (332 mg, 0.8 mmol) was dissolved in methanol (5 mL), and a solution of hydrogen chloride in dioxane (5 mL, 4M) was added. The mixture was reacted at room temperature for 4 hours and spun to dryness, to afford title compound 38C (267 mg, crude).

LC-MS (ESI): m/z=301.2 [M+H]⁺.

### Step 3:

1H (180 mg, 0.8 mmol) and 38C (267 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH=1:0-10:1), to afford compound 38 (141 mg, 36.2%).

¹H NMR (400 MHz, DMSO-d₆) δ 11.83 (s, 1H), 8.62-8.35 (m, 2H), 8.25 (d, 1H), 7.81 (d, 1H), 7.75 (s, 1H), 7.63 (d, 1H), 7.38 (dd, 1H), 4.26 (s, 1H), 3.65 (s, 2H), 3.34 (t, 4H), 2.61-2.52 (m, 6H), 2.32-2.24 (m, 2H), 2.13-1.98 (m, 4H), 1.90 (t, 2H), 1.79 (q, 2H), 1.18 (t, 3H).

LC-MS (ESI): m/z=487.2 [M+H]⁺.

### Example 39

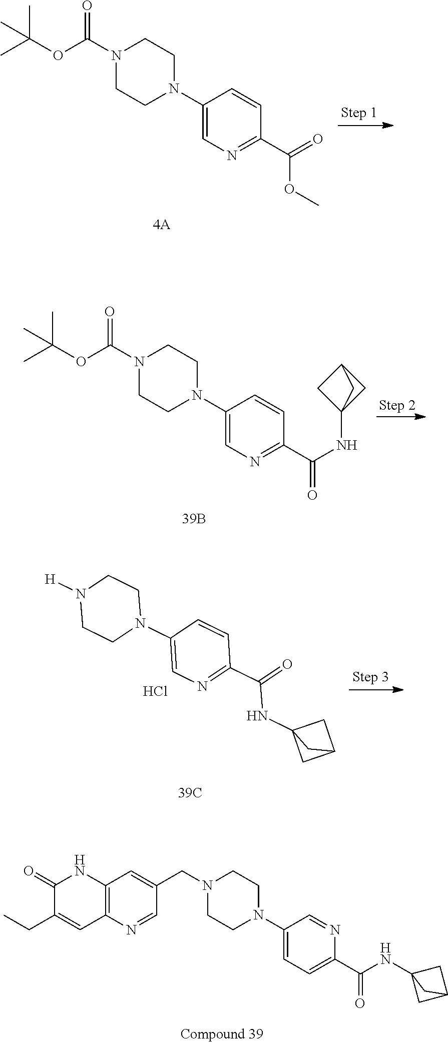 [SMILES: COC(=O)c1ccc(N2CCN(C(=O)OC(C)(C)C)CC2)cn1 ; CC(C)(C)OC(=O)N1CCN(c2ccc(C(=O)NC34CC(C3)C4)nc2)CC1 ; O=C(NC12CC(C1)C2)c1ccc(N2CCNCC2)cn1 ; Cl ; CCc1cc2ncc(CN3CCN(c4ccc(C(=O)NC56CC(C5)C6)nc4)CC3)cc2[nH]c1=O]

### Step 1:

Compound 4A (321 mg, 1 mmol) was dissolved in THF (10 mL) and H₂O (1 mL). LiOH·H₂O (45 mg, 1.1 mmol) was added, and the mixture was stirred and reacted at room temperature for 2 h. The solvent was removed by distillation under reduced pressure, to the resulting solid was added DMF (10 mL), HATU (570 mg, 1.5 mmol) was added under stirring, and the mixture was stirred at room temperature. After the solid was completely dissolved, DIEPA (1 mL) was added, bicyclo[1.1.1]pentan-1-amine (166 mg, 2 mmol) was finally added, and the mixture was stirred overnight at mom temperature. After the reaction was completed as monitored by LCMS, to the system was added water (50 mL) under stirring, at which time a large amount of white solid was observed to be precipitated out, and the mixture was stirred for another 10 min, filtered and dried under vacuum, to afford target compound 39B (292 mg, 78.5%) as a white solid.

LC-MS (ESI): m/z=373.2 [M+H]⁺.

### Step 2:

39B (292 mg, 0.78 mmol) was dissolved in methanol (5 mL), and a solution of hydrogen chloride in dioxane (5 mL, 4 M) was added. The mixture was reacted at room temperature for 4 hours and spun to dryness to obtain title compound 39C (239 mg, crude).

LC-MS (ESI): m/z=273.2 [M+H]⁺.

### Step 3:

1H (120 mg, 0.6 mmol) and 39C (239 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH=1:0-10:1), to afford compound 39 (87 mg, 26.2%).

¹H NMR (400 MHz, DMSO-d₆) δ 11.80 (s, 1H), 8.75 (s, 1H), 8.40 (d, 1H), 8.24 (d, 1H), 7.80 (d, 1H), 7.75 (s, 1H), 7.63 (d, 1H), 7.39 (dd, 1H), 3.65 (s, 2H), 3.34 (t, 4H), 2.60-2.52 (m, 6H), 2.43 (s, 1H), 2.07 (s, 6H), 1.18 (t, 3H).

LC-MS (ESI): m/z=459.2 [M+H]⁺.

### Example 40

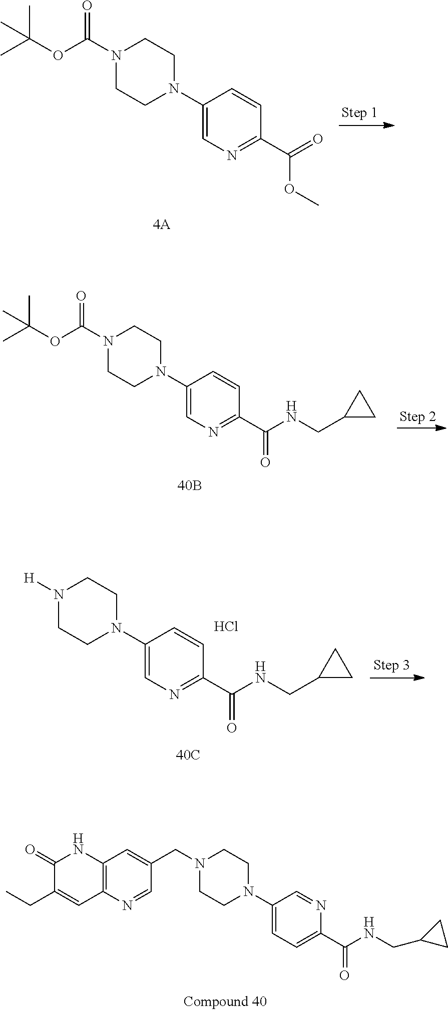 [SMILES: COC(=O)c1ccc(N2CCN(C(=O)OC(C)(C)C)CC2)cn1 ; CC(C)(C)OC(=O)N1CCN(c2ccc(C(=O)NCC3CC3)nc2)CC1 ; O=C(NCC1CC1)c1ccc(N2CCNCC2)cn1 ; Cl ; CCc1cc2ncc(CN3CCN(c4ccc(C(=O)NCC5CC5)nc4)CC3)cc2[nH]c1=O]

### Step 1:

Compound 4A (321 mg, 1 mmol) was dissolved in THF (10 mL) and H₂O (1 mL), LiOH·H₂O (45 mg, 1.1 mmol) was added, and the mixture was stirred and reacted at room temperature for 2 h. The solvent was removed by distillation under reduced pressure, to the resulting solid was added DMF (10 mL), HATU (570 mg, 1.5 mmol) was added under stirring, and the mixture was stirred at room temperature. After the solid was completely dissolved, DIEPA (1 mL) was added, cyclopropylmethylamine (140 mg, 2 mmol) was finally added, and the mixture was stirred overnight at room temperature. After the reaction was completed as monitored by LCMS, to the system was added water (50 mL) under stirring, at which time a large amount of white solid was observed to be precipitated out, and the mixture was stirred for another 10 min, filtered and dried under vacuum, to afford target compound 40B (332 mg, 92.2%) as a white solid.

LC-MS (ESI): m/z=361.2 [M+H]⁺.

### Step 2:

40B (332 mg, 0.92 mmol) was dissolved in methanol (5 mL), and a solution of hydrogen chloride in dioxane (5 mL, 4 M) was added. The mixture was reacted at room temperature for 4 hours and spun to dryness to obtain title compound 40C (281 mg, crude).

LC-MS (ESI): m/z=261.2 [M+H]⁺.

### Step 3:

1H (180 mg, 0.8 mmol) and 40C (281 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH=1:0-10:1), to afford compound 40 (143 mg, 39.7%).

¹H NMR (400 MHz, DMSO-d₆) δ 11.83 (s, 1H), 8.47-8.35 (m, 2H), 8.28 (d, 1H), 7.84 (d, 1H), 7.75 (s, 1H), 7.63 (d, 1H), 7.40 (dd, 1H), 3.66 (s, 2H), 3.34 (t, 4H), 3.14 (t, 2H), 2.66-2.52 (m, 6H), 1.19 (t, 3H), 1.12-0.95 (m, 1H), 0.49-0.33 (m, 2H), 0.30-0.16 (m, 2H).

LC-MS (ESI): m/z=447.2 [M+H]⁺.

### Example 41

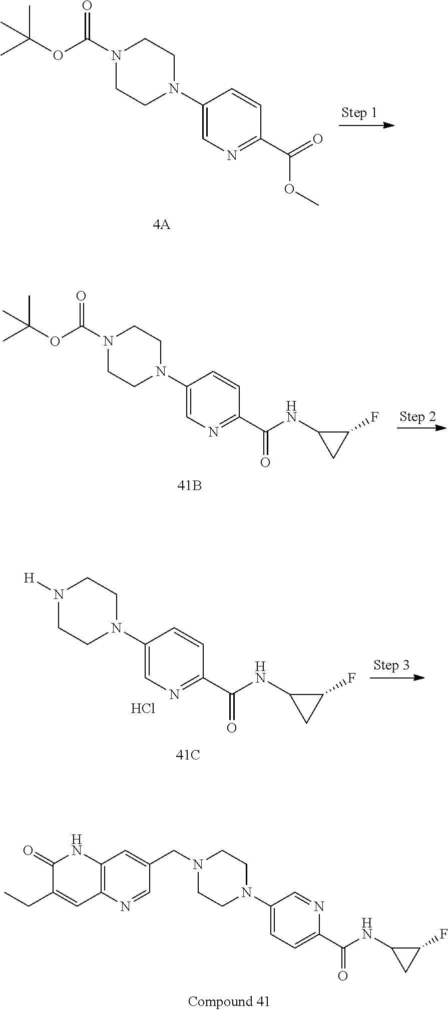 [SMILES: COC(=O)c1ccc(N2CCN(C(=O)OC(C)(C)C)CC2)cn1 ; CC(C)(C)OC(=O)N1CCN(c2ccc(C(=O)NC3C[C@H]3F)nc2)CC1 ; O=C(NC1C[C@H]1F)c1ccc(N2CCNCC2)cn1 ; Cl ; CCc1cc2ncc(CN3CCN(c4ccc(C(=O)NC5C[C@H]5F)nc4)CC3)cc2[nH]c1=O]

### Step 1:

Compound 4A (321 mg, 1 mmol) was dissolved in THF (10 mL) and H₂O (1 mL), LiOH·H₂O (45 mg, 1.1 mmol) was added, and the mixture was stirred and reacted at room temperature for 2 h. The solvent was removed by distillation under reduced pressure, to the resulting solid was added DMF (10 mL), HATU (570 mg, 1.5 mmol) was added under stirring, and the mixture was stirred at room temperature. After the solid was completely dissolved, DIEPA (1 mL) was added, cyclopropylmethylamine (150 mg, 2 mmol) was finally added, and the mixture was stirred overnight at room temperature. After the reaction was completed as monitored by LCMS, to the system was added water (50 mL) under stirring, at which time a large amount of white solid was observed to be precipitated out, and the mixture was stirred for another 10 min, filtered and dried under vacuum, to afford target compound 41B (327 mg, 89.8%) as a white solid.

LC-MS (ESI): m/z=365.2 [M+H]⁺.

### Step 2:

41B (327 mg, 0.9 mmol) was dissolved in methanol (5 mL), and a solution of hydrogen chloride in dioxane (5 mL, 4 M) was added. The mixture was reacted at room temperature for 4 hours and spun to dryness to obtain title compound 41C (271 mg, crude).

LC-MS (ESI): m/z=265.2 [M+H]⁺.

### Step 3:

1H (160 mg, 0.7 mmol) and 41C (271 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH=1:0-10:1), to afford compound 41 (118 mg, 37.4%).

¹H NMR (400 MHz, DMSO-d₆) δ 11.83 (s, 1H), 8.40 (d, 1H), 8.31 (d, 1H), 8.27 (d, 1H), 7.85 (d, 1H), 7.75 (s, 1H), 7.63 (d, 1H), 7.41 (dd, 1H), 4.89-4.65 (m, 1H), 3.65 (s, 2H), 3.35 (t, 4H), 2.90-2.82 (m, 1H), 2.62-2.52 (m, 6H), 1.24-1.19 (m, 1H), 1.18 (t, 3H), 1.15-1.03 (m, 1H).

LC-MS (ESI): m/z=451.2 [M+H]⁺.

### Example 42

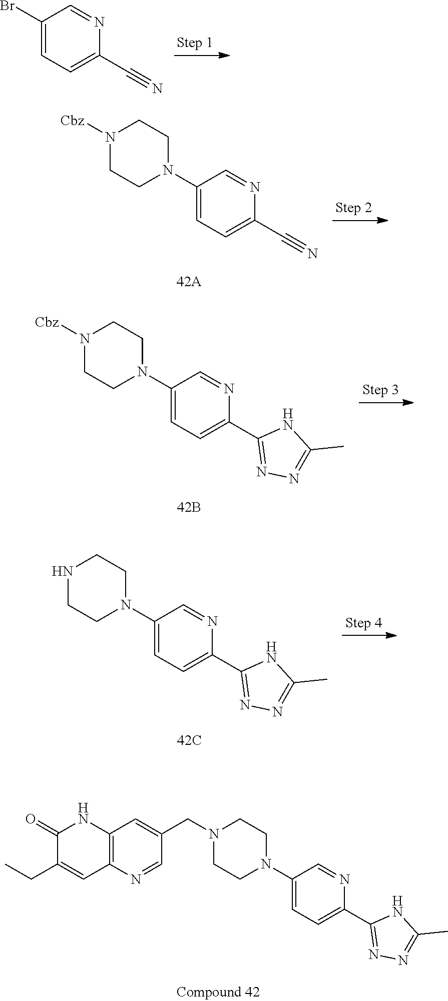

### Step 1:

5-Bromochloronitronitrile (1.0 g, 5.45 mmol) and benzyl piperazine-1-carboxylate (1.4 g, 6.54 mmol) were dissolved in 1,4-dioxane (10 mL), Cs₂CO₃ (5.3 g, 16.35 mmol) and RuPhos-Pd-G3 (182 mg, 0.22 mmol) were added, and the mixture was reacted overnight at 100° C. under nitrogen protection. Subsequently, the reaction liquid was quenched with water (15 mL) and extracted with ethyl acetate (20 mL×3). The organic phases were combined, dried over anhydrous Na₂SO₄ filtered, spun to dryness, and separated by silica gel chromatographic column (PE:EA=1:0-1:1), to afford title compound 42A (1.6 g, 91.1%) as a pale yellow solid.

LC-MS (ESI): m/z=323.1 [M+H]⁺.

### Step 2:

42A (500 mg, 1.55 mmol) was dissolved in ethanol (5 mL), sodium methoxide (0.29 mL, 5.4 mol/L) was then added dropwise, and the mixture was stirred at room temperature for 1 hour. After the reaction was completed as monitored by TLC, acetyl hydrazine (689 mg, 9.3 mmol) was added, and the mixture was refluxed overnight, concentrated and separated by silica gel chromatographic column (PE:EA=1:0-1:1), to afford title compound 42B (220 mg, 37.5%).

LC-MS (ESI): m/z=379.1 [M+H]⁺.

### Step 3:

42B (220 mg, 1.55 mmol) was dissolved in methanol (5 mL), Pd/C (44 mg) was added, and the mixture was reacted at room temperature in H₂ environment for two hours, filtered and spun to dryness, to afford title compound 42C (120 mg, 84.7%).

LC-MS (ESI): m/z=223.1 [M+H]⁺.

### Step 4:

1H (100 mg, 0.45 mmol) and 42C (110 mg, 0.45 mmol) were dissolved in anhydrous acetonitrile (5 mL), potassium iodide (8 mg, 0.05 mmol) and DIPEA (291 mg, 2.25 mmol) were added, and the mixture was subjected to nitrogen replacement and reacted at 80° C. for 2 hours. Upon complete depletion of raw materials monitored by LCMS, a product was generated, and the system was concentrated and purified by preparative HPLC with preparative HPLC separation methods: 1. Instruments: waters 2767 preparative liquid chromatographic instrument; chromatographic column: SunFire@ Prep C18 (19 mm×250 mm); 2. The sample was filtered with a 0.45 μm filter to prepare a sample liquid; 3. Preparative chromatography conditions: a. composition of mobile phases A and B: mobile phase A: acetonitrile; mobile phase B: water (containing 0.1% ammonia); b. gradient elution, mobile phase A: 5%-50%; c. flow rate: 12 mL/min; d. elution time: 20 min; and the preparative liquid was concentrated and lyophilized to obtain the compound 42 (30 mg, 15.5%).

¹H NMR (400 MHz, DMSO-d₆) δ 12.34 (s, 1H), 8.41 (d, 1H), 8.34 (d, 1H), 7.84 (d, 1H), 7.75 (s, 1H), 7.63 (s, 1H), 7.41 (dd, 1H), 3.66 (s, 2H), 3.36-3.32 (m, 4H, overlapped with H₂O peak), 2.61-2.52 (m, 6H), 2.31 (s, 3H), 1.19 (t, 3H).

LC-MS (ESI): m/z=431.2 [M+H]⁺.

### Example 43

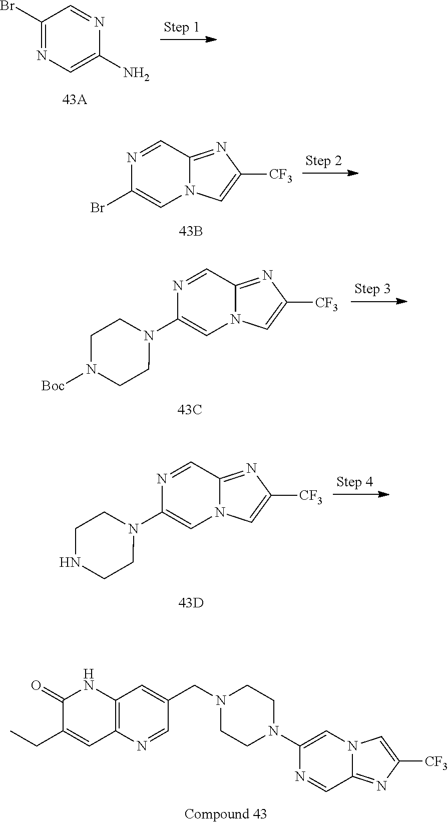 [SMILES: Nc1cnc(Br)cn1 ; FC(F)(F)c1cn2cc(Br)ncc2n1 ; CC(C)(C)OC(=O)N1CCN(c2cn3cc(C(F)(F)F)nc3cn2)CC1 ; FC(F)(F)c1cn2cc(N3CCNCC3)ncc2n1 ; CCc1cc2ncc(CN3CCN(c4cn5cc(C(F)(F)F)nc5cn4)CC3)cc2[nH]c1=O]

### Step 1:

43A (1.0 g, 5.71 mmol), 3-bromo-1,1,1-trifluoropropan-2-one (2.7 g, 14.26 mmol), and N,N-dimethylacetamide (4 mL) were added to a sealed tube, and the mixture was stirred at 80° C. for 16 h. After the reaction was completed, the reaction liquid was purified by reverse-phase column, to afford 43B (460 mg, 30%).

LCMS m/z=266.0 [M+1]⁺

### Step 2:

Compound 43B (800 mg, 3.01 mmol), tert-butyl piperazine-1-carboxylate (840 mg, 4.52 mmol), Pd₂(dba)₃ (287 mg, 0.30 mmol), JohnPhos (90 mg, 0.30 mmol), and sodium tert-butoxide (723 mg, 7.52 mmol) were added to a reaction flask, and the mixture was stirred at 110° C. under nitrogen protection for 4 h. After the reaction was completed, the reaction liquid was extracted three times with ethyl acetate, washed with saturated sodium chloride solution, dried over anhydrous sodium sulfate, concentrated under reduced pressure, and subjected to flash column chromatography (EA:PE=20%), to afford tert-butyl 4-(2-(trifluoromethyl)imidazole[1,2-a]pyrazin-6-yl)piperazine-1-carboxylate (43C) (330 mg, 29%).

LCMS m/z=372.1 [M+1]⁺

### Step 3:

Compound 43C (330 mg, 0.89 mmol) and CF₃COOH (3 mL) were added to a reaction flask, and the mixture was stirred at 25° C. for 1 h. After the reaction was completed, the reaction liquid was concentrated under reduced pressure, to afford crude 43D (240 mg, 100%), which was used directly in the next step.

### Step 4:

47D (240 mg, 0.88 mmol), 7-(chloromethyl)-3-ethyl-1,5-naphthyridine-2(1H)-one (235 mg, 1.06 mmol), DIPEA (341 mg, 2.64 mmol), KI (29 mg, 0.18 mmol) and acetonitrile (4 mL) were added to a reaction flask, and the mixture was stirred at 80° C. for 2 h. After the reaction was completed as monitored by LCMS, the reaction system was directly subjected to preparative HPLC with preparative HPLC separation methods: 1. Instruments: waters 2767 preparative liquid chromatographic instrument: chromatographic column: SunFire@ Prep C18 (19 mm×250 mm); 2. The sample was filtered with a 0.45 μm filter to prepare a sample liquid; 3. Preparative chromatography conditions: a. composition of mobile phases A and B: mobile phase A: acetonitrile; mobile phase B: water (containing 0.1% ammonium acetate); b. gradient elution, mobile phase A: 10%-55%; c. flow rate: 12 mL/min; retention time: 7.0 min, to obtain title compound 43 (27 mg, 7%).

LCMS m/z=458.3 [M+1]⁺

¹H NMR (400 MHz, DMSO-d₆) δ 11.84 (s, 1H), 8.99 (s, 1H), 8.46 (s, 1H), 8.42 (d, 1H), 7.93 (d, 1H), 7.76 (s, 1H), 7.64 (d, 1H), 3.67 (s, 2H), 3.33 (s, 4H), 2.62-2.54 (m, 6H), 1.19 (t, 3H).

### Example 44

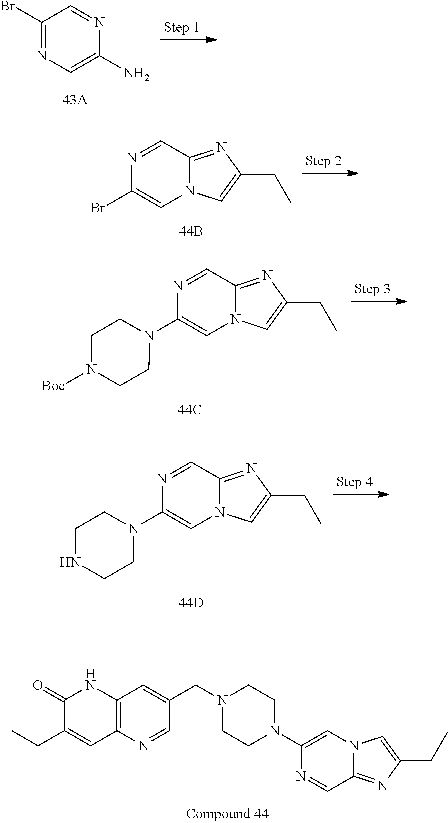

### Step 1:

43A (800 mg, 4.55 mmol), bromobutanone (687 mg, 4.55 mmol), and N,N-dimethylacetamide (4 mL) were added to a sealed tube, and the mixture was stirred at 80° C. for 16 h. After the reaction was completed, the reaction liquid was purified by reverse-phase column, to afford 44B (320 mg, 31%).

LCMS m/z=226.0 [M+1]⁺

### Step 2:

Compound 44B (220 mg, 0.97 mmol), tert-butyl piperazine-1-carboxylate (361 mg, 1.94 mmol), Pd₂(dba)₃ (89 mg, 0.10 mmol), JohnPhos (29 mg, 0.10 mmol), and sodium tert-butoxide (233 mg, 2.42 mmol) were added to a reaction flask, and the mixture was stirred at 110° C. under nitrogen protection for 4 h. After the reaction was completed, the reaction liquid was extracted three times with ethyl acetate, washed with saturated sodium chloride solution, dried over anhydrous sodium sulfate, concentrated under reduced pressure, and subjected to flash column chromatography (EA:PE=80%), to afford tert-butyl 4-(2-(trifluoromethyl)imidazole[1,2-a]pyrazin-6-yl)piperazine-1-carboxylate (44C) (71 mg, 22%).

LCMS m/z=332.2 [M+1]⁺

### Step 3:

Compound 44C (70 mg, 0.21 mmol) and CF₃COOH (3 mL) were added to a reaction flask, and the mixture was stirred at 25° C. for 1 h. After the reaction was completed, the reaction liquid was directly concentrated under reduced pressure, to afford crude 44D (48 mg, 100%), which was used directly in the next step.

### Step 4:

44D (48 mg, 0.21 mmol), 7-(chloromethyl)-3-ethyl-1,5-naphthyridine-2(1H)-one (71 mg, 0.32 mmol), DIPEA (81 mg, 0.63 mmol), KI (7 mg, 0.04 mmol) and acetonitrile (4 mL) were added to a reaction flask, and the mixture was stirred at 80° C. for 2 h. After the reaction was completed as monitored by LCMS, the reaction system was directly subjected to preparative HPLC with preparative HPLC separation methods: 1. Instruments: waters 2767 preparative liquid chromatographic instrument; chromatographic column: SunFire@ Prep C18 (19 mm×250 mm); 2. The sample was filtered with a 0.45 μm filter to prepare a sample liquid; 3. Preparative chromatography conditions: a. composition of mobile phases A and B: mobile phase A: acetonitrile; mobile phase B: water (containing 0.1% ammonium acetate); b. gradient elution, mobile phase A: 10%-55%; c. flow rate: 12 mL/min; retention time: 7.0 min, to obtain title compound 44 (4 mg, 5%).

LCMS m/z=418.3 [M+1]⁺

¹H NMR (400 MHz, DMSO-d₆) δ 11.83 (s, 1H), 8.68 (s, 1H), 8.42 (d, 1H), 7.85 (s, 1H), 7.74 (d, 2H), 7.64 (s, 1H), 3.66 (s, 2H), 3.28 (s, 4H), 2.72 (q, 2H), 2.62-2.53 (m, 6H), 1.25 (t, 3H), 1.19 (t, 3H).

### Example 45

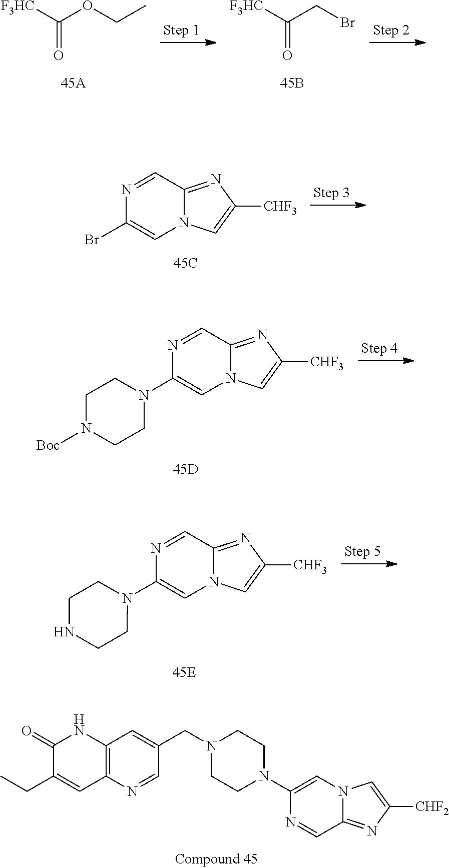

### Step 1:

45A (5 g, 40.27 mmol) was dissolved in anhydrous tetrahydrofuran, and the mixture was subjected to nitrogen replacement three times. Dibromomethane (14 g, 80.54 mmol) was added, the reaction system was cooled to −78° C., methyllithium (80.54 mmol) was added dropwise, and the mixture was reacted at −78° C. for 2 h. After the reaction was completed, saturated ammonium chloride solution was added at 0° C. to quench the reaction, and the mixture was extracted with ethyl acetate (50 ml*3). The organic phases were combined, dried over anhydrous sodium sulfate, filtered and concentrated, and the residue was separated and purified by flash column chromatography (eluent: EA/PE=0%-20%), to afford compound 45B (5.01 g, 72%).

LC-MS (ESI): m/z=173.2 [M+1]⁺

### Step 2:

45B (1 g, 5.78 mmol), 5-bromopyrazin-2-amine (0.33 g, 1.90 mmol), and N,N-dimethylacetamide (5 mL) were added to a sealed tube, and the mixture was stirred at 100° C. for 16 h. After the reaction was completed, the reaction liquid was purified by reverse-phase column, to afford 45C (250 mg, 53%).

LC-MS (ESI): m/z=248.0 [M+1]⁺

### Step 3:

Compound 45C (250 mg, 1.01 mmol), tert-butyl piperazine-1-carboxylate (280 mg, 1.5 mmol), Pd₂(dba)₃ (96 mg, 0.10 mmol), JohnPhos (30 mg, 0.10 mmol), and sodium tert-butoxide (290 mg, 3.03 mmol) were added to a reaction flask, and the mixture was stirred at 110° C. under nitrogen protection for 4 h. After the reaction was completed, the reaction liquid was extracted three times with ethyl acetate, washed with saturated sodium chloride solution, dried over anhydrous sodium sulfate, concentrated under reduced pressure, and subjected to flash column chromatography (EA:PE=20%), to afford target compound 45D (150 mg, 42%).

LC-MS (ESI): m/z=354.2 [M+1]⁺

### Step 4:

Compound 45D (150 mg, 0.42 mmol) was added to 50 mL of reaction flask, dichloromethane (3 ml)/trifluoroacetic acid (2 ml) were added, and the mixture was reacted at room temperature for 2 h. After the reaction was completed, the reaction liquid was directly concentrated, to afford crude compound 45E (150 mg).

LC-MS (ESI): m/z=254.2 [M+1]⁺

### Step 5:

45E (150 mg, 0.42 mmol), 7-(chloromethyl)-3-ethyl-1,5-naphthyridin-2(1H)-one (114 mg, 0.51 mmol), DIPEA (318 mg, 2.52 mmol), KI (16.6 mg, 0.1 mmol) and acetonitrile (4 mL) were added to a reaction flask, and the mixture was stirred at 80° C. for 2 h. After the reaction was completed as monitored by LCMS, the reaction system was directly subjected to preparative HPLC with preparative HPLC separation methods: 1. Instruments: waters 2767 preparative liquid chromatographic instrument; chromatographic column: SunFire@ Prep C18 (19 mm×250 mm); 2. The sample was filtered with a 0.45 μm filter to prepare a sample liquid; 3. Preparative chromatography conditions: a. composition of mobile phases A and B: mobile phase A: acetonitrile; mobile phase B: water (containing 0.1% ammonium acetate); b. gradient elution, mobile phase A: 10%-55%; c. flow rate: 12 mL/min; retention time: 7.0 min, to obtain title compound 45 (50 mg, 27%).

LCMS m/z=440.2 [M+1]⁺

¹H NMR (400 MHz, CDCl₃) δ=12.37 (s, 1H), 8.88 (s, 1H), 8.57 (s, 1H), 7.89 (s, 1H), 7.76 (d, 2H), 7.31 (s, 1H), 6.85 (t, 1H), 3.74 (s, 2H), 3.40 (s, 4H), 2.76 (d, 2H), 2.71 (s, 4H), 1.33 (d, 3H).

¹⁹F NMR (376 MHz, CDCl₃) δ=−111.39.

### Example 46

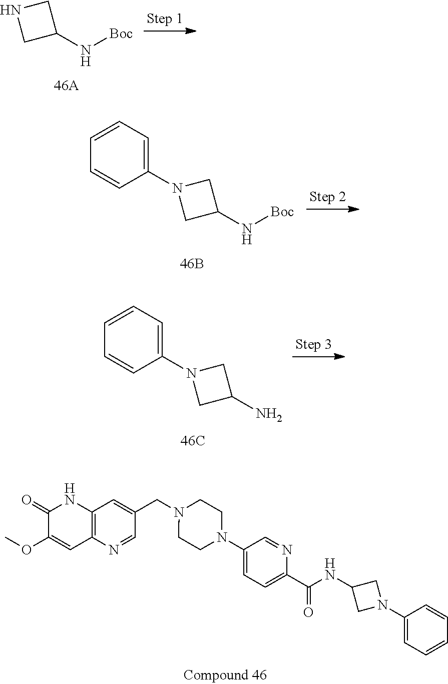 [SMILES: CC(C)(C)OC(=O)NC1CNC1 ; CC(C)(C)OC(=O)NC1CN(c2ccccc2)C1 ; NC1CN(c2ccccc2)C1 ; COc1cc2ncc(CN3CCN(c4ccc(C(=O)NC5CN(c6ccccc6)C5)nc4)CC3)cc2[nH]c1=O]

### Step 1:

Compound 46A (1 g, 5.79 mmol) was dissolved in 1,4-dioxane, iodobenzene (2.36 g, 11.58 mmol), Pd₂(dba)₃ (530 mg, 0.58 mmol), XantPhos (340 mg, 0.58 mmol) and cesium carbonate (5.66 g, 17.37 mmol) were added to a reaction flask under nitrogen, and the mixture was stirred overnight at 100° C. under nitrogen protection. After the reaction was completed, the reaction liquid was extracted three times with ethyl acetate, washed with saturated sodium chloride solution, dried over anhydrous sodium sulfate, concentrated under reduced pressure, and subjected to flash column chromatography (EA/PE=0-20%), to afford target compound 46B (350 mg, 18%).

LC-MS (ESI): m/z=249.2 [M+1]⁺

### Step 2:

Compound 46B (0.25 g, 1.01 mmol) was added to 50 mL of reaction flask, dichloromethane (3 ml)/trifluoroacetic acid (3 ml) were added, and the mixture was reacted at room temperature for 2 h. After the reaction was completed, the reaction liquid was directly concentrated, to afford crude compound 46C (0.2 g).

LC-MS (ESI): m/z=149.1 [M+1]⁺

### Step 3:

5-(4-((7-ethyl-6-oxo-5,6-dihydro-1,5-naphthyridin-3-yl)methyl)piperazin-1-yl)picolinic acid (100 mg, 0.25 mmol) was dissolved in N,N-dimethylformamide (2 ml), compound 46C (60 mg, 0.38 mmol), 2-(7-azobenzotriazole)-N,N,N′,N-tetramethyluronium hexafluorophosphate (190 mg, 0.5 mmol) and N,N-diisopropylethylamine (130 mg, 1 mmol) were added to the reaction system, and the mixture was reacted overnight at mom temperature. After the reaction was completed as monitored by LCMS, the reaction system was purified by preparative HPLC with preparative HPLC separation methods: 1. Instruments: waters 2767 preparative liquid chromatographic instrument; chromatographic column: SunFire@ Prep C18 (19 mm×250 mm); 2. The sample was filtered with a 0.45 μm filter to prepare a sample liquid; 3. Preparative chromatography conditions: a. composition of mobile phases A and B: mobile phase A: acetonitrile; mobile phase B: water (containing 0.1% ammonium acetate); b. gradient elution, mobile phase A: 10%-55%; c. flow rate: 12 mL/min; d. elution time: 20 min; retention time: 7.0 min, to afford compound 46 (10 mg, 5%).

LC-MS (ESI): m/z=524.2 [M+1]⁺

¹H NMR (400 MHz, CDCl₃) δ=10.93 (s, 1H), 8.51 (s, 1H), 8.24 (d, 1H), 8.14 (s, 1H), 8.04 (d, 1H), 7.82 (s, 1H), 7.22 (d, 3H), 6.77 (t, 1H), 6.50 (d, 2H), 5.13-4.95 (m, 1H), 4.32 (t, 2H), 3.94-3.66 (m, 4H), 3.41 (s, 4H), 2.70 (d, 5H), 1.29 (t, 3H).

### Example 47

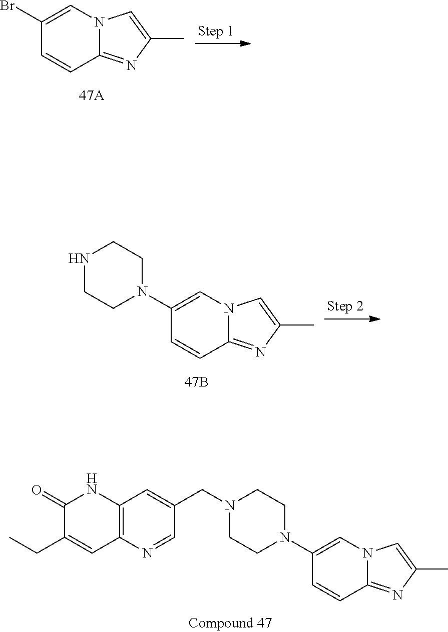 [SMILES: Cc1cn2cc(Br)ccc2n1 ; Cc1cn2cc(N3CCNCC3)ccc2n1 ; CCc1cc2ncc(CN3CCN(c4ccc5nc(C)cn5c4)CC3)cc2[nH]c1=O]

### Step 1:

6-Bromo-2-methylimidazole[1,2-a]pyridine (600 mg, 2.84 mmol) and piperazine (1.2 g, 14.2 mmol) were dissolved in toluene (10 mL), sodium tert-butoxide (546 mg, 5.69 mmol), JohnPhos (127 mg, 0.43 mmol) and palladium acetate (260 mg, 0.28 mmol) were added, and the mixture was reacted overnight at 100° C. under nitrogen protection, directly filtered, spun to dryness, and separated by silica gel chromatographic column (MeOH:DCM=0:1-1:1), to afford title compound 47B (610 mg, 99.3%).

LC-MS (ESI): m/z=217.1 [M+H]⁺.

### Step 2:

1H (50 mg, 0.22 mmol) and 47B (120 mg, 0.56 mmol) were dissolved in anhydrous acetonitrile (5 mL), potassium iodide (4 mg, 0.02 mmol) and DIPEA (144 mg, 1.12 mmol) were added, and the mixture was subjected to nitrogen replacement and reacted at 80° C. for 2 hours. Upon complete depletion of raw materials monitored by LCMS, a product was generated, and the system was concentrated and purified by preparative HPLC with preparative HPLC separation methods: 1. Instruments: waters 2767 preparative liquid chromatographic instrument; chromatographic column: SunFire@ Prep C18 (19 mm×250 mm); 2. The sample was filtered with a 0.45 μm filter to prepare a sample liquid; 3. Preparative chromatography conditions: a. composition of mobile phases A and B: mobile phase A: acetonitrile; mobile phase B: water (containing 0.1% ammonia); b. gradient elution, mobile phase A: 5%-50%; c. flow rate: 12 mL/min; d. elution time: 20 min; and the preparative liquid was concentrated and lyophilized, to afford compound 47 (12 mg, 13.3%).

¹H NMR (400 MHz, DMSO-d₆) δ 11.36 (s, 1H), 8.40 (d, 1H), 7.87 (d, 1H), 7.75 (s, 1H), 7.64-7.61 (m, 1H), 7.51 (s, 1H), 7.28 (d, 1H), 7.12 (dd, 1H), 3.66 (s, 2H), 3.08-2.99 (m, 4H), 2.60-2.52 (m, 6H), 2.27 (s, 3H), 1.18 (t, 3H).

LC-MS (ESI): m/z=403.2 [M+H]⁺.

### Example 48

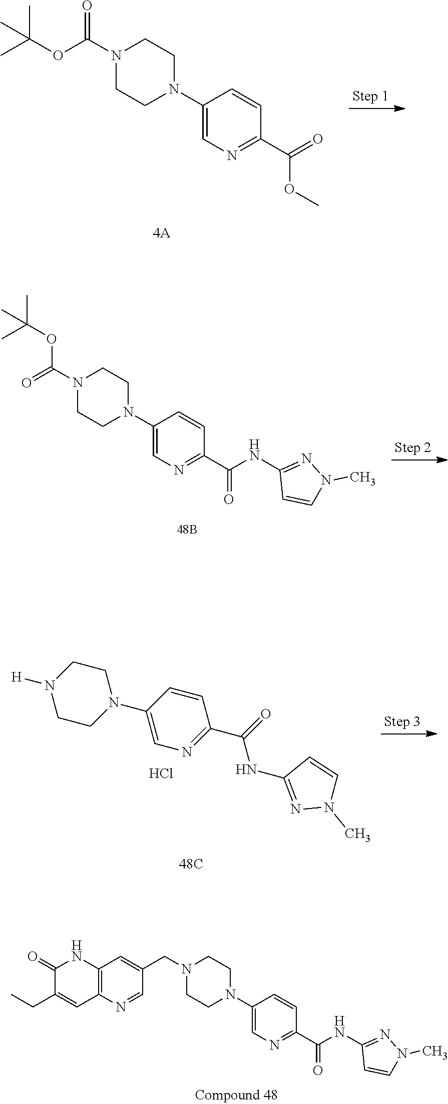

### Step 1:

Compound 4A (321 mg, 1 mmol) was dissolved in THF (10 mL) and H₂O (1 mL), LiOH·H₂O (45 mg, 1.1 mmol) was added, and the mixture was stirred and reacted at room temperature for 2 h. The solvent was removed by distillation under reduced pressure, to the resulting solid was added DMF (10 mL), HATU (570 mg, 1.5 mmol) was added under stirring, and the mixture was stirred at room temperature. After the solid was completely dissolved, DIEPA (1 mL) was added, 1-methyl-3-aminopyrazole (196 mg, 2 mmol) was finally added, and the mixture was stirred overnight at room temperature. After the reaction was completed as monitored by LCMS, to the system was added ethyl acetate (50 mL), and the mixture was washed with water (50 mL×4). The organic phase was collected, dried over anhydrous sodium sulfate, filtered, evaporated to dryness, and separated by silica gel chromatographic column (PE:EA=1:0-1:1), to afford title compound 48B (351 mg, 90.6%) as a pale yellow solid.

LC-MS (ESI): m/z=387.2 [M+H]⁺.

### Step 2:

48B (351 mg, 0.90 mmol) was dissolved in methanol (5 mL), and a solution of hydrogen chloride in dioxane (5 mL, 4M) was added. The mixture was reacted at room temperature for 4 hours and spun to dryness, to afford title compound 48C (276 mg, crude).

LC-MS (ESI): m/z=287.2 [M+H]⁺.

### Step 3:

1H (150 mg, 0.67 mmol) and 48C (276 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH=1:0-10:1), to afford compound 48 (144 mg, 50.1%).

¹H NMR (400 MHz, DMSO-d₆) δ 11.50 (s, 1H), 10.03 (s, 1H), 8.41 (d, 1H), 8.35 (d, 1H), 7.93 (d, 1H), 7.75 (s, 1H), 7.63 (d, 1H), 7.61 (d, 1H), 7.45 (dd, 1H), 6.58 (d, 1H), 3.76 (s, 3H), 3.66 (s, 2H), 3.40 (t, 4H), 2.60-2.52 (m, 6H), 1.19 (t, 3H).

LC-MS (ESI): m/z=473.2 [M+H]⁺.

### Example 49

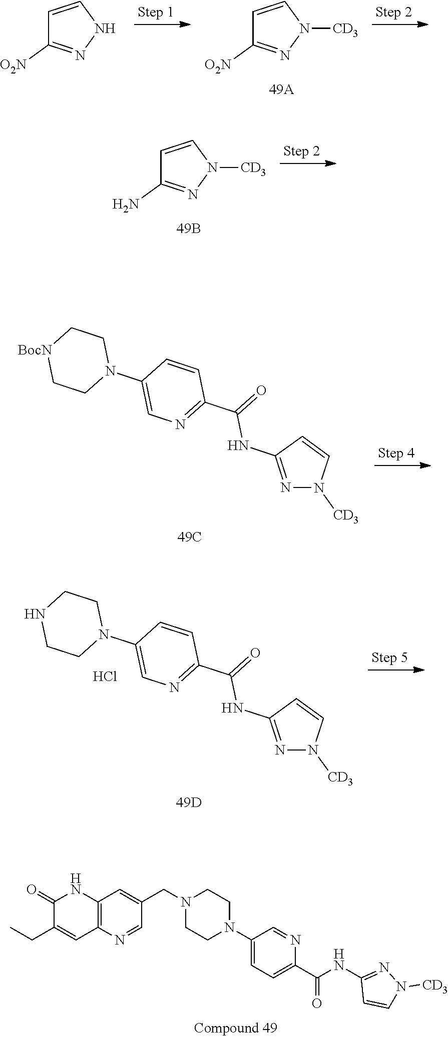

### Step 1:

Compound 3-nitro-1H-pyrazole (1.13 g, 10 mmol) was dissolved in DMF (20 mL), DBU (1.67 g, 11 mmol) and CD₃I (1.45 g, 10 mmol) were added, and the mixture was stirred and reacted at room temperature for 16 h. After the reaction was completed, to the system was added ethyl acetate (100 mL), the mixture was washed with aqueous saturated NaCl solution (4×80 mL), and the organic phase was dried over anhydrous sodium sulfate, concentrated, and separated by column chromatography (PE:EA=1:0-4:1), to afford target compound 49A (864 mg, 66.4%) as a white solid.

LC-MS (ESI): m/z=131.2 [M+H]⁺.

### Step 2:

49A (520 mg, 4 mmol) was dissolved in methanol (80 mL), Pd/C (100 mg, 10%) was added, the mixture was subjected to H₂ replacement, reacted at room temperature for 4 hours and filtered, and the filtrate was spun to dryness, to afford title compound 49B (387 mg, 96.7%), which was directly used without purification.

LC-MS (ESI): m/z=101.2 [M+H]⁺.

### Step 3:

Compound 4A (321 mg, 1 mmol) was dissolved in THF (10 mL) and H₂O (1 mL), LiOH·H₂O (45 mg, 1.1 mmol) was added, and the mixture was stirred and reacted at room temperature for 2 h. The solvent was removed by distillation under reduced pressure, to the resulting solid was added DMF (10 mL), HATU (570 mg, 1.5 mmol) was added under stirring, and the mixture was stirred at room temperature. After the solid was completely dissolved, DIEPA (1 mL) was added, 49B (200 mg, 2 mmol) was finally added, and the mixture was stirred overnight at room temperature. After the reaction was completed as monitored by LCMS, to the system was added ethyl acetate (50 mL), and the mixture was washed with water (50 mL×4). The organic phase was collected, dried over anhydrous sodium sulfate, filtered, evaporated to dryness, and separated by silica gel chromatographic column (PE:EA=1:0-1:1), to afford title compound 49C (338 mg, 87.2%) as a pale yellow solid.

LC-MS (ESI): m/z=390.2 [M+H]⁺.

### Step 4:

49C (338 mg, 0.87 mmol) was dissolved in methanol (5 mL), and a solution of hydrogen chloride in dioxane (5 mL, 4M) was added. The mixture was reacted at room temperature for 4 hours and spun to dryness, to afford title compound 49D (243 mg, crude).

LC-MS (ESI): m/z=290.2 [M+H]⁺.

### Step 5:

1H (150 mg, 0.67 mmol) and 49D (243 mg, crude) were dispersed in anhydrous acetonitrile (10 mL), potassium iodide (8 mg, 0.05 mmol) and DIPEA (0.5 mL) were added, and the mixture was subjected to nitrogen replacement and reacted at 80° C. for 4 hours. Upon complete depletion of raw materials monitored by LCMS, the system was concentrated, saturated sodium bicarbonate solution (20 mL) was added, and the mixture was extracted with a mixed solution of DCM:MeOH=10:1 (10 mL×3). The organic phases were combined, dried over anhydrous sodium sulfate, concentrated, and passed through column (DCM:MeOH=1:0-10:1), to afford compound 49 (122 mg, 46.9%).

¹H NMR (400 MHz, DMSO-d₆) δ 11.67 (s, 1H), 10.02 (s, 1H), 8.41 (d, 1H), 8.35 (d, 1H), 7.93 (d, 1H), 7.75 (s, 1H), 7.63 (d, 1H), 7.61 (d, 1H), 7.45 (dd, 1H), 6.58 (d, 1H), 3.66 (s, 2H), 3.40 (t, 4H), 2.60-2.52 (m, 6H), 1.19 (t, 3H).

LC-MS (ESI): m/z=476.2 [M+H]⁺.

### Example 50

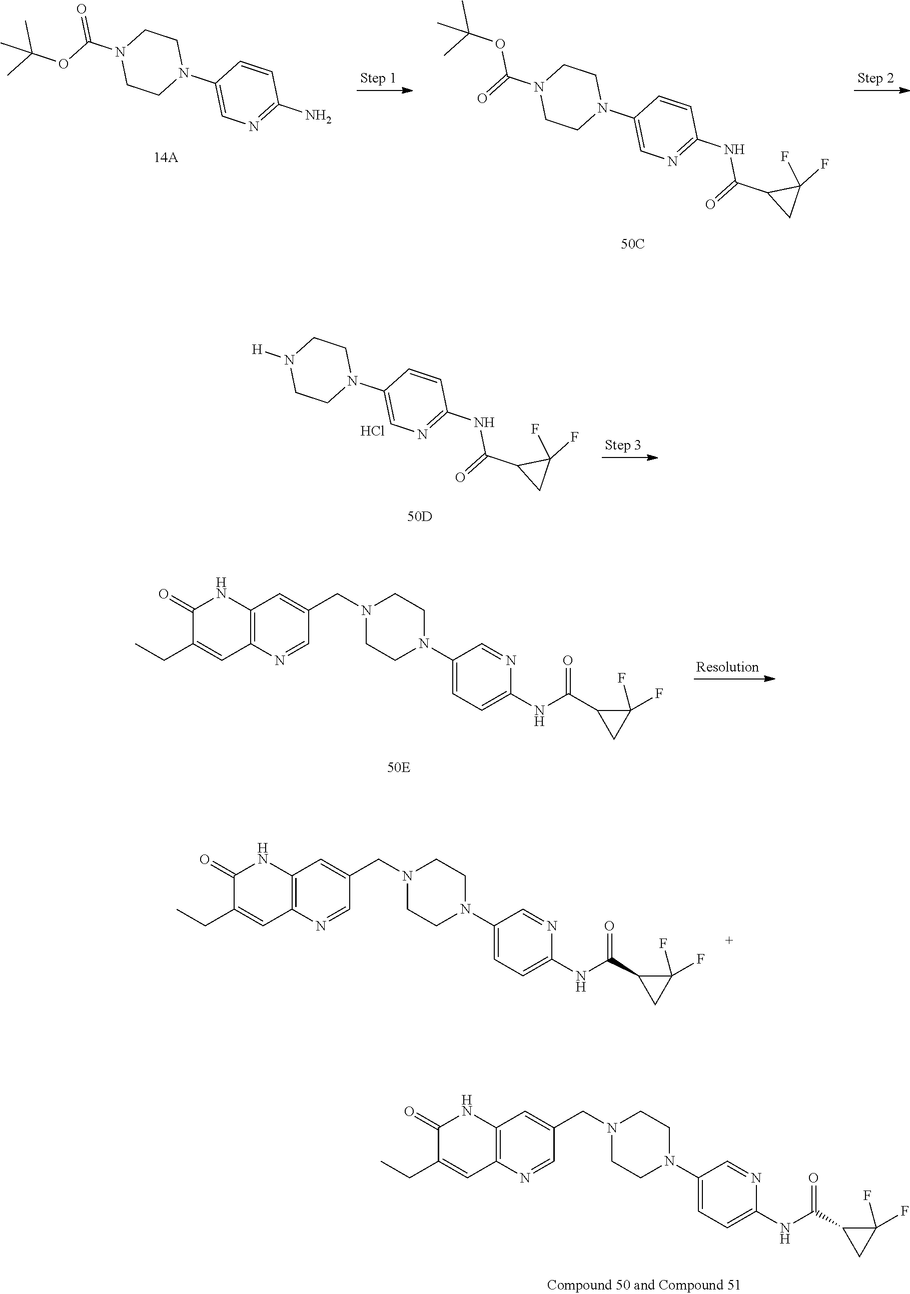

### Step 1:

2,2-Difluorocyclopropane-1-carboxylic acid (488 mg, 4 mmol) and tert-butyl 4-(6-aminopyridin-3-yl)piperazine-1-carboxylate (556 mg, 2 mmol) were dissolved in DMF (20 mL), HATU (2.28 g, 6 mmol) and DIEPA (3 mL) were added under stirring, and the mixture was stirred overnight at room temperature. After the reaction was completed as monitored by LCMS, to the system was added ethyl acetate (100 mL), and the mixture was washed with water (80 mL×4). The organic phase was collected, dried over anhydrous sodium sulfate, filtered, evaporated to dryness, and separated by silica gel chromatographic column (PE:EA=1:0-1:1), to afford title compound 50C (589 mg, 77.1%) as a yellow solid.

LC-MS (ESI): m/z=383.2 [M+H]⁺.

### Step 2:

50C (589 mg, 1.54 mmol) was dissolved in methanol (10 mL), and a solution of HCl in dioxane (8 mL, 4 M) was added. The mixture was reacted at room temperature for 4 hours and spun to dryness to obtain itle compound 50D (494 mg, crude).

LC-MS (ESI): m/z=283.2 [M+H]⁺.

### Step 3:

1H (260 mg, 1.2 mmol) and 50D (494 mg, crude) were dispersed in anhydrous acetonitrile (20 mL), potassium iodide (16 mg, 0.05 mmol) and DIPEA (2 mL) were added, and the mixture was subjected to nitrogen replacement and reacted at 80° C. for 4 hours. Upon complete depletion of raw materials monitored by LCMS, the system was concentrated, saturated sodium bicarbonate solution (30 mL) was added, and the mixture was extracted with a mixed solution of DCM:MeOH=10:1 (20 mL×3). The organic phases were combined, dried over anhydrous sodium sulfate, concentrated, and passed through column (DCM:MeOH=1:0-10:1), to afford 50E (246 mg, 46.2%).

50E was subjected to chiral resolution, to afford compound 50 (65 mg) and compound 51 (74 mg). Analytical method: instrument: Shimadzu LC-30AD sf; chromatographic column: Chiralpak OJ-3 50×4.6 mm I.D., 3 μm; mobile phase: A: CO₂, B: MeOH (0.05% DEA); gradient: B 5%-40%; flow rate: 3 mL/min, back pressure: 100 bar; column temperature: 35° C.; wavelength: 220 nm. Resolution method: instrument: Waters 150 SFC; chromatographic column: Chiralpak chromatographic column; mobile phase: A: CO₂, B: EtOH (0.1% NH3·H2O); gradient: 40% B, isocratic elution; flow rate: 100 mL/min; back pressure: 100 bar; column temperature: 25° C.; wavelength: 220 nm; cycle time: 4.5 min; sample preparation: 1 mg/ml compound, dissolved in acetonitrile. Injection: 5 ml/injection, post treatment: the separated components were dried in a water bath at 30° C. using a rotary evaporator, and then the solvent was dried using a freeze dryer at −80° C. to afford compound 50 and compound 51; retention time: Compound 50: 2.201 min; and compound 51: 2.500 min.

Compound 50: ¹H NMR (400 MHz, DMSO-d₆) δ 11.82 (s, 1H), 10.72 (s, 1H), 8.40 (d, 1H), 8.02 (d, 1H), 7.89 (d, 1H), 7.75 (s, 1H), 7.63 (d, 1H), 7.40 (dd, 1H), 3.65 (s, 2H), 3.24-3.05 (m, 4H), 2.98-2.85 (m, 1H), 2.55 (t, 6H), 2.05-1.87 (m, 2H), 1.18 (t, 3H).

LC-MS (ESI): m/z=469.2 [M+H]⁺

Compound 51: ¹H NMR (400 MHz, DMSO-d₆) δ 11.93-11.74 (m, 1H), 10.72 (s, 1H), 8.40 (d, 1H), 8.02 (d, 1H), 7.89 (d, 1H), 7.75 (s, 1H), 7.63 (d, 1H), 7.40 (dd, 11H), 3.65 (s, 2H), 3.16 (t, 4H), 2.95-2.87 (m, 1H), 2.60-2.52 (m, 6H), 2.05-1.87 (m, 2H), 1.18 (t, 3H).

LC-MS (ESI): m/z=469.2 [M+H]⁺

### Example 52

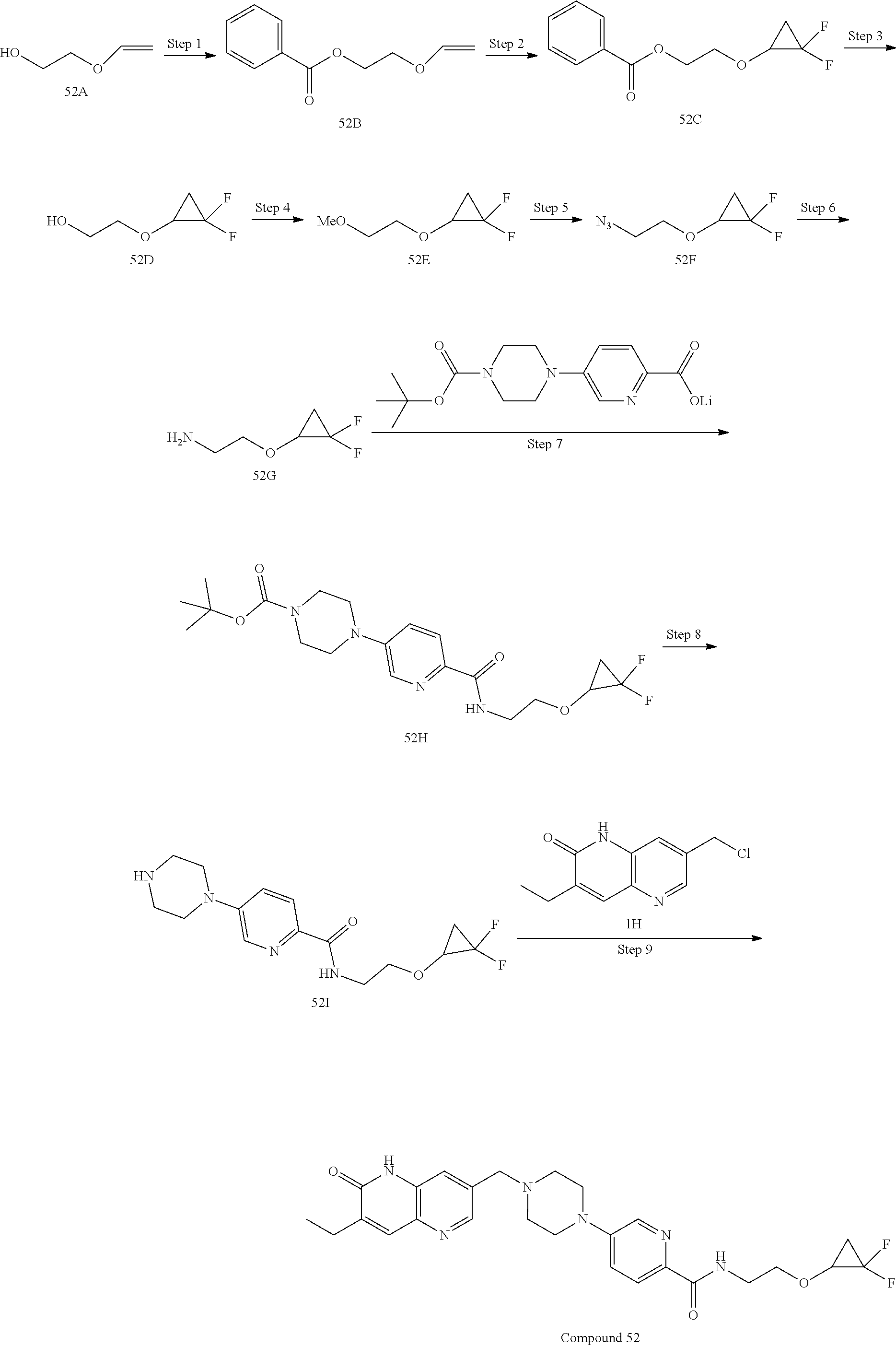

### Step 1:

Ethylene glycol vinyl ether 52A (22.0 g, 249.70 mmol) was dissolved in dichloromethane (330 mL), and then to the reaction liquid was added triethylamine (75.80 g, 749.10 mmol). After the addition was completed, the reaction liquid was cooled to about 0° C., benzoyl chloride (42.12 g, 299.64 mmol) was weighed and slowly added dropwise to the reaction liquid under stirring, and the mixture was stirred overnight at room temperature after the dropwise addition was completed. After the reaction was completed, to the reaction liquid were added water (300 mL) and dichloromethane (40 mL). The aqueous phase was separated, and the organic phase was dried over anhydrous sodium sulfate, filtered and concentrated, to afford a crude. The crude was purified by column chromatography (eluent: PE:EA=40:1 to 20:1 to 15:1), to afford target compound 52B (47.0 g, yield: 97.73%).

¹H NMR (400 MHz, CDCl₃) δ 8.21-7.93 (m, 2H), 7.66-7.50 (m, 1H), 7.50-7.32 (m, 2H), 6.54-6.49 (m, 1H), 4.57-4.55 (m, 2H), 4.27-4.22 (m, 1H), 4.08-4.01 (m, 1H), 4.04-3.98 (m, 2H).

### Step 2:

Compound 52B (19.2 g, 98.99 mmol) was dissolved in toluene (150 mL), and then a catalytic amount of potassium fluoride (460 mg) was added. After the addition was completed, the mixture was heated to 105° C. under N₂ protection, trimethylsilyl-2-(fluorosulfonyl)difluoroacetate (50.00 g, 199.78 mmol) was weighed and slowly added to the reaction liquid, and the mixture was reacted at this temperature for another 1 h after the addition was completed. The reaction process was monitored by TLC (ethyl acetate:petroleum ether=10:1). After the reaction was completed, the reaction liquid was cooled to room temperature, and to the reaction liquid were added ethyl acetate (500 mL) and water (300 mL). The aqueous phase was separated, and the organic phase was dried over anhydrous sodium sulfate, filtered and concentrated, to afford a crude. The crude was purified by column chromatography (ethyl acetate:petroleum ether=1:10), to afford target compound (6.90 g. yield: 28.52%).

¹H NMR (400 MHz, CDCl₃) δ 8.07-8.04 (m, 2H), 7.59-7.55 (m, 1H), 7.46-7.43 (m, 2H), 4.51-4.49 (m, 2H), 3.95-3.93 (m, 2H), 3.78-3.59 (m, 1H), 1.69-1.35 (m, 2H).

¹⁹F NMR (400 MHz, CDCl₃): δ=−128.47 (d), −146.94 (d).

### Step 3:

Compound 52C (6.90 g, 28.49 mmol) was dissolved in methanol (50 mL), sodium hydroxide aqueous solution (4.56 g, 113.96 mmol; 4 eq) [preparation of sodium hydroxide aqueous solution: weighing 4.56 g of solid sodium hydroxide and dissolving same in 10 ml of purified water, and then cooling the mixture to room temperature] was then slowly added, and the mixture was stirred at room temperature for 2 h after the dropwise addition was completed. After the reaction was completed as monitored by TLC (PE:EA=10:1), to the reaction liquid were added water (50 mL) and ethyl acetate (400 mL). The aqueous phase was separated, and the organic phase was washed with aqueous saturated NaCl solution (300 mL), dried over anhydrous sodium sulfate, filtered and concentrated, to afford target compound (2.42 g, yield: 61.50%).

¹H NMR (400 MHz, CDCl₃) δ 3.81-3.76 (m, 2H), 3.73-3.69 (m, 2H), 3.68-3.64 (m, 1H), 1.58-1.49 (m, 2H).

¹⁹F NMR (400 MHz, CDCl₃): δ=−128.62 (d), −146.91 (d).

### Step 4:

Compound 52D (2.42 g, 17.52 mmol, crude) was dissolved in dichloromethane (80 mL), and then triethylamine (3.55 g, 35.04 mmol) was added. After the addition was completed, the mixture was cooled to 0° C. under nitrogen protection, and then methanesulfonyl chloride (2.41 g, 21.02 mmol) was slowly added to the reaction liquid. After the addition was completed, the resulting mixture was stirred at room temperature for 20 h. After the reaction was completed, to the reaction liquid were added dichloromethane (100 ml) and purified water (100 mL). The aqueous phase was separated, and the organic phase was washed with dichloromethane (100 mL). The organic phases were combined, washed with aqueous saturated NaCl solution (150 mL, dried over anhydrous sodium sulfate, filtered and concentrated, to afford crude target compound 52E (3.48 g, 91.87%), which was directly used in the subsequent reaction.

¹H NMR (400 MHz, CDCl₃) δ 4.40-4.38 (m, 2H), 3.89-3.86 (m, 2H), 3.63-3.76 (m, 1H), 3.05 (s, 3H), 1.63-1.50 (m, 1H), 1.50-1.35 (m, 1H).

### Step 5:

Compound 52E (3.48 g, 16.10 nmol) was dissolved in DMF (20 mL), sodium azide (3.14 g, 48.30 mmol) was weighed and added to the reaction liquid, and the mixture was heated to 60° C. and reacted overnight after the addition was completed. After the reaction was completed, to the reaction liquid was added water (100 mL), and the mixture was extracted with ethyl acetate (200 mL×3). The organic phases were combined, washed with aqueous saturated NaCl solution (300 mL×2), and dried over anhydrous sodium sulfate, to afford crude target compound 52F (2.05 g, 78.05%), which was directly used in the next reaction.

¹H NMR (400 MHz, CDCl₃) δ 3.82-3.74 (m, 2H), 3.72-3.64 (m, 1H), 3.44-3.41 (m, 2H), 1.68-1.39 (m, 2H).

### Step 6:

Compound 52F (0.24 g, 1.47 mmol) was dissolved in ethyl acetate (6 mL), triphenylphosphine (0.579 g, 2.20 mmol) was then added, and the mixture was stirred at room temperature for 16 h after the addition was completed. After the reaction was completed, to the reaction liquid was added a solution of hydrogen chloride in 1,4-dioxane (2 mL), and the mixture was stirred for another 1 h. The reaction liquid was concentrated in vacuum under reduced pressure to afford an oil, ethyl acetate (5 mL) was then added for dissolution, and an anti-solvent petroleum ether (5 mL) was added following dissolution. After the addition was completed, the mixture was stirred at room temperature for 10 min and filtered, and the filter cake was washed with a mixed solvent (ethyl acetate:petroleum ether=1:1) (5 mL), filtered and dried, to afford crude target compound 52G hydrochloride (0.17 g), which was directly used in the next reaction.

### Step 7:

Compound 52G (0.39 g, 1.24 mmol) was dissolved in DMF (10 mL), HATU (0.67 g, 1.75 mmol) and diisopropylethylamine (0.45 g, 3.51 mmol) were then successively added, and the mixture was stirred at room temperature for 30 min after the addition was completed. Subsequently, to the reaction liquid was added lithium 5-(4-(tert-butoxycarbonyl)piperazin-1-yl)picolinate (0.16 g, 1.17 mmol) (synthesized with reference to patent US 2018141923), and then the mixture was stirred at room temperature for 18 h. After the reaction was completed, the reaction liquid was slowly added to water (50 mL), and then the mixture was extracted with ethyl acetate (100 mL×3). The organic phases were combined, washed with aqueous saturated NaCl solution (150 mL×2), dried over anhydrous sodium sulfate, filtered and concentrated, to afford a crude. The crude was purified by column chromatography (eluent: ethyl acetate:petroleum ether=3:1), to afford target compound 52H (0.19 g, yield: 38%).

LCMS m/z=427.2 [M+1]⁺

### Step 8:

Compound 52H (0.19 g, 0.45 mmol) was dissolved in dichloromethane (10 mL), and then a solution of hydrogen chloride in 1,4-dioxane (5 mL) was added under stirring at room temperature. After the addition was completed, the mixture was reacted and stirred overnight at room temperature. After the reaction was completed, the reaction liquid was directly concentrated to dryness, to afford target compound 52I hydrochloride (0.19 g). The hydrochloride was directly used in the next reaction.

LCMS m/z=327.1 [M+1]⁺

### Step 9:

Compound 52I (0.17 g, 0.52 mmol) and compound 1H (0.12 g, 0.52 mmol) were dissolved in DMF (5 mL), DIPEA (0.6 mL) and potassium iodide (0.17 g, 1.04 mmol) were then successively added, and the mixture was heated to 65° C. and reacted for 1 h after the addition was completed. After the reaction was completed, to the reaction liquid was added water (40 mL), and then the mixture was extracted with ethyl acetate (150 mL). The organic phase was separated, and the aqueous phase was extracted with ethyl acetate (100 mL×2). The organic phases were combined, washed with aqueous saturated NaCl solution (150 mL×2), dried over anhydrous sodium sulfate, filtered and concentrated, to afford a crude. The crude was purified by column chromatography (eluent: dichloromethane:methanol=10:1), to afford target compound (0.095 g, 35.64%).

LCMS m/z=513.2 [M+1]⁺

¹H NMR (400 MHz, DMSO) δ 11.83 (s, 1H), 8.57-8.38 (m, 2H), 8.29-8.28 (m, 1H), 7.85-7.83 (m, 1H), 7.75 (s, 1H), 7.63 (s, 1H), 7.41-7.38 (m, 1H), 4.11-3.82 (m, 1H), 3.77-3.62 (m, 4H), 3.50-3.46 (m, 2H), 3.41-3.33 (m, 4H), 2.57-2.54 (m, 6H), 1.73-1.62 (m, 1H), 1.57-1.48 (m, 1H), 1.20-1.17 (m, 3H).

### Example 53

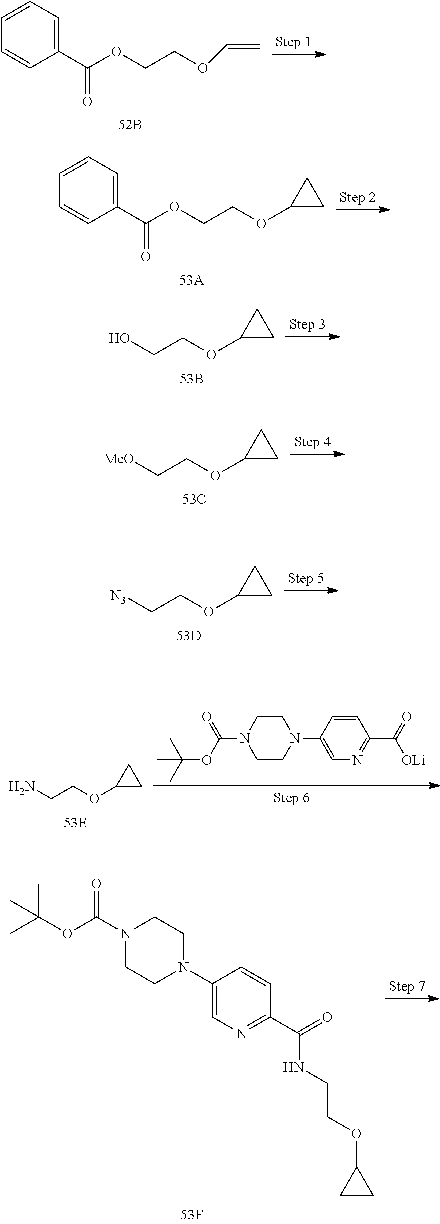

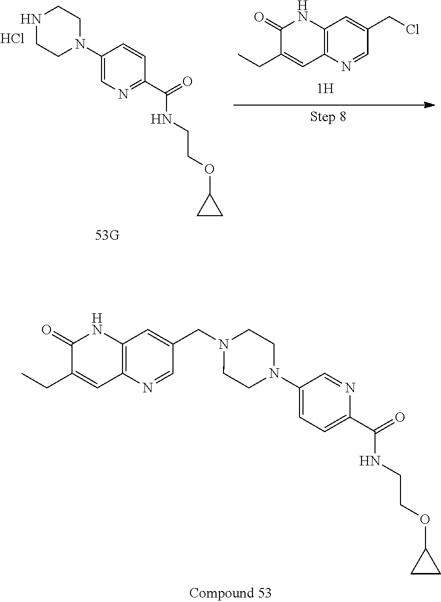 [SMILES: Cl ; O=C(NCCOC1CC1)c1ccc(N2CCNCC2)cn1 ; CCc1cc2ncc(CCl)cc2[nH]c1=O ; CCc1cc2ncc(CN3CCN(c4ccc(C(=O)NCCOC5CC5)nc4)CC3)cc2[nH]c1=O]

### Step 1:

Ultra-dry dichloromethane (50 mL) was placed in 500 mL three-necked flask and subjected to nitrogen replacement for protection, and then a solution of diethylzinc in n-hexane (1.0 M/L) (150 mL, 150 mmol) was added. After the addition was completed, the mixture was cooled with acetonitrile dry ice bath until the internal temperature dropped to −40° C., and then to the reaction liquid was slowly added a solution of diiodomethane in dichloromethane (26.78 g, 100 mmol) (26.78 g of diiodomethane was dissolved in 20 mL of dichloromethane). After the addition was completed, the mixture was stirred at a low temperature (about −40° C.) for 30 min, and then compound 52B (9.61 g, 50 mmol) (9.61 g of compound 52B was dissolved in dichloromethane (20 mL)) was slowly added to the reaction liquid. After the addition was completed, the mixture was slowly warmed to room temperature, and then stirred overnight at room temperature. After the reaction was completed, to the reaction liquid were added saturated ammonium chloride aqueous solution (200 mL) and ethyl acetate (600 mL), and the mixture was filtered to afford a filtrate. The aqueous phase was separated, and the organic phase was dried over anhydrous sodium sulfate, filtered and concentrated, to afford a crude. The crude was purified by column chromatography (eluent: ethyl acetate:petroleum ether=1:15), to afford target compound 53A (5.90 g, yield: 57.21%).

¹H NMR (400 MHz, CDCl₃) δ 8.07-8.05 (m, 2H), 7.57-7.53 (m, 1H), 7.45-7.42 (m, 2H), 4.47-4.45 (m, 2H), 3.85-3.83 (m, 2H), 3.40-3.37 (m, 1H), 0.65-0.54 (m, 2H), 0.52-0.48 (m, 2H).

### Step 2:

Compound 53A (5.90 g, 28.61 mmol) was dissolved in methanol (50 mL), the prepared sodium hydroxide aqueous solution (4.58 g, 114.44 mmol; 4 eq) [preparation of sodium hydroxide aqueous solution: weighing 4.58 g of solid sodium hydroxide and dissolving same in 10 ml of purified water, and then cooling the mixture to room temperature for later use] was then slowly added, and the mixture was stirred overnight at room temperature after the addition was completed. After the reaction was completed as monitored by TLC (PE:EA=10:1), to the reaction liquid were added water (50 mL) and ethyl acetate (150 mL×3). The aqueous phase was separated, and the organic phase was washed with aqueous saturated NaCl solution (300 mL), dried over anhydrous sodium sulfate, filtered and concentrated, to afford crude target compound 53B (1.29 g, 44.15%), which was directly used in the subsequent reaction.

¹H NMR (400 MHz, CDCl₃) δ 3.72-3.69 (m, 2H), 3.66-3.50 (m, 2H), 3.34-3.31 (m, 1H), 2.43 (s, 1H), 0.69-0.55 (m, 2H), 0.54-0.36 (m, 2H).

### Step 3:

Compound 53B (1.29 g, 12.63 mmol) was dissolved in dichloromethane (120 mL), and then triethylamine (3.83 g, 37.89 mmol) was added. After the addition was completed, the mixture was cooled to 0° C. under nitrogen protection, and then methanesulfonyl chloride (1.74 g, 15.12 mmol) was slowly added to the mixture. After the addition was completed, the resulting mixture was naturally warmed to room temperature and stirred overnight. After the reaction was completed, to the reaction liquid were added dichloromethane (200 mL) and water (200 mL). The organic phase was separated, and the aqueous phase was extracted once with dichloromethane (200 mL). The organic phases were combined, washed with aqueous saturated NaCl solution (300 mL), dried over anhydrous sodium sulfate, filtered and concentrated under reduced pressure (40° C.), to afford crude target compound 53C (1.77 g, yield: 77.76%).

¹H NMR (400 MHz, CDCl₃) δ 4.48-4.24 (m, 2H), 3.89-3.73 (m, 2H), 3.40-3.26 (m, 1H), 0.65-0.52 (m, 2H), 0.52-0.41 (m, 2H).

### Step 4:

Compound 53C (1.77 g, 9.82 mmol) was dissolved in DMF (10 mL), sodium azide (1.92 g, 29.46 mmol) was then weighed and added to the reaction liquid, and the mixture was heated to 60° C. and reacted overnight after the addition was completed. After the reaction was completed, to the reaction liquid was added water (50 mL), and the mixture was extracted with ethyl acetate (100 mL×3). The organic phases were combined, washed with aqueous saturated NaCl solution (100 mL×2), dried over anhydrous sodium sulfate, filtered and concentrated, to afford crude target compound 53D (0.48 g, yield: 38.45%).

¹H NMR (400 MHz, CDCl₃) δ 3.77-3.60 (m, 2H), 3.40-3.26 (m, 3H), 0.63-0.59 (m, 2H), 0.52-0.50 (m, 2H).

### Step 5:

Compound 53D (0.31 g, 2.44 mmol) was dissolved in ethyl acetate (6 mL), triphenylphosphine (0.770 g, 2.94 mmol) was then added, and the mixture was stirred at room temperature for 16 h after the addition was completed. After the reaction was completed, to the reaction liquid was added a solution of hydrogen chloride in 1,4-dioxane (2 mL), and the mixture was stirred for another 1 h. The reaction liquid was concentrated in vacuum under reduced pressure to afford an oil, ethyl acetate (5 mL) was then added for dissolution, and an anti-solvent petroleum ether (5 mL) was added following dissolution. After the addition was completed, the mixture was stirred at room temperature for 10 min and filtered, and the filter cake was washed with a mixed solvent (ethyl acetate:petroleum ether=1:1) (5 mL), filtered and dried, to afford crude target compound 53E hydrochloride (0.24 g), which was directly used in the next reaction.

LCMS m/z=102.2 [M+1]⁺

### Step 6:

Compound 53E (0.24 g, 2.08 mmol) was dissolved in DMF (10 mL), HATU (0.99 g, 2.49 mmol) and diisopropylethylamine (0.54 g, 4.16 mmol) were then successively added, and the mixture was stirred at room temperature for 30 min after the addition was completed. Subsequently, to the reaction liquid was added lithium 5-(4-(tert-butoxycarbonyl)piperazin-1-yl)picolinate (0.65 g, 2.08 mmol) (synthesized with reference to patent US 2018141923), and then the mixture was stirred at room temperature for 18 h. After the reaction was completed, the reaction liquid was slowly added to water (50 mL), and then the mixture was extracted with ethyl acetate (100 mL*3). The organic phases were combined, washed with aqueous saturated NaCl solution (050 mL*2), dried over anhydrous sodium sulfate, filtered and concentrated, to afford a crude. The crude was purified by column chromatography (eluent: ethyl acetate:petroleum ether=3:1), to afford target compound 53F (0.19 g, yield: 24%).

LCMS m/z=391.2 [M+1]⁺

### Step 7:

Compound 53F (0.19 g, 0.49 mmol) was dissolved in dichloromethane (10 mL), and then a solution of hydrogen chloride in 1,4-dioxane (5 mL) was added under stirring at room temperature. After the addition was completed, the mixture was reacted and stirred overnight at room temperature. After the reaction was completed, the reaction liquid was directly concentrated to dryness, to afford target compound 53G hydrochloride (0.15 g). The hydrochloride was directly used in the next reaction.

LCMS m/z=291.2 [M+1]⁺

### Step 8:

Compound 53G (0.15 g, 0.52 mmol) and compound 1H (0.10 g, 0.45 mmol) were dissolved in DMF (5 mL), DIPEA (0.5 mL) and potassium iodide (0.03 g, 0.21 mmol) were then successively added, and the mixture was heated to 80° C. and reacted for 3 h after the addition was completed. After the reaction was completed, to the reaction liquid was added water (40 mL), and then the mixture was extracted with ethyl acetate (150 mL). The organic phase was separated, and the aqueous phase was extracted with ethyl acetate (100 mL×2). The organic phases were combined, washed with aqueous saturated NaCl solution (150 mL×2), dried over anhydrous sodium sulfate, filtered and concentrated, and then purified by column chromatography (eluent: dichloromethane:methanol=10:1), to afford a crude, which was further purified by preparative HPLC to afford target compound 53 (25.1 mg, 10.20%).

LCMS m/z=477.3 [M+1]⁺

¹H NMR (400 MHz, CD₃OD) δ 8.51 (s, 1H), 8.31 (s, 1H), 7.92 (d, 1H), 7.85 (s, 1H), 7.79 (s, 1H), 7.38 (d, 1H), 4.55 (s, 1H), 3.76 (s, 2H), 3.70-3.68 (m, 2H), 3.58-3.57 (m, 2H), 3.43-3.33 (m, 41H), 2.70 (s, 61H), 1.32-1.28 (m, 3H), 0.56 (s, 2H), 0.49-0.48 (m, 2H).

### Preparation Examples

1. Preparation 1-1: Specification 50 mg/Tablet:

| | Dosage | | |
|---|---|---|---|
| Raw material name | (mg/tablet) | Proportion (%) | Function |
| Compound 4 | 50 | 10 | Active drug |
| Copovidone | 15 | 3 | Binder |
| Silica | 5 | 1 | Glidant |
| Croscarmellose sodium | 15 | 3 | Disintegrant |
| Microcrystalline | 150 | 30 | Filler |
| cellulose | | | |
| Mannitol | 260 | 52 | Filler |
| Sodium stearyl | 5 | 1 | Lubricant |
| fumarate | | | |
| Total | 500 | 100 | / |

2. Preparation 1-2: Specification 50 mg/Tablet:

| | Dosage | | |
|---|---|---|---|
| Raw material name | (mg/tablet) | Proportion (%) | Function |
| Compound 4 | 50 | 10 | Active drug |
| Copovidone | 15 | 3 | Binder |
| Silica | 5 | 1 | Glidant |
| Fumaric acid | 35 | 7 | PH regulator |
| Croscarmellose sodium | 15 | 3 | Disintegrant |
| Microcrystalline | 150 | 30 | Filler |
| cellulose | | | |
| Mannitol | 225 | 45 | Filler |
| Sodium stearyl | 5 | 1 | Lubricant |
| fumarate | | | |
| Total | 500 | 100 | / |

3. Preparation 1-3: Specification 50 mg/Tablet:

| | Dosage | | |
|---|---|---|---|
| Raw material name | (mg/tablet) | Proportion (%) | Function |
| Compound 4 (jet | 50 | 10 | Active drug |
| milling) | | | |
| Copovidone | 15 | 3 | Binder |
| Silica | 5 | 1 | Glidant |
| Fumaric acid | 35 | 7 | PH regulator |
| Croscarmellose sodium | 15 | 3 | Disintegrant |
| Microcrystalline | 150 | 30 | Filler |
| cellulose | | | |
| Mannitol | 225 | 45 | Filler |
| Sodium stearyl | 5 | 1 | Lubricant |
| fumarate | | | |
| Total | 500 | 100 | / |

Prescription preparations 1-3 above employ the following processes:

1) Weighing: weighing various raw materials according to the prescription (active pharmaceutical ingredient converted by the content).

2) Mixing: mixing the active drug, binder, glidant, pH regulator, disintegrant, and filler for 5 min, and adding the lubricant and mixing same for 2 min.

3) Tableting: tableting the powder in 2) using a shallow concave die with a diameter of 11 mm, with a controlled weight of 500 mg and a hardness of 70±30 N/mm².

4. Preparation 1-4: Specification 50 mg/Tablet

| | Dosage | | |
|---|---|---|---|
| Raw material name | (mg/tablet) | Proportion (%) | Function |
| Compound 4 | 50 | 12.5 | Active drug |
| Microcrystalline | 306 | 76.5 | Filler |
| cellulose | | | |
| Povidone K30 | 20 | 5.0 | Binder |
| Crospovidone | 20 | 5.0 | Disintegrant |
| Magnesium stearate | 4 | 1.0 | Lubricant |
| Total | 400 | 100 | / |

The prescription process is as follows:
Premixing: mixing compound 4 with microcrystalline cellulose for 1 minute and then screening same using a crushing granulator and then mixing same in a crospovidone wet granulator for 5 min to obtain a mixture;
Binder preparation: preparing 8.7% povidone solution;
Granulation: running the wet granulator, and slowly adding 8.7% povidone solution and stirring same for about 120 seconds to make soft material.

Whole granule drying: wetting the soft material through a 14 mesh screen using a rocking granulator to obtain wet granules; drying the wet granules in an oven at 80° C. controlled to a moisture level of less than 3.5%, and drying the whole granules by passing through a 14 mesh screen using a rocking granulator to obtain dry granules after granulation for later use.

Total mixing: after the granulation, adding magnesium stearate in the hopper mixer, and mixing same for 8 minutes, to obtain a total blend of granules.

Tableting: according to the theoretical tablet weight, using a shallow concave die with a diameter of 10 mm to press the tablet, with a controlled weight within ±5% of the theoretical tablet weight, and a hardness of 70N-130N, to obtain a plain tablet.

5. Preparation 1-5: Specification 10 mg/Tablet

| | Dosage | | |
|---|---|---|---|
| Raw material name | (mg/tablet) | Proportion (%) | Function |
| Compound 4 | 10 | 12.5 | Active drug |
| Povidone K30 | 4 | 5 | Binder |
| Crospovidone | 4 | 5 | Disintegrant |
| Microcrystalline | 61.2 | 76.5 | Filler |
| cellulose PH101 | | | |
| Magnesium stearate | 0.8 | 1 | Lubricant |
| Total | 80 | 100 | / |

6. Preparation 1-6: Specification 100 mg/Tablet

| | Dosage | | |
|---|---|---|---|
| Raw material name | (mg/tablet) | Proportion (%) | Function |
| Compound 4 | 100 | 12.5 | Active drug |
| Povidone K30 | 40 | 5 | Binder |
| Crospovidone | 40 | 5 | Disintegrant |
| Microcrystalline | 612 | 76.5 | Filler |
| cellulose PH101 | | | |
| Magnesium stearate | 8 | 1 | Lubricant |
| Total | 800 | 100 | / |

Prescription preparations 1-5 and 1-6 were prepared using a similar prescription process to prescription preparation 1-4. Purified water was used as a wetting agent during the preparation of a preparation.

### Biological Test Examples

### 1. PARP1 Enzyme Activity Test Experiment

PARP1 chemical fluorescence detection kit was purchased from BPS Bioscience. The histone solution in the kit was diluted 5× with 1×PBS, and 25 μL of the diluted histone solution was added to a microwell plate and incubated overnight at 4° C. After the incubation, the plate was washed three times with PBST (0.05% Tween-20). 100 μL of the blocking solution was added to the microwell plate and incubated at 25° C. for 90 minutes. After the incubation was completed, the plate was washed three times with PBST. 2.5 μL of compounds at different concentrations diluted in test buffer and 12.5 μL of substrate mixed solution (1.25 μL 10×PARP test buffer; 1.25 μL 10×PARP test mixed solution; 2.5 μL Activated DNA, 7.5 μL double-distilled water) were added to the microwell plate. The PARP1 enzyme was diluted to 2 ng/μL, 10 μL of the diluent was added to the microwell plate, and the reaction system was incubated at 25° C. for 60 minutes.

After the incubation was completed, the plate was washed three times with PBST. Streptavidin-HRP was diluted 50× with a blocking solution, and 25 μL of the diluent was added to the microwell plate and incubated at 25° C. for 30 minutes. After the incubation, the plate was washed three times with PBST. ELISA ECL substrate A and substrate B were mixed at a ratio of 1:1 (v/v), 50 μL of the mixture was added to the microwell plate, and the chemiluminescence value was read.

The inhibition rate was calculated according to formula [(1−(RLUₛₐₘₚₗₑ−RLUₘᵢₙ)/(RLUₘₐₓ−RLUₘᵢₙ))×100%], where RLUsample was the readout of the compound well, RLUmax was the readout of the solvent control well, and RLUmin was the readout of the control well without the PARP1 enzyme. Curve fitting was performed by four parameters (log(inhibitor) vs. response—Variable slope) using GraphPad Prism software, and the IC50 value was calculated.

Test results: the compound of the present disclosure had a significant inhibitory effect on PARP-1 enzyme activity in vitro, and the IC50 value of the example compounds on PARP-1 enzyme activity was less than 100 μM. The test results of some examples were shown in Table 1.

TABLE 1
PARP-1 enzyme activity

| compounds | IC₅₀ (nM) |
|---|---|
| 1 | 3.66 |
| 2 | 2.01 |
| 3 | 1.33 |
| 4 | 0.67 |
| 6 | 0.56 |
| 7 | 0.32 |
| 8 | 0.58 |
| 9 | 0.73 |
| 10 | 0.56 |
| 11 | 0.54 |
| 12 | 1.6 |
| 13 | 0.76 |
| 14 | 0.71 |
| 15 | 0.65 |
| 16 | 0.51 |
| 17 | 0.35 |
| 18 | 0.33 |
| 19 | 0.29 |
| 20 | 0.32 |
| 21 | 0.31 |
| 22 | 0.51 |
| 23 | 0.67 |
| 24 | 0.73 |
| 25 | 0.88 |
| 26 | 0.48 |
| 27 | 0.75 |
| 29 | 0.45 |
| 30 | 0.45 |
| 31 | 1.7 |
| 32 | 2.0 |
| 33 | 0.74 |
| 34 | 0.66 |
| 35 | 0.97 |
| 36 | 0.58 |
| 37 | 0.96 |
| 38 | 0.70 |
| 39 | 0.77 |
| 40 | 0.61 |
| 41 | 0.84 |
| 42 | 0.67 |
| 43 | 0.93 |
| 44 | 0.92 |
| 45 | 0.73 |
| 46 | 0.56 |
| 48 | 0.35 |
| 50 | 0.69 |
| 51 | 0.66 |

Conclusion: the compound of the present disclosure has a significant inhibitory effect on PARP-1 enzyme activity in vitro.

### 2. PARP2, PARP5A, PARP5B, PARP6, PARP7, PARP14 and PARP15 Enzyme Activity Test Experiments

PARP2, PARP5A, PARP5B, PARP6, PARP7. PARP14 and PARP15 chemical fluorescence detection kits were purchased from BPS Bioscience. The histone solution in the kit was diluted 5× with 1×PBS, and 25 μL of the diluted histone solution was added to a microwell plate and incubated overnight at 4° C. After the incubation, the plate was washed three times with PBST (0.05% Tween-20). 100 μL of the blocking solution was added to the microwell plate and incubated at 25° C. for 90 minutes. After the incubation was completed, the plate was washed three times with PBST. 2.5 μL of compound 4 diluted in test buffer and 5 μL of substrate mixed solution were added to the microwell plate. 5 μL of the diluted PARP enzyme was added to the microwell plate, and the reaction system was incubated at 25° C. for 60 minutes.

After the incubation was completed, the plate was washed three times with PBST. Streptavidin-HRP was diluted 50× with a blocking solution, and 25 μL of the diluent was added to the microwell plate and incubated at 25° C. for 30 minutes. After the incubation, the plate was washed three times with PBST. ELISA ECL substrate A and substrate B were mixed at a ratio of 1:1 (v/v), 25 μL of the mixture was added to the microwell plate, and the chemiluminescence value was read.

The inhibition rate was calculated according to formula [(1−(RLUₛₐₘₚₗₑ−RLUₘᵢₙ)/(RLUₘₐₓ−RLUₘᵢₙ))×100%], where RLUₛₐₘₚₗₑ was the readout of the compound well, RLUₘₐₓ was the readout of the solvent control well, and RLUₘᵢₙ was the readout of the control well without the PARP1 enzyme. Curve fitting was performed by four parameters (log(inhibitor) vs. response—Variable slope) using GraphPad Prism software, and the IC₅₀ value was calculated.

Test results: compound 4 of the present disclosure had a weak inhibitory effect on PARP2 enzyme activity in vitro, and its corresponding IC₅₀ value was 27.47 nM; compound 4 had a very weak inhibitory effect on PARP5A, PARP5B, PARP6, PARP7, PARP14 and PARP15 enzyme activities in vitro, and their corresponding IC₅₀ values were all greater than 500 nM. The specific test results were as shown in Table 2.

TABLE 2
PARP2, PARP5A, PARP5B, PARP6, PARP7,
PARP14 and PARP15 enzyme activities

| compounds | PARP enzyme | IC₅₀ (nM) |
|---|---|---|
| 4 | PARP2 | 27.47 |
| 4 | PARP5A | 6076 |
| 4 | PARP5B | 576 |
| 4 | PARP6 | 6860 |
| 4 | PARP7 | 5356 |
| 4 | PARP14 | 7064 |
| 4 | PARP15 | 4304 |

Conclusion: the inhibitory effect of compound 4 of the present disclosure on PARP2, PARP5A, PARP5B, PARP6, PARP7, PARP14 and PARP15 enzyme activities in vitro is much weaker than the inhibitory effect on PARP1, indicating that it has good PARP1 inhibitory selectivity.

### 3. MDA-MB-436 Cell Activity Test Experiment

Human breast cancer MDA-MB-436 cells were purchased from ATCC, placed in Leibovitz's L-15 medium (supplemented with 10 μg/mL insulin, 16 μg/mL glutathione, 10% fetal bovine serum and 1% penicillin-streptomycin solution) and cultured at 37° C. in a CO₂-free incubator. On day 1, the cells in the exponential growth phase were collected, and the cell suspension was adjusted to 4000 cells/135 μL with the culture medium. 135 μL of the cell suspension was added to each well of a 96-well cell culture plate and incubated overnight. On day 2, compounds at different concentrations were added, and the plate was placed in the incubator and incubated for 7 days. After the incubation was completed, according to operation instructions for a CellTiter-Glo kit (Promega, G7573), 75 μL of CTG solution, which was already pre-melted and equilibrated to room temperature, was added to each well, and the mixture was uniformly mixed for 2 min using a microplate shaker. The plate was placed at room temperature for 10 min, and then fluorescence signal values were measured using an Envision2104 plate reader (PerkinElmer). The inhibition rate was calculated according to the formula [(1−(RLU_{compound}−RLU_{blank})/(RLU_{control}−RLU_{blank}))×100%], where RLU_{compound} was the readout of the drug treated group, RLU_{control} was the average value of the solvent control group, and RLU_{blank} was the average value of the cell-free well. The IC₅₀ value was calculated using GraphPad Prism software.

Test results: the compound of the present disclosure had a significant inhibitory effect on breast tumor cells MDA-MB-436, with IC50 value of less than 100 nM, further IC50 value of less than 50 nM, furthermore IC50 value of less than 20 nM, and the most excellent IC50 value of less than 10 nM. At 10 μM, the maximum inhibition rate of the compound of the present disclosure on breast tumor cells MDA-MB-436 was up to 70% or more, further up to 80% or more, further up to 90%, with the optimal inhibition rate of 95% or more. The results of some examples were as shown in Table 3.

TABLE 3
MDA-MB-436 cell inhibitory activity

| compounds | IC₅₀ (nM) | Max inh. % 10 μM |
|---|---|---|
| 4 | 2 | 89.4 |
| 6 | 10 | 85.7 |
| 9 | 6 | 86.2 |
| 10 | 17 | 94.2 |
| 11 | 6 | 85.2 |
| 22 | 12 | 89.4 |
| 23 | 1.4 | 84.2 |
| 24 | 9 | 82.3 |
| 25 | 16 | 99.0 |
| 26 | 18 | 86.3 |
| 33 | 3.1 | 90.0 |
| 34 | 16.5 | 81.9 |
| 35 | 9.8 | 90.4 |
| 37 | 18.9 | 93.7 |
| 38 | 17.6 | 98.8 |
| 39 | 1.2 | 99.1 |
| 40 | 5.6 | 99.3 |
| 41 | 2.3 | 92.1 |
| 42 | 24.1 | 84.1 |
| 50 | 3.0 | 92.9 |
| 51 | 8.5 | 95.6 |
| 52 | 3.1 | 92.4 |
| 53 | 2.2 | 90.6 |

Conclusion: the compound of the present disclosure has good inhibitory activity on breast tumor cells MDA-MB-436.

### 4. Mouse MDA-MB-436 Subcutaneous In Vivo Transplanted Tumor Model

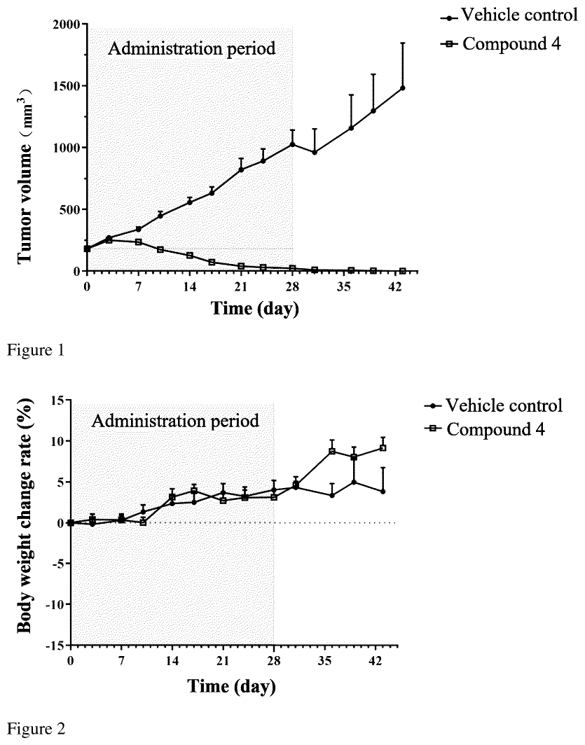

Human breast cancer MDA-MB-436 cells were placed in Leibovitz's L-15 medium (supplemented with 10 μg/mL insulin, 16 μg/mL glutathione, 10% fetal bovine serum and 1% penicillin-streptomycin solution) and cultured at 37° C. Conventional digestion treatment with trypsin was performed twice a week for passage. When the cell saturation was 80%-90%, and the number reached the requirement, the cells were harvested, counted, and inoculated. BALB/c nude mice (from Beijing Vital River Laboratory Animal Technology Co., Ltd.) were subcutaneously inoculated with 0.2 mL (10×10⁶) of MDA-MB-436 cells (plus matrigel, with the volume ratio of 1:1) on the right back. When the average tumor volume reached about 180 mms, grouping and administration were performed (marked as Day 0). The vehicle group was given 5% DMSO, 30% PEG400 and 65% of 20% sulfobutyl-β-cyclodextrin solution, and the administration group was given compound 4 (Day 0-Day 10: 1 mg/kg; Day 11-Day 28: 0.1 mg/kg). The administration frequency was once a day, the administration cycle was 29 days, and the drug withdrawal observation period was set to 14 days. After grouping, the tumor diameter was measured twice a week with a vernier caliper. The formula for calculating the tumor volume was: V=0.5×a×b², where a and b represented the long and short diameters of the tumor, respectively. The tumor inhibitory effect of compound 4 was evaluated by TGI (%)=[1−(average tumor volume at the end of administration in the treatment group−average tumor volume at the beginning of administration in the treatment group)/(average tumor volume at the end of treatment in the solvent control group−average tumor volume at the beginning of treatment in the solvent control group)]×100%. The tumor growth curve and the animal body weight change curve were as shown in FIG. 1 and FIG. 2, respectively.

Test results: after 28 days of administration, the TGI of the group given compound 4 was 119%; after the drug withdrawal, the tumor of the animals in the group given compound 4 did not grow again; There was no significant decrease in the body weight of the animals in the group given compound 4.

Conclusion: in the mouse MDA-MB-436 subcutaneous in vivo transplanted tumor model compound 4 of the present disclosure has good efficacy in inhibiting tumor growth and inducing tumor regression, and is well tolerated.

### 5. Pharmacokinetic Test in Rats

1.1 Experimental animals: male SD rats, about 220 g, 6-8 weeks old, 6 rats/compound. Purchased from Chengdu Ddossy Experimental Animals Co., Ltd.

1.2 Experimental design: on the day of the experiment, 6 SD rats were randomly grouped according to their body weights. The animals were fasted with water available for 12 to 14 h one day before the administration and were fed 4 h after the administration.

TABLE 4
Administration information

| | | Administration information | | | | | |
|---|---|---|---|---|---|---|---|
| | | | Administration | Administration | Administration | | |
| | Number | Test | dosage | concentration | volume | Collected | Mode of |
| Group | Male | compound | (mg/kg) | (mg/mL) | (mL/kg) | samples | administration |
| G1 | 3 | Compound | 2.5 | 0.5 | 5 | Plasma | Intravenous |
| | | 4 | | | | | administration |
| G2 | 3 | | 10 | 1 | 10 | Plasma | Intragastric |
| | | | | | | | administration |

Note:
vehicle for intravenous administration: 10% DMA + 10% Solutol + 80% Saline; vehicle for intragastric administration: 5% DMSO + 30% PEG400 + 65% (20% SBE-CD)
(DMA: dimethylacetamide; Solutol: polyethylene glycol-15-hydroxystearate; Saline; physiological saline; DMSO: dimethyl sulfoxide; SBE-CD: β cyclodextrin)

Before and after the administration, 0.15 mL of blood was taken from the orbit of the animals under isoflurane anesthesia and placed in an EDTAK2 centrifuge tube. The blood was centrifuged at 5000 rpm and 4° C. for 10 min to collect plasma. The blood collection time points for the intravenous administration group and intragastric administration group were: 0, 5 min, 15 min, 30 min, 1 h, 2 h, 4 h, 6 h, 8 h and 24 h. Before analysis and detection, all samples were stored at −80° C. The samples were analyzed quantitatively by LC-MS/MS. The results of some examples were as shown in Table 5.

TABLE 5
Pharmacokinetic parameters of test compound in plasma of rats

| Test | Mode of | CL | | | |
|---|---|---|---|---|---|
| compound | administration | (mL/min/kg) | Vdss (L/kg) | AUC0-t (hr*ng/mL) | F (%) |
| Compound 4 | i.v. (2.5 mg/kg) | 0.896 ± 0.097 | 0.236 ± 0.022 | 46601 ± 4753 | — |
| | i.g. (10 mg/kg) | — | — | 184171 ± 72260 | 98.8 ± 39 |

—: not applicable.

Conclusion: Preparations of compound 4 of different specifications all had good pharmacokinetic characteristics in rats.

### 6. Pharmacokinetic Test of Dogs

Experimental animals: male beagle dogs, 9-11 kg, 0.5-3.0 years old, 3 dogs/group, purchased from Beijing Marshall Biotechnology Co. Ltd.

Experimental method: On the day of the experiment, 9 beagle dogs were randomly grouped according to their body weights. The animals were fasted with water available for 14 to 18 h one day before the administration, and were fed 4 h after the administration.

TABLE 6
Administration information
Administration information

| | | | Administration | | Collect- |
|---|---|---|---|---|---|
| | | Test | concentration* | Dosing | ed |
| Group | Male | compound | (mg/dog) | quantity | samples |
| G1 | 3 | Preparation 1-1 | 50 | 1 tablet/dog | Plasma |
| G2 | 3 | Preparation 1-2 | 50 | 1 tablet/dog | Plasma |
| G3 | 3 | Preparation 1-3 | 50 | 1 tablet/dog | Plasma |

Before and after the administration, 1.0 mL of blood samples were drawn from the limb veins and placed in an EDTAK₂ centrifuge tube. Centrifugation was carried out at 5000 rpm at 4° C. for 10 min, and the plasma was collected. The sample collection time points for the intragastric administration group were 0 min, 15 min, 30 min, 1 h, 2 h, 4 h, 6 h, 8 h, 10 h, 12 h, 24 h and 48 h. Before analysis and detection, all samples were stored at −80° C., and a quantitative analysis of samples was performed using LC-MS/MS.

TABLE 7
Pharmacokinetic parameters of test compound in plasma of dogs

| | Mode of | AUC₀₋ₜ | | |
|---|---|---|---|---|
| Test compound | administration | (hr*ng/mL) | T_{1/2} (h) | F (%) |
| Preparation 1-1 | i.g. (50 mg/dog) | 45801 ± 11333 | 7.66 ± 1.1 | 62.7 ± 17 |
| Preparation 1-2 | i.g. (50 mg/dog) | 36720 ± 2243 | 7.42 ± 1.6 | 53.6 ± 18 |
| Preparation 1-3 | i.g. (50 mg/dog) | 39676 ± 5961 | 10.0 ± 0.19 | 58.3 ± 8.2 |

Conclusion: Preparations of the compound of the present disclosure of different specifications all had good pharmacokinetic characteristics in dogs.

### 7. Preparation Stability Investigation

### 7.1 High Temperature (60° C.) Investigation Test

Taking a bare sample of preparation 1-4, placing same under high temperature of 60° C. for investigation, and taking samples for testing at day 5 and day 10, respectively. The test results are shown as follows:

| Detection | | | | |
|---|---|---|---|---|
| item | Limit requirements | Day 0 | Day 5 | Day 10 |
| Character | This sample being an off- | Off-white | Off-white | Off-white |
| | white to white circular | circular tablet | circular tablet | circular tablet |
| | tablet | | | |
| Related | Impurity with RRT of | 0.25 | 0.24 | 0.24 |
| Substances | about 0.7 ≤ 0.5% | | | |
| (%) | Other maximum single | 0.05 | 0.06 | 0.07 |
| | impurity ≤ 0.2% | | | |
| | Total impurities ≤ 2.0% | 0.54 | 0.57 | 0.58 |
| Dissolution | ≥75% of the indicated | 97 | 95 | ≥95 |
| rate (%) | value | | | |
| Content | Should be 90.0%-110.0% | 101.1 | 99.1 | 100.3 |
| determination | of the indicated value | | | |
| (%) | | | | |

### 7.2 High Humidity (RH 92.5%) Investigation Test

Taking a bare sample of preparation 1-4, placing same under high humidity of RH 92.5% for investigation, and taking samples for testing at day 5 and day 10, respectively. The test results are shown as follows:

| Detection | | | | |
|---|---|---|---|---|
| item | Limit requirements | Day 0 | Day 5 | Day 10 |
| Character | This sample being an off- | Off-white | Off-white | Off-white |
| | white to white circular | circular tablet | circular tablet | circular tablet |
| | tablet | | | |
| Related | Impurity with RRT of | 0.25 | 0.24 | 0.23 |
| Substances | about 0.7 ≤ 0.5% | | | |
| (%) | Other maximum single | 0.05 | 0.05 | 0.05 |
| | impurity ≤ 0.2% | | | |
| | Total impurities ≤ 2.0% | 0.54 | 0.52 | 0.50 |
| Dissolution | ≥75% of the indicated | 97 | 97 | ≥95 |
| rate (%) | value | | | |
| Content | Should be 90.0%-110.0% | 101.1 | 101.6 | 102.9 |
| determination | of the indicated value | | | |
| (%) | | | | |

### 7.3 Accelerated Test

Taking a sample of preparation 1-4 to simulate the commercially available packaging and placing same at 40° C.±2° C. and RH 75%±5% for investigation, and taking a sample for testing. The test results are shown as follows:

| Detection | | | |
|---|---|---|---|
| item | Limit requirements | Day 0 | Day 28 |
| Character | This sample being an off-white | Off-white | Off-white |
| | to white circular tablet | circular tablet | circular tablet |
| Related | RRT0.7 ≤ 0.5% | 0.246 | 0.271 |
| Substances | Other maximum single | 0.047 | 0.064 |
| (%) | impurity ≤ 0.2% | | |
| | Total impurities ≤2.0% | 0.535 | 0.640 |
| Dissolution | ≥80% | 101.7 | ≥97 |
| rate (%) | | | |
| Content | Should be 90.0%-110.0% of the | 101.1 | 97.4 |
| determination | indicated value | | |
| (%) | | | |

Conclusion: The compound and preparation compositions of the present disclosure all had good stability under high temperature, high humidity, and accelerated conditions.

1. A pharmaceutical composition or a pharmaceutical preparation, wherein the pharmaceutical composition or the pharmaceutical preparation comprises an active ingredient M and a pharmaceutically acceptable excipient, wherein the active ingredient M is selected from among a compound having general formula (I) or a stereoisomer, a tautomer, a deuterated substance, a solvate, a prodrug, a metabolite, a pharmaceutically acceptable salt or a co-crystal thereof, 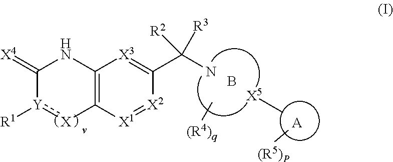
wherein X is selected from CR^{x}, C(R^{x})₂, O, N or NR^{x};
Y is selected from N, C or CH; [symbol]
represents a single bond or a double bond;
v is selected from 1, 2 or 3;
X¹, X² and X³ are each independently selected from N or CR^{x}; provided that when [symbol] represents a double bond, and v is selected from 1, X, X¹, X² and X³ are not all selected from CR^{x};
X⁴ is selected from O or S;
X⁵ is independently selected from N or CR^{x};
each R^{x} is independently selected from H, D, halogen, cyano, amino, hydroxyl, —SF₅, C₁₋₆ alkyl, halo C₁₋₆ alkyl, halo C₁₋₆ alkoxy, deuterated C₁₋₆ alkyl, deuterated C₁₋₆ alkoxy, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl); or two R^{x} on the same carbon atom together form ═O;
R¹ is selected from halogen, nitro, cyano, amino, hydroxyl, —SF₅, C₁₋₆ alkyl, C₁₋₆ alkoxy, C₂₋₆ alkenyl, C₂₋₆ alkynyl, C₁₋₆ alkyl-O—C₁₋₆ alkyl, —(CH₂)ᵣ—C₃₋₁₂ cycloalkyl or —(CH₂)ᵣ-(3- to 12-membered heterocycloalkyl), wherein the alkyl, alkoxy, alkenyl, alkynyl, cycloalkyl or heterocycloalkyl is optionally further substituted with 1-3 groups selected from D, halogen, cyano, amino, hydroxyl, C₁₋₆ alkyl or C₁₋₆ alkoxy;
each r is independently selected from 0, 1, 2 or 3;
R² and R³ are each independently selected from H, D, halogen, cyano, amino, hydroxyl, C₁₋₆ alkyl-O—C₁₋₆ alkyl, 4ydroxyl C₁₋₆ alkyl, C₁₋₆ alkoxy, halo C₁₋₆ alkyl, halo C₁₋₆ alkoxy, deuterated C₁₋₆ alkyl, deuterated C₁₋₆ alkoxy or C₁₋₆ alkyl; or R² and R³ together with the carbon atom to which they are attached form C₃₋₅ cycloalkyl or 4- to 5-membered heterocycloalkyl;
R⁴ is selected from D, halogen, cyano, amino, hydroxyl, —SF₅, C₁₋₆ alkyl, C₁₋₆ alkoxy, halo C₁₋₆ alkyl, halo C₁₋₆ alkoxy, deuterated C₁₋₆ alkyl or deuterated C₁₋₆ alkoxy; or two R⁴ on the same carbon atom together with the carbon atom to which they are attached form ═O;
R⁵ is selected from D, halogen, cyano, amino, hydroxyl, —SF₅, C₁₋₆ alkyl, C₁₋₆ alkoxy, halo C₁₋₆ alkyl, halo C₁₋₆ alkoxy, deuterated C₁₋₆ alkyl, or deuterated C₁₋₆ alkoxy;
q is selected from 0, 1, 2 or 3; p is selected from 0, 1, 2 or 3;
ring B is 5- to 6-membered saturated monocyclic heterocycloalkane containing 1-2 nitrogen atoms, 5- to 6-membered partially unsaturated monocyclic heterocycloalkane containing 1-2 nitrogen atoms, 6- to 8-membered saturated bridged heterocycle containing 1-4 nitrogen atoms, 5- to 10-membered saturated fused heterocycle containing 1-4 nitrogen atoms, or 5- to 11-membered saturated spiro heterocycle containing 1-4 nitrogen atoms;
ring A is selected from 5-membered monocyclic heteroaromatic ring containing 1-5 nitrogen, oxygen or sulfur atoms, 6-membered monocyclic heteroaromatic ring containing 2-5 nitrogen, oxygen or sulfur atoms, or 2-pyridyl, wherein the heteroaromatic ring or 2-pyridyl is further substituted with 1 substituent selected from Rₐ; or
ring A is selected from 7- to 10-membered bicyclic heteroaromatic ring containing 1-5 nitrogen, oxygen or sulfur atoms, or 7- to 10-membered bicyclic aromatic ring, wherein the heteroaromatic ring or aromatic ring is optionally further substituted with 1-3 substituents selected from R_{b}; or 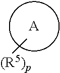
is selected from 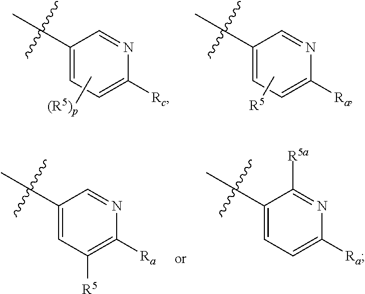
R^{5a} is selected from cyano, amino, hydroxyl, —SF₅, C₁₋₆ alkoxy, halo C₁₋₆ alkoxy, deuterated C₁₋₆ alkyl, or deuterated C₁₋₆ alkoxy;
Rₐ is selected from —C(O)N(R^{a1})₂, —NR^{a1}C(O)OR^{a1}, —NR^{a1}C(O)R^{a1}, —NR^{a1}C(O)N(R^{a1})₂, —C(═S)N(R^{a1})₂, —S(O)₂N(R^{a1})₂, 5- to 6-membered monocyclic heteroaryl containing 1-5 nitrogen, oxygen or sulfur atoms, 4- to 7-membered monocyclic heterocycloalkyl containing 1-4 nitrogen, oxygen or sulfur atoms, or 3- to 7-membered monocyclic cycloalkyl, wherein the heteroaryl, heterocycloalkyl, or cycloalkyl is optionally further substituted with 1-3 substituents selected from D, halogen, cyano, hydroxyl, amino, —NHC₁₋₆ alkyl, —N(C₁₋₆ alkyl)₂, C₁₋₆ alkyl, halo C₁₋₆ alkyl, C₁₋₆ alkoxy, halo C₁₋₆ alkoxy, deuterated C₁₋₆ alkyl, or deuterated C₁₋₆ alkoxy;
R_{b} is selected from —C(O)N(R^{a1})₂, —NR^{a1}C(O)OR^{a1}, —NR^{a1}C(O)R^{a1}, —NR^{a1}C(O)N(R^{a1})₂, —C(═S)N(R^{a1})₂, —S(O)₂N(R^{a1})₂, ═O, D, halogen, cyano, hydroxyl, amino, —NHC₁₋₆ alkyl, —N(C₁₋₆ alkyl)₂, C₁₋₆ alkyl, C₃₋₁₂ cycloalkyl, 3- to 12-membered heterocycloalkyl, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl, or deuterated C₁₋₆ alkoxy;
R_{c} is selected from —C(O)R^{a2}, —NHR^{a2}, —C(O)N(R^{a2})₂, —C(O)NHR^{a2}, —NR^{a1}C(O)OR^{a1}, —NR^{a1}C(O)R^{a1}, —NR^{a1}C(O)R^{a2}, —NR^{a1}R^{a2}, —NR^{a1}C(O)N(R^{a1})₂, —C(═S)N(R^{a1})₂, —S(O)₂N(R^{a1})₂, 5- to 6-membered monocyclic heteroaryl containing 1-5 nitrogen, oxygen or sulfur atoms, 4- to 7-membered monocyclic heterocycloalkyl containing 1-4 nitrogen, oxygen or sulfur atoms, or 3- to 7-membered monocyclic cycloalkyl, wherein the heteroaryl, heterocycloalkyl, or cycloalkyl is optionally further substituted with 1-3 substituents selected from D, halogen, cyano, hydroxyl, amino, —NHC₁₋₆ alkyl, —N(C₁₋₆ alkyl)₂, C₁₋₆ alkyl, halo C₁₋₆ alkyl, C₁₋₆ alkoxy, halo C₁₋₆ alkoxy, deuterated C₁₋₆ alkyl, or deuterated C₁₋₆ alkoxy;
each R^{a1} is independently selected from H, D, C₁₋₆ alkyl, C₃₋₁₂ cycloalkyl, 3- to 12-membered heterocycloalkyl, 5- to 6-membered monocyclic heteroaryl containing 1-5 nitrogen, oxygen or sulfur atoms, C₁₋₆ alkoxy, C₁₋₆ alkyl-O—C₁₋₆ alkyl, halo C₁₋₆ alkyl, halo C₁₋₆ alkoxy, deuterated C₁₋₆ alkyl, or deuterated C₁₋₆ alkoxy, wherein the cycloalkyl, heterocycloalkyl, or heteroaryl is optionally substituted with 1-3 substituents selected from halogen, deuterium or C₁₋₆ alkyl;
each R^{a2} is independently selected from C₃₋₁₂ cycloalkyl, 3- to 12-membered heterocycloalkyl, C₁₋₆ alkyl-C₃₋₁₂ cycloalkyl, 5- to 6-membered monocyclic heteroaryl containing 1-5 nitrogen, oxygen or sulfur atoms, C₁₋₆ alkoxy, C₁₋₆ alkyl-O—C₁₋₆ alkyl, C₁₋₆ alkyl-O—C₃₋₄ cycloalkyl, halo C₁₋₆ alkyl, halo C₁₋₆ alkoxy, deuterated C₁₋₆ alkyl, or deuterated C₁₋₆ alkoxy, wherein the cycloalkyl, heterocycloalkyl, or heteroaryl is optionally substituted with 1-3 substituents selected from halogen, deuterium, C₁₋₆ alkyl, deuterated C₁₋₆ alkyl or phenyl;
alternatively, two R^{a2} together with the nitrogen atom to which they are attached form 4- to 6-membered heterocycloalkyl, wherein the heterocycloalkyl is optionally substituted with 1-3 substituents selected from halogen, deuterium, or C₁₋₆ alkyl;
unless otherwise specified, the above-mentioned heterocycloalkane, heterocycloalkyl, heteroaryl, or heteroaromatic ring contains 1-5 heteroatoms selected from nitrogen, oxygen or sulfur;
the pharmaceutical composition or the pharmaceutical preparation comprises 1-600 mg of the active ingredient M and the excipient includes one or both of a filler and a disintegrant.

2. The pharmaceutical composition or the pharmaceutical preparation according to claim 1, wherein the pharmaceutical composition or the pharmaceutical preparation comprises 5-300 mg of the active ingredient M.

3. The pharmaceutical composition or the pharmaceutical preparation according to claim 2, wherein the pharmaceutical composition or the pharmaceutical preparation comprises 5-200 mg of the active ingredient M.

4. The pharmaceutical composition or the pharmaceutical preparation according to claim 3, wherein the pharmaceutical composition or the pharmaceutical preparation comprises 5-100 mg of the active ingredient M.

5. The pharmaceutical composition or the pharmaceutical preparation according to claim 4, wherein the pharmaceutical composition or the pharmaceutical preparation comprises 5 mg of the active ingredient M; or comprises 10 mg of the active ingredient M; or comprises 50 mg of the active ingredient M; or comprises 100 mg of the active ingredient M; or comprises 200 mg of the active ingredient M.

6-9. (canceled)

10. The pharmaceutical composition or the pharmaceutical preparation according to claim 1, wherein the pharmaceutical composition or the pharmaceutical preparation comprises an active ingredient M and a pharmaceutically acceptable excipient, wherein the active ingredient M is selected from among a compound having general formula (I) or a stereoisomer, a tautomer, a deuterated substance, a solvate, a prodrug, a metabolite, a pharmaceutically acceptable salt or a co-crystal thereof, having a structure of formula (II), (III), (IV), (V) or (VI): 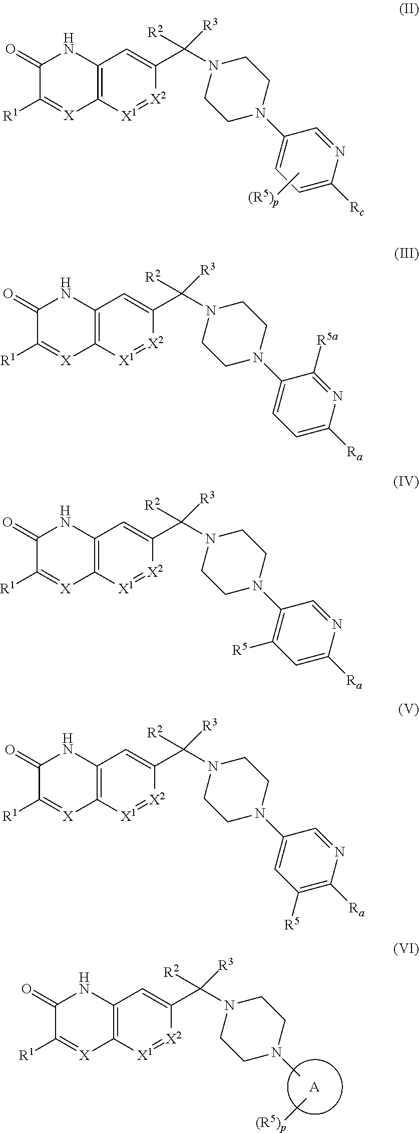
wherein X is selected from CR^{x} or N, provided that X, X¹ and X² are not all selected from CR^{x}.

11. The pharmaceutical composition or the pharmaceutical preparation according to claim 1, wherein the pharmaceutical composition or the pharmaceutical preparation comprises an active ingredient M and a pharmaceutically acceptable excipient, wherein the active ingredient M is selected from among a compound having general formula (I), (II), (III), (IV), (V) or (VI) or a stereoisomer, a tautomer, a deuterated substance, a solvate, a prodrug, a metabolite, a pharmaceutically acceptable salt or a co-crystal thereof, 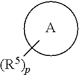
is selected from: 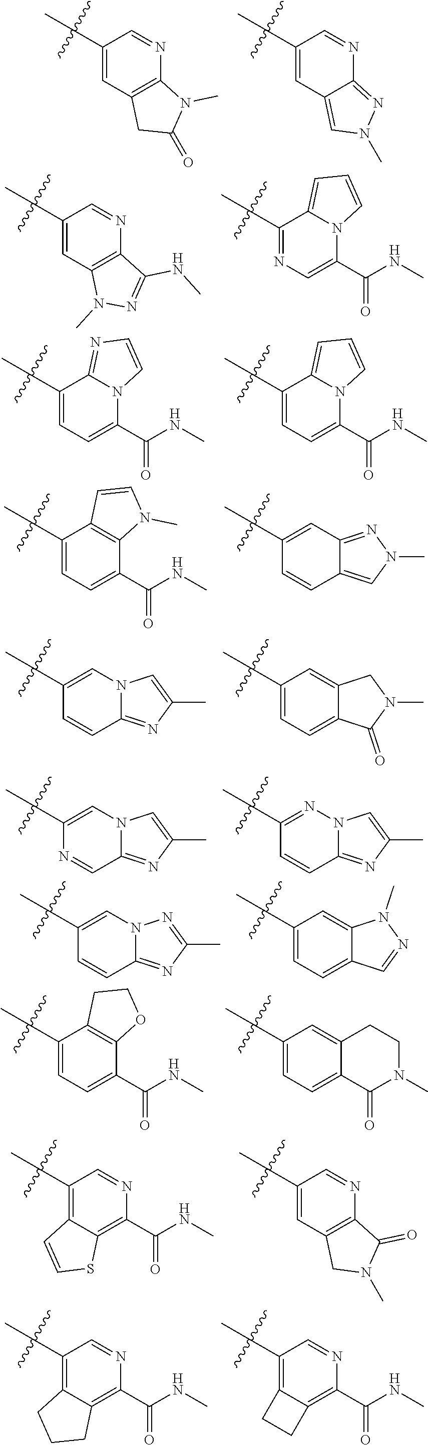 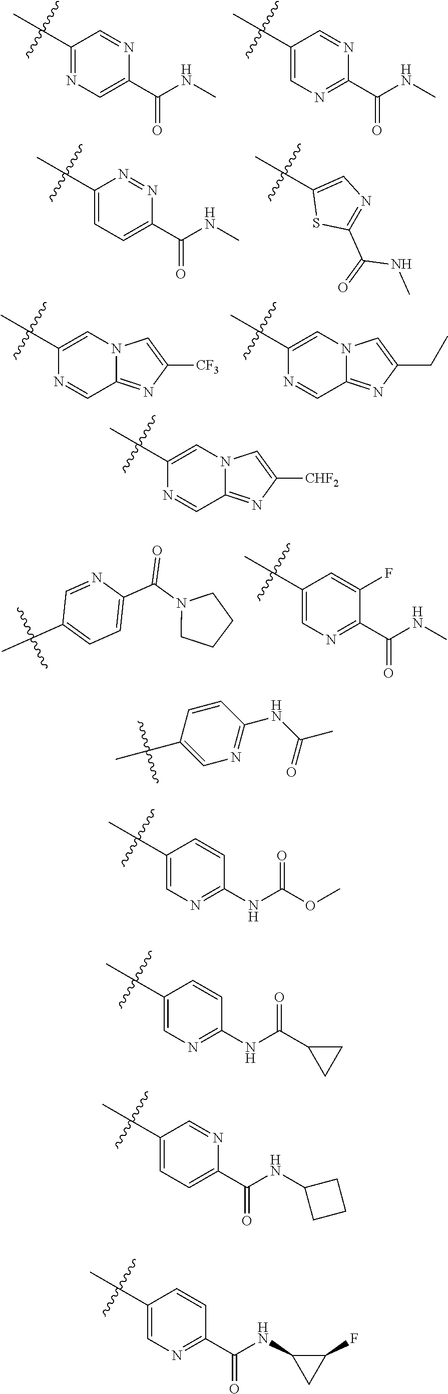 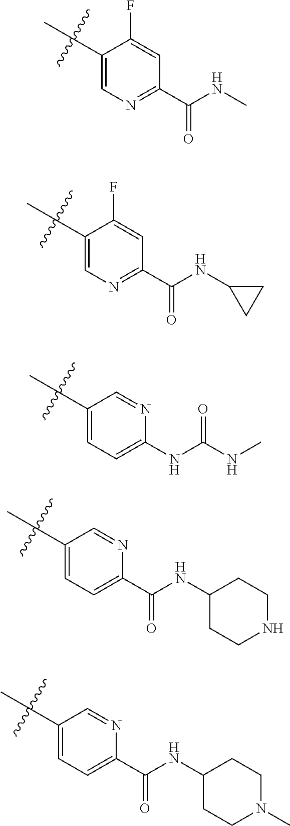 [SMILES: *c1cnc(C(=O)NC)cc1F ; *c1cnc(C(=O)NC2CC2)cc1F ; *c1ccc(NC(=O)NC)nc1 ; *c1ccc(C(=O)NC2CCNCC2)nc1 ; *c1ccc(C(=O)NC2CCN(C)CC2)nc1] 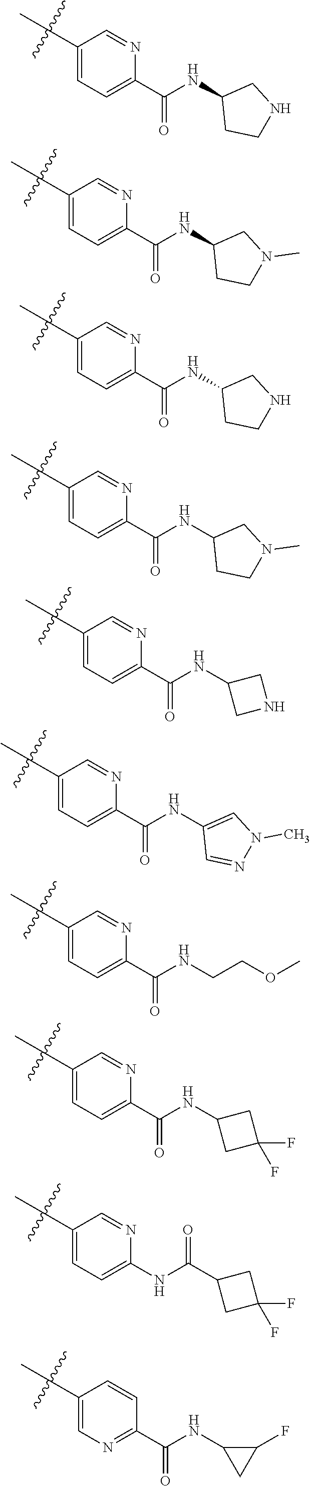 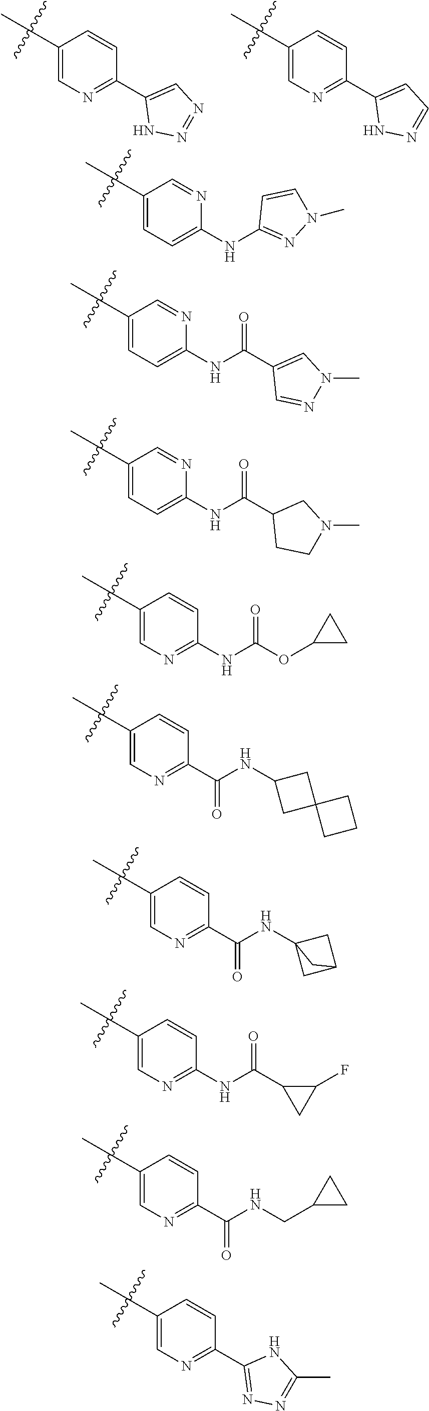 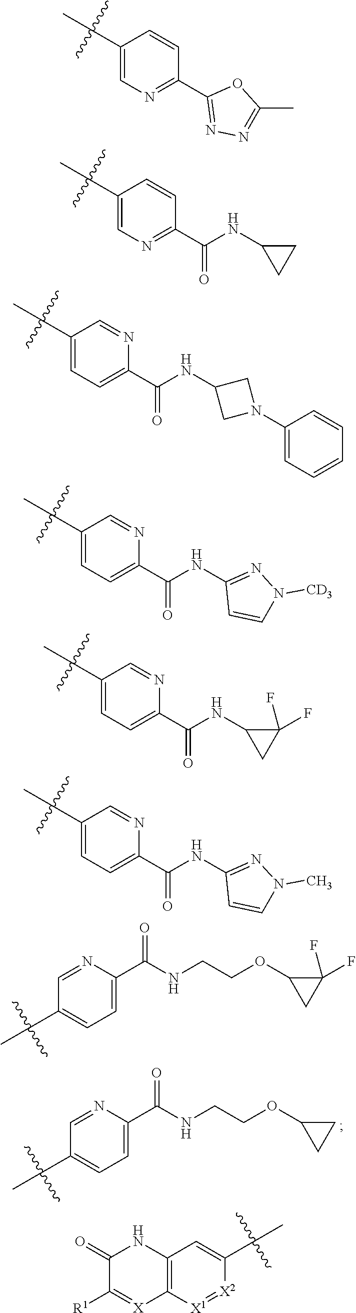
is selected from:

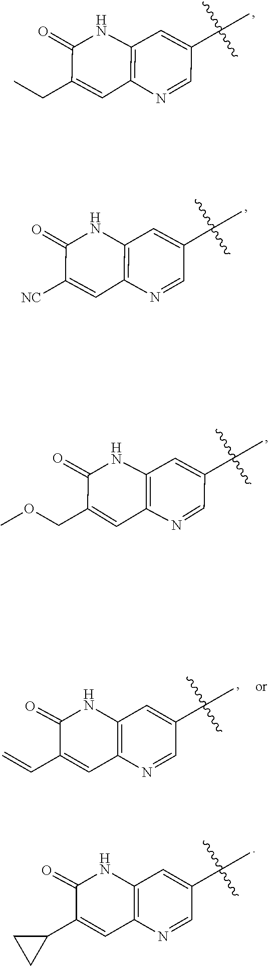 [SMILES: *c1cnc2cc(CC)c(=O)[nH]c2c1 ; *c1cnc2cc(C#N)c(=O)[nH]c2c1 ; *c1cnc2cc(COC)c(=O)[nH]c2c1 ; *c1cnc2cc(C=C)c(=O)[nH]c2c1 ; *c1cnc2cc(C3CC3)c(=O)[nH]c2c1]

12. The pharmaceutical composition or the pharmaceutical preparation according to claim 1, wherein the pharmaceutical composition or the pharmaceutical preparation comprises an active ingredient M and a pharmaceutically acceptable excipient, wherein the active ingredient M is selected from among a compound having general formula (I) or a stereoisomer, a tautomer, a deuterated substance, a solvate, a prodrug, a metabolite, a pharmaceutically acceptable salt or a co-crystal thereof, and the compound has a structure selected from one of the structures shown in Table S-1.

13. The pharmaceutical composition or the pharmaceutical preparation according to claim 1, wherein the active ingredient M is selected from the following structure:

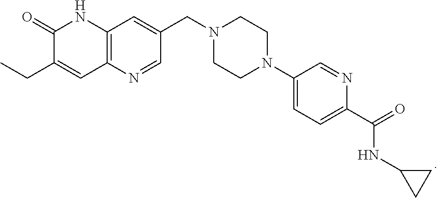 [SMILES: CCc1cc2ncc(CN3CCN(c4ccc(C(=O)NC5CC5)nc4)CC3)cc2[nH]c1=O]

14. The pharmaceutical composition or the pharmaceutical preparation according to claim 1, wherein the active ingredient M is present in an amount of 0.5%-90%.

15. The pharmaceutical composition or the pharmaceutical preparation according to claim 14, wherein the pharmaceutical composition or the pharmaceutical preparation comprises the active ingredient M and the pharmaceutically acceptable excipient according to claim 1, the pharmaceutically acceptable excipient includes a filler and a disintegrant.

16. The pharmaceutical composition or the pharmaceutical preparation according to claim 15, wherein the pharmaceutically acceptable excipient further includes one or more of a binder, a glidant, a lubricant, and a pH regulator.

17. A pharmaceutical composition or a pharmaceutical preparation comprising the active ingredient M and the pharmaceutically acceptable excipient according to claim 1, wherein the pharmaceutically acceptable excipient includes a filler and a disintegrant.

18. The pharmaceutical composition or the pharmaceutical preparation according to claim 17, wherein the active ingredient M is present in an amount of 5%-20%.

19. The pharmaceutical composition or the pharmaceutical preparation according to claim 17, wherein the filler is present in an amount of 50%-90%.

20. The pharmaceutical composition or the pharmaceutical preparation according to claim 19, wherein the filler is a combination of microcrystalline cellulose and mannitol.

21. The pharmaceutical composition or the pharmaceutical preparation according to claim 17, wherein the pharmaceutical composition or the pharmaceutical preparation comprises:
(i) the active ingredient M in an amount of 0.5%-99%;
(ii) the filler in an amount of 50%-90% which is a combination of microcrystalline cellulose and mannitol;
(iii) the disintegrant, croscarmellose sodium, in an amount of 1%-5%;
(iv) the binder, copovidone, in an amount of 1%-5%;
(v) the lubricant, sodium stearyl fumarate, in an amount of 0.1%-3%;
(vi) the glidant, silica, in an amount of 0.1%-3%.

22. The pharmaceutical composition or the pharmaceutical preparation according to claim 21, wherein the pharmaceutical composition or the pharmaceutical preparation further comprises the pH regulator fumaric acid in an amount of 1%-10%.

23. The pharmaceutical composition or the pharmaceutical preparation according to claim 17, wherein the pharmaceutical composition or the pharmaceutical preparation comprises:
(i) the active ingredient M in an amount of 0.5%-99%;
(ii) the pharmaceutically acceptable excipient including one or more of a filler, a binder, a wetting agent, a disintegrant, a glidant, and a lubricant;
optionally,
the filler including, but not limited to, one or more of microcrystalline cellulose, mannitol, lactose, sucrose, sorbitol, dextran, pregelatinized starch, calcium dihydrogen phosphate, and starch;
the binder including, but not limited to, one or more of povidone, hydroxypropylcellulose, hypromellose, and methylcellulose;
the wetting agent including, but not limited to, one or more of water and ethanol;
the disintegrant including, but not limited to, one or more of sodium carboxymethyl starch, low-substituted hydroxypropyl cellulose, crospovidone, croscarmellose sodium, and carboxymethylcellulose calcium;
the glidant including, but not limited to, one or more of talc, silica, colloidal silicon dioxide, polyethylene glycol, and dodecyl magnesium sulfate;
the lubricant including, but not limited to, magnesium stearate, calcium stearate, stearic acid, and sodium stearyl fumarate;
and may further comprise one or more of a flavoring agent, an antioxidant, a preservative, an opacifier, and a film coating premixer.

24. (canceled)

25. A method for the treatment of a disease in a mammal, comprising administering to a subject a therapeutically effective amount of the active ingredient M according to claim 1.
